(19) United States
(12) Patent Application Publication
(10) Pub. No.: US 2025/0145999 A1
(43) Pub. Date: May 8, 2025

(54) COMPOSITIONS AND METHODS FOR INHIBITING EXPRESSION OF PREKALLIKREIN (PKK) PROTEIN

(71) Applicant: Shanghai Argo Biopharmaceutical Co., Ltd., Shanghai (CN)

(72) Inventors: Dongxu Shu, Ningbo (CN); Pengcheng Patrick Shao, Warrington Township, PA (US); Shiwei Xia, Hangzhou (CN)

(73) Assignee: Shanghai Argo Biopharmaceutical Co., Ltd., Shanghai (CN)

(21) Appl. No.: 18/833,727

(22) PCT Filed: Jan. 28, 2023
(86) PCT No.: PCT/CN2023/073501

§ 371 Date: Jul. 26, 2024

(30) Foreign Application Priority Data
Jan. 28, 2022 (WO) PCT/CN2022/074535

(51) Int. Cl. C12N 15/113

(52) U.S. Cl. CPC C12N 15/1137; C12Y 304/21034; C12N 2310/14; C12N 2310/315; C12N 2310/351; C12N 2320/32

(57) ABSTRACT

Compositions and methods useful to reduce expression of a kallikrein B1 (KLKB1 or PKK) gene and for treatment of PKK-associated diseases and conditions are provided. Provided are PKK dsRNA agents, and compositions comprising PKK dsRNA agents that can be used to reduce PKK expression in cells and subjects.

### FIELD OF THE INVENTION

The invention relates, in part, to compositions and methods that can be used to inhibit prekallikrein (PKK) expression.

### BACKGROUND

The glycoprotein plasma prekallikrein (PKK) is encoded by the KLKB1 gene and its expression in cells and tissues is important in physiological events such as blood coagulation, fibrinolysis, kinin generation, and inflammation. PKK is converted to PK by Factor Xlla as in the kinin-kallikrein pathway. The level of PKK expression and its subsequent conversion to PK are important in the development of vascular diseases and inflammatory conditions. Vascular diseases and inflammatory conditions such as hereditary angioedema (HAB), edema, angioedema, swelling, angioedema of the lids, ocular edema, macular edema, cerebral edema, thrombosis, embolism, thromboembolism, deep vein thrombosis, pulmonary embolism, myocardial infarction, infarct, stroke, peripheral vascular disease, and others are significant health concerns in many countries. Vascular diseases result in reduced quality of life and can result in death and there is a need for improved means of preventing and treating such diseases and conditions.

### SUMMARY OF THE DISCLOSURE

In some aspects of the invention, a double-stranded ribonucleic acid (dsRNA) agent for inhibiting expression of prekallikrein (PKK) in a cell is provided, wherein the dsRNA agent includes a sense strand and an antisense strand, nucleotide positions 2 to 18 in the antisense strand comprising a region of complementarity to a PKK RNA transcript, wherein the region of complementarity includes at least 15 contiguous nucleotides that differ by 0, 1, 2, or 3 nucleotides from one of the antisense sequences listed in one of Tables 1-3, and optionally comprising a targeting ligand. In certain embodiments, the region of complementarity to the PKK RNA transcript includes at least 15, 16, 17, 18, or 19 contiguous nucleotides that differ by no more than 3 nucleotides from one of the antisense sequences listed in one of Tables 1-3. In some embodiments, the antisense strand of dsRNA is at least substantially complementary to a target region of SEQ ID NO: 767 and is provided in any one of Tables 1-3. In some embodiments, the antisense strand of dsRNA is fully complementary to a target region of SEQ ID NO: 767 and is provided in any one of Tables 1-3. In some embodiments, the dsRNA agent includes a sense strand sequence set forth in any one of Tables 1-3, wherein the sense strand sequence is at least substantially complementary to the antisense strand sequence in the dsRNA agent. In certain embodiments, the dsRNA agent includes a sense strand sequence set forth in any one of Tables 1-3, wherein the sense strand sequence is fully complementary to the antisense strand sequence in the dsRNA agent. In some embodiments, the dsRNA agent includes an antisense strand sequence set forth in any one of Tables 1-3. In some embodiments, the dsRNA agent includes the sequences set forth as a duplex sequence in any of Tables 1-3.

In some embodiments, the antisense strand of dsRNA comprises a nucleotide sequence of at least 15 contiguous nucleotides that differ by 0, 1, 2, or 3 nucleotides from formula (I): 5′-Z₁GAAUAAGUGAAAAACUZ₂-3′ (I), wherein Z₁ is selected from one of C, G, A, U or absent, Z₂ is a nucleotide sequence is 0-15 nucleotides in length. In certain embodiments, Z₁ is U. In certain embodiments, Z₁ is A. In certain embodiments, Z₂ is selected from G, GU, GA, GC, GG, GAU, GAA, GAC, GAG, GACC, GACU, GACG, GACA, GACAG, GACAGC, GACAGCG, GACAGCGA, GACAGCGAA, GACAGCGAAU, GACAGCGAAUC, GACAGCGAAUCA, GACAGCGAAUCAU, GACAGCGAAUCAUC, GACAGCGAAUCAUCU, GACAUU, GACAGA or absent. In certain embodiments, Z₂ is a nucleotide sequence is 1, 2, 3 or 4 nucleotides in length, the nucleotide sequence is selected from G, GU, GA, GC, GG, GAU, GAA, GAC, GAG, GACC, GACU, GACG, GACA. In certain embodiments, Z₁ is absent, Z₂ is G. In some embodiments, the sense strand of dsRNA comprises a nucleotide sequence at least 15 contiguous nucleotides that differ by 0, 1, 2, or 3 nucleotides from formula (II): 5′-Z₃AGUUUUUCACUUAUUCZ₄-3′ (II), wherein Z₃ is a nucleotide sequence is 0-15 nucleotides in length, Z₄ is selected from one of C, G, A, U or absent. In certain embodiments, Z₄ is U. In certain embodiments, Z₄ is A. In certain embodiments, Z₃ is selected from C, AC, UC, GC, CC, AUC, UUC, GUC, CUC, UGUC, GGUC, AGUC, CGUC, CUGUC, GCUGUC, CGCUGUC, UCGCUGUC, UUCGCUGUC, AUUCGCUGUC, GAUUCGCUGUC, UGAUUCGCUGUC, AUGAUUCGCUGUC, GAUGAUUCGCUGUC, AGAUGAUUCGCUGUC or absent. In certain embodiments, Z₃ is a nucleotide sequence is 1, 2, 3 or 4 nucleotides in length, the nucleotide sequence is selected from C, AC, UC, GC, CC, AUC, UUC, GUC, CUC, UGUC, GGUC, AGUC, CGUC. In certain embodiments, Z₄ is absent, Z₃ is C. In some embodiments, the dsRNA agent includes a sense strand and an antisense strand, wherein the antisense strand of dsRNA comprises nucleotide sequence of at least 15 contiguous nucleotides that differ by 0, 1, 2, or 3 nucleotides from formula (I) and the sense strand of dsRNA comprises nucleotide sequence of at least 15 contiguous nucleotides that differ by 0, 1, 2, or 3 nucleotides from formula (II), wherein the nucleotide formula (I) and (II) are as described above. In some embodiments, each strand is no more than 40 nucleotides in length. In certain embodiments, the sense strand is complementary or substantially complementary to the antisense strand, and the region of complementarity is between 16 and 23 nucleotides in length. In some embodiments, the region of complementarity is 19-21 nucleotides in length. In some embodiments, wherein the sense strand is no more than 35 nucleotides in length comprising a region of complementarity to the antisense strand including at least 15, 16, 17, 18, or 19 nucleotides. In some embodiments, Z₁ is a nucleotide complementary to Z₄. In some embodiments, Z₂ is a nucleotide sequence complementary to Z₃.

In some embodiments, the antisense strand of dsRNA comprises a nucleotide sequence of at least 15 contiguous nucleotides that differ by 0, 1, 2, or 3 nucleotides from formula (III): 5′-Z₅UAUUUGUGAGAAAGGUZ₆-3′ (III), wherein Z₅ is selected from one of C, G, A, U or absent, Z₆ is a nucleotide sequence is 0-15 nucleotides in length. In certain embodiments, Z₅ is U. In certain embodiments, Z₆ is selected from G, GU, GA, GC, GG, GUU, GUA, GUC, GUG, GUAA, GUAU, GUAC, GUAG, GUAUC, GUAUCU, GUAUCUU, GUAUCUUU, GUAUCUUUU, GUAUCUUUUG, GUAUCUUUUGU, GUAUCUUUUGUA, GUAUCUUUUGUAA, GUAUCUUUUGUAAU, GUAUCUUUUGUAAUG, GUAUUU, GUAUGA, or absent. In certain embodiments, Z₆ is a nucleotide sequence is 1, 2, 3 or 4 nucleotides in length, the nucleotide sequence is selected from G, GU, GA, GC, GG, GUU, GUA, GUC, GUG, GUAA, GUAU, GUAC, GUAG. In certain embodiments, Z₅ is absent, Z₆ is G. In some embodiments, the sense strand of dsRNA comprises a nucleotide sequence at least 15 contiguous nucleotides that differ by 0, 1, 2, or 3 nucleotides from formula (IV): 5′-Z₇ACCUUUCUCACAAAUAZ₈-3′ (IV), wherein Z₇ is a nucleotide sequence is 0-15 nucleotides in length, Z₈ is selected from one of C, G, A, U or absent. In certain embodiments, Z₈ is A. In certain embodiments, Z₇ is selected from C, AC, UC, GC, CC, AAC, UAC, GAC, CAC, GUAC, CUAC, UUAC, AUAC, GAUAC, AGAUAC, AAGAUAC, AAAGAUAC, AAAAGAUAC, CAAAAGAUAC, ACAAAAGAUAC, UACAAAAGAUAC, UUACAAAAGAUAC, AUUACAAAAGAUAC, CAUUACAAAAGAUAC, or absent. In certain embodiments, Z₇ is a nucleotide sequence is 1, 2, 3 or 4 nucleotides in length, the nucleotide sequence is selected from C, AC, UC, GC, CC, AAC, UAC, GAC, CAC, GUAC, CUAC, UUAC, AUAC. In certain embodiments, Z₈ is absent, Z₇ is C. In some embodiments, the dsRNA agent includes a sense strand and an antisense strand, wherein the antisense strand of dsRNA comprises nucleotide sequence of at least 15 contiguous nucleotides that differ by 0, 1, 2, or 3 nucleotides from formula (III) and the sense strand of dsRNA comprises nucleotide sequence of at least 15 contiguous nucleotides that differ by 0, 1, 2, or 3 nucleotides from formula (IV), wherein the nucleotide formula (III) and (IV) are as described above. In some embodiments, each strand is no more than 40 nucleotides in length. In certain embodiments, the sense strand is complementary or substantially complementary to the antisense strand, and the region of complementarity is between 16 and 23 nucleotides in length. In some embodiments, the region of complementarity is 19-21 nucleotides in length. In some embodiments, wherein the sense strand is no more than 35 nucleotides in length comprising a region of complementarity to the antisense strand including at least 15, 16, 17, 18, or 19 nucleotides. In some embodiments, Z₅ is a nucleotide complementary to Z₈. In some embodiments, Z₆ is a nucleotide sequence complementary to Z₇.

In some embodiments, the antisense strand of dsRNA comprises a nucleotide sequence of at least 15 contiguous nucleotides that differ by 0, 1, 2, or 3 nucleotides from formula (V): 5′-ZAGUGAAAAACUGACAGZ₁₀-3′ (V), wherein Z₉ is selected from one of C, G, A, U or absent, Z₁₀ is a nucleotide sequence is 0-15 nucleotides in length. In certain embodiments, Z₉ is U. In certain embodiments, Z₉ is A. In certain embodiments, Z₁₀ is selected from C, CC, CU, CA, CG, CGU, CGG, CGC, CGA, CGAC, CGAU, CGAA, CGAG, CGAAU, CGAAUC, CGAAUCA, CGAAUCAU, CGAAUCAUC, CGAAUCAUCU, CGAAUCAUCUU, CGAAUCAUCUUU, CGAAUCAUCUUUG, CGAAUCAUCUUUGU, CGAAUCAUCUUUGUG, CGAAUU, CGAAGA, or absent. In certain embodiments, Z₁₀ is a nucleotide sequence is 1, 2, 3 or 4 nucleotides in length, the nucleotide sequence is selected from C, CC, CU, CA, CG, CGU, CGG, CGC, CGA, CGAC, CGAU, CGAA, CGAG. In certain embodiments, Z₉ is absent, Z₁₀ is C. In some embodiments, the sense strand of dsRNA comprises a nucleotide sequence at least 15 contiguous nucleotides that differ by 0, 1, 2, or 3 nucleotides from formula (VI): 5′-Z₁₁CUGUCAGUUUUUCACUZ₁₂-3′ (VI), wherein Z₁₁ is a nucleotide sequence is 0-15 nucleotides in length, Z₁₂ is selected from one of C, G, A, U or absent. In certain embodiments, Z₁₂ is A. In certain embodiments, Z₁₂ is U. In certain embodiments, Z₁₁ is selected from G, AG, UG, GG, CG, ACG, UCG, GCG, CCG, GUCG, AUCG, CUCG, UUCG, AUUCG, GAUUCG, UGAUUCG, AUGAUUCG, GAUGAUUCG, AGAUGAUUCG, AAGAUGAUUCG, AAAGAUGAUUCG, CAAAGAUGAUUCG, ACAAAGAUGAUUCG, CACAAAGAUGAUUCG, or absent. In certain embodiments, Z₁₁ is a nucleotide sequence is 1, 2, 3 or 4 nucleotides in length, the nucleotide sequence is selected from G, AG, UG, GG, CG, ACG, UCG, GCG, CCG, GUCG, AUCG, CUCG, UUCG. In certain embodiments, Z₁₂ is absent, Z₁₁ is G. In some embodiments, the dsRNA agent includes a sense strand and an antisense strand, wherein the antisense strand of dsRNA comprises nucleotide sequence of at least 15 contiguous nucleotides that differ by 0, 1, 2, or 3 nucleotides from formula (V) and the sense strand of dsRNA comprises nucleotide sequence of at least 15 contiguous nucleotides that differ by 0, 1, 2, or 3 nucleotides from formula (VI), wherein the nucleotide formula (V) and (VI) are as described above. In some embodiments, each strand is no more than 40 nucleotides in length. In certain embodiments, the sense strand is complementary or substantially complementary to the antisense strand, and the region of complementarity is between 16 and 23 nucleotides in length. In some embodiments, the region of complementarity is 19-21 nucleotides in length. In some embodiments, wherein the sense strand is no more than 35 nucleotides in length comprising a region of complementarity to the antisense strand including at least 15, 16, 17, 18, or 19 nucleotides. In some embodiments, Z₉ is a nucleotide complementary to Z₁₂. In some embodiments, Z₁₀ is a nucleotide sequence complementary to Z₁₁.

In some embodiments, the antisense strand of dsRNA comprises a nucleotide sequence of at least 15 contiguous nucleotides that differ by 0, 1, 2, or 3 nucleotides from formula (IX): 5′-Z₁₃GUAAUUCAAAGGAGCCZ₁₄-3′ (IX), wherein Z₁₃ is selected from one of C, G, A, U or absent, Z₁₄ is a nucleotide sequence is 0-15 nucleotides in length. In certain embodiments, Z₁₃ is U. In certain embodiments, Z₁₄ is selected from U, UC, UU, UA, UG, UGU, UGG, UGC, UGA, UGGC, UGGU, UGGA, UGGG, UGGAG, UGGAGU, UGGAGUU, UGGAGUUU, UGGAGUUUU, UGGAGUUUUA, UGGAGUUUUAU, UGGAGUUUUAUC, UGGAGUUUUAUCA, UGGAGUUUUAUCAA, UGGAGUUUUAUCAAG, UGGAUU, UGGAUU, or absent. In certain embodiments, Z₁₄ is a nucleotide sequence is 1, 2, 3 or 4 nucleotides in length, the nucleotide sequence is selected from U, UC, UU, UA, UG, UGU, UGG, UGC, UGA, UGGC, UGGU, UGGA, UGGG. In certain embodiments, Z₁₃ is absent, Z₁₄ is U. In some embodiments, the sense strand of dsRNA comprises a nucleotide sequence at least 15 contiguous nucleotides that differ by 0, 1, 2, or 3 nucleotides from formula (X): 5′-Z₁₅GGCUCCUUUGAAUUACZ₁₆-3′ (X), wherein Z₁₅ is a nucleotide sequence is 0-15 nucleotides in length, Z₁₆ is selected from one of C, G, A, U or absent. In certain embodiments, Z₁₆ is A. In certain embodiments, Z₁₅ is selected from A, AA, UA, GA, CA, ACA, UCA, GCA, CCA, GCCA, ACCA, CCCA, UCCA, CUCCA, ACUCCA, AACUCCA, AAACUCCA, AAAACUCCA, UAAAACUCCA, AUAAAACUCCA, GAUAAAACUCCA, UGAUAAAACUCCA, UUGAUAAAACUCCA, CUUGAUAAAACUCCA, or absent. In certain embodiments, Z₁₅ is a nucleotide sequence is 1, 2, 3 or 4 nucleotides in length, the nucleotide sequence is selected from A, AA, UA, GA, CA, ACA, UCA, GCA, CCA, GCCA, ACCA, CCCA, UCCA. In certain embodiments, Z₁₆ is absent, Z₁₅ is A. In some embodiments, the dsRNA agent includes a sense strand and an antisense strand, wherein the antisense strand of dsRNA comprises nucleotide sequence of at least 15 contiguous nucleotides that differ by 0, 1, 2, or 3 nucleotides from formula (IX) and the sense strand of dsRNA comprises nucleotide sequence of at least 15 contiguous nucleotides that differ by 0, 1, 2, or 3 nucleotides from formula (X), wherein the nucleotide formula (IX) and (X) are as described above. In some embodiments, each strand is no more than 40 nucleotides in length. In certain embodiments, the sense strand is complementary or substantially complementary to the antisense strand, and the region of complementarity is between 16 and 23 nucleotides in length. In some embodiments, the region of complementarity is 19-21 nucleotides in length. In some embodiments, wherein the sense strand is no more than 35 nucleotides in length comprising a region of complementarity to the antisense strand including at least 15, 16, 17, 18, or 19 nucleotides. In some embodiments, Z₁₃ is a nucleotide complementary to Z₁₆. In some embodiments, Z₁₄ is a nucleotide sequence complementary to Z₁₅.

In some embodiments, the antisense strand of dsRNA comprises a nucleotide sequence of at least 15 contiguous nucleotides that differ by 0, 1, 2, or 3 nucleotides from formula (XI): 5′-Z₁₇UGGCAAACAUUCACUCZ₁₈-3′ (XI), wherein Z₁₇ is selected from one of C, G, A, U or absent, Z₁₈ is a nucleotide sequence is 0-15 nucleotides in length. In certain embodiments, Z₁₇ is U. In certain embodiments, Z₁₈ is selected from C, CC, CU, CA, CG, CUU, CUG, CUC, CUA, CUUC, CUUU, CUUA, CUUG, CUUUA, CUUUAA, CUUUAAC, CUUUAACA, CUUUAACAA, CUUUAACAAA, CUUUAACAAAA, CUUUAACAAAAG, CUUUAACAAAAGU, CUUUAACAAAAGUC, CUUUAACAAAAGUCA, CUUCUU, CUUCGA, or absent. In certain embodiments, Z₁₈ is a nucleotide sequence is 1, 2, 3 or 4 nucleotides in length, the nucleotide sequence is selected from C, CC, CU, CA, CG, CUU, CUG, CUC, CUA, CUUC, CUUU, CUUA, CUUG. In certain embodiments, Z₁₇ is absent, Z₁₈ is C. In some embodiments, the sense strand of dsRNA comprises a nucleotide sequence at least 15 contiguous nucleotides that differ by 0, 1, 2, or 3 nucleotides from formula (XII): 5′-Z₁₉GAGUGAAUGUUUGCCAZ₂₀-3′ (XII), wherein Z₁₉ is a nucleotide sequence is 0-15 nucleotides in length, Z₂₀ is selected from one of C, G, A, U or absent. In certain embodiments, Z₂₀ is A. In certain embodiments, Z₁₉ is selected from G, AG, UG, GG, CG, AAG, UAG, GAG, CAG, CAAG, AAAG, UAAG, GAAG, UAAAG, UUAAAG, GUUAAAG, UGUUAAAG, UUGUUAAAG, UUUGUUAAAG, UUUUGUUAAAG, CUUUUGUUAAAG, ACUUUUGUUAAAG, GACUUUUGUUAAAG, UGACUUUUGUUAAAG or absent. In certain embodiments, Z₁₉ is a nucleotide sequence is 1, 2, 3 or 4 nucleotides in length, the nucleotide sequence is selected from G, AG, UG, GG, CG, AAG, UAG, GAG, CAG, CAAG, AAAG, UAAG, GAAG. In certain embodiments, Z₂₀ is absent, Z₁₉ is G. In some embodiments, the dsRNA agent includes a sense strand and an antisense strand, wherein the antisense strand of dsRNA comprises nucleotide sequence of at least 15 contiguous nucleotides that differ by 0, 1, 2, or 3 nucleotides from formula (XI) and the sense strand of dsRNA comprises nucleotide sequence of at least 15 contiguous nucleotides that differ by 0, 1, 2, or 3 nucleotides from formula (XII), wherein the nucleotide formula (XI) and (XII) are as described above. In some embodiments, each strand is no more than 40 nucleotides in length. In certain embodiments, the sense strand is complementary or substantially complementary to the antisense strand, and the region of complementarity is between 16 and 23 nucleotides in length. In some embodiments, the region of complementarity is 19-21 nucleotides in length. In some embodiments, wherein the sense strand is no more than 35 nucleotides in length comprising a region of complementarity to the antisense strand including at least 15, 16, 17, 18, or 19 nucleotides. In some embodiments, Z₁₇ is a nucleotide complementary to Z₂₀. In some embodiments, Z₁₈ is a nucleotide sequence complementary to Z₁₉.

In certain embodiments, a double-stranded ribonucleic acid (dsRNA) agent for inhibiting expression of prekallikrein (PKK) is provided, wherein the dsRNA agent includes a sense strand and an antisense strand, wherein the sense strand comprises at least 15 contiguous nucleotides that differ by 0, 1, 2, or 3 nucleotides from any one of the nucleotide sequences of nucleotides 1014-1044; 1054-1084; 1059-1089; 1106-1138; 1456-1486; 1543-1573; or 2029-2059 of SEQ ID: 767, and the antisense strand is complementary or substantially complementary to the sense strand sequence in the dsRNA agent, and optionally comprising a targeting ligand. In some embodiments, each strand is no more than 40 nucleotides in length. In certain embodiments, the dsRNA agent includes at least one modified nucleotide. In certain embodiments, all or substantially all of the nucleotides of the antisense strand are modified nucleotides. In some embodiments, the at least one modified nucleotide includes: a 2′-O-methyl nucleotide, 2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-OMe nucleotide, inverted 2′-deoxy nucleotide, 2′-amino-modified nucleotide, 2′-alkyl-modified nucleotide, morpholino nucleotide, and 3′-OMe nucleotide, a nucleotide comprising a 5′-phosphorothioate group, or a terminal nucleotide linked to a cholesteryl derivative or dodecanoic acid bisdecylamide group, a 2′-amino-modified nucleotide, a phosphoramidate, or a non-natural base comprising nucleotide. In some embodiments, the dsRNA agent includes an E-vinylphosphonate nucleotide at the 5′ end of the antisense strand. In some embodiments, the dsRNA agent includes at least one phosphorothioate internucleoside linkage. In certain embodiments, the sense strand includes at least one phosphorothioate internucleoside linkage. In certain embodiments, the antisense strand includes at least one phosphorothioate internucleoside linkage. In some embodiments, the sense strand includes 1, 2, 3, 4, 5, or 6, phosphorothioate internucleoside linkages. In some embodiments, the antisense strand includes 1, 2, 3, 4, 5, or 6, phosphorothioate internucleoside linkages. In some embodiments, all or substantially all of the nucleotides of the sense strand and the antisense strand are modified nucleotides. In some embodiments, the modified sense strand is a modified sense strand sequence set forth in one of Tables 2 and 3. In certain embodiments, the modified antisense strand is a modified antisense strand sequence set forth in one of Tables 2 and 3. In some embodiments, the antisense strand comprises 15 or more modified nucleotides independently selected from a 2′-O-methyl nucleotide and a 2′-fluoro nucleotide, wherein less than 6 modified nucleotides are 2′-fluoro nucleotides. In certain embodiments, the antisense strand comprises 3 or 5 2′-fluoro nucleotides, preferably, the antisense strand comprises 5 2′-fluoro nucleotides. In some embodiments, the sense strand comprises 15 or more modified nucleotides independently selected from a 2′-O-methyl nucleotide and a 2′-fluoro nucleotide, wherein less than 4 modified nucleotides are 2′-fluoro nucleotides. In certain embodiments, the sense strand comprises 3 2′-fluoro nucleotides. In some embodiments, the antisense strand comprises 15 or more modified nucleotides independently selected from a 2′-O-methyl nucleotide and a 2′-fluoro nucleotide, wherein at least 16 modified nucleotides are 2′-O-methyl nucleotide and the nucleotides at position 2, 7, 12, 14 and/or 16 from the 5′ end of the antisense strand are a 2′-fluoro nucleotide. In some embodiments, the sense strand comprises 15 or more modified nucleotides independently selected from a 2′-O-methyl nucleotide and a 2′-fluoro nucleotide, wherein at least 18 modified nucleotides are 2′-O-methyl nucleotide and the nucleotides at position 9, 11 and/or 13 from the 3′ end of the sense strand are 2′-fluoro nucleotide. In some embodiments, the antisense strand comprises in the direction from 5′ terminal to 3′ terminal, the nucleotides at position 2, 7, 12, 14, and 16 of the antisense strand counting from the first paired nucleotide from the 5′ end of the antisense strand, are a 2′-fluoro modified nucleotides, each of the nucleotides at the other positions in the antisense strand is independently a non-fluoro modified nucleotide. In some embodiments, the antisense strand comprises in the direction from 5′ terminal to 3′ terminal, the nucleotides at position 2, 5, 12, 14, and 18 of the antisense strand counting from the first paired nucleotide from the 5′ end of the antisense strand, are a 2′-fluoro modified nucleotides, each of the nucleotides at the other positions in the antisense strand is independently a non-fluoro modified nucleotide. In some embodiments, the sense strand comprises in the direction from 3′ terminal to 5′ terminal, the nucleotides at position 9, 11, and 13 of the sense strand counting from the first paired nucleotide from the 3′ end of the sense strand, are a 2′-fluoro modified nucleotides, each of the nucleotides at the other positions in the sense strand is independently a non-fluoro modified nucleotide. In certain embodiments, the sense strand is complementary or substantially complementary to the antisense strand, and the region of complementarity is between 16 and 23 nucleotides in length. In some embodiments, the region of complementarity is 19-21 nucleotides in length. In some embodiments, each strand is no more than 40 nucleotides in length. In some embodiments, each strand is no more than 30 nucleotides in length. In certain embodiments, each strand is no more than 25 nucleotides in length. In certain embodiments, each strand is no more than 23 nucleotides in length. In certain embodiments, each strand is more than 17 nucleotides in length.

In some embodiments, the dsRNA agent includes at least one modified nucleotide and further includes one or more targeting groups or linking groups. In some embodiments, the one or more targeting groups or linking groups are conjugated to the sense strand. In some embodiments, the targeting group or linking group includes N-acetyl-galactosamine (GalNAc). In some embodiments, the targeting group has a structure:

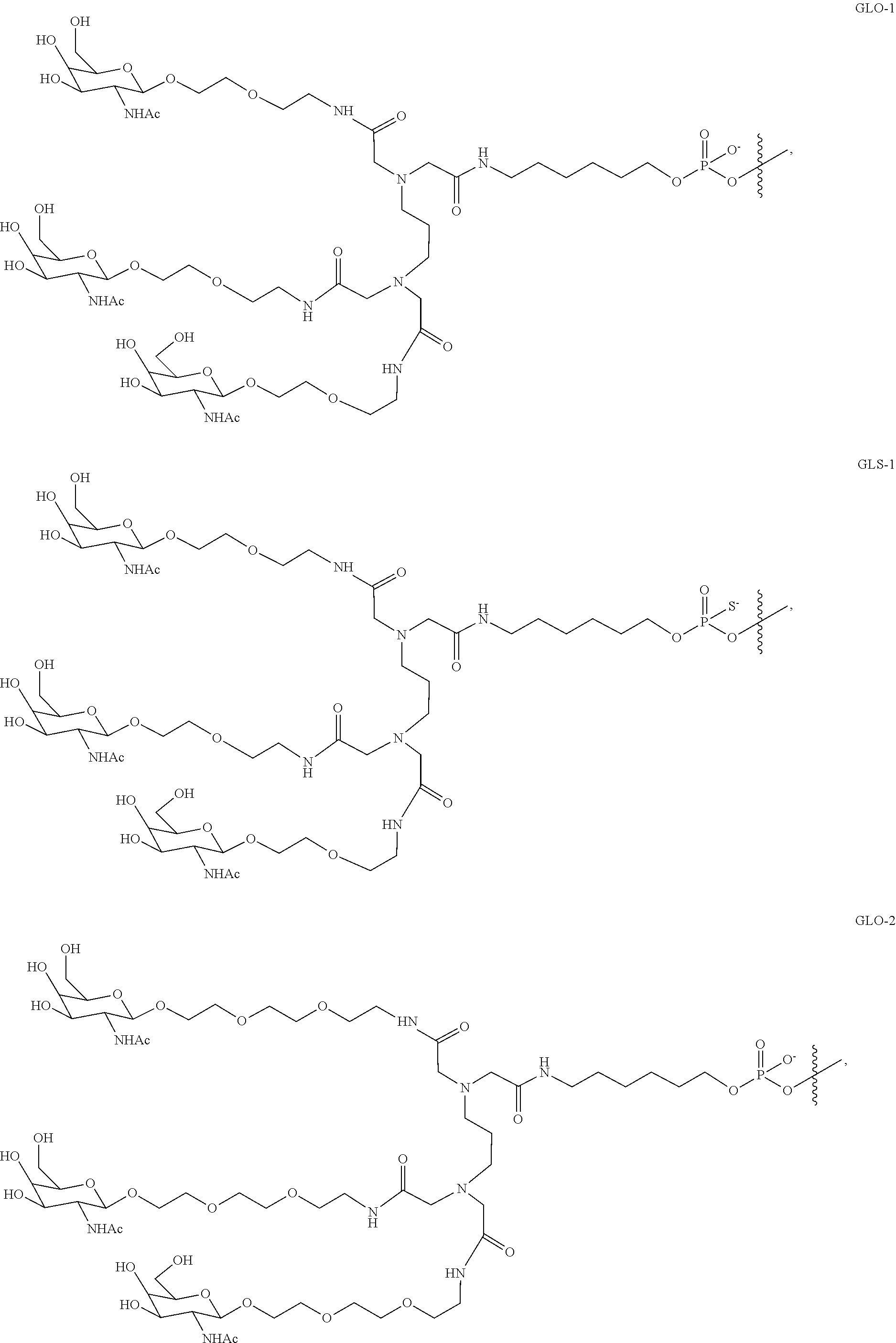 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

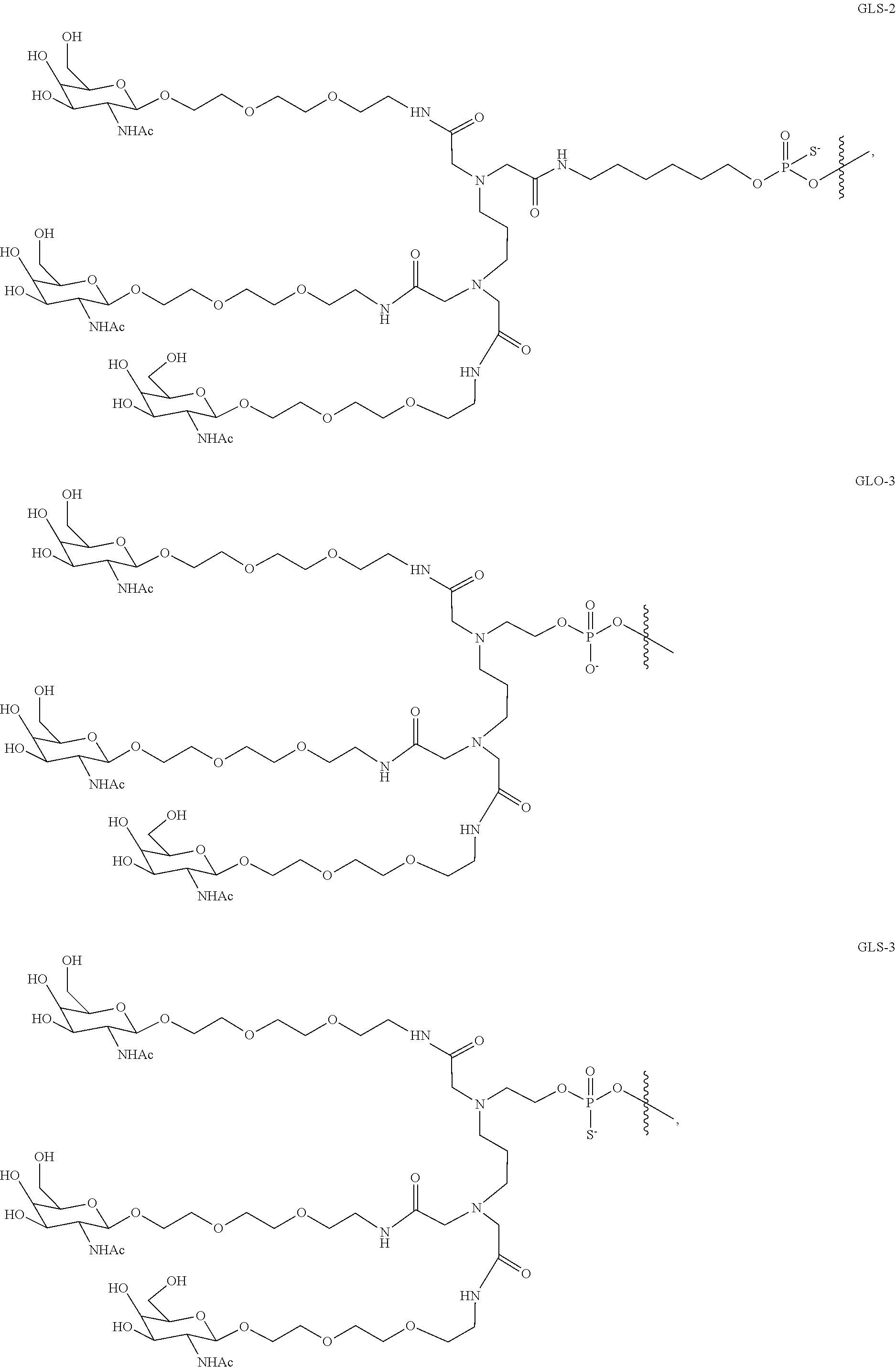 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

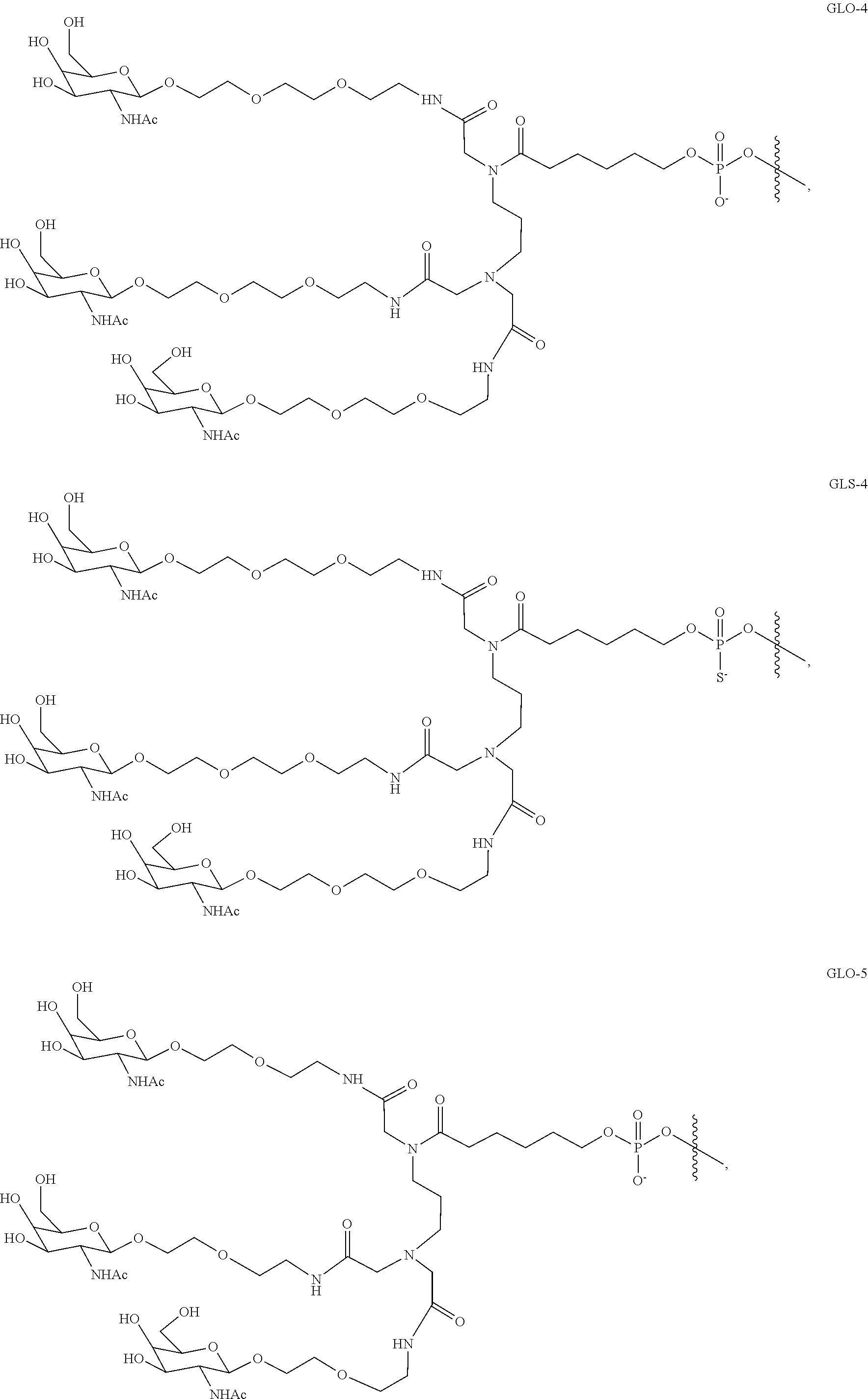 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

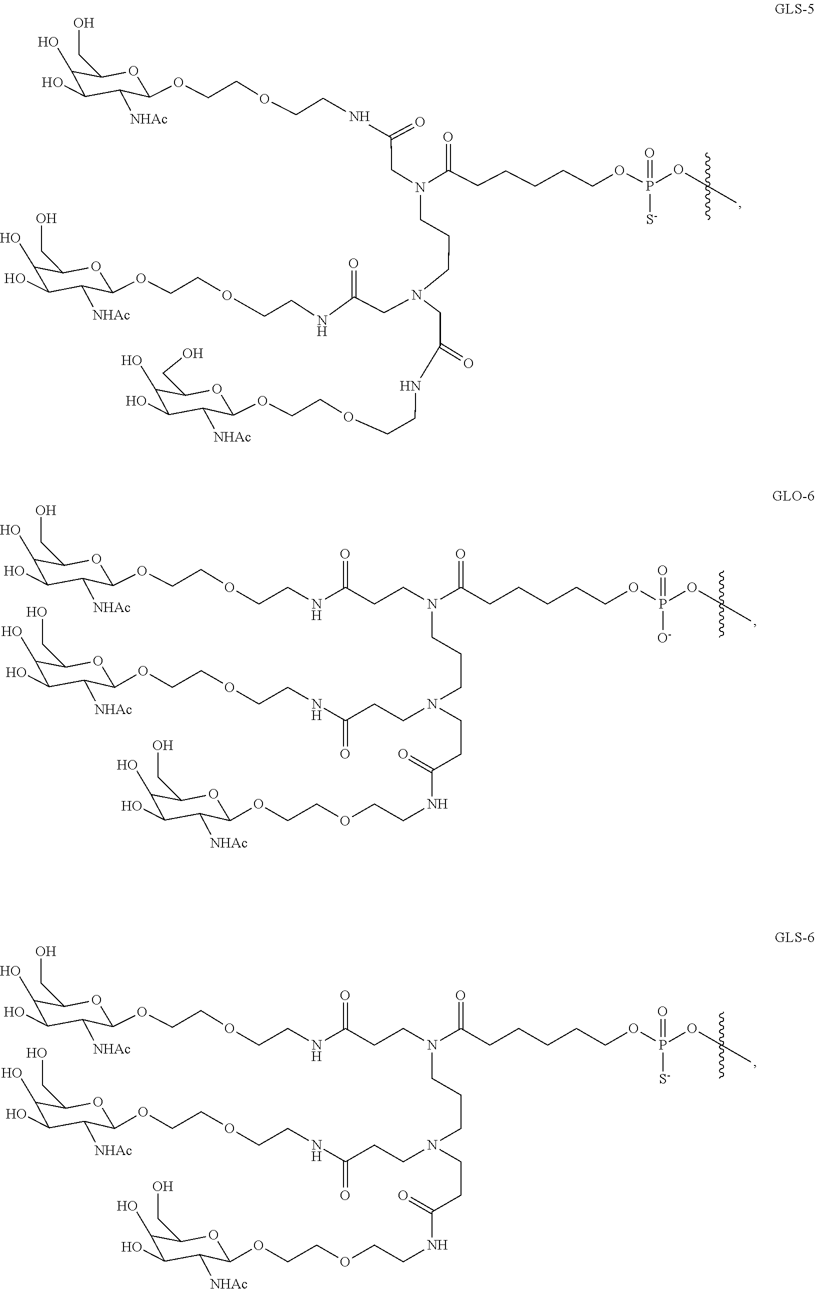 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

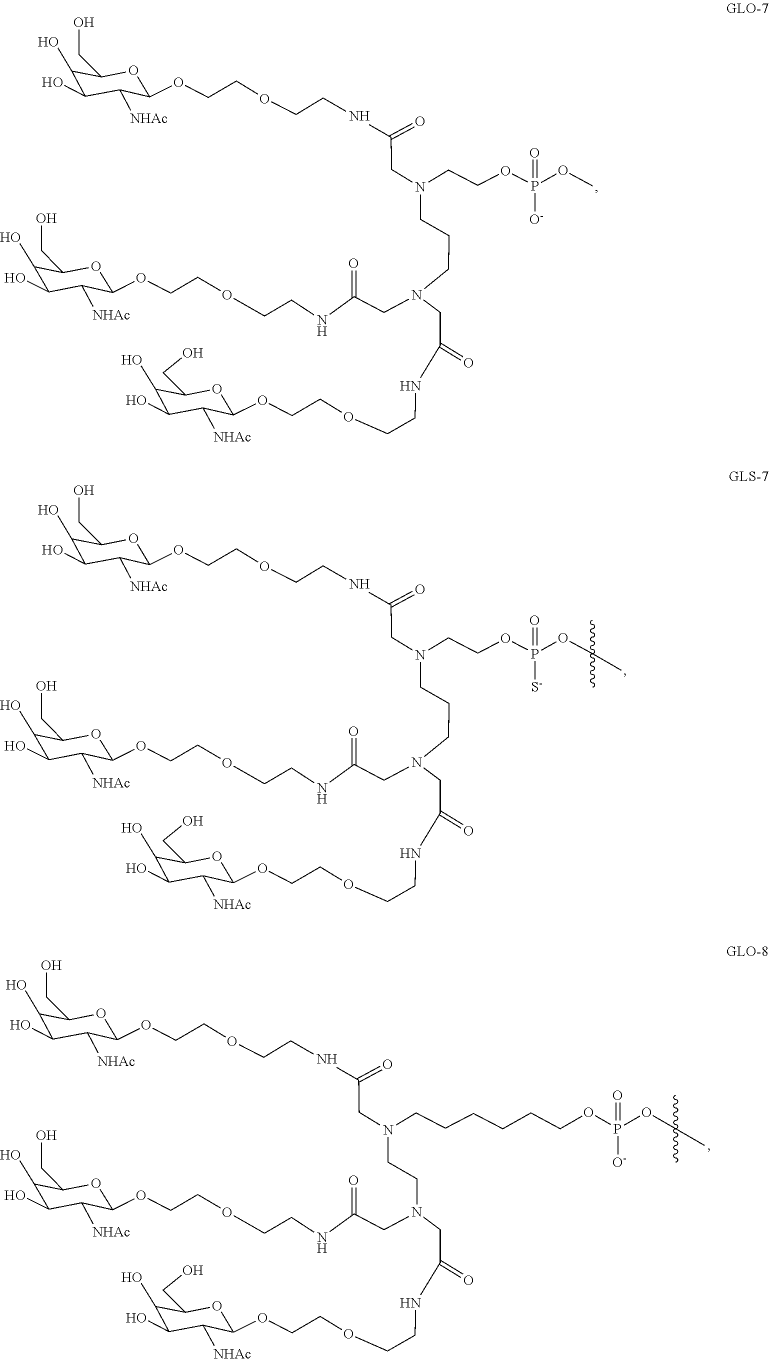 [SMILES: COP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

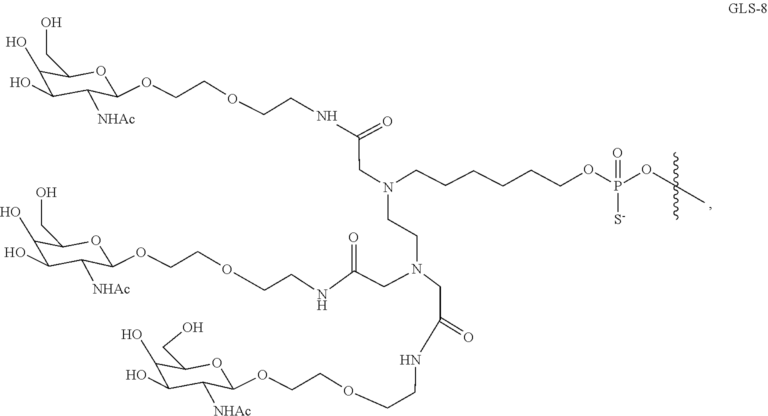 [SMILES: *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

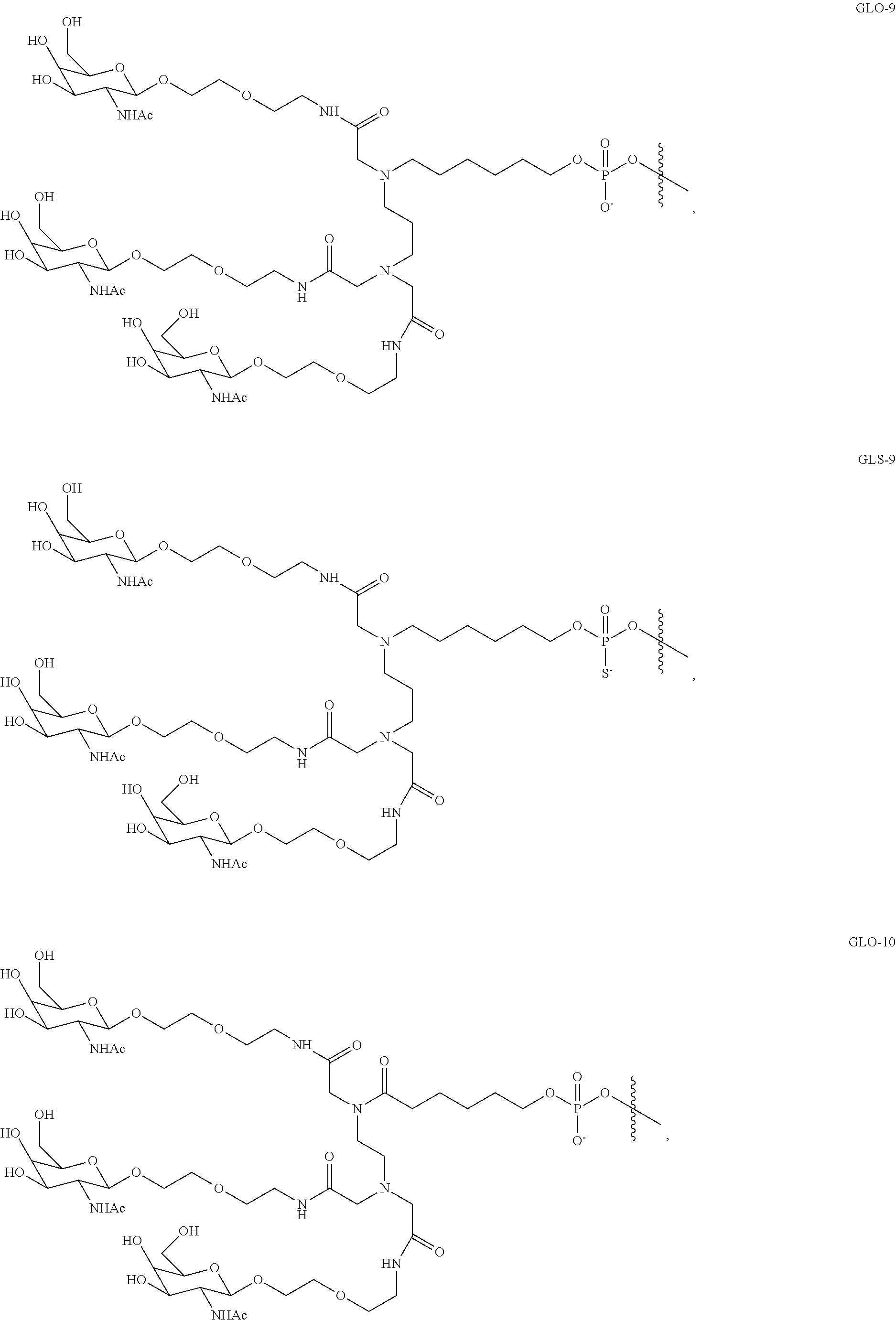 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

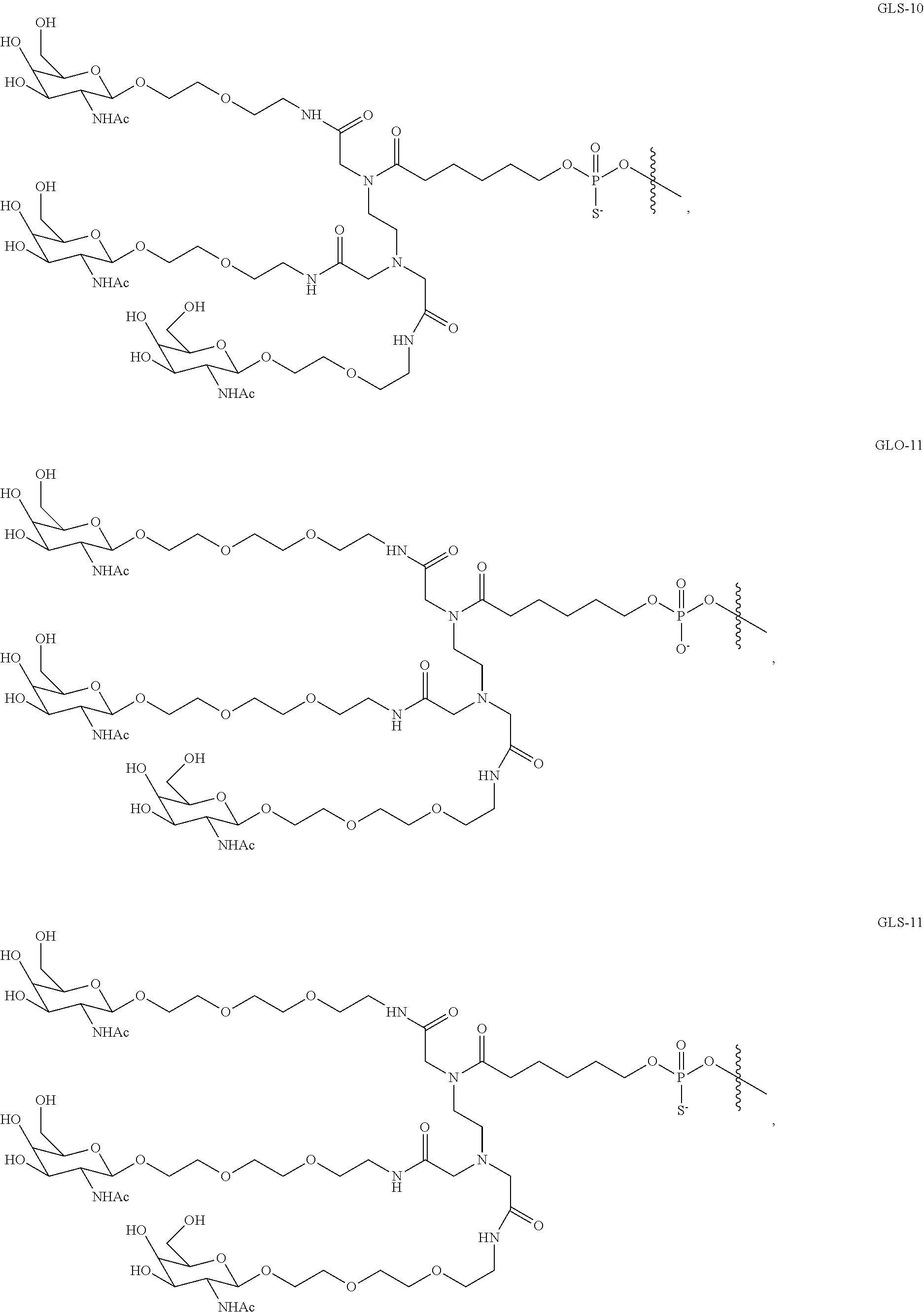 [SMILES: *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

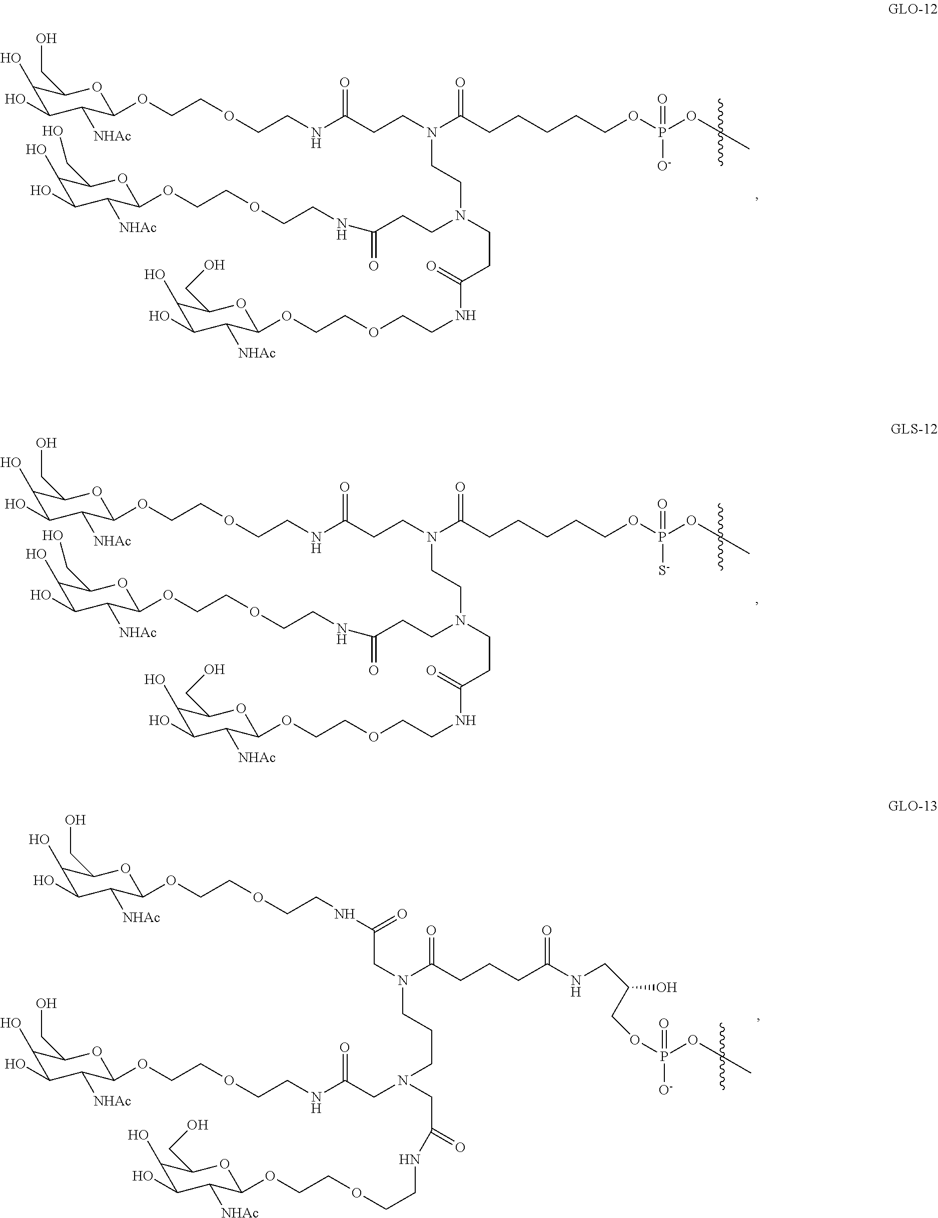 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

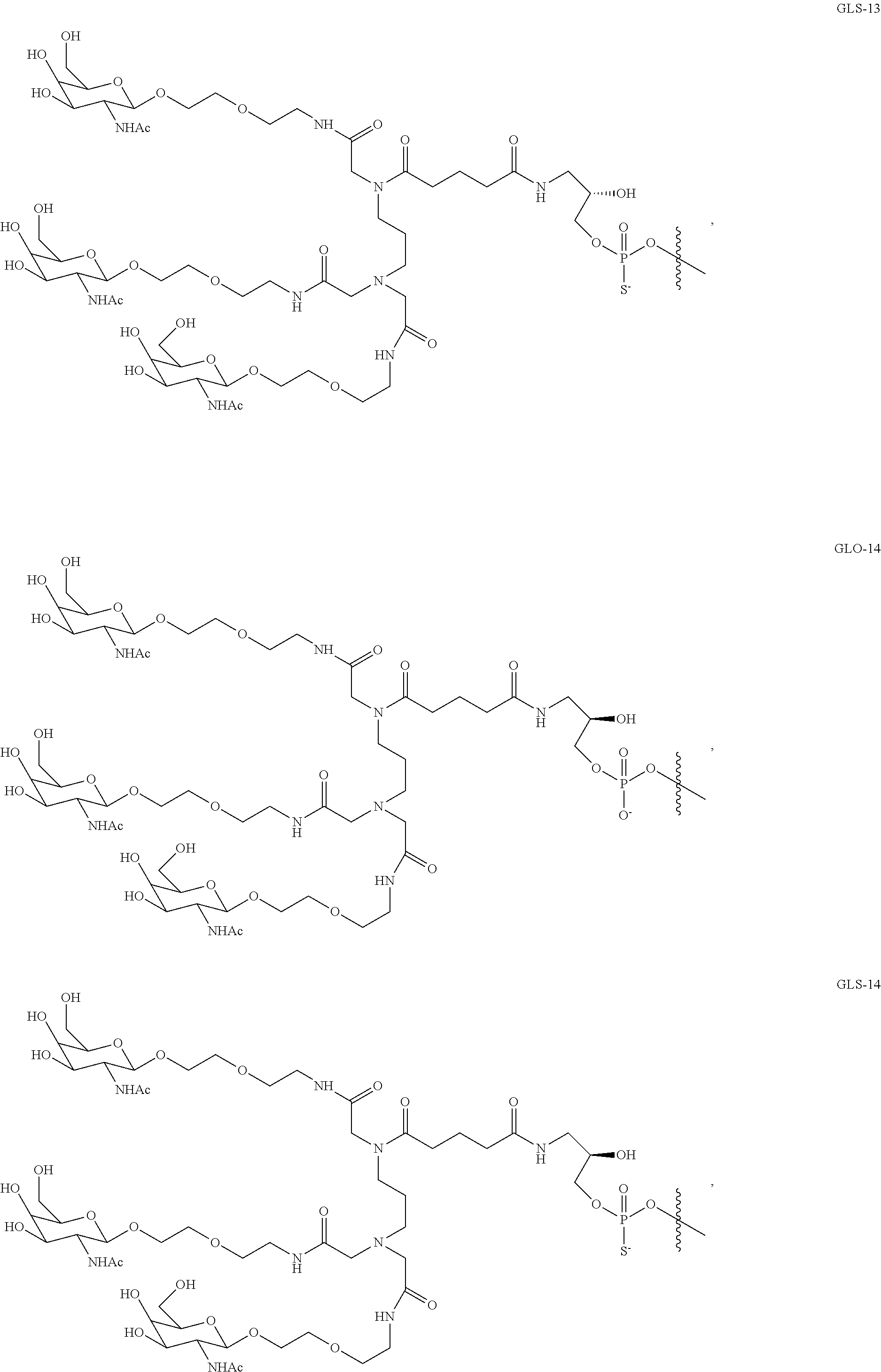 [SMILES: *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

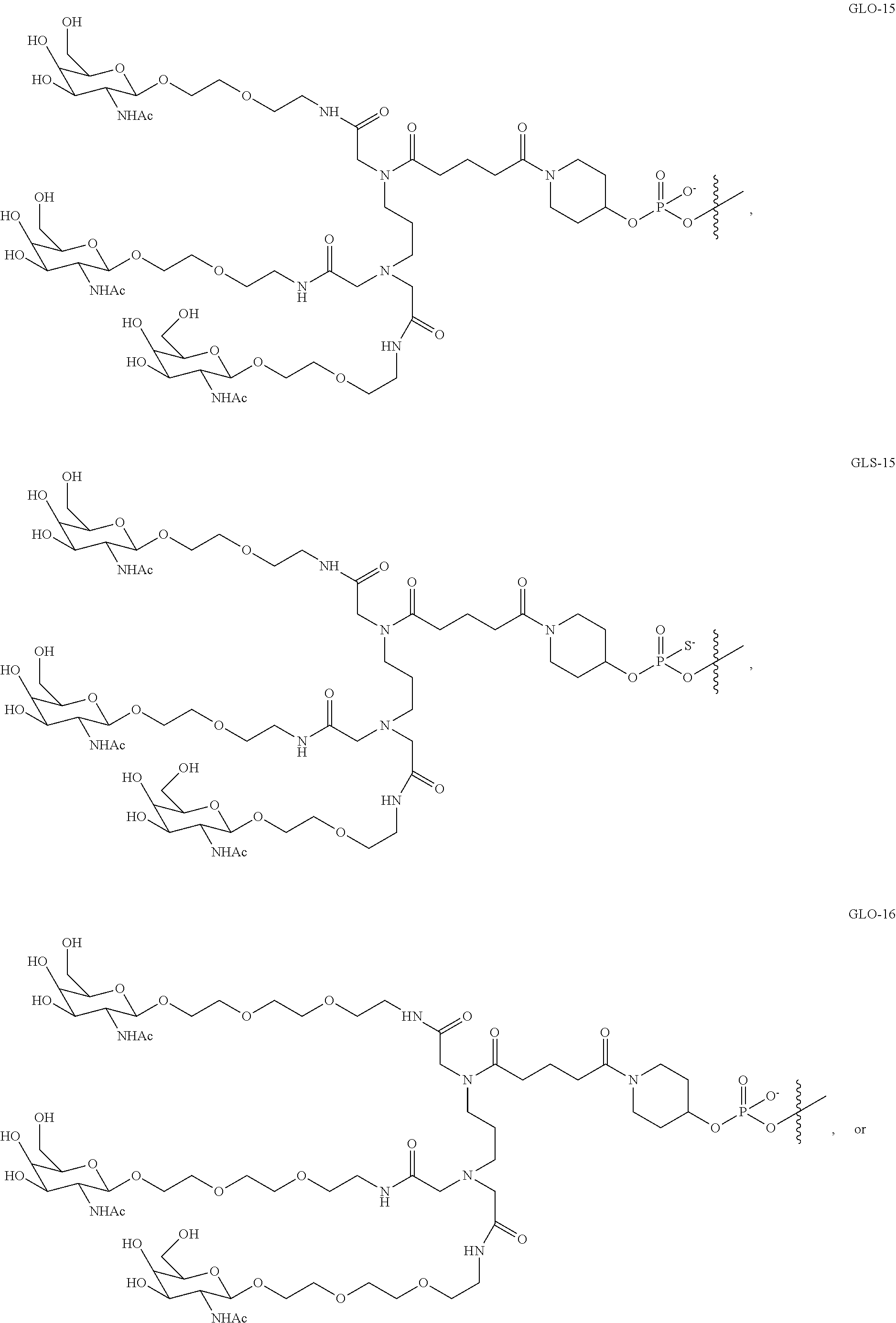 [SMILES: *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

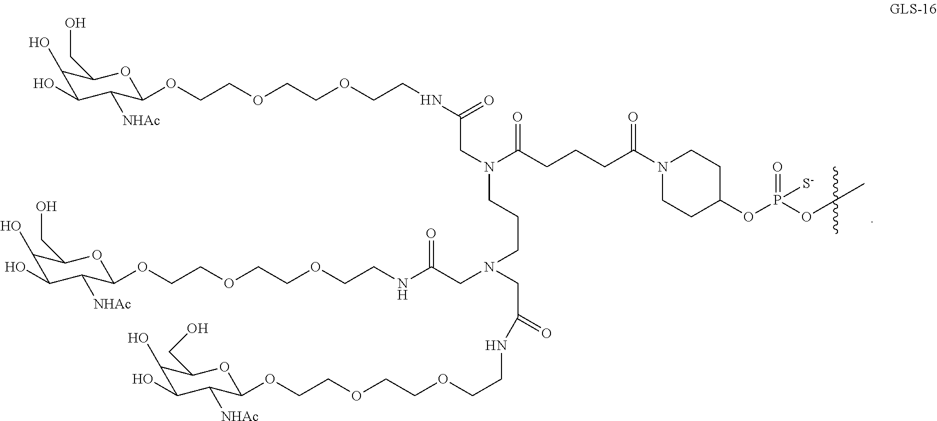 [SMILES: *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In certain embodiments, the dsRNA agent includes a targeting group that is conjugated to the 5′-terminal end of the sense strand. In some embodiments, the dsRNA agent includes a targeting group that is conjugated to the 3′-terminal end of the sense strand. In some embodiments, the antisense strand includes one inverted abasic residue at 3′-terminal end. In certain embodiments, the sense strand includes one or two inverted abasic residues at the 3′ and/or the 5′ terminal end. In certain embodiments, the dsRNA agent has two blunt ends. In certain embodiments, at least one strand includes a 3′ overhang of at least 1 nucleotide. In some embodiments, at least one strand includes a 3′ overhang of at least 2 nucleotides. In some embodiments, the antisense strand includes a 2 3′ overhang of nucleotides, that overhang of nucleotide sequence selected from UU, GA. In some embodiments, the PKK RNA transcript is SEQ ID NO: 767.

According to another aspect of the invention, a composition including one, two, three, or more dsRNA agents of any embodiment of the aforementioned aspect of the invention is provided. In some embodiments, the composition also includes a pharmaceutically acceptable carrier. In some embodiments, the composition also includes one or more additional therapeutic agents. In certain embodiments, composition is packaged in a kit, container, pack, dispenser, pre-filled syringe, or vial. In certain embodiments, the composition is formulated for subcutaneous administration or is formulated for intravenous (IV) administration.

According to another aspect of the invention, a cell including 1, 2, 3, or more of a dsRNA agent of any embodiment of the aforementioned dsRNA agents is provided. In some embodiments, the cell is a mammalian cell. In certain embodiments, the cell is a human cell.

According to another aspect of the invention, a method of inhibiting the expression of a prekallikrein (PKK) gene in a cell is provided, the method including: (i) preparing a cell or plurality of cells comprising an effective amount of one or more double-stranded ribonucleic acid (dsRNA) agents of any one embodiment of an aforementioned dsRNA agent or any embodiment of an aforementioned composition. In certain embodiments, the method also includes (ii) maintaining the cell or plurality of cells prepared for a time sufficient to obtain degradation of the mRNA transcript of a PKK gene, thereby inhibiting expression of the PKK gene in the cell or plurality of cells and reducing a level of the PKK polypeptide in the cell or plurality of cells. In some embodiments, the cell or plurality of cells is in a subject and the dsRNA agent is administered to the subject subcutaneously or by IV administration, or the cell or plurality of cells is outside a subject and is contacted with the dsRNA agent. In some embodiments, wherein 2, 3, 4, or more dsRNA agents are administered to the subject or contacted with the cell or plurality of cells. In some embodiments, the method also includes assessing inhibition of the PKK gene in the subject, following the administration of the dsRNA agent(s) to the subject, wherein a means for the assessing includes: (i) determining one or more physiological characteristics of a PKK-associated disease or condition in the subject and (ii) comparing the determined physiological characteristic(s) to a baseline pre-administration physiological characteristic of the PKK-associated disease or condition and/or to a control physiological characteristic of the PKK-associated disease or condition, wherein the comparison indicates one or more of a presence or absence of inhibition of expression of the PKK gene in the subject. In certain embodiments, the method also includes assessing inhibition of the PKK gene in the cell or plurality of cells, following contacting the dsRNA agent(s) to the cell or plurality of cells, wherein a means for the assessing includes: (i) determining one or more physiological characteristics of a PKK-associated disease or condition in the cell or plurality of cells and (ii) comparing the determined physiological characteristic(s) to a baseline pre-contact physiological characteristic of the PKK-associated disease or condition and/or to a control physiological characteristic of the PKK-associated disease or condition, wherein the comparison indicates one or more of a presence or absence of inhibition of expression of the PKK gene in the cell or plurality of cells. In some embodiments, the control physiological characteristic is the physiological characteristic in a subject with a PKK-associated disease or condition and not administered the dsRNA agent(s) or is the physiological characteristic in a cell with a PKK-associated disease or condition and not contacted with the dsRNA agent(s). In some embodiments, (i) the determined physiological characteristic in the subject is one or more of: a level of plasma PKK in the subject; presence, absence, and/or a level of one or more of plasma kallikrein, high-molecular-weight kininogen (HK), and bradykinin in the subject; or is one or more of a physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject, and (ii) the determined physiological characteristic in the cell or plurality of cells is a level of PKK expression in the cell or plurality of cells, presence, absence, and/or a level of one or more of kallikrein expression, high-molecular-weight kininogen (HK) expression, and bradykinin expression in the cell or plurality of cells. In certain embodiments, the physiological characteristic is determined in a biological sample obtained from the subject. In certain embodiments, the biological sample includes one or more of: a blood sample, a serum sample, a tissue sample, a cell sample, and a liver sample. In some embodiments, the determined physiological characteristic in the subject is abnormal compared to a control level of the physiological characteristic. In some embodiments, wherein the control physiological characteristic in the subject is the physiological characteristic in a subject with the PKK-associated disease or condition and not administered the anti-PKK dsRNA agent(s) and the control physiological characteristic in the cell or plurality of cells is the physiological characteristic in a cell with the PKK-associated disease or condition and not contacted with the anti-PKK dsRNA agent(s). In some embodiments, a change in a level of plasma PKK in the subject; presence, absence, and/or a level of one or more of plasma kallikrein, high-molecular-weight kininogen (HK), and bradykinin in the subject indicates a reduction of PKK gene expression in the subject and change in a level of PKK expression in the cell; presence, absence, and/or a level of one or more of kallikrein, high-molecular-weight kininogen (HK), and bradykinin in the cell or plurality of cells indicates a reduction of PKK gene expression in the cell or plurality of cells. In certain embodiments, the PKK-associated disease or condition is hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), or varicose veins. In certain embodiments, the method also includes administering an additional therapeutic regimen to the subject.

According to another aspect of the invention, a method of inhibiting expression of a PKK gene in a subject is provided, the method including administering to the subject an effective amount of one or more double-stranded ribonucleic acid (dsRNA) agents of any one embodiment of an aforementioned dsRNA agent or any embodiment of an aforementioned composition. In certain embodiments, two, three, four, or more of the dsRNA agents are administered to the subject. In some embodiments, the dsRNA agent is administered to the subject subcutaneously or by IV administration. In some embodiments, the method also includes assessing inhibition of the PKK gene in the subject, following the administration of the one or more dsRNA agents, wherein a means for the assessing includes: (i) determining one or more physiological characteristics of a PKK-associated disease or condition in the subject and (ii) comparing the determined physiological characteristic(s) to a baseline pre-treatment physiological characteristic of the PKK-associated disease or condition and/or to a control physiological characteristic of the PKK-associated disease or condition, wherein the comparison indicates one or more of a presence or absence of inhibition of expression of the PKK gene in the subject. In certain embodiments, the determined physiological characteristic in the subject is one or more of: a level of plasma PKK in the subject; presence, absence, and/or a level of one or more of plasma kallikrein, high-molecular-weight kininogen (HK), and bradykinin in the subject; a physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject. In certain embodiments, the physiological characteristic is determined in a biological sample obtained from the subject. In some embodiments, the biological sample includes one or more of: a blood sample, a serum sample, a cell sample, a tissue sample, and a liver sample. In some embodiments, the determined physiological characteristic in the subject is more normal compared to a baseline pre-treatment physiological characteristic of the PKK-associated disease or condition in the subject and/or to a control physiological characteristic of the PKK-associated disease or condition. In certain embodiments, a level of one or more of: plasma PKK in the subject; plasma kallikrein, high-molecular-weight kininogen (HK), and bradykinin in the subject; determined to be statistically significantly higher, lower, or unchanged compared to a baseline pre-treatment physiological characteristic of the PKK-associated disease or condition and/or to a control physiological characteristic of the PKK-associated disease or condition indicates a status of PKK gene expression in the subject. In certain embodiments, a reduction in one or more of the physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject indicates a reduction of PKK gene expression in the subject. In some embodiments, the PKK-associated disease or condition is one or more of: hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins. In some embodiments, the method also includes administering an additional therapeutic regimen to the subject.

According to another aspect of the invention, a method of treating a disease or condition in a subject is provided, wherein the disease or condition is associated with the presence of a prekallikrein (PKK) protein, and the method includes administering to the subject an effective amount of one or more of any embodiment of an aforementioned double-stranded ribonucleic acid (dsRNA) agent or any embodiment of an aforementioned composition to inhibit expression of the PKK gene encoding the PKK protein. In certain embodiments, the method includes administering two, three, four, or more dsRNA agents to the subject. In certain embodiments, the disease or condition is one or more of: hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins. In some embodiments, the method also includes administering an additional therapeutic regimen to the subject. In some embodiments, the additional therapeutic regimen includes: administering to the subject one or more: PKK antisense polynucleotides, additional PKK dsRNA therapeutic agent, a non-PKK dsRNA therapeutic agent, a PKK non-dsRNA therapeutic agent, and a behavioral modification. In certain embodiments, the non-PKK dsRNA therapeutic agent is one or more of: a blood thinner, an NSAID, aspirin, an anticoagulant, tissue plasminogen activator (tPA); recombinant tissue plasminogen activator (tPA), a statin, a stent, a cholesterol-lowering therapeutic agent, a vasodilator, cilostazol, Plavix®, an anti-coagulation drug, and an antiplatelet agent. In certain embodiments, one or more dsRNA agent(s) is administered to the subject by one of both of subcutaneous administration and intravenous (IV) administration. In some embodiments, the administration results in a reduction in a level and/or severity of the physiological characteristic.

In some embodiments, the method also includes determining an efficacy of the administered one or more double-stranded ribonucleic acid (dsRNA) agent(s) in the subject. In certain embodiments, a means of determining an efficacy of the treatment in the subject includes: (i) determining one or more physiological characteristics of the PKK-associated disease or condition in the subject and (ii) comparing the determined physiological characteristic(s) to a baseline pre-treatment physiological characteristic of the PKK-associated disease or condition, wherein the comparison indicates one or more of a presence, absence, and level of efficacy of the administration of the double-stranded ribonucleic acid (dsRNA) agent to the subject. In certain embodiments, the determined physiological characteristic in the subject is one or more of: a level of plasma PKK in the subject; presence, absence, and/or a level of one or more of plasma kallikrein, high-molecular-weight kininogen (HK), and bradykinin in the subject; a physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject. In some embodiments, the physiological characteristic is determined in a biological sample obtained from the subject. In some embodiments, the biological sample includes one or more of: a blood sample, a serum sample, a cell sample, a tissue sample, and a liver sample. In certain embodiments, the determined physiological characteristic in the subject is different compared to the baseline pre-treatment physiological characteristic of the PKK-associated disease or condition in the subject or to a control level of the physiological characteristic. In certain embodiments, a level of one or more of the plasma PKK; plasma kallikrein, high-molecular-weight kininogen (HK), and bradykinin; determined to be statistically significantly higher, lower, or unchanged compared to a baseline pre-treatment physiological characteristic of the PKK-associated disease or condition and/or to a control physiological characteristic of the PKK-associated disease or condition indicates a status of PKK gene expression in the subject. In some embodiments, the PKK-associated disease or condition is one or more of: hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins.

According to another aspect of the invention, a method of decreasing a level of PKK protein in a subject compared to a baseline pre-treatment level of PKK protein in the subject is provided, the method including administering to the subject an effective amount of one or more of any embodiment of an aforementioned double-stranded ribonucleic acid (dsRNA) agent(s) or any embodiment of an aforementioned composition of the invention, to decrease the level of PKK gene expression. In some embodiments, the dsRNA agent is administered to the subject subcutaneously or is administered to the subject by IV administration.

According to yet another aspect of the invention, a method of altering a physiological characteristic of a prekallikrein (PKK)-associated disease or condition in a subject compared to a baseline pre-treatment physiological characteristic of the PKK-associated disease or condition in the subject is provided, the method including administering to the subject an effective amount of one or more of any embodiment of an aforementioned double-stranded ribonucleic acid (dsRNA) agent(s) or any embodiment of an aforementioned composition of the invention to alter the physiological characteristic of the PKK-associated disease or condition in the subject. In certain embodiments, the altering is reducing a level or severity of the physiological characteristic. In some embodiments, the one or more dsRNA agent(s) is administered to the subject subcutaneously or is administered to the subject by IV administration. In some embodiments, wherein the physiological characteristic is one or more of a level of plasma PKK in the subject; presence, absence, and/or a level of one or more of plasma kallikrein, high-molecular-weight kininogen (HK), and bradykinin in the subject; a physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject.

### BRIEF DESCRIPTION OF THE SEQUENCES

SEQ ID NOs: 1-224 and 768-972 are shown in Table 1 and are sense strand sequences.
SEQ ID NOs: 225-448 and 973-1177 are shown in Table 1 and are antisense strand sequences.
SEQ ID NOs: 449-560, 1178-1382 are sense strand sequences and SEQ ID NOs: 561-672, 1383-1587 are antisense strand sequences and are shown in Table 2 with chemical modifications indicated as: upper case: 2′-Fluoro; lower case: 2′-OMe; and thiophosphate: *.
SEQ ID NOs: 673-719, 1588-1635 are sense strand sequences and SEQ ID NO: 720-766, 1636-1683 are antisense strand sequences and are shown in Table 3. Delivery molecules are indicated as “GLX-0” at the 3′ end of sense strands SEQ ID NOs: 673-697; “GLS-5” at the 5′ end of sense strands SEQ ID NOs: 698-709; and GLS-15 at the 5′ end of sense strands 710-719, 1588-1635. Chemical modifications are indicated as: upper case: 2′-Fluoro; lower case: 2′-OMe; thiophosphate: *; and Invab: inverted abasic.

SEQ ID NO: 767 is Homo sapiens kallikrein B1
(KLKB1 or PKK) mRNA sequence [NCBI Reference
Sequence: NM_000892.5]:
agtgccacattagaacagcttgaagaccgttcatttttaagtgaca
agagactcacctccaagaagcaattgtgttttcagaatgatttta
ttcaagcaagcaacttatttcatttccttgtttgctacagtttcc
tgtggatgtctgactcaactctatgaaaacgccttcttcagaggt
ggggatgtagcttccatgtacaccccaaatgcccaatactgccag
atgaggtgcacattccacccaaggtgtttgctattcagttttctt
ccagcaagttcaatcaatgacatggagaaaaggtttggttgcttc
ttgaaagatagtgttacaggaaccctgccaaaagtacatcgaaca
ggtgcagtttctggacattccttgaagcaatgtggtcatcaaata
agtgcttgccatcgagacatttataaaggagttgatatgagagga
gtcaattttaatgtgtctaaggttagcagtgttgaagaatgccaa
aaaaggtgcaccagtaacattcgctgccagtttttttcatatgcc
acgcaaacatttcacaaggcagagtaccggaacaattgcctatta
aagtacagtcccggaggaacacctaccgctataaaggtgctgagt
aacgtggaatctggattctcactgaagccctgtgccctttcagaa
attggttgccacatgaacatcttccagcatcttgcgttctcagat
gtggatgttgccagggttctcactccagatgcttttgtgtgtcgg
accatctgcacctatcaccccaactgcctcttctttacattctat
acaaatgtatggaaaatcgagtcacaaagaaatgtttgtcttctt
aaaacatctgaaagtggcacaccaagttcctctactcctcaagaa
aacaccatatctggatatagccttttaacctgcaaaagaacttta
cctgaaccctgccattctaaaatttacccgggagttgactttgga
ggagaagaattgaatgtgacttttgttaaaggagtgaatgtttgc
caagagacttgcacaaagatgattcgctgtcagtttttcacttat
tctttactcccagaagactgtaaggaagagaagtgtaagtgtttc
ttaagattatctatggatggttctccaactaggattgcgtatggg
acacaagggagctctggttactctttgagattgtgtaacactggg
gacaactctgtctgcacaacaaaaacaagcacacgcattgttgga
ggaacaaactcttcttggggagagtggccctggcaggtgagcctg
caggtgaagctgacagctcagaggcacctgtgtggagggtcactc
ataggacaccagtgggtcctcactgctgcccactgctttgatggg
cttcccctgcaggatgtttggcgcatctatagtggcattttaaat
ctgtcagacattacaaaagatacacctttctcacaaataaaagag
attattattcaccaaaactataaagtctcagaagggaatcatgat
atcgccttgataaaactccaggctcctttgaattacactgaattc
caaaaaccaatatgcctaccttccaaaggtgacacaagcacaatt
tataccaactgttgggtaaccggatggggcttctcgaaggagaaa
ggtgaaatccaaaatattctacaaaaggtaaatattcctttggta
acaaatgaagaatgccagaaaagatatcaagattataaaataacc
caacggatggtctgtgctggctataaagaagggggaaaagatgct
tgtaagggagattcaggtggtcccttagtttgcaaacacaatgga
atgtggcgtttggtgggcatcaccagctggggtgaaggctgtgcc
cgcagggagcaacctggtgtctacaccaaagtcgctgagtacatg
gactggattttagagaaaacacagagcagtgatggaaaagctcag
atgcagtcaccagcatgagaagcagtccagagtctaggcaatttt
tacaacctgagttcaagtcaaattctgagcctggggggtcctcat
ctgcaaagcatggagagtggcatcttctttgcatcctaaggacga
aaaacacagtgcactcagagctgctgaggacaatgtctggctgaa
gcccgctttcagcacgccgtaaccaggggctgacaatgcgaggtc
gcaactgagatctccatgactgtgtgttgtgaaataaaatggtga
aagatca

### BRIEF DESCRIPTION OF THE DRAWINGS

FIG. 1 is a graph showing PKK level in monkey serum after a single subcutaneous dose of siRNA compounds at 3 mg/kg.

FIG. 2 is a graph showing PKK level in monkey serum after a single subcutaneous dose of siRNA compounds at 3 mg/kg.

FIG. 3 is a graph showing PKK level in monkey serum after a single subcutaneous dose of siRNA compounds at 3 mg/kg.

### DETAILED DESCRIPTION

The invention in part, includes RNAi agents, for example, though not limited to double stranded (ds) RNAi agents capable of inhibiting kallikrein B1 (KLKB1 or PKK) gene expression. Anti-PKK dsRNA agents of the invention can target a PKK transcript, leading to suppression of PKK protein expression. Compounds, compositions, and methods of the invention provide anti-PKK RNAi therapeutic agents and treatments for hereditary angioedema, angioedema, bradykinin-mediated angioedema, diabetic retinopathy, diabetic macular edema, hypertension, nephropathy, vascular disease, cardiovascular disease, thrombosis, inflammation, and/or other PKK-associated diseases and conditions. Anti-PKK RNAi agents and compounds delivered to cells using methods of the invention are capable of inhibiting PKK gene expression, thereby reducing activity in the cell of the PKK protein product of the gene. Anti-PKK dsRNAi agents of the invention can be administered to a subject to treat a PKK-associated disease and/or condition in the subject.

In certain embodiments of the invention, agents, compounds, compositions and methods of the invention are used to achieve efficacy in preventing/treating a PKK-associated disease or condition. For example, though not intended to be limiting: a subject with angioedema that is administered a treatment/therapeutic agent or composition of the invention may have one or more of a reduction in severity and frequency of instances of angioedema, a reduction of pain, a reduction of swelling, fewer hospital visits, shorter-length hospital stays, and improved life quality compared to these characteristics in a subject not administered the treatment/therapeutic agent or composition of the invention. In another non limiting example, a subject with thrombosis that is administered a treatment/therapeutic agent or composition of the invention may have one or more of: fewer blood clots, eradication of one or more blood clots, and a reduction in severity and frequency of instances of thrombosis.

A PKK RNAi as described herein is capable of inhibiting expression of PKK protein. In some embodiments of the invention reducing PKK expression in a cell or subject treats a disease or condition associated with PKK expression in the cell or subject, respectively. Non-limiting examples of diseases and conditions that may be treated by reducing PKK activity are: hereditary angioedema, angioedema, hypertension, nephropathy, vascular disease [non-limiting examples of which are: stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins, cardiovascular disease, thrombosis, inflammation, and/or other diseases for which reducing a level and activity of PKK protein is medically beneficial to a subject.

The invention, in part, includes compositions comprising anti-PKK RNAi agents and methods of use of such compositions. Some aspects of the invention include pharmaceutical compositions comprising one or more PKK dsRNA agents and a pharmaceutically acceptable carrier. In certain embodiments, an anti-PKK RNAi agent of the invention is attached to a delivery compound capable of delivering the RNAi agent to a cell, including but not limited to a hepatocyte. In some embodiments, a delivery compound is a GalNAc-containing delivery compound.

Certain pharmaceutical compositions of the invention include one, two, three, or more independently selected anti-PKK dsRNA agents, and may also include one or more independently selected delivery compounds. In some embodiments, two, three, four, or more anti-PKK dsRNAs capable of targeting one, two, three, four, or more different positions/regions of PKK mRNA, respectively, are administered to a cell and/or a subject.

The following describes ways to make and use compositions comprising anti-PKK dsRNA agents (duplexes) capable of inhibiting PKK gene expression, as well as compositions and methods for treating diseases and conditions caused and/or modulated by PKK gene expression. As used herein diseases and/or conditions caused or modulated by a presence and/or level of PKK gene expression are referred to as “PKK-associated diseases and/or conditions.”

As used herein, the term “RNAi” refers to an agent comprising RNA that is capable of mediating targeted cleavage of an RNA transcript via an RNA-induced silencing complex (RISC) pathway. The terms “RNAi” and RNAi agent” may be used interchangeably herein with the terms “anti-PKK RNAi” and “anti-PKK RNAi agent”; “PKK RNAi” and “PKK RNAi agent”; “dsRNA” and “dsRNA agent”; “PKK dsRNA” and “PKK dsRNA agent”; and “anti-PKK dsRNA” and “anti-PKK dsRNA agent”, respectively. As is known in the art, an RNAi target region refers to a contiguous portion of the nucleotide sequence of an mRNA molecule formed during the transcription of a gene, including messenger RNA (mRNA) that is a product of RNA processing of a primary transcription product. The target portion of the mRNA molecule (also referred herein to as the “target sequence”) will be at least long enough to serve as a substrate for RNAi-directed cleavage at or near that portion. A target sequence may be from 8-30 nucleotides long (inclusive), from 10-30 nucleotides long (inclusive), from 12-25 nucleotides long (inclusive), from 15-23 nucleotides long (inclusive), from 16-23 nucleotides long (inclusive), or from 18-23 nucleotides long (inclusive), including all shorter lengths within each stated range. In certain embodiment a target sequence is between 9 and 26 nucleotides long (inclusive), including all sub-ranges and integers there between. For example, though not intended to be limiting, in certain embodiments of the invention a target sequence is one of 8, 9, 10, 11, 12, 13, 14, 15, 16, 17, 18, 19, 20, 21, 22, 23, 24, 25, 26, 27, 28, 29, or 30 nucleotides long, with the sequence fully or at least substantially complementary to at least part of a PKK RNA transcript.

As used herein, a “dsRNA agent” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. Although not wishing to be limited to a particular theory, dsRNA agents of the invention may operate through the RNA interference mechanism [i.e., inducing RNA interference through interaction with the RNA interference pathway machinery (RNA-induced silencing complex or RISC) of mammalian cells], or by any alternative mechanism(s) or pathway(s). Methods for silencing genes in plant, invertebrate, and vertebrate cells are well known in the art [see, for example, (Sharp et al., Genes Dev. 2001, 15:485; Bernstein, et al., (2001) Nature 409:363; Nykanen, et al., (2001) Cell 107:309; and Elbashir, et al., (2001) Genes Dev. 15:188)], the disclosure of each of which is incorporated herein by reference in its entirety]. Art-known gene silencing procedures can be used in conjunction with the disclosure provided herein to inhibit expression of PKK.

dsRNA agents disclosed herein are comprised of a sense strand and an antisense strand, and include, but are not limited to: short interfering RNAs (siRNAs), RNAi agents, micro RNAs (miRNAs), short hairpin RNAs (shRNA), and dicer substrates. An antisense strand of a dsRNA agents described herein is at least partially complementary to the mRNA being targeted. It is understood in the art that different lengths of dsRNA duplex structure can be used to inhibit target gene expression. For example, dsRNAs having a duplex structure of 19, 20, 21, 22, and 23 base pairs are known to be effective to induce RNA interference (Elbashir et al., EMBO 2001, 20:6877-6888) and shorter or longer RNA duplex structures are also known in the art capable of inducing RNA interference. PKK dsRNAs in certain embodiments of the invention can include at least one strand of a length of minimally 21 nt or may have shorter duplexes. In certain embodiments a dsRNA agent of the invention is a duplex disclosed in any of Tables 1-3. In some embodiments, a dsRNA agent of the invention is a duplex disclosed in Tables 1-3, but minus 1, 2, 3, or 4 nucleotides on one or both ends of the duplex, and is also capable of reducing PKK expression. In some embodiments of the invention, PKK dsRNA agents may have a partial sequence of at least 15, 16, 17, 18, 19, 20, or more contiguous nucleotides from one or more sequences of Tables 1-3, and differ in their ability to inhibit the expression of the PKK gene by not more than 5%, 10%, 15%, 20%, 25%, or 30% from the level of inhibition resulting from a dsRNA comprising the full sequence. A sense sequence, an antisense sequence and a duplex disclosed in Tables 1-3 may be referred to herein as a “parent” sequence, meaning that the sequences disclosed in Tables 1-3 may be modified, shorten, lengthened, include substitutions, etc. as set forth herein, with the resulting sequences retaining all or at least a portion of the efficacy of their parent sequences in methods and compositions of the invention. Sense and antisense strands included in a dsRNA of the invention are independently selected. As used herein the term “independently selected” means each of two or more like elements can be selected independent of the selection of the other elements. For example, though not intended to be limiting, in preparing a dsRNA of the invention, one may select the “elements” of the two strands to include in the duplex. One selected element, the sense sequence may be SEQ ID NO: 675 (shown in Table 3) and the other selected element, the antisense sequence, may be SEQ ID NO: 722, or may be SEQ ID NO: 722 that is modified, shortened, lengthened, and/or includes 1, 2, or 3 substitutions as compared to its parent sequence SEQ ID NO: 722. It will be understood that a duplex of the invention need not include both sense and antisense sequences shown as paired in duplexes in Tables 1-3. Each sense and antisense strand sequence in the tables is immediately followed by its SEQ ID NO.

Tables 1-3 show certain PKK dsRNA agent antisense strand and sense strand core stretch base sequences. The term “base sequence” is used herein in reference to a polynucleotide sequence without chemical modifications or delivery compounds. For example, the sense strand ugguugcuucuugaaagauaa (SEQ ID NO: 2) shown in Table 1 is the base sequence for SEQ ID NO: 450 in Table 2 with SEQ ID NO: 450 shown with its chemical modifications. Sequences disclosed herein may be assigned identifiers. For example, a single-stranded sense sequence may be identified with a “Sense strand SS #”; a single stranded antisense sequence may be identified with an “Antisense strand AS #” and a duplex that includes a sense strand and an antisense strand may be identified with a “Duplex AV #” or a “Duplex AD #”.

Table 1 discloses sense and antisense strand sequences and provides the identification number of duplexes formed from the sense and antisense strand on the same line in Table 1. As used herein, the term “matching position” in a sense and an antisense strands are the positions in each strand that “pair” when the two strands are duplexed strands. For example, in a 21 nucleobase sense strand and a 21 nucleobase antisense strand, nucleobase in position 1 of the sense strand and the nucleobase in position 21 in the antisense strand are in “matching positions”. In yet another non-limiting example in a 23 nucleobase sense strand and a 23 nucleobase antisense strand, nucleobase 2 of the sense strand and the nucleobase in position 22 of the antisense strand are in matching positions. In another non-limiting example, in an 18 nucleobase sense strand and an 18 nucleobase antisense strand, the nucleobase in position 1 of the sense strand and the nucleobase in position 18 in the antisense strand are in matching positions, and the nucleobase in position 4 in the sense strand and the nucleobase in position 15 in the antisense strand are in matching positions. A skilled artisan will understand how to identify matching positions in sense and antisense strands that are or will be duplexed strands and paired strands.

The first column in Table 1 indicates the Duplex AV # for a duplex that includes the sense and antisense sequences in the same table row. For example, Table 1 discloses the duplex assigned Duplex AV #AV00001, which includes sense strand SEQ ID NO: 1 and antisense strand SEQ ID NO: 225. Thus, each row in Table 1 discloses duplexes of the invention, each comprising the sense and antisense sequences shown in the same row.

In some embodiments of methods of the invention, an RNAi agent comprising a double stranded sequence disclosed in Table 1 is administered to a subject. In some embodiments of the invention an RNAi agent administered to a subject comprises a duplex comprising at least one of the strand sequences set forth in Table 1, that comprises 0, 1, 2, 3, 4, 5, 6, 7, 8, 9, 10, 11, 12, 13, 14, 15, 16, 17, 18, 19, 20, 21, 22, 23, or 24 sequence modifications. In some embodiments of methods of the invention an RNAi agent comprising a dsRNA polynucleotide sequence shown in Table 1 is attached to a delivery molecule, also referred to herein as a delivery compound. A non-limiting example of a delivery compound is a delivery compound comprising a GalNAc compound, or a GLS-15 compound.

TABLE 1
Unmodified PKK RNAi agent antisense strand and sense strand sequences.
All sequences shown 5' to 3' direction. Duplex AV#s are the number
assigned to the duplex of the two strands in the same row in the table.

| | | SEQ | | SEQ |
|---|---|---|---|---|
| | | ID | | ID |
| Duplex ID | Sense Strand | NO | Antisense Strand | NO |
| AV00001 | GGGAUGUAGCUUCCAUGUACA | 1 | UGUACAUGGAAGCUACAUCCC | 225 |
| AV00003 | UGGUUGCUUCUUGAAAGAUAA | 2 | UUAUCUUUCAAGAAGCAACCA | 226 |
| AV00004 | CUUCUUGAAAGAUAGUGUUAA | 3 | UUAACACUAUCUUUCAAGAAG | 227 |
| AV00005 | CAAUGUGGUCAUCAAAUAAGA | 4 | UCUUAUUUGAUGACCACAUUG | 228 |
| AV00006 | GUGGUCAUCAAAUAAGUGCUA | 5 | UAGCACUUAUUUGAUGACCAC | 229 |
| AV00007 | CAUCGAGACAUUUAUAAAGGA | 6 | UCCUUUAUAAAUGUCUCGAUG | 230 |
| AV00008 | AUCGAGACAUUUAUAAAGGAA | 7 | UUCCUUUAUAAAUGUCUCGAU | 231 |
| AV00009 | GUUGAUAUGAGAGGAGUCAAA | 8 | UUUGACUCCUCUCAUAUCAAC | 232 |
| AV00010 | CUAAGGUUAGCAGUGUUGAAA | 9 | UUUCAACACUGCUAACCUUAG | 233 |
| AV00011 | GGUUAGCAGUGUUGAAGAAUA | 10 | UAUUCUUCAACACUGCUAACC | 234 |
| AV00012 | GUUAGCAGUGUUGAAGAAUGA | 11 | UCAUUCUUCAACACUGCUAAC | 235 |
| AV00013 | GUAACGUGGAAUCUGGAUUCA | 12 | UGAAUCCAGAUUCCACGUUAC | 236 |
| AV00014 | AACGUGGAAUCUGGAUUCUCA | 13 | UGAGAAUCCAGAUUCCACGUU | 237 |
| AV00015 | AAUCUGGAUUCUCACUGAAGA | 14 | UCUUCAGUGAGAAUCCAGAUU | 238 |
| AV00016 | ACUCCAGAUGCUUUUGUGUGA | 15 | UCACACAAAAGCAUCUGGAGU | 239 |
| AV00017 | UUCUUAAAACAUCUGAAAGUA | 16 | UACUUUCAGAUGUUUUAAGAA | 240 |
| AV00018 | UUCCUCUACUCCUCAAGAAAA | 17 | UUUUCUUGAGGAGUAGAGGAA | 241 |
| AV00019 | UCCUCUACUCCUCAAGAAAAA | 18 | UUUUUCUUGAGGAGUAGAGGA | 242 |
| AV00020 | GCCUUUUAACCUGCAAAAGAA | 19 | UUCUUUUGCAGGUUAAAAGGC | 243 |
| AV00021 | AACCUGCAAAAGAACUUUACA | 20 | UGUAAAGUUCUUUUGCAGGUU | 244 |
| AV00022 | ACCUGCAAAAGAACUUUACCA | 21 | UGGUAAAGUUCUUUUGCAGGU | 245 |
| AV00023 | CCUGCAAAAGAACUUUACCUA | 22 | UAGGUAAAGUUCUUUUGCAGG | 246 |
| AV00024 | GCAAAAGAACUUUACCUGAAA | 23 | UUUCAGGUAAAGUUCUUUUGC | 247 |
| AV00025 | ACUUUGGAGGAGAAGAAUUGA | 24 | UCAAUUCUUCUCCUCCAAAGU | 248 |
| AV00026 | GAGGAGAAGAAUUGAAUGUGA | 25 | UCACAUUCAAUUCUUCUCCUC | 249 |
| AV00027 | UGUUAAAGGAGUGAAUGUUUA | 26 | UAAACAUUCACUCCUUUAACA | 250 |
| AV00028 | UUCGCUGUCAGUUUUUCACUA | 27 | UAGUGAAAAACUGACAGCGAA | 251 |
| AV00029 | CGCUGUCAGUUUUUCACUUAA | 28 | UUAAGUGAAAAACUGACAGCG | 252 |
| AV00030 | UUUUCACUUAUUCUUUACUCA | 29 | UGAGUAAAGAAUAAGUGAAAA | 253 |
| AV00031 | GAAGAGAAGUGUAAGUGUUUA | 30 | UAAACACUUACACUUCUCUUC | 254 |
| AV00032 | AGAGAAGUGUAAGUGUUUCUA | 31 | UAGAAACACUUACACUUCUCU | 255 |
| AV00033 | AGAAGUGUAAGUGUUUCUUAA | 32 | UUAAGAAACACUUACACUUCU | 256 |
| AV00034 | AGUGUAAGUGUUUCUUAAGAA | 33 | UUCUUAAGAAACACUUACACU | 257 |
| AV00035 | CUGGUUACUCUUUGAGAUUGA | 34 | UCAAUCUCAAAGAGUAACCAG | 258 |
| AV00036 | UGGUUACUCUUUGAGAUUGUA | 35 | UACAAUCUCAAAGAGUAACCA | 259 |
| AV00037 | ACUCUUUGAGAUUGUGUAACA | 36 | UGUUACACAAUCUCAAAGAGU | 260 |
| AV00038 | CUCUUUGAGAUUGUGUAACAA | 37 | UUGUUACACAAUCUCAAAGAG | 261 |
| AV00039 | UCUGUCUGCACAACAAAAACA | 38 | UGUUUUUGUUGUGCAGACAGA | 262 |
| AV00040 | GAGGAACAAACUCUUCUUGGA | 39 | UCCAAGAAGAGUUUGUUCCUC | 263 |
| AV00041 | UGUGUGGAGGGUCACUCAUAA | 40 | UUAUGAGUGACCCUCCACACA | 264 |
| AV00042 | UAUAGUGGCAUUUUAAAUCUA | 41 | UAGAUUUAAAAUGCCACUAUA | 265 |
| AV00043 | UCUGUCAGACAUUACAAAAGA | 42 | UCUUUUGUAAUGUCUGACAGA | 266 |
| AV00044 | CUGUCAGACAUUACAAAAGAA | 43 | UUCUUUUGUAAUGUCUGACAG | 267 |
| AV00045 | AUACACCUUUCUCACAAAUAA | 44 | UUAUUUGUGAGAAAGGUGUAU | 268 |
| AV00046 | GAAGGGAAUCAUGAUAUCGCA | 45 | UGCGAUAUCAUGAUUCCCUUC | 269 |
| AV00047 | CUCCUUUGAAUUACACUGAAA | 46 | UUUCAGUGUAAUUCAAAGGAG | 270 |
| AV00048 | UUACACUGAAUUCCAAAAACA | 47 | UGUUUUUGGAAUUCAGUGUAA | 271 |
| AV00049 | CAAGCACAAUUUAUACCAACA | 48 | UGUUGGUAUAAAUUGUGCUUG | 272 |
| AV00050-2 | CCAUCGAGACAUUUAUAAAGA | 49 | UCUUUAUAAAUGUCUCGAUGG | 273 |
| AV00051-2 | UACCUGAACCCUGCCAUUCUA | 50 | UAGAAUGGCAGGGUUCAGGUA | 274 |
| AV00052-2 | AAGGUUAGCAGUGUUGAAGAA | 51 | UUCUUCAACACUGCUAACCUU | 275 |
| AV00732 | CUCUAUGAAAACGCCUUCUUA | 52 | UAAGAAGGCGUUUUCAUAGAG | 276 |
| AV00733 | CACCCAAGGUGUUUGCUAUUA | 53 | UAAUAGCAAACACCUUGGGUG | 277 |
| AV00734 | UCAGUUUUCUUCCAGCAAGUA | 54 | UACUUGCUGGAAGAAAACUGA | 278 |
| AV00735 | AAUGACAUGGAGAAAAGGUUA | 55 | UAACCUUUUCUCCAUGUCAUU | 279 |
| AV00736 | GGUUGCUUCUUGAAAGAUAGA | 56 | UCUAUCUUUCAAGAAGCAACC | 280 |
| AV00737 | UCUUGAAAGAUAGUGUUACAA | 57 | UUGUAACACUAUCUUUCAAGA | 281 |
| AV00738 | UGUGGUCAUCAAAUAAGUGCA | 58 | UGCACUUAUUUGAUGACCACA | 282 |
| AV00739 | UGAUAUGAGAGGAGUCAAUUA | 59 | UAAUUGACUCCUCUCAUAUCA | 283 |
| AV00740 | UAUGAGAGGAGUCAAUUUUAA | 60 | UUAAAAUUGACUCCUCUCAUA | 284 |
| AV00741 | AGUGUUGAAGAAUGCCAAAAA | 61 | UUUUUGGCAUUCUUCAACACU | 285 |
| AV00742 | AGUAACGUGGAAUCUGGAUUA | 62 | UAAUCCAGAUUCCACGUUACU | 286 |
| AV00743 | CCUGUGCCCUUUCAGAAAUUA | 63 | UAAUUUCUGAAAGGGCACAGG | 287 |
| AV00744 | CCUUUUAACCUGCAAAAGAAA | 64 | UUUCUUUUGCAGGUUAAAAGG | 288 |
| AV00745 | GAACCCUGCCAUUCUAAAAUA | 65 | UAUUUUAGAAUGGCAGGGUUC | 289 |
| AV00746 | CCCUGCCAUUCUAAAAUUUAA | 66 | UUAAAUUUUAGAAUGGCAGGG | 290 |
| AV00747 | GAGAAGAAUUGAAUGUGACUA | 67 | UAGUCACAUUCAAUUCUUCUC | 291 |
| AV00748 | UUUUGUUAAAGGAGUGAAUGA | 68 | UCAUUCACUCCUUUAACAAAA | 292 |
| AV00749 | GUUAAAGGAGUGAAUGUUUGA | 69 | UCAAACAUUCACUCCUUUAAC | 293 |
| AV00750 | UUAAAGGAGUGAAUGUUUGCA | 70 | UGCAAACAUUCACUCCUUUAA | 294 |
| AV00751 | AAAGGAGUGAAUGUUUGCCAA | 71 | UUGGCAAACAUUCACUCCUUU | 295 |
| AV00752 | UUGCACAAAGAUGAUUCGCUA | 72 | UAGCGAAUCAUCUUUGUGCAA | 296 |
| AV00753 | AAGAUGAUUCGCUGUCAGUUA | 73 | UAACUGACAGCGAAUCAUCUU | 297 |
| AV00754 | UGAUUCGCUGUCAGUUUUUCA | 74 | UGAAAAACUGACAGCGAAUCA | 298 |
| AV00755 | CAUUCGCUGUCAGUUUUUCAA | 75 | UUGAAAAACUGACAGCGAAUG | 299 |
| AV00756 | CUUCGCUGUCAGUUUUUCACA | 76 | UGUGAAAAACUGACAGCGAAG | 300 |
| AV00757 | CCGCUGUCAGUUUUUCACUUA | 77 | UAAGUGAAAAACUGACAGCGG | 301 |
| AV00758 | CCUGUCAGUUUUUCACUUAUA | 78 | UAUAAGUGAAAAACUGACAGG | 302 |
| AV00759 | CUGUCAGUUUUUCACUUAUUA | 79 | UAAUAAGUGAAAAACUGACAG | 303 |
| AV00760 | CGUCAGUUUUUCACUUAUUCA | 80 | UGAAUAAGUGAAAAACUGACG | 304 |
| AV00761 | GUCAGUUUUUCACUUAUUCUA | 81 | UAGAAUAAGUGAAAAACUGAC | 305 |
| AV00762 | GUUUUUCACUUAUUCUUUACA | 82 | UGUAAAGAAUAAGUGAAAAAC | 306 |
| AV00763 | UUUUUCACUUAUUCUUUACUA | 83 | UAGUAAAGAAUAAGUGAAAAA | 307 |
| AV00764 | UUUCACUUAUUCUUUACUCCA | 84 | UGGAGUAAAGAAUAAGUGAAA | 308 |
| AV00765 | UAUUCUUUACUCCCAGAAGAA | 85 | UUCUUCUGGGAGUAAAGAAUA | 309 |
| AV00766 | UUACUCCCAGAAGACUGUAAA | 86 | UUUACAGUCUUCUGGGAGUAA | 310 |
| AV00767 | CUCCCAGAAGACUGUAAGGAA | 87 | UUCCUUACAGUCUUCUGGGAG | 311 |
| AV00768 | GAGAAGUGUAAGUGUUUCUUA | 88 | UAAGAAACACUUACACUUCUC | 312 |
| AV00769 | AAGUGUAAGUGUUUCUUAAGA | 89 | UCUUAAGAAACACUUACACUU | 313 |
| AV00770 | UGUAAGUGUUUCUUAAGAUUA | 90 | UAAUCUUAAGAAACACUUACA | 314 |
| AV00771 | CUAAGUGUUUCUUAAGAUUAA | 91 | UUAAUCUUAAGAAACACUUAG | 315 |
| AV00772 | UAAGUGUUUCUUAAGAUUAUA | 92 | UAUAAUCUUAAGAAACACUUA | 316 |
| AV00773 | AAGUGUUUCUUAAGAUUAUCA | 93 | UGAUAAUCUUAAGAAACACUU | 317 |
| AV00774 | GAGCUCUGGUUACUCUUUGAA | 94 | UUCAAAGAGUAACCAGAGCUC | 318 |
| AV00775 | GGUUACUCUUUGAGAUUGUGA | 95 | UCACAAUCUCAAAGAGUAACC | 319 |
| AV00776 | CAUUGUUGGAGGAACAAACUA | 96 | UAGUUUGUUCCUCCAACAAUG | 320 |
| AV00777 | CUAUAGUGGCAUUUUAAAUCA | 97 | UGAUUUAAAAUGCCACUAUAG | 321 |
| AV00778 | UUUAAAUCUGUCAGACAUUAA | 98 | UUAAUGUCUGACAGAUUUAAA | 322 |
| AV00779 | AUUAUUAUUCACCAAAACUAA | 99 | UUAGUUUUGGUGAAUAAUAAU | 323 |
| AV00780 | AGAAGGGAAUCAUGAUAUCGA | 100 | UCGAUAUCAUGAUUCCCUUCU | 324 |
| AV00781 | UCCAGGCUCCUUUGAAUUACA | 101 | UGUAAUUCAAAGGAGCCUGGA | 325 |
| AV00782 | ACUGAAUUCCAAAAACCAAUA | 102 | UAUUGGUUUUUGGAAUUCAGU | 326 |
| AV00783 | AUUCCAAAAACCAAUAUGCCA | 103 | UGGCAUAUUGGUUUUUGGAAU | 327 |
| AV00784 | GCACAAUUUAUACCAACUGUA | 104 | UACAGUUGGUAUAAAUUGUGC | 328 |
| AV00785 | UUAUACCAACUGUUGGGUAAA | 105 | UUUACCCAACAGUUGGUAUAA | 329 |
| AV00786 | UACACCAAAGUCGCUGAGUAA | 106 | UUACUCAGCGACUUUGGUGUA | 330 |
| AV00787 | AGUACAUGGACUGGAUUUUAA | 107 | UUAAAAUCCAGUCCAUGUACU | 331 |
| AV00788 | UGGACUGGAUUUUAGAGAAAA | 108 | UUUUCUCUAAAAUCCAGUCCA | 332 |
| AV00789 | GGACUGGAUUUUAGAGAAAAA | 109 | UUUUUCUCUAAAAUCCAGUCC | 333 |
| AV00790 | ACACAGAGCAGUGAUGGAAAA | 110 | UUUUCCAUCACUGCUCUGUGU | 334 |
| AV00791 | UGAGUUCAAGUCAAAUUCUGA | 111 | UCAGAAUUUGACUUGAACUCA | 335 |
| AV00792 | GAGUUCAAGUCAAAUUCUGAA | 112 | UUCAGAAUUUGACUUGAACUC | 336 |
| AV00001-19-1 | GAUGUAGCUUCCAUGUACA | 113 | UGUACAUGGAAGCUACAUC | 337 |
| AV00003-19-1 | GUUGCUUCUUGAAAGAUAA | 114 | UUAUCUUUCAAGAAGCAAC | 338 |
| AV00004-19-1 | UCUUGAAAGAUAGUGUUAA | 115 | UUAACACUAUCUUUCAAGA | 339 |
| AV00005-19-1 | AUGUGGUCAUCAAAUAAGA | 116 | UCUUAUUUGAUGACCACAU | 340 |
| AV00006-19-1 | GGUCAUCAAAUAAGUGCUA | 117 | UAGCACUUAUUUGAUGACC | 341 |
| AV00007-19-1 | UCGAGACAUUUAUAAAGGA | 118 | UCCUUUAUAAAUGUCUCGA | 342 |
| AV00008-19-1 | CGAGACAUUUAUAAAGGAA | 119 | UUCCUUUAUAAAUGUCUCG | 343 |
| AV00009-19-1 | UGAUAUGAGAGGAGUCAAA | 120 | UUUGACUCCUCUCAUAUCA | 344 |
| AV00010-19-1 | AAGGUUAGCAGUGUUGAAA | 121 | UUUCAACACUGCUAACCUU | 345 |
| AV00011-19-1 | UUAGCAGUGUUGAAGAAUA | 122 | UAUUCUUCAACACUGCUAA | 346 |
| AV00012-19-1 | UAGCAGUGUUGAAGAAUGA | 123 | UCAUUCUUCAACACUGCUA | 347 |
| AV00013-19-1 | AACGUGGAAUCUGGAUUCA | 124 | UGAAUCCAGAUUCCACGUU | 348 |
| AV00014-19-1 | CGUGGAAUCUGGAUUCUCA | 125 | UGAGAAUCCAGAUUCCACG | 349 |
| AV00015-19-1 | UCUGGAUUCUCACUGAAGA | 126 | UCUUCAGUGAGAAUCCAGA | 350 |
| AV00016-19-1 | UCCAGAUGCUUUUGUGUGA | 127 | UCACACAAAAGCAUCUGGA | 351 |
| AV00017-19-1 | CUUAAAACAUCUGAAAGUA | 128 | UACUUUCAGAUGUUUUAAG | 352 |
| AV00018-19-1 | CCUCUACUCCUCAAGAAAA | 129 | UUUUCUUGAGGAGUAGAGG | 353 |
| AV00019-19-1 | CUCUACUCCUCAAGAAAAA | 130 | UUUUUCUUGAGGAGUAGAG | 354 |
| AV00020-19-1 | CUUUUAACCUGCAAAAGAA | 131 | UUCUUUUGCAGGUUAAAAG | 355 |
| AV00021-19-1 | CCUGCAAAAGAACUUUACA | 132 | UGUAAAGUUCUUUUGCAGG | 356 |
| AV00022-19-1 | CUGCAAAAGAACUUUACCA | 133 | UGGUAAAGUUCUUUUGCAG | 357 |
| AV00023-19-1 | UGCAAAAGAACUUUACCUA | 134 | UAGGUAAAGUUCUUUUGCA | 358 |
| AV00024-19-1 | AAAAGAACUUUACCUGAAA | 135 | UUUCAGGUAAAGUUCUUUU | 359 |
| AV00025-19-1 | UUUGGAGGAGAAGAAUUGA | 136 | UCAAUUCUUCUCCUCCAAA | 360 |
| AV00026-19-1 | GGAGAAGAAUUGAAUGUGA | 137 | UCACAUUCAAUUCUUCUCC | 361 |
| AV00027-19-1 | UUAAAGGAGUGAAUGUUUA | 138 | UAAACAUUCACUCCUUUAA | 362 |
| AV00028-19-1 | CGCUGUCAGUUUUUCACUA | 139 | UAGUGAAAAACUGACAGCG | 363 |
| AV00029-19-1 | CUGUCAGUUUUUCACUUAA | 140 | UUAAGUGAAAAACUGACAG | 364 |
| AV00030-19-1 | UUCACUUAUUCUUUACUCA | 141 | UGAGUAAAGAAUAAGUGAA | 365 |
| AV00031-19-1 | AGAGAAGUGUAAGUGUUUA | 142 | UAAACACUUACACUUCUCU | 366 |
| AV00032-19-1 | AGAAGUGUAAGUGUUUCUA | 143 | UAGAAACACUUACACUUCU | 367 |
| AV00033-19-1 | AAGUGUAAGUGUUUCUUAA | 144 | UUAAGAAACACUUACACUU | 368 |
| AV00034-19-1 | UGUAAGUGUUUCUUAAGAA | 145 | UUCUUAAGAAACACUUACA | 369 |
| AV00035-19-1 | GGUUACUCUUUGAGAUUGA | 146 | UCAAUCUCAAAGAGUAACC | 370 |
| AV00036-19-1 | GUUACUCUUUGAGAUUGUA | 147 | UACAAUCUCAAAGAGUAAC | 371 |
| AV00037-19-1 | UCUUUGAGAUUGUGUAACA | 148 | UGUUACACAAUCUCAAAGA | 372 |
| AV00038-19-1 | CUUUGAGAUUGUGUAACAA | 149 | UUGUUACACAAUCUCAAAG | 373 |
| AV00039-19-1 | UGUCUGCACAACAAAAACA | 150 | UGUUUUUGUUGUGCAGACA | 374 |
| AV00040-19-1 | GGAACAAACUCUUCUUGGA | 151 | UCCAAGAAGAGUUUGUUCC | 375 |
| AV00041-19-1 | UGUGGAGGGUCACUCAUAA | 152 | UUAUGAGUGACCCUCCACA | 376 |
| AV00042-19-1 | UAGUGGCAUUUUAAAUCUA | 153 | UAGAUUUAAAAUGCCACUA | 377 |
| AV00043-19-1 | UGUCAGACAUUACAAAAGA | 154 | UCUUUUGUAAUGUCUGACA | 378 |
| AV00044-19-1 | GUCAGACAUUACAAAAGAA | 155 | UUCUUUUGUAAUGUCUGAC | 379 |
| AV00045-19-1 | ACACCUUUCUCACAAAUAA | 156 | UUAUUUGUGAGAAAGGUGU | 380 |
| AV00046-19-1 | AGGGAAUCAUGAUAUCGCA | 157 | UGCGAUAUCAUGAUUCCCU | 381 |
| AV00047-19-1 | CCUUUGAAUUACACUGAAA | 158 | UUUCAGUGUAAUUCAAAGG | 382 |
| AV00048-19-1 | ACACUGAAUUCCAAAAACA | 159 | UGUUUUUGGAAUUCAGUGU | 383 |
| AV00049-19-1 | AGCACAAUUUAUACCAACA | 160 | UGUUGGUAUAAAUUGUGCU | 384 |
| AV00050-19-1 | AUCGAGACAUUUAUAAAGA | 161 | UCUUUAUAAAUGUCUCGAU | 385 |
| AV00051-19-1 | CCUGAACCCUGCCAUUCUA | 162 | UAGAAUGGCAGGGUUCAGG | 386 |
| AV00052-19-1 | GGUUAGCAGUGUUGAAGAA | 163 | UUCUUCAACACUGCUAACC | 387 |
| AV00732-19-1 | CUAUGAAAACGCCUUCUUA | 164 | UAAGAAGGCGUUUUCAUAG | 388 |
| AV00733-19-1 | CCCAAGGUGUUUGCUAUUA | 165 | UAAUAGCAAACACCUUGGG | 389 |
| AV00734-19-1 | AGUUUUCUUCCAGCAAGUA | 166 | UACUUGCUGGAAGAAAACU | 390 |
| AV00735-19-1 | UGACAUGGAGAAAAGGUUA | 167 | UAACCUUUUCUCCAUGUCA | 391 |
| AV00736-19-1 | UUGCUUCUUGAAAGAUAGA | 168 | UCUAUCUUUCAAGAAGCAA | 392 |
| AV00737-19-1 | UUGAAAGAUAGUGUUACAA | 169 | UUGUAACACUAUCUUUCAA | 393 |
| AV00738-19-1 | UGGUCAUCAAAUAAGUGCA | 170 | UGCACUUAUUUGAUGACCA | 394 |
| AV00739-19-1 | AUAUGAGAGGAGUCAAUUA | 171 | UAAUUGACUCCUCUCAUAU | 395 |
| AV00740-19-1 | UGAGAGGAGUCAAUUUUAA | 172 | UUAAAAUUGACUCCUCUCA | 396 |
| AV00741-19-1 | UGUUGAAGAAUGCCAAAAA | 173 | UUUUUGGCAUUCUUCAACA | 397 |
| AV00742-19-1 | UAACGUGGAAUCUGGAUUA | 174 | UAAUCCAGAUUCCACGUUA | 398 |
| AV00743-19-1 | UGUGCCCUUUCAGAAAUUA | 175 | UAAUUUCUGAAAGGGCACA | 399 |
| AV00744-19-1 | UUUUAACCUGCAAAAGAAA | 176 | UUUCUUUUGCAGGUUAAAA | 400 |
| AV00745-19-1 | ACCCUGCCAUUCUAAAAUA | 177 | UAUUUUAGAAUGGCAGGGU | 401 |
| AV00746-19-1 | CUGCCAUUCUAAAAUUUAA | 178 | UUAAAUUUUAGAAUGGCAG | 402 |
| AV00747-19-1 | GAAGAAUUGAAUGUGACUA | 179 | UAGUCACAUUCAAUUCUUC | 403 |
| AV00748-19-1 | UUGUUAAAGGAGUGAAUGA | 180 | UCAUUCACUCCUUUAACAA | 404 |
| AV00749-19-1 | UAAAGGAGUGAAUGUUUGA | 181 | UCAAACAUUCACUCCUUUA | 405 |
| AV00750-19-1 | AAAGGAGUGAAUGUUUGCA | 182 | UGCAAACAUUCACUCCUUU | 406 |
| AV00751-19-1 | AGGAGUGAAUGUUUGCCAA | 183 | UUGGCAAACAUUCACUCCU | 407 |
| AV00752-19-1 | GCACAAAGAUGAUUCGCUA | 184 | UAGCGAAUCAUCUUUGUGC | 408 |
| AV00753-19-1 | GAUGAUUCGCUGUCAGUUA | 185 | UAACUGACAGCGAAUCAUC | 409 |
| AV00754-19-1 | AUUCGCUGUCAGUUUUUCA | 186 | UGAAAAACUGACAGCGAAU | 410 |
| AV00755-19-1 | UUCGCUGUCAGUUUUUCAA | 187 | UUGAAAAACUGACAGCGAA | 411 |
| AV00756-19-1 | UCGCUGUCAGUUUUUCACA | 188 | UGUGAAAAACUGACAGCGA | 412 |
| AV00757-19-1 | GCUGUCAGUUUUUCACUUA | 189 | UAAGUGAAAAACUGACAGC | 413 |
| AV00758-19-1 | UGUCAGUUUUUCACUUAUA | 190 | UAUAAGUGAAAAACUGACA | 414 |
| AV00759-19-1 | GUCAGUUUUUCACUUAUUA | 191 | UAAUAAGUGAAAAACUGAC | 415 |
| AV00760-19-1 | UCAGUUUUUCACUUAUUCA | 192 | UGAAUAAGUGAAAAACUGA | 416 |
| AV00761-19-1 | CAGUUUUUCACUUAUUCUA | 193 | UAGAAUAAGUGAAAAACUG | 417 |
| AV00762-19-1 | UUUUCACUUAUUCUUUACA | 194 | UGUAAAGAAUAAGUGAAAA | 418 |
| AV00763-19-1 | UUUCACUUAUUCUUUACUA | 195 | UAGUAAAGAAUAAGUGAAA | 419 |
| AV00764-19-1 | UCACUUAUUCUUUACUCCA | 196 | UGGAGUAAAGAAUAAGUGA | 420 |
| AV00765-19-1 | UUCUUUACUCCCAGAAGAA | 197 | UUCUUCUGGGAGUAAAGAA | 421 |
| AV00766-19-1 | ACUCCCAGAAGACUGUAAA | 198 | UUUACAGUCUUCUGGGAGU | 422 |
| AV00767-19-1 | CCCAGAAGACUGUAAGGAA | 199 | UUCCUUACAGUCUUCUGGG | 423 |
| AV00768-19-1 | GAAGUGUAAGUGUUUCUUA | 200 | UAAGAAACACUUACACUUC | 424 |
| AV00769-19-1 | GUGUAAGUGUUUCUUAAGA | 201 | UCUUAAGAAACACUUACAC | 425 |
| AV00770-19-1 | UAAGUGUUUCUUAAGAUUA | 202 | UAAUCUUAAGAAACACUUA | 426 |
| AV00771-19-1 | AAGUGUUUCUUAAGAUUAA | 203 | UUAAUCUUAAGAAACACUU | 427 |
| AV00772-19-1 | AGUGUUUCUUAAGAUUAUA | 204 | UAUAAUCUUAAGAAACACU | 428 |
| AV00773-19-1 | GUGUUUCUUAAGAUUAUCA | 205 | UGAUAAUCUUAAGAAACAC | 429 |
| AV00774-19-1 | GCUCUGGUUACUCUUUGAA | 206 | UUCAAAGAGUAACCAGAGC | 430 |
| AV00775-19-1 | UUACUCUUUGAGAUUGUGA | 207 | UCACAAUCUCAAAGAGUAA | 431 |
| AV00776-19-1 | UUGUUGGAGGAACAAACUA | 208 | UAGUUUGUUCCUCCAACAA | 432 |
| AV00777-19-1 | AUAGUGGCAUUUUAAAUCA | 209 | UGAUUUAAAAUGCCACUAU | 433 |
| AV00778-19-1 | UAAAUCUGUCAGACAUUAA | 210 | UUAAUGUCUGACAGAUUUA | 434 |
| AV00779-19-1 | UAUUAUUCACCAAAACUAA | 211 | UUAGUUUUGGUGAAUAAUA | 435 |
| AV00780-19-1 | AAGGGAAUCAUGAUAUCGA | 212 | UCGAUAUCAUGAUUCCCUU | 436 |
| AV00781-19-1 | CAGGCUCCUUUGAAUUACA | 213 | UGUAAUUCAAAGGAGCCUG | 437 |
| AV00782-19-1 | UGAAUUCCAAAAACCAAUA | 214 | UAUUGGUUUUUGGAAUUCA | 438 |
| AV00783-19-1 | UCCAAAAACCAAUAUGCCA | 215 | UGGCAUAUUGGUUUUUGGA | 439 |
| AV00784-19-1 | ACAAUUUAUACCAACUGUA | 216 | UACAGUUGGUAUAAAUUGU | 440 |
| AV00785-19-1 | AUACCAACUGUUGGGUAAA | 217 | UUUACCCAACAGUUGGUAU | 441 |
| AV00786-19-1 | CACCAAAGUCGCUGAGUAA | 218 | UUACUCAGCGACUUUGGUG | 442 |
| AV00787-19-1 | UACAUGGACUGGAUUUUAA | 219 | UUAAAAUCCAGUCCAUGUA | 443 |
| AV00788-19-1 | GACUGGAUUUUAGAGAAAA | 220 | UUUUCUCUAAAAUCCAGUC | 444 |
| AV00789-19-1 | ACUGGAUUUUAGAGAAAAA | 221 | UUUUUCUCUAAAAUCCAGU | 445 |
| AV00790-19-1 | ACAGAGCAGUGAUGGAAAA | 222 | UUUUCCAUCACUGCUCUGU | 446 |
| AV00791-19-1 | AGUUCAAGUCAAAUUCUGA | 223 | UCAGAAUUUGACUUGAACU | 447 |
| AV00792-19-1 | GUUCAAGUCAAAUUCUGAA | 224 | UUCAGAAUUUGACUUGAAC | 448 |
| AV01712 | UGUCAGUUUUUCACUUAUUCA | 768 | UGAAUAAGUGAAAAACUGACA | 973 |
| AV01713 | UGUCAGUUUUUCACUUAUUCU | 769 | AGAAUAAGUGAAAAACUGACA | 974 |
| AV01714 | UGUCAGUUUUUCACUUAUUCC | 770 | GGAAUAAGUGAAAAACUGACA | 975 |
| AV01715 | UGUCAGUUUUUCACUUAUUCG | 771 | CGAAUAAGUGAAAAACUGACA | 976 |
| AV01716 | AGUUUUUCACUUAUUCA | 772 | UGAAUAAGUGAAAAACU | 977 |
| AV01717 | CAGUUUUUCACUUAUUCA | 773 | UGAAUAAGUGAAAAACUG | 978 |
| AV01718 | ACAGUUUUUCACUUAUUCA | 774 | UGAAUAAGUGAAAAACUGU | 979 |
| AV01719 | UCAGUUUUUCACUUAUUCA | 775 | UGAAUAAGUGAAAAACUGA | 980 |
| AV01720 | GCAGUUUUUCACUUAUUCA | 776 | UGAAUAAGUGAAAAACUGC | 981 |
| AV01721 | CCAGUUUUUCACUUAUUCA | 777 | UGAAUAAGUGAAAAACUGG | 982 |
| AV01722 | AUCAGUUUUUCACUUAUUCA | 778 | UGAAUAAGUGAAAAACUGAU | 983 |
| AV01723 | UUCAGUUUUUCACUUAUUCA | 779 | UGAAUAAGUGAAAAACUGAA | 984 |
| AV01724 | GUCAGUUUUUCACUUAUUCA | 780 | UGAAUAAGUGAAAAACUGAC | 985 |
| AV01725 | CUCAGUUUUUCACUUAUUCA | 781 | UGAAUAAGUGAAAAACUGAG | 986 |
| AV01726 | GGUCAGUUUUUCACUUAUUCA | 782 | UGAAUAAGUGAAAAACUGACC | 987 |
| AV01727 | AGUCAGUUUUUCACUUAUUCA | 783 | UGAAUAAGUGAAAAACUGACU | 988 |
| AV01728 | CGUCAGUUUUUCACUUAUUCA | 784 | UGAAUAAGUGAAAAACUGACG | 989 |
| AV01729 | CUGUCAGUUUUUCACUUAUUCA | 785 | UGAAUAAGUGAAAAACUGACAG | 990 |
| AV01730 | GCUGUCAGUUUUUCACUUAUUCA | 786 | UGAAUAAGUGAAAAACUGACAGC | 991 |
| AV01731 | CGCUGUCAGUUUUUCACUUAUUCA | 787 | UGAAUAAGUGAAAAACUGACAGCG | 992 |
| AV01732 | UCGCUGUCAGUUUUUCACUUAUUCA | 788 | UGAAUAAGUGAAAAACUGACAGCGA | 993 |
| AV01733 | UUCGCUGUCAGUUUUUCACUUAUUCA | 789 | UGAAUAAGUGAAAAACUGACAGCGAA | 994 |
| AV01734 | AGAUGAUUCGCUGUCAGUUUUUCAC | 790 | UGAAUAAGUGAAAAACUGACAGCGAAUCAU | 995 |
| | UUAUUCA | | CU | |
| AV01735 | UGUCAGUUUUUCACUUAUUCA | 791 | UGAAUAAGUGAAAAACUGACAUU | 996 |
| AV01736 | UGUCAGUUUUUCACUUAUUCA | 792 | UGAAUAAGUGAAAAACUGACAGA | 997 |
| AV01737 | UGUCAGUUUUUCACUUAUUCA | 793 | UGAAUAAGUGAAAAACUGACA | 998 |
| AV01738 | UGUCAGUUUUUCACUUAUUCA | 794 | UGAAUAAGUGAAAAACUGACA | 999 |
| AV01739 | UGUCAGUUUUUCACUUAUUCA | 795 | UGAAUAAGUGAAAAACUGACA | 1000 |
| AV01740 | UGUCAGUUUUUCACUCAUUCA | 796 | UGAAUAAGUGAAAAACUGACA | 1001 |
| AV01741 | UGUCAGUUUUUCACUUAUUUA | 797 | UAAAUAAGUGAAAAACUGACA | 1002 |
| AV01742 | UGUCAGUUUUUCACUUAUUCA | 798 | UGAAUAAGUGAAAAACUGACA | 1003 |
| AV01743 | AUACACCUUUCUCACAAAUAA | 799 | UUAUUUGUGAGAAAGGUGUAU | 1004 |
| AV01744 | AUACACCUUUCUCACAAAUAU | 800 | AUAUUUGUGAGAAAGGUGUAU | 1005 |
| AV01745 | AUACACCUUUCUCACAAAUAC | 801 | GUAUUUGUGAGAAAGGUGUAU | 1006 |
| AV01746 | AUACACCUUUCUCACAAAUAG | 802 | CUAUUUGUGAGAAAGGUGUAU | 1007 |
| AV01747 | ACCUUUCUCACAAAUAA | 803 | UUAUUUGUGAGAAAGGU | 1008 |
| AV01748 | CACCUUUCUCACAAAUAA | 804 | UUAUUUGUGAGAAAGGUG | 1009 |
| AV01749 | ACACCUUUCUCACAAAUAA | 805 | UUAUUUGUGAGAAAGGUGU | 1010 |
| AV01750 | UCACCUUUCUCACAAAUAA | 806 | UUAUUUGUGAGAAAGGUGA | 1011 |
| AV01751 | GCACCUUUCUCACAAAUAA | 807 | UUAUUUGUGAGAAAGGUGC | 1012 |
| AV01752 | CCACCUUUCUCACAAAUAA | 808 | UUAUUUGUGAGAAAGGUGG | 1013 |
| AV01753 | AACACCUUUCUCACAAAUAA | 809 | UUAUUUGUGAGAAAGGUGUU | 1014 |
| AV01754 | UACACCUUUCUCACAAAUAA | 810 | UUAUUUGUGAGAAAGGUGUA | 1015 |
| AV01755 | GACACCUUUCUCACAAAUAA | 811 | UUAUUUGUGAGAAAGGUGUC | 1016 |
| AV01756 | CACACCUUUCUCACAAAUAA | 812 | UUAUUUGUGAGAAAGGUGUG | 1017 |
| AV01757 | GUACACCUUUCUCACAAAUAA | 813 | UUAUUUGUGAGAAAGGUGUAC | 1018 |
| AV01758 | CUACACCUUUCUCACAAAUAA | 814 | UUAUUUGUGAGAAAGGUGUAG | 1019 |
| AV01759 | UUACACCUUUCUCACAAAUAA | 815 | UUAUUUGUGAGAAAGGUGUAA | 1020 |
| AV01760 | GAUACACCUUUCUCACAAAUAA | 816 | UUAUUUGUGAGAAAGGUGUAUC | 1021 |
| AV01761 | AGAUACACCUUUCUCACAAAUAA | 817 | UUAUUUGUGAGAAAGGUGUAUCU | 1022 |
| AV01762 | AAGAUACACCUUUCUCACAAAUAA | 818 | UUAUUUGUGAGAAAGGUGUAUCUU | 1023 |
| AV01763 | AAAGAUACACCUUUCUCACAAAUAA | 819 | UUAUUUGUGAGAAAGGUGUAUCUUU | 1024 |
| AV01764 | AAAAGAUACACCUUUCUCACAAAUAA | 820 | UUAUUUGUGAGAAAGGUGUAUCUUUU | 1025 |
| AV01765 | CAUUACAAAAGAUACACCUUUCUCACAAAU | 821 | UUAUUUGUGAGAAAGGUGUAUCUUUUG | 1026 |
| | AA | | UAAUG | |
| AV01766 | AUACACCUUUCUCACAAAUAA | 822 | UUAUUUGUGAGAAAGGUGUAUUU | 1027 |
| AV01767 | AUACACCUUUCUCACAAAUAA | 823 | UUAUUUGUGAGAAAGGUGUAUGA | 1028 |
| AV01768 | AUACACCUUUCUCACAAAUAA | 824 | UUAUUUGUGAGAAAGGUGUAU | 1029 |
| AV01769 | AUACACCUUUCUCACAAAUAA | 825 | UUAUUUGUGAGAAAGGUGUAU | 1030 |
| AV01770 | AUACACCUUUCUCACAAAUAA | 826 | UUAUUUGUGAGAAAGGUGUAU | 1031 |
| AV01771 | AUACACCUUUCUCACGAAUAA | 827 | UUAUUUGUGAGAAAGGUGUAU | 1032 |
| AV01772 | AUACACCUUUCUCACAAAUGA | 828 | UCAUUUGUGAGAAAGGUGUAU | 1033 |
| AV01773 | AUACACCUUUCUCACAAAUAA | 829 | UUAUUUGUGAGAAAGGUGUAU | 1034 |
| AV01774 | UUCGCUGUCAGUUUUUCACUA | 830 | UAGUGAAAAACUGACAGCGAA | 1035 |
| AV01775 | UUCGCUGUCAGUUUUUCACUU | 831 | AAGUGAAAAACUGACAGCGAA | 1036 |
| AV01776 | UUCGCUGUCAGUUUUUCACUC | 832 | GAGUGAAAAACUGACAGCGAA | 1037 |
| AV01777 | UUCGCUGUCAGUUUUUCACUG | 833 | CAGUGAAAAACUGACAGCGAA | 1038 |
| AV01778 | CUGUCAGUUUUUCACUA | 834 | UAGUGAAAAACUGACAG | 1039 |
| AV01779 | GCUGUCAGUUUUUCACUA | 835 | UAGUGAAAAACUGACAGC | 1040 |
| AV01780 | AGCUGUCAGUUUUUCACUA | 836 | UAGUGAAAAACUGACAGCU | 1041 |
| AV01781 | UGCUGUCAGUUUUUCACUA | 837 | UAGUGAAAAACUGACAGCA | 1042 |
| AV01782 | GGCUGUCAGUUUUUCACUA | 838 | UAGUGAAAAACUGACAGCC | 1043 |
| AV01783 | CGCUGUCAGUUUUUCACUA | 839 | UAGUGAAAAACUGACAGCG | 1044 |
| AV01784 | ACGCUGUCAGUUUUUCACUA | 840 | UAGUGAAAAACUGACAGCGU | 1045 |
| AV01785 | UCGCUGUCAGUUUUUCACUA | 841 | UAGUGAAAAACUGACAGCGA | 1046 |
| AV01786 | GCGCUGUCAGUUUUUCACUA | 842 | UAGUGAAAAACUGACAGCGC | 1047 |
| AV01787 | CCGCUGUCAGUUUUUCACUA | 843 | UAGUGAAAAACUGACAGCGG | 1048 |
| AV01788 | GUCGCUGUCAGUUUUUCACUA | 844 | UAGUGAAAAACUGACAGCGAC | 1049 |
| AV01789 | CUCGCUGUCAGUUUUUCACUA | 845 | UAGUGAAAAACUGACAGCGAG | 1050 |
| AV01790 | AUCGCUGUCAGUUUUUCACUA | 846 | UAGUGAAAAACUGACAGCGAU | 1051 |
| AV01791 | AUUCGCUGUCAGUUUUUCACUA | 847 | UAGUGAAAAACUGACAGCGAAU | 1052 |
| AV01792 | GAUUCGCUGUCAGUUUUUCACUA | 848 | UAGUGAAAAACUGACAGCGAAUC | 1053 |
| AV01793 | UGAUUCGCUGUCAGUUUUUCACUA | 849 | UAGUGAAAAACUGACAGCGAAUCA | 1054 |
| AV01794 | AUGAUUCGCUGUCAGUUUUUCACUA | 850 | UAGUGAAAAACUGACAGCGAAUCAU | 1055 |
| AV01795 | GAUGAUUCGCUGUCAGUUUUUCACUA | 851 | UAGUGAAAAACUGACAGCGAAUCAUC | 1056 |
| AV01796 | CACAAAGAUGAUUCGCUGUCAGUUUUUCAC | 852 | UAGUGAAAAACUGACAGCGAAUCAUCU | 1057 |
| | UA | | UUGUG | |
| AV01797 | UUCGCUGUCAGUUUUUCACUA | 853 | UAGUGAAAAACUGACAGCGAA | 1058 |
| AV01798 | UUCGCUGUCAGUUUUUCACUA | 854 | UAGUGAAAAACUGACAGCGAA | 1059 |
| AV01799 | UUCGCUGUCAGUUUUUCACUA | 855 | UAGUGAAAAACUGACAGCGAA | 1060 |
| AV01800 | UUCGCUGUCAGUUUUUCACUA | 856 | UAGUGAAAAACUGACAGCGAA | 1061 |
| AV01801 | UUCGCUGUCAGUUUUUCACUA | 857 | UAGUGAAAAACUGACAGCGAA | 1062 |
| AV01802 | UUCGCUGUCAGUUUUCCACUA | 858 | UAGUGAAAAACUGACAGCGAA | 1063 |
| AV01803 | UUCGCUGUCAGUUUUUCACCA | 859 | UGGUGAAAAACUGACAGCGAA | 1064 |
| AV01804 | UUCGCUGUCAGUUUUUCACUA | 860 | UAGUGAAAAACUGACAGCGAA | 1065 |
| AV01805 | UCCAGGCUCCUUUGAAUUACA | 861 | UGUAAUUCAAAGGAGCCUGGA | 1066 |
| AV01806 | UCCAGGCUCCUUUGAAUUACU | 862 | AGUAAUUCAAAGGAGCCUGGA | 1067 |
| AV01807 | UCCAGGCUCCUUUGAAUUACC | 863 | GGUAAUUCAAAGGAGCCUGGA | 1068 |
| AV01808 | UCCAGGCUCCUUUGAAUUACG | 864 | CGUAAUUCAAAGGAGCCUGGA | 1069 |
| AV01809 | GGCUCCUUUGAAUUACA | 865 | UGUAAUUCAAAGGAGCC | 1070 |
| AV01810 | AGGCUCCUUUGAAUUACA | 866 | UGUAAUUCAAAGGAGCCU | 1071 |
| AV01811 | AAGGCUCCUUUGAAUUACA | 867 | UGUAAUUCAAAGGAGCCUU | 1072 |
| AV01812 | UAGGCUCCUUUGAAUUACA | 868 | UGUAAUUCAAAGGAGCCUA | 1073 |
| AV01813 | GAGGCUCCUUUGAAUUACA | 869 | UGUAAUUCAAAGGAGCCUC | 1074 |
| AV01814 | CAGGCUCCUUUGAAUUACA | 870 | UGUAAUUCAAAGGAGCCUG | 1075 |
| AV01815 | ACAGGCUCCUUUGAAUUACA | 871 | UGUAAUUCAAAGGAGCCUGU | 1076 |
| AV01816 | UCAGGCUCCUUUGAAUUACA | 872 | UGUAAUUCAAAGGAGCCUGA | 1077 |
| AV01817 | GCAGGCUCCUUUGAAUUACA | 873 | UGUAAUUCAAAGGAGCCUGC | 1078 |
| AV01818 | CCAGGCUCCUUUGAAUUACA | 874 | UGUAAUUCAAAGGAGCCUGG | 1079 |
| AV01819 | GCCAGGCUCCUUUGAAUUACA | 875 | UGUAAUUCAAAGGAGCCUGGC | 1080 |
| AV01820 | CCCAGGCUCCUUUGAAUUACA | 876 | UGUAAUUCAAAGGAGCCUGGG | 1081 |
| AV01821 | ACCAGGCUCCUUUGAAUUACA | 877 | UGUAAUUCAAAGGAGCCUGGU | 1082 |
| AV01822 | CUCCAGGCUCCUUUGAAUUACA | 878 | UGUAAUUCAAAGGAGCCUGGAG | 1083 |
| AV01823 | ACUCCAGGCUCCUUUGAAUUACA | 879 | UGUAAUUCAAAGGAGCCUGGAGU | 1084 |
| AV01824 | AACUCCAGGCUCCUUUGAAUUACA | 880 | UGUAAUUCAAAGGAGCCUGGAGUU | 1085 |
| AV01825 | AAACUCCAGGCUCCUUUGAAUUACA | 881 | UGUAAUUCAAAGGAGCCUGGAGUUU | 1086 |
| AV01826 | AAAACUCCAGGCUCCUUUGAAUUACA | 882 | UGUAAUUCAAAGGAGCCUGGAGUUUU | 1087 |
| AV01827 | CUUGAUAAAACUCCAGGCUCCUUUGAAUUA | 883 | UGUAAUUCAAAGGAGCCUGGAGUUUUA | 1088 |
| | CA | | UCAAG | |
| AV01828 | UCCAGGCUCCUUUGAAUUACA | 884 | UGUAAUUCAAAGGAGCCUGGAUU | 1089 |
| AV01829 | UCCAGGCUCCUUUGAAUUACA | 885 | UGUAAUUCAAAGGAGCCUGGAGA | 1090 |
| AV01830 | UCCAGGCUCCUUUGAAUUACA | 886 | UGUAAUUCAAAGGAGCCUGGA | 1091 |
| AV01831 | UCCAGGCUCCUUUGAAUUACA | 887 | UGUAAUUCAAAGGAGCCUGGA | 1092 |
| AV01832 | UCCAGGCUCCUUUGAAUUACA | 888 | UGUAAUUCAAAGGAGCCUGGA | 1093 |
| AV01833 | UCCAGGCUCCUUUGAGUUACA | 889 | UGUAAUUCAAAGGAGCCUGGA | 1094 |
| AV01834 | UCCAGGCUCCUUUGAAUUAUA | 890 | UAUAAUUCAAAGGAGCCUGGA | 1095 |
| AV01835 | UCCAGGCUCCUUUGAAUUACA | 891 | UGUAAUUCAAAGGAGCCUGGA | 1096 |
| AV01836 | GAAGGAGUGAAUGUUUGCCAA | 892 | UUGGCAAACAUUCACUCCUUC | 1097 |
| AV01837 | GAAGGAGUGAAUGUUUGCCAU | 893 | AUGGCAAACAUUCACUCCUUC | 1098 |
| AV01838 | GAAGGAGUGAAUGUUUGCCAC | 894 | GUGGCAAACAUUCACUCCUUC | 1099 |
| AV01839 | GAAGGAGUGAAUGUUUGCCAG | 895 | CUGGCAAACAUUCACUCCUUC | 1100 |
| AV01840 | GAGUGAAUGUUUGCCAA | 896 | UUGGCAAACAUUCACUC | 1101 |
| AV01841 | GGAGUGAAUGUUUGCCAA | 897 | UUGGCAAACAUUCACUCC | 1102 |
| AV01842 | AGGAGUGAAUGUUUGCCAA | 898 | UUGGCAAACAUUCACUCCU | 1103 |
| AV01843 | UGGAGUGAAUGUUUGCCAA | 899 | UUGGCAAACAUUCACUCCA | 1104 |
| AV01844 | GGGAGUGAAUGUUUGCCAA | 900 | UUGGCAAACAUUCACUCCC | 1105 |
| AV01845 | CGGAGUGAAUGUUUGCCAA | 901 | UUGGCAAACAUUCACUCCG | 1106 |
| AV01846 | AAGGAGUGAAUGUUUGCCAA | 902 | UUGGCAAACAUUCACUCCUU | 1107 |
| AV01847 | UAGGAGUGAAUGUUUGCCAA | 903 | UUGGCAAACAUUCACUCCUA | 1108 |
| AV01848 | GAGGAGUGAAUGUUUGCCAA | 904 | UUGGCAAACAUUCACUCCUC | 1109 |
| AV01849 | CAGGAGUGAAUGUUUGCCAA | 905 | UUGGCAAACAUUCACUCCUG | 1110 |
| AV01850 | UAAGGAGUGAAUGUUUGCCAA | 906 | UUGGCAAACAUUCACUCCUUA | 1111 |
| AV01851 | CAAGGAGUGAAUGUUUGCCAA | 907 | UUGGCAAACAUUCACUCCUUG | 1112 |
| AV01852 | AAAGGAGUGAAUGUUUGCCAA | 908 | UUGGCAAACAUUCACUCCUUU | 1113 |
| AV01853 | UAAAGGAGUGAAUGUUUGCCAA | 909 | UUGGCAAACAUUCACUCCUUUA | 1114 |
| AV01854 | UUAAAGGAGUGAAUGUUUGCCAA | 910 | UUGGCAAACAUUCACUCCUUUAA | 1115 |
| AV01855 | GUUAAAGGAGUGAAUGUUUGCCAA | 911 | UUGGCAAACAUUCACUCCUUUAAC | 1116 |
| AV01856 | UGUUAAAGGAGUGAAUGUUUGCCAA | 912 | UUGGCAAACAUUCACUCCUUUAACA | 1117 |
| AV01857 | UUGUUAAAGGAGUGAAUGUUUGCCAA | 913 | UUGGCAAACAUUCACUCCUUUAACAA | 1118 |
| AV01858 | UGACUUUUGUUAAAGGAGUGAAUGUUUGCC | 914 | UUGGCAAACAUUCACUCCUUUAACAAAA | 1119 |
| | AA | | GUCA | |
| AV01859 | GAAGGAGUGAAUGUUUGCCAA | 915 | UUGGCAAACAUUCACUCCUUCUU | 1120 |
| AV01860 | GAAGGAGUGAAUGUUUGCCAA | 916 | UUGGCAAACAUUCACUCCUUCGA | 1121 |
| AV01861 | GAAGGAGUGAAUGUUUGCCAA | 917 | UUGGCAAACAUUCACUCCUUC | 1122 |
| AV01862 | GAAGGAGUGAAUGUUUGCCAA | 918 | UUGGCAAACAUUCACUCCUUC | 1123 |
| AV01863 | GAAGGAGUGAAUGUUUGCCAA | 919 | UUGGCAAACAUUCACUCCUUC | 1124 |
| AV01864 | GAAGGAGUGAAUGUUCGCCAA | 920 | UUGGCAAACAUUCACUCCUUC | 1125 |
| AV01865 | GAAGGAGUGAAUGUUUGCCGA | 921 | UCGGCAAACAUUCACUCCUUC | 1126 |
| AV01866 | GAAGGAGUGAAUGUUUGCCAA | 922 | UUGGCAAACAUUCACUCCUUC | 1127 |
| AV01960 | UUCGCUGUCAGUUUUUCACUA | 923 | UAGUGAAAAACUGACAGCGAA | 1128 |
| AV01961 | UCGCUGUCAGUUUUUCACUUA | 924 | UAAGUGAAAAACUGACAGCGA | 1129 |
| AV01962 | CGCUGUCAGUUUUUCACUUAA | 925 | UUAAGUGAAAAACUGACAGCG | 1130 |
| AV01963 | GCUGUCAGUUUUUCACUUAUA | 926 | UAUAAGUGAAAAACUGACAGC | 1131 |
| AV01964 | CUGUCAGUUUUUCACUUAUUA | 927 | UAAUAAGUGAAAAACUGACAG | 1132 |
| AV01965 | GUCAGUUUUUCACUUAUUCUA | 928 | UAGAAUAAGUGAAAAACUGAC | 1133 |
| AV01966 | UCAGUUUUUCACUUAUUCUUA | 929 | UAAGAAUAAGUGAAAAACUGA | 1134 |
| AV01967 | CAGUUUUUCACUUAUUCUUUA | 930 | UAAAGAAUAAGUGAAAAACUG | 1135 |
| AV01968 | AGUUUUUCACUUAUUCUUUAA | 931 | UUAAAGAAUAAGUGAAAAACU | 1136 |
| AV01969 | GUUUUUCACUUAUUCUUUACA | 932 | UGUAAAGAAUAAGUGAAAAAC | 1137 |
| AV01970 | AAAAGAUACACCUUUCUCACA | 933 | UGUGAGAAAGGUGUAUCUUUU | 1138 |
| AV01971 | AAAGAUACACCUUUCUCACAA | 934 | UUGUGAGAAAGGUGUAUCUUU | 1139 |
| AV01972 | AAGAUACACCUUUCUCACAAA | 935 | UUUGUGAGAAAGGUGUAUCUU | 1140 |
| AV01973 | AGAUACACCUUUCUCACAAAA | 936 | UUUUGUGAGAAAGGUGUAUCU | 1141 |
| AV01974 | GAUACACCUUUCUCACAAAUA | 937 | UAUUUGUGAGAAAGGUGUAUC | 1142 |
| AV01975 | UACACCUUUCUCACAAAUAAA | 938 | UUUAUUUGUGAGAAAGGUGUA | 1143 |
| AV01976 | ACACCUUUCUCACAAAUAAAA | 939 | UUUUAUUUGUGAGAAAGGUGU | 1144 |
| AV01977 | CACCUUUCUCACAAAUAAAAA | 940 | UUUUUAUUUGUGAGAAAGGUG | 1145 |
| AV01978 | ACCUUUCUCACAAAUAAAAGA | 941 | UCUUUUAUUUGUGAGAAAGGU | 1146 |
| AV01979 | CCUUUCUCACAAAUAAAAGAA | 942 | UUCUUUUAUUUGUGAGAAAGG | 1147 |
| AV01980 | GAUGAUUCGCUGUCAGUUUUA | 943 | UAAAACUGACAGCGAAUCAUC | 1148 |
| AV01981 | AUGAUUCGCUGUCAGUUUUUA | 944 | UAAAAACUGACAGCGAAUCAU | 1149 |
| AV01982 | UGAUUCGCUGUCAGUUUUUCA | 945 | UGAAAAACUGACAGCGAAUCA | 1150 |
| AV01983 | GAUUCGCUGUCAGUUUUUCAA | 946 | UUGAAAAACUGACAGCGAAUC | 1151 |
| AV01984 | AUUCGCUGUCAGUUUUUCACA | 947 | UGUGAAAAACUGACAGCGAAU | 1152 |
| AV01985 | UCGCUGUCAGUUUUUCACUUA | 948 | UAAGUGAAAAACUGACAGCGA | 1153 |
| AV01986 | CGCUGUCAGUUUUUCACUUAA | 949 | UUAAGUGAAAAACUGACAGCG | 1154 |
| AV01987 | GCUGUCAGUUUUUCACUUAUA | 950 | UAUAAGUGAAAAACUGACAGC | 1155 |
| AV01988 | CUGUCAGUUUUUCACUUAUUA | 951 | UAAUAAGUGAAAAACUGACAG | 1156 |
| AV01989 | UGUCAGUUUUUCACUUAUUCA | 952 | UGAAUAAGUGAAAAACUGACA | 1157 |
| AV01990 | AAAACUCCAGGCUCCUUUGAA | 953 | UUCAAAGGAGCCUGGAGUUUU | 1158 |
| AV01991 | AAACUCCAGGCUCCUUUGAAA | 954 | UUUCAAAGGAGCCUGGAGUUU | 1159 |
| AV01992 | AACUCCAGGCUCCUUUGAAUA | 955 | UAUUCAAAGGAGCCUGGAGUU | 1160 |
| AV01993 | ACUCCAGGCUCCUUUGAAUUA | 956 | UAAUUCAAAGGAGCCUGGAGU | 1161 |
| AV01994 | CUCCAGGCUCCUUUGAAUUAA | 957 | UUAAUUCAAAGGAGCCUGGAG | 1162 |
| AV01995 | CCAGGCUCCUUUGAAUUACAA | 958 | UUGUAAUUCAAAGGAGCCUGG | 1163 |
| AV01996 | CAGGCUCCUUUGAAUUACACA | 959 | UGUGUAAUUCAAAGGAGCCUG | 1164 |
| AV01997 | AGGCUCCUUUGAAUUACACUA | 960 | UAGUGUAAUUCAAAGGAGCCU | 1165 |
| AV01998 | GGCUCCUUUGAAUUACACUGA | 961 | UCAGUGUAAUUCAAAGGAGCC | 1166 |
| AV01999 | GCUCCUUUGAAUUACACUGAA | 962 | UUCAGUGUAAUUCAAAGGAGC | 1167 |
| AV02000 | UUGUUAAAGGAGUGAAUGUUA | 963 | UAACAUUCACUCCUUUAACAA | 1168 |
| AV02001 | UGUUAAAGGAGUGAAUGUUUA | 964 | UAAACAUUCACUCCUUUAACA | 1169 |
| AV02002 | GUUAAAGGAGUGAAUGUUUGA | 965 | UCAAACAUUCACUCCUUUAAC | 1170 |
| AV02003 | UUAAAGGAGUGAAUGUUUGCA | 966 | UGCAAACAUUCACUCCUUUAA | 1171 |
| AV02004 | UAAAGGAGUGAAUGUUUGCCA | 967 | UGGCAAACAUUCACUCCUUUA | 1172 |
| AV02005 | AAGGAGUGAAUGUUUGCCAAA | 968 | UUUGGCAAACAUUCACUCCUU | 1173 |
| AV02006 | AGGAGUGAAUGUUUGCCAAGA | 969 | UCUUGGCAAACAUUCACUCCU | 1174 |
| AV02007 | GGAGUGAAUGUUUGCCAAGAA | 970 | UUCUUGGCAAACAUUCACUCC | 1175 |
| AV02008 | GAGUGAAUGUUUGCCAAGAGA | 971 | UCUCUUGGCAAACAUUCACUC | 1176 |
| AV02009 | AGUGAAUGUUUGCCAAGAGAA | 972 | UUCUCUUGGCAAACAUUCACU | 1177 |

Table 2 shows certain chemically modified PKK RNAi agent sense strand and antisense strand sequences of the invention. In some embodiments of methods of the invention, an RNAi agent with a polynucleotide sequence shown in Table 2 is administered to a cell and/or subject. In some embodiments of the invention an RNAi agent administered to a subject comprises is a duplex identified in a row in Table 2, column one and includes the sequence modifications shown in the sense and antisense strand sequences in the same row in Table 2, columns four and seven, respectively. In some embodiments of methods of the invention, a sequence shown in Table 2 may be attached to (also referred to herein as “conjugated to”) a compound capable of delivering the RNAi agent to a cell and/or tissue in a subject. Non-limiting examples of delivery compounds that may be used in certain embodiments of the invention are a GalNAc-containing compound and a GLS-15-containing compound. In Table 2, the first column indicates the Duplex AV # of the base sequences as shown in Table 1. Table 2 discloses Duplex AV # and also shows chemical modifications included in sense and antisense sequence of the duplexes. For example, Table 1 shows base single-strand sequences SEQ ID NO: 1 (sense) and SEQ ID NO: 225 (antisense), which together are the double-stranded duplex identified as: Duplex AV #AV00001. Table 2 lists Duplex AV #AV00001, and shows the duplex of SEQ ID NO: 449 and SEQ ID NO: 561 includes base sequences SEQ ID NO: 1 and SEQ ID NO: 225, respectively, but with the chemical modifications shown in the sense and antisense sequences shown in Table 2, columns four and seven, respectively. The “Sense strand SS #” in Table 2 column two is the assigned identifier for the Sense Sequence (including modifications) shown column four in the same row. The “Antisense strand AS #” in Table 2 column five is the assigned identifier for the Antisense sequence (including modifications) shown in column seven.

TABLE 2
Chemically modified PKK RNAi agent antisense strand and sense strand sequences.
All sequences shown 5′to 3′. These sequences were used in certain in vitro
testing studies described herein. The chemical modifications are indicated by:
Upper case: 2′-Fluoro; lower case: 2′-OMe;
thiophosphate: *. 2′,3′-Sec-adenosine unlocked nucleic acid: aUNA

| | Sense | SEQ | | Antisense | SEQ | |
|---|---|---|---|---|---|---|
| Duplex | strand | ID | | strand | ID | |
| ID | SS# | NO | Sense Strand | AS# | NO | Antisense Strand |
| AV00001 | AV00001- | 449 | g*g*gauguaGcUuCcaugu | AV00001- | 561 | u*G*uacaUggaaGcUaCau |
| | SS | | ac*a | AS | | c*c*c |
| AV00003 | AV00003- | 450 | u*g*guugcuUcUuGaaaga | AV00003- | 562 | u*U*aucuUucaaGaAgCaa |
| | SS | | ua*a | AS | | c*c*a |
| AV00004 | AV00004- | 451 | c*u*ucuugaAaGaUagugu | AV00004- | 563 | u*U*aacaCuaucUuUcAag |
| | SS | | ua*a | AS | | a*a*g |
| AV00005 | AV00005- | 452 | c*a*auguggUcAuCaaaua | AV00005- | 564 | u*C*uuauUugauGaCcAca |
| | SS | | ag*a | AS | | u*u*g |
| AV00006 | AV00006- | 453 | g*u*ggucauCaAaUaagug | AV00006- | 565 | u*A*gcacUuauuUgAuGac |
| | SS | | cu*a | AS | | c*a*c |
| AV00007 | AV00007- | 454 | c*a*ucgagaCaUuUauaaa | AV00007- | 566 | u*C*cuuuAuaaaUgUcUcg |
| | SS | | gg*a | AS | | a*u*g |
| AV00008 | AV00008- | 455 | a*u*cgagacAuUuAuaaag | AV00008- | 567 | u*U*ccuuUauaaAuGuCuc |
| | SS | | ga*a | AS | | g*a*u |
| AV00009 | AV00009- | 456 | g*u*ugauauGaGaGgaguc | AV00009- | 568 | u*U*ugacUccucUcAuAuc |
| | SS | | aa*a | AS | | a*a*c |
| AV00010 | AV00010- | 457 | c*u*aagguuAgCaGuguug | AV00010- | 569 | u*U*ucaaCacugCuAaCcu |
| | SS | | aa*a | AS | | u*a*g |
| AV00011 | AV00011- | 458 | g*g*uuagcaGuGuUgaaga | AV00011- | 570 | u*A*uucuUcaacAcUgCua |
| | SS | | au*a | AS | | a*c*c |
| AV00012 | AV00012- | 459 | g*u*uagcagUgUuGaagaa | AV00012- | 571 | u*C*auucUucaaCaCuGcu |
| | SS | | ug*a | AS | | a*a*c |
| AV00013 | AV00013- | 460 | g*u*aacgugGaAuCuggau | AV00013- | 572 | u*G*aaucCagauUcCaCgu |
| | SS | | uc*a | AS | | u*a*c |
| AV00014 | AV00014- | 461 | a*a*cguggaAuCuGgauuc | AV00014- | 573 | u*G*agaaUccagAuUcCac |
| | SS | | uc*a | AS | | g*u*u |
| AV00015 | AV00015- | 462 | a*a*ucuggaUuCuCacuga | AV00015- | 574 | u*C*uucaGugagAaUcCag |
| | SS | | ag*a | AS | | a*u*u |
| AV00016 | AV00016- | 463 | a*c*uccagaUgCuUuugug | AV00016- | 575 | u*C*acacAaaagCaUcUgg |
| | SS | | ug*a | AS | | a*g*u |
| AV00017 | AV00017- | 464 | u*u*cuuaaaAcAuCugaaa | AV00017- | 576 | u*A*cuuuCagauGuUuUaa |
| | SS | | gu*a | AS | | g*a*a |
| AV00018 | AV00018- | 465 | u*u*ccucuaCuCcUcaaga | AV00018- | 577 | u*U*uucuUgaggAgUaGag |
| | SS | | aa*a | AS | | g*a*a |
| AV00019 | AV00019- | 466 | u*c*cucuacUcCuCaagaa | AV00019- | 578 | u*U*uuucUugagGaGuAga |
| | SS | | aa*a | AS | | g*g*a |
| AV00020 | AV00020- | 467 | g*c*cuuuuaAcCuGcaaaa | AV00020- | 579 | u*U*cuuuUgcagGuUaAaa |
| | SS | | ga*a | AS | | g*g*c |
| AV00021 | AV00021- | 468 | a*a*ccugcaAaAgAacuuu | AV00021- | 580 | u*G*uaaaGuucuUuUgCag |
| | SS | | ac*a | AS | | g*u*u |
| AV00022 | AV00022- | 469 | a*c*cugcaaAaGaAcuuua | AV00022- | 581 | u*G*guaaAguucUuUuGca |
| | SS | | cc*a | AS | | g*g*u |
| AV00023 | AV00023- | 470 | c*c*ugcaaaAgAaCuuuac | AV00023- | 582 | u*A*gguaAaguuCuUuUgc |
| | SS | | cu*a | AS | | a*g*g |
| AV00024 | AV00024- | 471 | g*c*aaaagaAcUuUaccug | AV00024- | 583 | u*U*ucagGuaaaGuUcUuu |
| | SS | | aa*a | AS | | u*g*c |
| AV00025 | AV00025- | 472 | a*c*uuuggaGgAgAagaau | AV00025- | 584 | u*C*aauuCuucuCcUcCaa |
| | SS | | ug*a | AS | | a*g*u |
| AV00026 | AV00026- | 473 | g*a*ggagaaGaAuUgaaug | AV00026- | 585 | u*C*acauUcaauUcUuCuc |
| | SS | | ug*a | AS | | c*u*c |
| AV00027 | AV00027- | 474 | u*g*uuaaagGaGuGaaugu | AV00027- | 586 | u*A*aacaUucacUcCuUua |
| | SS | | uu*a | AS | | a*c*a |
| AV00028 | AV00028- | 475 | u*u*cgcuguCaGuUuuuca | AV00028- | 587 | u*A*gugaAaaacUgAcAgc |
| | SS | | cu*a | AS | | g*a*a |
| AV00029 | AV00029- | 476 | c*g*cugucaGuUuUucacu | AV00029- | 588 | u*U*aaguGaaaaAcUgAca |
| | SS | | ua*a | AS | | g*c*g |
| AV00030 | AV00030- | 477 | u*u*uucacuUaUuCuuuac | AV00030- | 589 | u*G*aguaAagaaUaAgUga |
| | SS | | uc*a | AS | | a*a*a |
| AV00031 | AV00031- | 478 | g*a*agagaaGuGuAagugu | AV00031- | 590 | u*A*aacaCuuacAcUuCuc |
| | SS | | uu*a | AS | | u*u*c |
| AV00032 | AV00032- | 479 | a*g*agaaguGuAaGuguuu | AV00032- | 591 | u*A*gaaaCacuuAcAcUuc |
| | SS | | cu*a | AS | | u*c*u |
| AV00033 | AV00033- | 480 | a*g*aaguguAaGuGuuucu | AV00033- | 592 | u*U*aagaAacacUuAcAcu |
| | SS | | ua*a | AS | | u*c*u |
| AV00034 | AV00034- | 481 | a*g*uguaagUgUuUcuuaa | AV00034- | 593 | u*U*cuuaAgaaaCaCuUac |
| | SS | | ga*a | AS | | a*c*u |
| AV00035 | AV00035- | 482 | c*u*gguuacUcUuUgagau | AV00035- | 594 | u*C*aaucUcaaaGaGuAac |
| | SS | | ug*a | AS | | c*a*g |
| AV00036 | AV00036- | 483 | u*g*guuacuCuUuGagauu | AV00036- | 595 | u*A*caauCucaaAgAgUaa |
| | SS | | gu*a | AS | | c*c*a |
| AV00037 | AV00037- | 484 | a*c*ucuuugAgAuUgugua | AV00037- | 596 | u*G*uuacAcaauCuCaAag |
| | SS | | ac*a | AS | | a*g*u |
| AV00038 | AV00038- | 485 | c*u*cuuugaGaUuGuguaa | AV00038- | 597 | u*U*guuaCacaaUcUcAaa |
| | SS | | ca*a | AS | | g*a*g |
| AV00039 | AV00039- | 486 | u*c*ugucugCaCaAcaaaa | AV00039- | 598 | u*G*uuuuUguugUgCaGac |
| | SS | | ac*a | AS | | a*g*a |
| AV00040 | AV00040- | 487 | g*a*ggaacaAaCuCuucuu | AV00040- | 599 | u*C*caagAagagUuUgUuc |
| | SS | | gg*a | AS | | c*u*c |
| AV00041 | AV00041- | 488 | u*g*uguggaGgGuCacuca | AV00041- | 600 | u*U*augaGugacCcUcCac |
| | SS | | ua*a | AS | | a*c*a |
| AV00042 | AV00042- | 489 | u*a*uaguggCaUuUuaaau | AV00042- | 601 | u*A*gauuUaaaaUgCcAcu |
| | SS | | cu*a | AS | | a*u*a |
| AV00043 | AV00043- | 490 | u*c*ugucagAcAuUacaaa | AV00043- | 602 | u*C*uuuuGuaauGuCuGac |
| | SS | | ag*a | AS | | a*g*a |
| AV00044 | AV00044- | 491 | c*u*gucagaCaUuAcaaaa | AV00044- | 603 | u*U*cuuuUguaaUgUcUga |
| | SS | | ga*a | AS | | c*a*g |
| AV00045 | AV00045- | 492 | a*u*acaccuUuCuCacaaa | AV00045- | 604 | u*U*auuuGugagAaAgGug |
| | SS | | ua*a | AS | | u*a*u |
| AV00046 | AV00046- | 493 | g*a*agggaaUcAuGauauc | AV00046- | 605 | u*G*cgauAucauGaUuCcc |
| | SS | | gc*a | AS | | u*u*c |
| AV00047 | AV00047- | 494 | c*u*ccuuugAaUuAcacug | AV00047- | 606 | u*U*ucagUguaaUuCaAag |
| | SS | | aa*a | AS | | g*a*g |
| AV00048 | AV00048- | 495 | u*u*acacugAaUuCcaaaa | AV00048- | 607 | u*G*uuuuUggaaUuCaGug |
| | SS | | ac*a | AS | | u*a*a |
| AV00049 | AV00049- | 496 | c*a*agcacaAuUuAuacca | AV00049- | 608 | u*G*uuggUauaaAuUgUgc |
| | SS | | ac*a | AS | | u*u*g |
| AV00050- | AV00050- | 497 | c*c*aucgagAcAuUuauaa | AV00050- | 609 | u*C*uuuaUaaauGuCuCga |
| 2 | 2-SS | | ag*a | 2-AS | | u*g*g |
| AV00051- | AV00051- | 498 | u*a*ccugaaCcCuGccauu | AV00051- | 610 | u*A*gaauGgcagGgUuCag |
| 2 | 2-SS | | cu*a | 2-AS | | g*u*a |
| AV00052- | AV00052- | 499 | a*a*gguuagCaGuGuugaa | AV00052- | 611 | u*U*cuucAacacUgCuAac |
| 2 | 2-SS | | ga*a | 2-AS | | c*u*u |
| AV00732 | AV00732- | 500 | c*u*cuaugaAaAcGccuuc | AV00732- | 612 | u*A*agaaGgcguUuUcAua |
| | SS | | uu*a | AS | | g*a*g |
| AV00733 | AV00733- | 501 | c*a*cccaagGuGuUugcua | AV00733- | 613 | u*A*auagCaaacAcCuUgg |
| | SS | | uu*a | AS | | g*u*g |
| AV00734 | AV00734- | 502 | u*c*aguuuuCuUcCagcaa | AV00734- | 614 | u*A*cuugCuggaAgAaAac |
| | SS | | gu*a | AS | | u*g*a |
| AV00735 | AV00735- | 503 | a*a*ugacauGgAgAaaagg | AV00735- | 615 | u*A*accuUuucuCcAuGuc |
| | SS | | uu*a | AS | | a*u*u |
| AV00736 | AV00736- | 504 | g*g*uugcuuCuUgAaagau | AV00736- | 616 | u*C*uaucUuucaAgAaGca |
| | SS | | ag*a | AS | | a*c*c |
| AV00737 | AV00737- | 505 | u*c*uugaaaGaUaGuguua | AV00737- | 617 | u*U*guaaCacuaUcUuUca |
| | SS | | ca*a | AS | | a*g*a |
| AV00738 | AV00738- | 506 | u*g*uggucaUcAaAuaagu | AV00738- | 618 | u*G*cacuUauuuGaUgAcc |
| | SS | | gc*a | AS | | a*c*a |
| AV00739 | AV00739- | 507 | u*g*auaugaGaGgAgucaa | AV00739- | 619 | u*A*auugAcuccUcUcAua |
| | SS | | uu*a | AS | | u*c*a |
| AV00740 | AV00740- | 508 | u*a*ugagagGaGuCaauuu | AV00740- | 620 | u*U*aaaaUugacUcCuCuc |
| | SS | | ua*a | AS | | a*u*a |
| AV00741 | AV00741- | 509 | a*g*uguugaAgAaUgccaa | AV00741- | 621 | u*U*uuugGcauuCuUcAac |
| | SS | | aa*a | AS | | a*c*u |
| AV00742 | AV00742- | 510 | a*g*uaacguGgAaUcugga | AV00742- | 622 | u*A*auccAgauuCcAcGuu |
| | SS | | uu*a | AS | | a*c*u |
| AV00743 | AV00743- | 511 | c*c*ugugccCuUuCagaaa | AV00743- | 623 | u*A*auuuCugaaAgGgCac |
| | SS | | uu*a | AS | | a*g*g |
| AV00744 | AV00744- | 512 | c*c*uuuuaaCcUgCaaaag | AV00744- | 624 | u*U*ucuuUugcaGgUuAaa |
| | SS | | aa*a | AS | | a*g*g |
| AV00745 | AV00745- | 513 | g*a*acccugCcAuUcuaaa | AV00745- | 625 | u*A*uuuuAgaauGgCaGgg |
| | SS | | au*a | AS | | u*u*c |
| AV00746 | AV00746- | 514 | c*c*cugccaUuCuAaaauu | AV00746- | 626 | u*U*aaauUuuagAaUgGca |
| | SS | | ua*a | AS | | g*g*g |
| AV00747 | AV00747- | 515 | g*a*gaagaaUuGaAuguga | AV00747- | 627 | u*A*gucaCauucAaUuCuu |
| | SS | | cu*a | AS | | c*u*c |
| AV00748 | AV00748- | 516 | u*u*uuguuaAaGgAgugaa | AV00748- | 628 | u*C*auucAcuccUuUaAca |
| | SS | | ug*a | AS | | a*a*a |
| AV00749 | AV00749- | 517 | g*u*uaaaggAgUgAauguu | AV00749- | 629 | u*C*aaacAuucaCuCcUuu |
| | SS | | ug*a | AS | | a*a*c |
| AV00750 | AV00750- | 518 | u*u*aaaggaGuGaAuguuu | AV00750- | 630 | u*G*caaaCauucAcUcCuu |
| | SS | | gc*a | AS | | u*a*a |
| AV00751 | AV00751- | 519 | a*a*aggaguGaAuGuuugc | AV00751- | 631 | u*U*ggcaAacauUcAcUcc |
| | SS | | ca*a | AS | | u*u*u |
| AV00752 | AV00752- | 520 | u*u*gcacaaAgAuGauucg | AV00752- | 632 | u*A*gegaAucauCuUuGug |
| | SS | | cu*a | AS | | c*a*a |
| AV00753 | AV00753- | 521 | a*a*gaugauUcGcUgucag | AV00753- | 633 | u*A*acugAcagcGaAuCau |
| | SS | | uu*a | AS | | c*u*u |
| AV00754 | AV00754- | 522 | u*g*auucgcUgUcAguuuu | AV00754- | 634 | u*G*aaaaAcugaCaGcGaa |
| | SS | | uc*a | AS | | u*c*a |
| AV00755 | AV00755- | 523 | c*a*uucgcuGuCaGuuuuu | AV00755- | 635 | u*U*gaaaAacugAcAgCga |
| | SS | | ca*a | AS | | a*u*g |
| AV00756 | AV00756- | 524 | c*u*ucgcugUcAgUuuuuc | AV00756- | 636 | u*G*ugaaAaacuGaCaGcg |
| | SS | | ac*a | AS | | a*a*g |
| AV00757 | AV00757- | 525 | c*c*gcugucAgUuUuucac | AV00757- | 637 | u*A*agugAaaaaCuGaCag |
| | SS | | uu*a | AS | | c*g*g |
| AV00758 | AV00758- | 526 | c*c*ugucagUuUuUcacuu | AV00758- | 638 | u*A*uaagUgaaaAaCuGac |
| | SS | | au*a | AS | | a*g*g |
| AV00759 | AV00759- | 527 | c*u*gucaguUuUuCacuua | AV00759- | 639 | u*A*auaaGugaaAaAcUga |
| | SS | | uu*a | AS | | c*a*g |
| AV00760 | AV00760- | 528 | c*g*ucaguuUuUcAcuuau | AV00760- | 640 | u*G*aauaAgugaAaAaCug |
| | SS | | uc*a | AS | | a*c*g |
| AV00761 | AV00761- | 529 | g*u*caguuuUuCaCuuauu | AV00761- | 641 | u*A*gaauAagugAaAaAcu |
| | SS | | cu*a | AS | | g*a*c |
| AV00762 | AV00762- | 530 | g*u*uuuucaCuUaUucuuu | AV00762- | 642 | u*G*uaaaGaauaAgUgAaa |
| | SS | | ac*a | AS | | a*a*c |
| AV00763 | AV00763- | 531 | u*u*uuucacUuAuUcuuua | AV00763- | 643 | u*A*guaaAgaauAaGuGaa |
| | SS | | cu*a | AS | | a*a*a |
| AV00764 | AV00764- | 532 | u*u*ucacuuAuUcUuuacu | AV00764- | 644 | u*G*gaguAaagaAuAaGug |
| | SS | | cc*a | AS | | a*a*a |
| AV00765 | AV00765- | 533 | u*a*uucuuuAcUcCcagaa | AV00765- | 645 | u*U*cuucUgggaGuAaAga |
| | SS | | ga*a | AS | | a*u*a |
| AV00766 | AV00766- | 534 | u*u*acucccAgAaGacugu | AV00766- | 646 | u*U*uacaGucuuCuGgGag |
| | SS | | aa*a | AS | | u*a*a |
| AV00767 | AV00767- | 535 | c*u*cccagaAgAcUguaag | AV00767- | 647 | u*U*ccuuAcaguCuUcUgg |
| | SS | | ga*a | AS | | g*a*g |
| AV00768 | AV00768- | 536 | g*a*gaagugUaAgUguuuc | AV00768- | 648 | u*A*agaaAcacuUaCaCuu |
| | SS | | uu*a | AS | | c*u*c |
| AV00769 | AV00769- | 537 | a*a*guguaaGuGuUucuua | AV00769- | 649 | u*C*uuaaGaaacAcUuAca |
| | SS | | ag*a | AS | | c*u*u |
| AV00770 | AV00770- | 538 | u*g*uaagugUuUcUuaaga | AV00770- | 650 | u*A*aucuUaagaAaCaCuu |
| | SS | | uu*a | AS | | a*c*a |
| AV00771 | AV00771- | 539 | c*u*aaguguUuCuUaagau | AV00771- | 651 | u*U*aaucUuaagAaAcAcu |
| | SS | | ua*a | AS | | u*a*g |
| AV00772 | AV00772- | 540 | u*a*aguguuUcUuAagauu | AV00772- | 652 | u*A*uaauCuuaaGaAaCac |
| | SS | | au*a | AS | | u*u*a |
| AV00773 | AV00773- | 541 | a*a*guguuuCuUaAgauua | AV00773- | 653 | u*G*auaaUcuuaAgAaAca |
| | SS | | uc*a | AS | | c*u*u |
| AV00774 | AV00774- | 542 | g*a*gcucugGuUaCucuuu | AV00774- | 654 | u*U*caaaGaguaAcCaGag |
| | SS | | ga*a | AS | | c*u*c |
| AV00775 | AV00775- | 543 | g*g*uuacucUuUgAgauug | AV00775- | 655 | u*C*acaaUcucaAaGaGua |
| | SS | | ug*a | AS | | a*c*c |
| AV00776 | AV00776- | 544 | c*a*uuguugGaGgAacaaa | AV00776- | 656 | u*A*guuuGuuccUcCaAca |
| | SS | | cu*a | AS | | a*u*g |
| AV00777 | AV00777- | 545 | c*u*auagugGcAuUuuaaa | AV00777- | 657 | u*G*auuuAaaauGcCaCua |
| | SS | | uc*a | AS | | u*a*g |
| AV00778 | AV00778- | 546 | u*u*uaaaucUgUcAgacau | AV00778- | 658 | u*U*aaugUcugaCaGaUuu |
| | SS | | ua*a | AS | | a*a*a |
| AV00779 | AV00779- | 547 | a*u*uauuauUcAcCaaaac | AV00779- | 659 | u*U*aguuUugguGaAuAau |
| | SS | | ua*a | AS | | a*a*u |
| AV00780 | AV00780- | 548 | a*g*aagggaAuCaUgauau | AV00780- | 660 | u*C*gauaUcaugAuUcCcu |
| | SS | | cg*a | AS | | u*c*u |
| AV00781 | AV00781- | 549 | u*c*caggcuCcUuUgaauu | AV00781- | 661 | u*G*uaauUcaaaGgAgCcu |
| | SS | | ac*a | AS | | g*g*a |
| AV00782 | AV00782- | 550 | a*c*ugaauuCcAaAaacca | AV00782- | 662 | u*A*uuggUuuuuGgAaUuc |
| | SS | | au*a | AS | | a*g*u |
| AV00783 | AV00783- | 551 | a*u*uccaaaAaCcAauaug | AV00783- | 663 | u*G*gcauAuuggUuUuUgg |
| | SS | | cc*a | AS | | a*a*u |
| AV00784 | AV00784- | 552 | g*c*acaauuUaUaCcaacu | AV00784- | 664 | u*A*caguUgguaUaAaUug |
| | SS | | gu*a | AS | | u*g*c |
| AV00785 | AV00785- | 553 | u*u*auaccaAcUgUugggu | AV00785- | 665 | u*U*uaccCaacaGuUgGua |
| | SS | | aa*a | AS | | u*a*a |
| AV00786 | AV00786- | 554 | u*a*caccaaAgUcGcugag | AV00786- | 666 | u*U*acucAgcgaCuUuGgu |
| | SS | | ua*a | AS | | g*u*a |
| AV00787 | AV00787- | 555 | a*g*uacaugGaCuGgauuu | AV00787- | 667 | u*U*aaaaUccagUcCaUgu |
| | SS | | ua*a | AS | | a*c*u |
| AV00788 | AV00788- | 556 | u*g*gacuggAuUuUagaga | AV00788- | 668 | u*U*uucuCuaaaAuCcAgu |
| | SS | | aa*a | AS | | c*c*a |
| AV00789 | AV00789- | 557 | g*g*acuggaUuUuAgagaa | AV00789- | 669 | u*U*uuucUcuaaAaUcCag |
| | SS | | aa*a | AS | | u*c*c |
| AV00790 | AV00790- | 558 | a*c*acagagCaGuGaugga | AV00790- | 670 | u*U*uuccAucacUgCuCug |
| | SS | | aa*a | AS | | u*g*u |
| AV00791 | AV00791- | 559 | u*g*aguucaAgUcAaauuc | AV00791- | 671 | u*C*agaaUuugaCuUgAac |
| | SS | | ug*a | AS | | u*c*a |
| AV00792 | AV00792- | 560 | g*a*guucaaGuCaAauucu | AV00792- | 672 | u*U*cagaAuuugAcUuGaa |
| | SS | | ga*a | AS | | c*u*c |
| AV01712 | AV01712- | 1178 | u*g*ucaguuUuUcAcuuau | AV01712- | 1383 | u*G*aauaAgugaAaAaCug |
| | SS | | uc*a | AS | | a*c*a |
| AV01713 | AV01713- | 1179 | u*g*ucaguuUuUcAcuuau | AV01713- | 1384 | a*G*aauaAgugaAaAaCug |
| | SS | | uc*u | AS | | a*c*a |
| AV01714 | AV01714- | 1180 | u*g*ucaguuUuUcAcuuau | AV01714- | 1385 | g*G*aauaAgugaAaAaCug |
| | SS | | uc*c | AS | | a*c*a |
| AV01715 | AV01715- | 1181 | u*g*ucaguuUuUcAcuuau | AV01715- | 1386 | c*G*aauaAgugaAaAaCug |
| | SS | | uc*g | AS | | a*c*a |
| AV01716 | AV01716- | 1182 | a*g*uuUuUcAcuuauuc*a | AV01716- | 1387 | u*G*aauaAgugaAaAa*C* |
| | SS | | | AS | | u |
| AV01717 | AV01717- | 1183 | c*a*guuUuUcAcuuauuc* | AV01717- | 1388 | u*G*aauaAgugaAaAaC*u |
| | SS | | a | AS | | *g |
| AV01718 | AV01718- | 1184 | a*c*aguuUuUcAcuuauuc | AV01718- | 1389 | u*G*aauaAgugaAaAaCu* |
| | SS | | *a | AS | | g*u |
| AV01719 | AV01719- | 1185 | u*c*aguuUuUcAcuuauuc | AV01719- | 1390 | u*G*aauaAgugaAaAaCu* |
| | SS | | *a | AS | | g*a |
| AV01720 | AV01720- | 1186 | g*c*aguuUuUcAcuuauuc | AV01720- | 1391 | u*G*aauaAgugaAaAaCu* |
| | SS | | *a | AS | | g*c |
| AV01721 | AV01721- | 1187 | c*c*aguuUuUcAcuuauuc | AV01721- | 1392 | u*G*aauaAgugaAaAaCu* |
| | SS | | *a | AS | | g*g |
| AV01722 | AV01722- | 1188 | a*u*caguuUuUcAcuuauu | AV01722- | 1393 | u*G*aauaAgugaAaAaCug |
| | SS | | c*a | AS | | *a*u |
| AV01723 | AV01723- | 1189 | u*u*caguuUuUcAcuuauu | AV01723- | 1394 | u*G*aauaAgugaAaAaCug |
| | SS | | c*a | AS | | *a*a |
| AV01724 | AV01724- | 1190 | g*u*caguuUuUcAcuuauu | AV01724- | 1395 | u*G*aauaAgugaAaAaCug |
| | SS | | c*a | AS | | *a*c |
| AV01725 | AV01725- | 1191 | c*u*caguuUuUcAcuuauu | AV01725- | 1396 | u*G*aauaAgugaAaAaCug |
| | SS | | c*a | AS | | *a*g |
| AV01726 | AV01726- | 1192 | g*g*ucaguuUuUcAcuuau | AV01726- | 1397 | u*G*aauaAgugaAaAaCug |
| | SS | | uc*a | AS | | a*c*c |
| AV01727 | AV01727- | 1193 | a*g*ucaguuUuUcAcuuau | AV01727- | 1398 | u*G*aauaAgugaAaAaCug |
| | SS | | uc*a | AS | | a*c*u |
| AV01728 | AV01728- | 1194 | c*g*ucaguuUuUcAcuuau | AV01728- | 1399 | u*G*aauaAgugaAaAaCug |
| | SS | | uc*a | AS | | a*c*g |
| AV01729 | AV01729- | 1195 | c*u*gucaguuUuUcAcuua | AV01729- | 1400 | u*G*aauaAgugaAaAaCug |
| | SS | | uuc*a | AS | | ac*a*g |
| AV01730 | AV01730- | 1196 | g*c*ugucaguuUuUcAcuu | AV01730- | 1401 | u*G*aauaAgugaAaAaCug |
| | SS | | auuc*a | AS | | aca*g*c |
| AV01731 | AV01731- | 1197 | c*g*cugucaguuUuUcAcu | AV01731- | 1402 | u*G*aauaAgugaAaAaCug |
| | SS | | uauuc*a | AS | | acag*c*g |
| AV01732 | AV01732- | 1198 | u*c*gcugucaguuUuUcAc | AV01732- | 1403 | u*G*aauaAgugaAaAaCug |
| | SS | | uuauuc*a | AS | | acagc*g*a |
| AV01733 | AV01733- | 1199 | u*u*cgcugucaguuUuUcA | AV01733- | 1404 | u*G*aauaAgugaAaAaCug |
| | SS | | cuuauuc*a | AS | | acagcg*a*a |
| AV01734 | AV01734- | 1200 | a*g*augauucgcugucagu | AV01734- | 1405 | u*G*aauaAgugaAaAaCug |
| | SS | | uUuUcAcuuauuc*a | AS | | acagcgaaucau*c*u |
| AV01735 | AV01735- | 1201 | u*g*ucaguuUuUcAcuuau | AV01735- | 1406 | u*G*aauaAgugaAaAaCug |
| | SS | | uc*a | AS | | aca*u*u |
| AV01736 | AV01736- | 1202 | u*g*ucaguuUuUcAcuuau | AV01736- | 1407 | u*G*aauaAgugaAaAaCug |
| | SS | | uc*a | AS | | aca*g*a |
| AV01737 | AV01737- | 1203 | u*g*ucaguuUuUcAcuuau | AV01737- | 1408 | u*G*aau(aUNA)AgugaAa |
| | SS | | uc*a | AS | | AaCuga*c*a |
| AV01738 | AV01738- | 1204 | u*g*ucaguuUuUcAcuuau | AV01738- | 1409 | u*G*aaua(aUNA)gugaAa |
| | SS | | uc*a | AS | | AaCuga*c*a |
| AV01739 | AV01739- | 1205 | u*g*ucaguuUUUcacuuau | AV01739- | 1410 | u*G*aauAagugaaaAaCug |
| | SS | | uc*a | AS | | a*c*a |
| AV01740 | AV01740- | 1206 | u*g*ucaguuUuUcAcucau | AV01740- | 1411 | u*G*aauaAgugaAaAaCug |
| | SS | | uc*a | AS | | a*c*a |
| AV01741 | AV01741- | 1207 | u*g*ucaguuUuUcAcuuau | AV01741- | 1412 | u*A*aauaAgugaAaAaCug |
| | SS | | uu*a | AS | | a*c*a |
| AV01742 | AV01742- | 1208 | u*g*ucaguuUuUcAcuuau | AV01742- | 1413 | u*G*aauaAgugaAaAaCug |
| | SS | | u*c*a | AS | | a*c*a |
| AV01743 | AV01743- | 1209 | a*u*acaccuUuCuCacaaa | AV01743- | 1414 | u*U*auuuGugagAaAgGug |
| | SS | | ua*a | AS | | u*a*u |
| AV01744 | AV01744- | 1210 | a*u*acaccuUuCuCacaaa | AV01744- | 1415 | a*U*auuuGugagAaAgGug |
| | SS | | ua*u | AS | | u*a*u |
| AV01745 | AV01745- | 1211 | a*u*acaccuUuCuCacaaa | AV01745- | 1416 | g*U*auuuGugagAaAgGug |
| | SS | | ua*c | AS | | u*a*u |
| AV01746 | AV01746- | 1212 | a*u*acaccuUuCuCacaaa | AV01746- | 1417 | c*U*auuuGugagAaAgGug |
| | SS | | ua*g | AS | | u*a*u |
| AV01747 | AV01747- | 1213 | a*c*cuUuCuCacaaaua*a | AV01747- | 1418 | u*U*auuuGugagAaAg*G* |
| | SS | | | AS | | u |
| AV01748 | AV01748- | 1214 | c*a*ccuUuCuCacaaaua* | AV01748- | 1419 | u*U*auuuGugagAaAgG*u |
| | SS | | a | AS | | *g |
| AV01749 | AV01749- | 1215 | a*c*accuUuCuCacaaaua | AV01749- | 1420 | u*U*auuuGugagAaAgGu* |
| | SS | | *a | AS | | g*u |
| AV01750 | AV01750- | 1216 | u*c*accuUuCuCacaaaua | AV01750- | 1421 | u*U*auuuGugagAaAgGu* |
| | SS | | *a | AS | | g*a |
| AV01751 | AV01751- | 1217 | g*c*accuUuCuCacaaaua | AV01751- | 1422 | u*U*auuuGugagAaAgGu* |
| | SS | | *a | AS | | g*c |
| AV01752 | AV01752- | 1218 | c*c*accuUuCuCacaaaua | AV01752- | 1423 | u*U*auuuGugagAaAgGu* |
| | SS | | *a | AS | | g*g |
| AV01753 | AV01753- | 1219 | a*a*caccuUuCuCacaaau | AV01753- | 1424 | u*U*auuuGugagAaAgGug |
| | SS | | a*a | AS | | *u*u |
| AV01754 | AV01754- | 1220 | u*a*caccuUuCuCacaaau | AV01754- | 1425 | u*U*auuuGugagAaAgGug |
| | SS | | a*a | AS | | *u*a |
| AV01755 | AV01755- | 1221 | g*a*caccuUuCuCacaaau | AV01755- | 1426 | u*U*auuuGugagAaAgGug |
| | SS | | a*a | AS | | *u*c |
| AV01756 | AV01756- | 1222 | c*a*caccuUuCuCacaaau | AV01756- | 1427 | u*U*auuuGugagAaAgGug |
| | SS | | a*a | AS | | *u*g |
| AV01757 | AV01757- | 1223 | g*u*acaccuUuCuCacaaa | AV01757- | 1428 | u*U*auuuGugagAaAgGug |
| | SS | | ua*a | AS | | u*a*c |
| AV01758 | AV01758- | 1224 | c*u*acaccuUuCuCacaaa | AV01758- | 1429 | u*U*auuuGugagAaAgGug |
| | SS | | ua*a | AS | | u*a*g |
| AV01759 | AV01759- | 1225 | u*u*acaccuUuCuCacaaa | AV01759- | 1430 | u*U*auuuGugagAaAgGug |
| | SS | | ua*a | AS | | u*a*a |
| AV01760 | AV01760- | 1226 | g*a*uacaccuUuCuCacaa | AV01760- | 1431 | u*U*auuuGugagAaAgGug |
| | SS | | aua*a | AS | | ua*u*c |
| AV01761 | AV01761- | 1227 | a*g*auacaccuUuCuCaca | AV01761- | 1432 | u*U*auuuGugagAaAgGug |
| | SS | | aaua*a | AS | | uau*c*u |
| AV01762 | AV01762- | 1228 | a*a*gauacaccuUuCuCac | AV01762- | 1433 | u*U*auuuGugagAaAgGug |
| | SS | | aaaua*a | AS | | uauc*u*u |
| AV01763 | AV01763- | 1229 | a*a*agauacaccuUuCuCa | AV01763- | 1434 | u*U*auuuGugagAaAgGug |
| | SS | | caaaua*a | AS | | uaucu*u*u |
| AV01764 | AV01764- | 1230 | a*a*aagauacaccuUuCuC | AV01764- | 1435 | u*U*auuuGugagAaAgGug |
| | SS | | acaaaua*a | AS | | uaucuu*u*u |
| AV01765 | AV01765- | 1231 | c*a*uuacaaaagauacacc | AV01765- | 1436 | u*U*auuuGugagAaAgGug |
| | SS | | uUuCuCacaaaua*a | AS | | uaucuuuuguaa*u*g |
| AV01766 | AV01766- | 1232 | a*u*acaccuUuCuCacaaa | AV01766- | 1437 | u*U*auuuGugagAaAgGug |
| | SS | | ua*a | AS | | uau*u*u |
| AV01767 | AV01767- | 1233 | a*u*acaccuUuCuCacaaa | AV01767- | 1438 | u*U*auuuGugagAaAgGug |
| | SS | | ua*a | AS | | uau*g*a |
| AV01768 | AV01768- | 1234 | a*u*acaccuUuCuCacaaa | AV01768- | 1439 | u*U*auu(uUNA)GugagAa |
| | SS | | ua*a | AS | | AgGugu*a*u |
| AV01769 | AV01769- | 1235 | a*u*acaccuUuCuCacaaa | AV01769- | 1440 | u*U*auuu(gUNA)ugagAa |
| | SS | | ua*a | AS | | AgGugu*a*u |
| AV01770 | AV01770- | 1236 | a*u*acaccuUUCucacaaa | AV01770- | 1441 | u*U*auuUgugagaaAgGug |
| | SS | | ua*a | AS | | u*a*u |
| AV01771 | AV01771- | 1237 | a*u*acaccuUuCuCacgaa | AV01771- | 1442 | u*U*auuuGugagAaAgGug |
| | SS | | ua*a | AS | | u*a*u |
| AV01772 | AV01772- | 1238 | a*u*acaccuUuCuCacaaa | AV01772- | 1443 | u*C*auuuGugagAaAgGug |
| | SS | | ug*a | AS | | u*a*u |
| AV01773 | AV01773- | 1239 | a*u*acaccuUuCuCacaaa | AV01773- | 1444 | u*U*auuuGugagAaAgGug |
| | SS | | u*a*a | AS | | u*a*u |
| AV01774 | AV01774- | 1240 | u*u*cgcuguCaGuUuuuca | AV01774- | 1445 | u*A*gugaAaaacUgAcAgc |
| | SS | | cu*a | AS | | g*a*a |
| AV01775 | AV01775- | 1241 | u*u*cgcuguCaGuUuuuca | AV01775- | 1446 | a*A*gugaAaaacUgAcAgc |
| | SS | | cu*u | AS | | g*a*a |
| AV01776 | AV01776- | 1242 | u*u*cgcuguCaGuUuuuca | AV01776- | 1447 | g*A*gugaAaaacUgAcAgc |
| | SS | | cu*c | AS | | g*a*a |
| AV01777 | AV01777- | 1243 | u*u*cgcuguCaGuUuuuca | AV01777- | 1448 | c*A*gugaAaaacUgAcAgc |
| | SS | | cu*g | AS | | g*a*a |
| AV01778 | AV01778- | 1244 | c*u*guCaGuUuuucacu*a | AV01778- | 1449 | u*A*gugaAaaacUgAc*A* |
| | SS | | | AS | | g |
| AV01779 | AV01779- | 1245 | g*c*uguCaGuUuuucacu* | AV01779- | 1450 | u*A*gugaAaaacUgAcA*g |
| | SS | | a | AS | | *c |
| AV01780 | AV01780- | 1246 | a*g*cuguCaGuUuuucacu | AV01780- | 1451 | u*A*gugaAaaacUgAcAg* |
| | SS | | *a | AS | | c*u |
| AV01781 | AV01781- | 1247 | u*g*cuguCaGuUuuucacu | AV01781- | 1452 | u*A*gugaAaaacUgAcAg* |
| | SS | | *a | AS | | c*a |
| AV01782 | AV01782- | 1248 | g*g*cuguCaGuUuuucacu | AV01782- | 1453 | u*A*gugaAaaacUgAcAg* |
| | SS | | *a | AS | | c*c |
| AV01783 | AV01783- | 1249 | c*g*cuguCaGuUuuucacu | AV01783- | 1454 | u*A*gugaAaaacUgAcAg* |
| | SS | | *a | AS | | c*g |
| AV01784 | AV01784- | 1250 | a*c*gcuguCaGuUuuucac | AV01784- | 1455 | u*A*gugaAaaacUgAcAgc |
| | SS | | u*a | AS | | *g*u |
| AV01785 | AV01785- | 1251 | u*c*gcuguCaGuUuuucac | AV01785- | 1456 | u*A*gugaAaaacUgAcAgc |
| | SS | | u*a | AS | | *g*a |
| AV01786 | AV01786- | 1252 | g*c*gcuguCaGuUuuucac | AV01786- | 1457 | u*A*gugaAaaacUgAcAgc |
| | SS | | u*a | AS | | *g*c |
| AV01787 | AV01787- | 1253 | c*c*gcuguCaGuUuuucac | AV01787- | 1458 | u*A*gugaAaaacUgAcAgc |
| | SS | | u*a | AS | | *g*g |
| AV01788 | AV01788- | 1254 | g*u*cgcuguCaGuUuuuca | AV01788- | 1459 | u*A*gugaAaaacUgAcAgc |
| | SS | | cu*a | AS | | g*a*c |
| AV01789 | AV01789- | 1255 | c*u*cgcuguCaGuUuuuca | AV01789- | 1460 | u*A*gugaAaaacUgAcAgc |
| | SS | | cu*a | AS | | g*a*g |
| AV01790 | AV01790- | 1256 | a*u*cgcuguCaGuUuuuca | AV01790- | 1461 | u*A*gugaAaaacUgAcAgc |
| | SS | | cu*a | AS | | g*a*u |
| AV01791 | AV01791- | 1257 | a*u*ucgcuguCaGuUuuuc | AV01791- | 1462 | u*A*gugaAaaacUgAcAgc |
| | SS | | acu*a | AS | | ga*a*u |
| AV01792 | AV01792- | 1258 | g*a*uucgcuguCaGuUuuu | AV01792- | 1463 | u*A*gugaAaaacUgAcAgc |
| | SS | | cacu*a | AS | | gaa*u*c |
| AV01793 | AV01793- | 1259 | u*g*auucgcuguCaGuUuu | AV01793- | 1464 | u*A*gugaAaaacUgAcAgc |
| | SS | | ucacu*a | AS | | gaau*c*a |
| AV01794 | AV01794- | 1260 | a*u*gauucgcuguCaGuUu | AV01794- | 1465 | u*A*gugaAaaacUgAcAgc |
| | SS | | uucacu*a | AS | | gaauc*a*u |
| AV01795 | AV01795- | 1261 | g*a*ugauucgcuguCaGuU | AV01795- | 1466 | u*A*gugaAaaacUgAcAgc |
| | SS | | uuucacu*a | AS | | gaauca*u*C |
| AV01796 | AV01796- | 1262 | c*a*caaagaugauucgcug | AV01796- | 1467 | u*A*gugaAaaacUgAcAge |
| | SS | | uCaGuUuuucacu*a | AS | | gaaucaucuuug*u*g |
| AV01797 | AV01797- | 1263 | u*u*cgcuguCaGuUuuuca | AV01797- | 1468 | u*A*gugaAaaacUgAcAgc |
| | SS | | cu*a | AS | | gaa*u*u |
| AV01798 | AV01798- | 1264 | u*u*cgcuguCaGuUuuuca | AV01798- | 1469 | u*A*gugaAaaacUgAcAgc |
| | SS | | cu*a | AS | | gaa*g*a |
| AV01799 | AV01799- | 1265 | u*u*cgcuguCaGuUuuuca | AV01799- | 1470 | u*A*gug(aUNA)AaaacUg |
| | SS | | cu*a | AS | | AcAgcg*a*a |
| AV01800 | AV01800- | 1266 | u*u*cgcuguCaGuUuuuca | AV01800- | 1471 | u*A*guga(aUNA)aaacUg |
| | SS | | cu*a | AS | | AcAgcg*a*a |
| AV01801 | AV01801- | 1267 | u*u*cgcuguCAGuuuuuca | AV01801- | 1472 | u*A*gugAaaaacugAcAgc |
| | SS | | cu*a | AS | | g*a*a |
| AV01802 | AV01802- | 1268 | u*u*cgcuguCaGuUuucca | AV01802- | 1473 | u*A*gugaAaaacUgAcAgc |
| | SS | | cu*a | AS | | g*a*a |
| AV01803 | AV01803- | 1269 | u*u*cgcuguCaGuUuuuca | AV01803- | 1474 | u*G*gugaAaaacUgAcAgc |
| | SS | | cc*a | AS | | g*a*a |
| AV01804 | AV01804- | 1270 | u*u*cgcuguCaGuUuuuca | AV01804- | 1475 | u*A*gugaAaaacUgAcAgc |
| | SS | | c*u*a | AS | | g*a*a |
| AV01805 | AV01805- | 1271 | u*c*caggcuCcUuUgaauu | AV01805- | 1476 | u*G*uaauUcaaaGgAgCcu |
| | SS | | ac*a | AS | | g*g*a |
| AV01806 | AV01806- | 1272 | u*c*caggcuCcUuUgaauu | AV01806- | 1477 | a*G*uaauUcaaaGgAgCcu |
| | SS | | ac*u | AS | | g*g*a |
| AV01807 | AV01807- | 1273 | u*c*caggcuCcUuUgaauu | AV01807- | 1478 | g*G*uaauUcaaaGgAgCcu |
| | SS | | ac*c | AS | | g*g*a |
| AV01808 | AV01808- | 1274 | u*c*caggcuCcUuUgaauu | AV01808- | 1479 | c*G*uaauUcaaaGgAgCcu |
| | SS | | ac*g | AS | | g*g*a |
| AV01809 | AV01809- | 1275 | g*g*cuCcUuUgaauuac*a | AV01809- | 1480 | u*G*uaauUcaaaGgAg*C* |
| | SS | | | AS | | c |
| AV01810 | AV01810- | 1276 | a*g*gcuCcUuUgaauuac* | AV01810- | 1481 | u*G*uaauUcaaaGgAgC*c |
| | SS | | a | AS | | *u |
| AV01811 | AV01811- | 1277 | a*a*ggcuCcUuUgaauuac | AV01811- | 1482 | u*G*uaauUcaaaGgAgCc* |
| | SS | | *a | AS | | u*u |
| AV01812 | AV01812- | 1278 | u*a*ggcuCcUuUgaauuac | AV01812- | 1483 | u*G*uaauUcaaaGgAgCc* |
| | SS | | *a | AS | | u*a |
| AV01813 | AV01813- | 1279 | g*a*ggcuCcUuUgaauuac | AV01813- | 1484 | u*G*uaauUcaaaGgAgCc* |
| | SS | | *a | AS | | u*c |
| AV01814 | AV01814- | 1280 | c*a*ggcuCcUuUgaauuac | AV01814- | 1485 | u*G*uaauUcaaaGgAgCc* |
| | SS | | *a | AS | | u*g |
| AV01815 | AV01815- | 1281 | a*c*aggcuCcUuUgaauua | AV01815- | 1486 | u*G*uaauUcaaaGgAgCcu |
| | SS | | c*a | AS | | *g*u |
| AV01816 | AV01816- | 1282 | u*c*aggcuCcUuUgaauua | AV01816- | 1487 | u*G*uaauUcaaaGgAgCcu |
| | SS | | c*a | AS | | *g*a |
| AV01817 | AV01817- | 1283 | g*c*aggcuCcUuUgaauua | AV01817- | 1488 | u*G*uaauUcaaaGgAgCcu |
| | SS | | c*a | AS | | *g*c |
| AV01818 | AV01818- | 1284 | c*c*aggcuCcUuUgaauua | AV01818- | 1489 | u*G*uaauUcaaaGgAgCcu |
| | SS | | c*a | AS | | *g*g |
| AV01819 | AV01819- | 1285 | g*c*caggcuCcUuUgaauu | AV01819- | 1490 | u*G*uaauUcaaaGgAgCcu |
| | SS | | ac*a | AS | | g*g*c |
| AV01820 | AV01820- | 1286 | c*c*caggcuCcUuUgaauu | AV01820- | 1491 | u*G*uaauUcaaaGgAgCcu |
| | SS | | ac*a | AS | | g*g*g |
| AV01821 | AV01821- | 1287 | a*c*caggcuCcUuUgaauu | AV01821- | 1492 | u*G*uaauUcaaaGgAgCcu |
| | SS | | ac*a | AS | | g*g*u |
| AV01822 | AV01822- | 1288 | c*u*ccaggcuCcUuUgaau | AV01822- | 1493 | u*G*uaauUcaaaGgAgCcu |
| | SS | | uac*a | AS | | gg*a*g |
| AV01823 | AV01823- | 1289 | a*c*uccaggcuCcUuUgaa | AV01823- | 1494 | u*G*uaauUcaaaGgAgCcu |
| | SS | | uuac*a | AS | | gga*g*u |
| AV01824 | AV01824- | 1290 | a*a*cuccaggcuCcUuUga | AV01824- | 1495 | u*G*uaauUcaaaGgAgCcu |
| | SS | | auuac*a | AS | | ggag*u*u |
| AV01825 | AV01825- | 1291 | a*a*acuccaggcuCcUuUg | AV01825- | 1496 | u*G*uaauUcaaaGgAgCcu |
| | SS | | aauuac*a | AS | | ggagu*u*u |
| AV01826 | AV01826- | 1292 | a*a*aacuccaggcuCcUuU | AV01826- | 1497 | u*G*uaauUcaaaGgAgCcu |
| | SS | | gaauuac*a | AS | | ggaguu*u*u |
| AV01827 | AV01827- | 1293 | c*u*ugauaaaacuccaggc | AV01827- | 1498 | u*G*uaauUcaaaGgAgCcu |
| | SS | | uCcUuUgaauuac*a | AS | | ggaguuuuauca*a*g |
| AV01828 | AV01828- | 1294 | u*c*caggcuCcUuUgaauu | AV01828- | 1499 | u*G*uaauUcaaaGgAgCcu |
| | SS | | ac*a | AS | | gga*u*u |
| AV01829 | AV01829- | 1295 | u*c*caggcuCcUuUgaauu | AV01829- | 1500 | u*G*uaauUcaaaGgAgCcu |
| | SS | | ac*a | AS | | gga*g*a |
| AV01830 | AV01830- | 1296 | u*c*caggcuCcUuUgaauu | AV01830- | 1501 | u*G*uaa(uUNA)UcaaaGg |
| | SS | | ac*a | AS | | AgCcug*g*a |
| AV01831 | AV01831- | 1297 | u*c*caggcuCcUuUgaauu | AV01831- | 1502 | u*G*uaau(uUNA)caaaGg |
| | SS | | ac*a | AS | | AgCcug*g*a |
| AV01832 | AV01832- | 1298 | u*c*caggcuCCUuugaauu | AV01832- | 1503 | u*G*uaaUucaaaggAgCcu |
| | SS | | ac*a | AS | | g*g*a |
| AV01833 | AV01833- | 1299 | u*c*caggcuCcUuUgaguu | AV01833- | 1504 | u*G*uaauUcaaaGgAgCcu |
| | SS | | ac*a | AS | | g*g*a |
| AV01834 | AV01834- | 1300 | u*c*caggcuCcUuUgaauu | AV01834- | 1505 | u*A*uaauUcaaaGgAgCcu |
| | SS | | au*a | AS | | g*g*a |
| AV01835 | AV01835- | 1301 | u*c*caggcuCcUuUgaauu | AV01835- | 1506 | u*G*uaauUcaaaGgAgCcu |
| | SS | | a*c*a | AS | | g*g*a |
| AV01836 | AV01836- | 1302 | g*a*aggaguGaAuGuuugc | AV01836- | 1507 | u*U*ggcaAacauUcAcUcc |
| | SS | | ca*a | AS | | u*u*c |
| AV01837 | AV01837- | 1303 | g*a*aggaguGaAuGuuugc | AV01837- | 1508 | a*U*ggcaAacauUcAcUcc |
| | SS | | ca*u | AS | | u*u*c |
| AV01838 | AV01838- | 1304 | g*a*aggaguGaAuGuuugc | AV01838- | 1509 | g*U*ggcaAacauUcAcUcc |
| | SS | | ca*c | AS | | u*u*c |
| AV01839 | AV01839- | 1305 | g*a*aggaguGaAuGuuugc | AV01839- | 1510 | c*U*ggcaAacauUcAcUcc |
| | SS | | ca*g | AS | | u*u*c |
| AV01840 | AV01840- | 1306 | g*a*guGaAuGuuugcca*a | AV01840- | 1511 | u*U*ggcaAacauUcAc*U* |
| | SS | | | AS | | c |
| AV01841 | AV01841- | 1307 | g*g*aguGaAuGuuugcca* | AV01841- | 1512 | u*U*ggcaAacauUcAcU*c |
| | SS | | a | AS | | *c |
| AV01842 | AV01842- | 1308 | a*g*gaguGaAuGuuugcca | AV01842- | 1513 | u*U*ggcaAacauUcAcUc* |
| | SS | | *a | AS | | c*u |
| AV01843 | AV01843- | 1309 | u*g*gaguGaAuGuuugcca | AV01843- | 1514 | u*U*ggcaAacauUcAcUc* |
| | SS | | *a | AS | | c*a |
| AV01844 | AV01844- | 1310 | g*g*gaguGaAuGuuugcca | AV01844- | 1515 | u*U*ggcaAacauUcAcUc* |
| | SS | | *a | AS | | c*c |
| AV01845 | AV01845- | 1311 | c*g*gaguGaAuGuuugcca | AV01845- | 1516 | u*U*ggcaAacauUcAcUc* |
| | SS | | *a | AS | | c*g |
| AV01846 | AV01846- | 1312 | a*a*ggaguGaAuGuuugcc | AV01846- | 1517 | u*U*ggcaAacauUcAcUcc |
| | SS | | a*a | AS | | *u*u |
| AV01847 | AV01847- | 1313 | u*a*ggaguGaAuGuuugcc | AV01847- | 1518 | u*U*ggcaAacauUcAcUcc |
| | SS | | a*a | AS | | *u*a |
| AV01848 | AV01848- | 1314 | g*a*ggaguGaAuGuuugcc | AV01848- | 1519 | u*U*ggcaAacauUcAcUcc |
| | SS | | a*a | AS | | *u*c |
| AV01849 | AV01849- | 1315 | c*a*ggaguGaAuGuuugcc | AV01849- | 1520 | u*U*ggcaAacauUcAcUcc |
| | SS | | a*a | AS | | *u*g |
| AV01850 | AV01850- | 1316 | u*a*aggaguGaAuGuuugc | AV01850- | 1521 | u*U*ggcaAacauUcAcUcc |
| | SS | | ca*a | AS | | u*u*a |
| AV01851 | AV01851- | 1317 | c*a*aggaguGaAuGuuugc | AV01851- | 1522 | u*U*ggcaAacauUcAcUcc |
| | SS | | ca*a | AS | | u*u*g |
| AV01852 | AV01852- | 1318 | a*a*aggaguGaAuGuuugc | AV01852- | 1523 | u*U*ggcaAacauUcAcUcc |
| | SS | | ca*a | AS | | u*u*u |
| AV01853 | AV01853- | 1319 | u*a*aaggaguGaAuGuuug | AV01853- | 1524 | u*U*ggcaAacauUcAcUcc |
| | SS | | cca*a | AS | | uu*u*a |
| AV01854 | AV01854- | 1320 | u*u*aaaggaguGaAuGuuu | AV01854- | 1525 | u*U*ggcaAacauUcAcUcc |
| | SS | | gcca*a | AS | | uuu*a*a |
| AV01855 | AV01855- | 1321 | g*u*uaaaggaguGaAuGuu | AV01855- | 1526 | u*U*ggcaAacauUcAcUcc |
| | SS | | ugcca*a | AS | | uuua*a*c |
| AV01856 | AV01856- | 1322 | u*g*uuaaaggaguGaAuGu | AV01856- | 1527 | u*U*ggcaAacauUcAcUcc |
| | SS | | uugcca*a | AS | | uuuaa*c*a |
| AV01857 | AV01857- | 1323 | u*u*guuaaaggaguGaAuG | AV01857- | 1528 | u*U*ggcaAacauUcAcUcc |
| | SS | | uuugcca*a | AS | | uuuaac*a*a |
| AV01858 | AV01858- | 1324 | u*g*acuuuuguuaaaggag | AV01858- | 1529 | u*U*ggcaAacauUcAcUcc |
| | SS | | uGaAuGuuugcca*a | AS | | uuuaacaaaagu*c*a |
| AV01859 | AV01859- | 1325 | g*a*aggaguGaAuGuuugc | AV01859- | 1530 | u*U*ggcaAacauUcAcUcc |
| | SS | | ca*a | AS | | uuc*u*u |
| AV01860 | AV01860- | 1326 | g*a*aggaguGaAuGuuugc | AV01860- | 1531 | u*U*ggcaAacauUcAcUcc |
| | SS | | ca*a | AS | | uuc*g*a |
| AV01861 | AV01861- | 1327 | g*a*aggaguGaAuGuuugc | AV01861- | 1532 | u*U*ggc(aUNA)AacauUc |
| | SS | | ca*a | AS | | AcUccu*u*c |
| AV01862 | AV01862- | 1328 | g*a*aggaguGaAuGuuugc | AV01862- | 1533 | u*U*ggca(aUNA)acauUc |
| | SS | | ca*a | AS | | AcUccu*u*c |
| AV01863 | AV01863- | 1329 | g*a*aggaguGAAuguuugc | AV01863- | 1534 | u*U*ggcAaacauucAcUcc |
| | SS | | ca*a | AS | | u*u*c |
| AV01864 | AV01864- | 1330 | g*a*aggaguGaAuGuucgc | AV01864- | 1535 | u*U*ggcaAacauUcAcUcc |
| | SS | | ca*a | AS | | u*u*c |
| AV01865 | AV01865- | 1331 | g*a*aggaguGaAuGuuugc | AV01865- | 1536 | u*C*ggcaAacauUcAcUcc |
| | SS | | cg*a | AS | | u*u*c |
| AV01866 | AV01866- | 1332 | g*a*aggaguGaAuGuuugc | AV01866- | 1537 | u*U*ggcaAacauUcAcUcc |
| | SS | | c*a*a | AS | | u*u*c |
| AV01960 | AV01960- | 1333 | u*u*cgcuguCaGuUuuuca | AV01960- | 1538 | u*A*gugaAaaacUgAcAgc |
| | SS | | cu*a | AS | | g*a*a |
| AV01961 | AV01961- | 1334 | u*c*gcugucAgUuUuucac | AV01961- | 1539 | u*A*agugAaaaaCuGaCag |
| | SS | | uu*a | AS | | c*g*a |
| AV01962 | AV01962- | 1335 | c*g*cugucaGuUuUucacu | AV01962- | 1540 | u*U*aaguGaaaaAcUgAca |
| | SS | | ua*a | AS | | g*c*g |
| AV01963 | AV01963- | 1336 | g*c*ugucagUuUuUcacuu | AV01963- | 1541 | u*A*uaagUgaaaAaCuGac |
| | SS | | au*a | AS | | a*g*c |
| AV01964 | AV01964- | 1337 | c*u*gucaguUuUuCacuua | AV01964- | 1542 | u*A*auaaGugaaAaAcUga |
| | SS | | uu*a | AS | | c*a*g |
| AV01965 | AV01965- | 1338 | g*u*caguuuUuCaCuuauu | AV01965- | 1543 | u*A*gaauAagugAaAaAcu |
| | SS | | cu*a | AS | | g*a*c |
| AV01966 | AV01966- | 1339 | u*c*aguuuuUcAcUuauuc | AV01966- | 1544 | u*A*agaaUaaguGaAaAac |
| | SS | | uu*a | AS | | u*g*a |
| AV01967 | AV01967- | 1340 | c*a*guuuuuCaCuUauucu | AV01967- | 1545 | u*A*aagaAuaagUgAaAaa |
| | SS | | uu*a | AS | | c*u*g |
| AV01968 | AV01968- | 1341 | a*g*uuuuucAcUuAuucuu | AV01968- | 1546 | u*U*aaagAauaaGuGaAaa |
| | SS | | ua*a | AS | | a*c*u |
| AV01969 | AV01969- | 1342 | g*u*uuuucaCuUaUucuuu | AV01969- | 1547 | u*G*uaaaGaauaAgUgAaa |
| | SS | | ac*a | AS | | a*a*c |
| AV01970 | AV01970- | 1343 | a*a*aagauaCaCcUuucuc | AV01970- | 1548 | u*G*ugagAaaggUgUaUcu |
| | SS | | ac*a | AS | | u*u*u |
| AV01971 | AV01971- | 1344 | a*a*agauacAcCuUucuca | AV01971- | 1549 | u*U*gugaGaaagGuGuAuc |
| | SS | | ca*a | AS | | u*u*u |
| AV01972 | AV01972- | 1345 | a*a*gauacaCcUuUcucac | AV01972- | 1550 | u*U*ugugAgaaaGgUgUau |
| | SS | | aa*a | AS | | c*u*u |
| AV01973 | AV01973- | 1346 | a*g*auacacCuUuCucaca | AV01973- | 1551 | u*U*uuguGagaaAgGuGua |
| | SS | | aa*a | AS | | u*c*u |
| AV01974 | AV01974- | 1347 | g*a*uacaccUuUcUcacaa | AV01974- | 1552 | u*A*uuugUgagaAaGgUgu |
| | SS | | au*a | AS | | a*u*c |
| AV01975 | AV01975- | 1348 | u*a*caccuuUcUcAcaaau | AV01975- | 1553 | u*U*uauuUgugaGaAaGgu |
| | SS | | aa*a | AS | | g*u*a |
| AV01976 | AV01976- | 1349 | a*c*accuuuCuCaCaaaua | AV01976- | 1554 | u*U*uuauUugugAgAaAgg |
| | SS | | aa*a | AS | | u*g*u |
| AV01977 | AV01977- | 1350 | c*a*ccuuucUcAcAaauaa | AV01977- | 1555 | u*U*uuuaUuuguGaGaAag |
| | SS | | aa*a | AS | | g*u*g |
| AV01978 | AV01978- | 1351 | a*c*cuuucuCaCaAauaaa | AV01978- | 1556 | u*C*uuuuAuuugUgAgAaa |
| | SS | | ag*a | AS | | g*g*u |
| AV01979 | AV01979- | 1352 | c*c*uuucucAcAaAuaaaa | AV01979- | 1557 | u*U*cuuuUauuuGuGaGaa |
| | SS | | ga*a | AS | | a*g*g |
| AV01980 | AV01980- | 1353 | g*a*ugauucGcUgUcaguu | AV01980- | 1558 | u*A*aaacUgacaGcGaAuc |
| | SS | | uu*a | AS | | a*u*c |
| AV01981 | AV01981- | 1354 | a*u*gauucgCuGuCaguuu | AV01981- | 1559 | u*A*aaaaCugacAgCgAau |
| | SS | | uu*a | AS | | c*a*u |
| AV01982 | AV01982- | 1355 | u*g*auucgcUgUcAguuuu | AV01982- | 1560 | u*G*aaaaAcugaCaGcGaa |
| | SS | | uc*a | AS | | u*c*a |
| AV01983 | AV01983- | 1356 | g*a*uucgcuGuCaGuuuuu | AV01983- | 1561 | u*U*gaaaAacugAcAgCga |
| | SS | | ca*a | AS | | a*u*c |
| AV01984 | AV01984- | 1357 | a*u*ucgcugUcAgUuuuuc | AV01984- | 1562 | u*G*ugaaAaacuGaCaGcg |
| | SS | | ac*a | AS | | a*a*u |
| AV01985 | AV01985- | 1358 | u*c*gcugucAgUuUuucac | AV01985- | 1563 | u*A*agugAaaaaCuGaCag |
| | SS | | uu*a | AS | | c*g*a |
| AV01986 | AV01986- | 1359 | c*g*cugucaGuUuUucacu | AV01986- | 1564 | u*U*aaguGaaaaAcUgAca |
| | SS | | ua*a | AS | | g*c*g |
| AV01987 | AV01987- | 1360 | g*c*ugucagUUUuucacuu | AV01987- | 1565 | u*A*uaagUgaaaAaCuGac |
| | SS | | au*a | AS | | a*g*c |
| AV01988 | AV01988- | 1361 | c*u*gucaguUuUuCacuua | AV01988- | 1566 | u*A*auaaGugaaAaAcUga |
| | SS | | uu*a | AS | | c*a*g |
| AV01989 | AV01989- | 1362 | u*g*ucaguuUuUcAcuuau | AV01989- | 1567 | u*G*aauaAgugaAaAaCug |
| | SS | | uc*a | AS | | a*c*a |
| AV01990 | AV01990- | 1363 | a*a*aacuccAgGcUccuuu | AV01990- | 1568 | u*U*caaaGgagcCuGgAgu |
| | SS | | ga*a | AS | | u*u*u |
| AV01991 | AV01991- | 1364 | a*a*acuccaGgCuCcuuug | AV01991- | 1569 | u*U*ucaaAggagCcUgGag |
| | SS | | aa*a | AS | | u*u*u |
| AV01992 | AV01992- | 1365 | a*a*cuccagGcUcCuuuga | AV01992- | 1570 | u*A*uucaAaggaGcCuGga |
| | SS | | au*a | AS | | g*u*u |
| AV01993 | AV01993- | 1366 | a*c*uccaggCuCcUuugaa | AV01993- | 1571 | u*A*auucAaaggAgCcUgg |
| | SS | | uu*a | AS | | a*g*u |
| AV01994 | AV01994- | 1367 | c*u*ccaggcUcCuUugaau | AV01994- | 1572 | u*U*aauuCaaagGaGcCug |
| | SS | | ua*a | AS | | g*a*g |
| AV01995 | AV01995- | 1368 | c*c*aggcucCuUuGaauua | AV01995- | 1573 | u*U*guaaUucaaAgGaGcc |
| | SS | | ca*a | AS | | u*g*g |
| AV01996 | AV01996- | 1369 | c*a*ggcuccUuUgAauuac | AV01996- | 1574 | u*G*uguaAuucaAaGgAgc |
| | SS | | ac*a | AS | | c*u*g |
| AV01997 | AV01997- | 1370 | a*g*gcuccuUuGaAuuaca | AV01997- | 1575 | u*A*guguAauucAaAgGag |
| | SS | | cu*a | AS | | c*c*u |
| AV01998 | AV01998- | 1371 | g*g*cuccuuUgAaUuacac | AV01998- | 1576 | u*C*agugUaauuCaAaGga |
| | SS | | ug*a | AS | | g*c*c |
| AV01999 | AV01999- | 1372 | g*c*uccuuuGaAuUacacu | AV01999- | 1577 | u*U*caguGuaauUcAaAgg |
| | SS | | ga*a | AS | | a*g*c |
| AV02000 | AV02000- | 1373 | u*u*guuaaaGgAgUgaaug | AV02000- | 1578 | u*A*acauUcacuCcUuUaa |
| | SS | | uu*a | AS | | c*a*a |
| AV02001 | AV02001- | 1374 | u*g*uuaaagGaGuGaaugu | AV02001- | 1579 | u*A*aacaUucacUcCuUua |
| | SS | | uu*a | AS | | a*c*a |
| AV02002 | AV02002- | 1375 | g*u*uaaaggAgUgAauguu | AV02002- | 1580 | u*C*aaacAuucaCuCcUuu |
| | SS | | ug*a | AS | | a*a*c |
| AV02003 | AV02003- | 1376 | u*u*aaaggaGuGaAuguuu | AV02003- | 1581 | u*G*caaaCauucAcUcCuu |
| | SS | | gc*a | AS | | u*a*a |
| AV02004 | AV02004- | 1377 | u*a*aaggagUgAaUguuug | AV02004- | 1582 | u*G*gcaaAcauuCaCuCcu |
| | SS | | cc*a | AS | | u*u*a |
| AV02005 | AV02005- | 1378 | a*a*ggagugAaUgUuugcc | AV02005- | 1583 | u*U*uggcAaacaUuCaCuc |
| | SS | | aa*a | AS | | c*u*u |
| AV02006 | AV02006- | 1379 | a*g*gagugaAuGuUugcca | AV02006- | 1584 | u*C*uuggCaaacAuUcAcu |
| | SS | | ag*a | AS | | c*c*u |
| AV02007 | AV02007- | 1380 | g*g*agugaaUgUuUgccaa | AV02007- | 1585 | u*U*cuugGcaaaCaUuCac |
| | SS | | ga*a | AS | | u*c*c |
| AV02008 | AV02008- | 1381 | g*a*gugaauGuUuGccaag | AV02008- | 1586 | u*C*ucuuGgcaaAcAuUca |
| | SS | | ag*a | AS | | c*u*c |
| AV02009 | AV02009- | 1382 | a*g*ugaaugUuUgCcaaga | AV02009- | 1587 | u*U*cucuUggcaAaCaUuc |
| | SS | | ga*a | AS | | a*c*u |

In some embodiments of table 2 Chemically modified PKK RNAi agent sense strand sequences of nucleotides from SEQ ID NO: 767 of Homo sapiens kallikrein B1 (KLKB1 or PKK) mRNA sequence [NCBI Reference Sequence: NM_000892.5], Range in NM_000892.5 as shown in table 2-1

TABLE 2-1
Sense strand sequences of nucleotides range from SEQ ID NO: 767

| | Sense | | | Unmodified |
|---|---|---|---|---|
| Duplex | strand | SEQ ID NO | Range in | SEQ ID NO |
| ID | SS# | in table 2 | NM_000892.5 | in table 1 |
| AV01980 | AV01980-SS | 1353 | 1054-1074 | 943 |
| AV01981 | AV01981-SS | 1354 | 1055-1075 | 944 |
| AV01982 | AV01982-SS | 1355 | 1056-1076 | 945 |
| AV01983 | AV01983-SS | 1356 | 1057-1077 | 946 |
| AV01984 | AV01984-SS | 1357 | 1058-1078 | 947 |
| AV01960 | AV01960-SS | 1333 | 1059-1079 | 923 |
| AV01985 | AV01985-SS | 1358 | 1060-1080 | 948 |
| AV01986 | AV01986-SS | 1359 | 1061-1081 | 949 |
| AV01987 | AV01987-SS | 1360 | 1062-1082 | 950 |
| AV01988 | AV01988-SS | 1361 | 1063-1083 | 951 |
| AV01989 | AV01989-SS | 1362 | 1064-1084 | 952 |
| AV01965 | AV01965-SS | 1338 | 1065-1085 | 928 |
| AV01966 | AV01966-SS | 1339 | 1066-1086 | 929 |
| AV01967 | AV01967-SS | 1340 | 1067-1087 | 930 |
| AV01968 | AV01968-SS | 1341 | 1068-1088 | 931 |
| AV01969 | AV01969-SS | 1342 | 1069-1089 | 932 |
| AV02000 | AV02000-SS | 1373 | 1014-1034 | 963 |
| AV02001 | AV02001-SS | 1374 | 1015-1035 | 964 |
| AV02002 | AV02002-SS | 1375 | 1016-1036 | 965 |
| AV02003 | AV02003-SS | 1376 | 1017-1037 | 966 |
| AV02004 | AV02004-SS | 1377 | 1018-1038 | 967 |
| AV00751 | AV00751-SS | 519 | 1019-1039 | 71 |
| AV02005 | AV02005-SS | 1378 | 1020-1040 | 968 |
| AV02006 | AV02006-SS | 1379 | 1021-1041 | 969 |
| AV02007 | AV02007-SS | 1380 | 1022-1042 | 970 |
| AV02008 | AV02008-SS | 1381 | 1023-1043 | 971 |
| AV02009 | AV02009-SS | 1382 | 1024-1044 | 972 |
| AV01970 | AV01970-SS | 1236 | 1456-1476 | 933 |
| AV01971 | AV01971-SS | 1237 | 1457-1477 | 934 |
| AV01972 | AV01972-SS | 1238 | 1458-1478 | 935 |
| AV01973 | AV01973-SS | 1239 | 1459-1479 | 936 |
| AV01974 | AV01974-SS | 1240 | 1460-1480 | 937 |
| AV00045 | AV0045-SS | 492 | 1461-1481 | 44 |
| AV01975 | AV01975-SS | 1241 | 1462-1482 | 938 |
| AV01976 | AV01976-SS | 1242 | 1463-1483 | 939 |
| AV01977 | AV01977-SS | 1243 | 1464-1484 | 940 |
| AV01978 | AV01978-SS | 1244 | 1465-1485 | 941 |
| AV01979 | AV01979-SS | 1245 | 1466-1486 | 942 |
| AV01990 | AV01990-SS | 1256 | 1543-1563 | 953 |
| AV01991 | AV01991-SS | 1257 | 1544-1564 | 954 |
| AV01992 | AV01992-SS | 1258 | 1545-1565 | 955 |
| AV01993 | AV01993-SS | 1259 | 1546-1566 | 956 |
| AV01994 | AV01994-SS | 1260 | 1547-1567 | 957 |
| AV00781 | AV00781-SS | 549 | 1548-1568 | 101 |
| AV01995 | AV01995-SS | 1261 | 1549-1569 | 958 |
| AV01996 | AV01996-SS | 1262 | 1550-1570 | 959 |
| AV01997 | AV01997-SS | 1263 | 1551-1571 | 960 |
| AV01998 | AV01998-SS | 1264 | 1552-1572 | 961 |
| AV01999 | AV01999-SS | 1265 | 1553-1573 | 962 |
| AV00031 | AV00031-SS | 478 | 1106-1126 | 29 |
| AV00032 | AV00032-SS | 479 | 1108-1128 | 30 |
| AV00768 | AV00768-SS | 536 | 1109-1129 | 88 |
| AV00033 | AV00033-SS | 480 | 1110-1130 | 32 |
| AV00769 | AV00769-SS | 537 | 1112-1132 | 89 |
| AV00034 | AV00734-SS | 502 | 1113-1133 | 33 |
| AV00770 | AV00770-SS | 538 | 1115-1135 | 90 |
| AV00771 | AV00771-SS | 539 | 1116-1136 | 91 |
| AV00772 | AV00772-SS | 540 | 1117-1137 | 92 |
| AV00773 | AV00773-SS | 541 | 1118-1138 | 93 |
| AV00791 | AV00791-SS | 559 | 2034-3054 | 111 |
| AV00792 | AV00792-SS | 560 | 2035-2055 | 112 |

Table 3 discloses certain chemically modified PKK RNAi agent antisense strand and sense strand sequences and dsRNAs of the invention. In some embodiments of methods of the invention, RNAi agents shown in Table 3 are administered to a cell and/or subject. In some embodiments of methods of the invention, an RNAi agent comprising a polynucleotide sequence shown in Table 3 is administered to a subject. In some embodiments of the invention an RNAi agent administered to a subject comprises a duplex identified in column one in Table 3, wherein the duplex comprises the sequence modifications and/or delivery compound show in the sense and antisense strand sequences in columns four and seven, respectively, in the same row in Table 3. The sequences were used in certain in vivo testing studies described elsewhere herein. A sequence shown in Table 3 may in some embodiments, be attached to (also referred to herein as “conjugated to”) a compound for delivery, a non-limiting example of which is a GalNAc-containing compound. Certain embodiments of delivery compounds are identified in Table 3 as “GLS-5”, “GLS-15” or “GLX-0” on sense strands in column four. As used herein and shown in Table 3, “GLS-5”, “GLS-15” and “GLX-0” indicate a GalNAc-containing compound. As used herein, “GLX” is used to represent either a “GLS” or a GLO” delivery compound (“X” can be either “S” or “O”) and GLX-0 can be any of the GLS and GLO delivery compounds that can be attached to 3′-end of oligonucleotide during synthesis. For example, though not intended to be limiting: GLX-13 and GLX-14 can be attached to the 3′ end of an oligonucleotide of the invention during synthesis. In some embodiments of the invention, the delivery compound shown in Table 3 as “GLS-5”, “GLS-15” or “GLX-0” is replaced with any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is provided elsewhere herein. One skilled in the art will be able to prepare and use a dsRNA compound of the invention in which the attached delivery compound is one of GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16. Each row of Table 3 provides a Duplex AD # assigned to the duplex of the sense and antisense sequences in that row of the table. For example, Duplex AD #AD00311 is the duplex comprising sense strand SEQ ID NO: 693 and antisense strand SEQ ID NO: 740. Each line in Table 3 provides a sense strand and an antisense strand, and discloses the duplex of the sense and antisense strands shown. The “Sense strand SS #” in Table 3 column two is the assigned identifier for the Sense Sequence (including modifications) shown column four in the same row. The “Antisense strand AS #” in Table 3 column five is the assigned identifier for the Antisense sequence (including modifications) shown in column seven. An identifier for certain attached GalNAc-containing GLO or GLS compounds is shown as GLS-5, GLS-15 or GLX-0, and it will be understood that another of the GLO or GLS compounds may substitute the compounds shown as GLS-5, GLS-15 or GLX-0, with the resulting compound included in an embodiment of a method and/or a composition of the invention. In Table 3 SEQ ID NOs: 673-719, 1588-1637 are sense strand sequences and SEQ ID NO: 720-766, 1638-1687 are antisense strand sequences. In Table 3 delivery molecules are indicated as “GLX-0” at the 3′ end of sense strands SEQ ID NOs: 673-697; “GLS-5” at the 5′ end of sense strands SEQ ID NOs: 698-709; and GLS-15 at the 5′ end of sense strands 710-719. Chemical modifications are indicated as: upper case: 2′-Fluoro; lower case: 2′-OMe; thiophosphate: *; and Invab: inverted abasic.

TABLE 3
Chemically modified PKK RNAi agent antisense strand and sense strand sequences.
Sequences were used in certain in vivo studies described elsewhere herein.
All sequences are shown 5′ to 3′. Chemical modifications are indicated as:
upper case: 2′-Fluoro; lower case: 2′-OMe; thiophosphate: *;
Invab = inverted abasic.

| | Sense | SEQ | | Antisense | SEQ | |
|---|---|---|---|---|---|---|
| Duplex | strand | ID | | strand | ID | |
| ID | SS# | NO | Sense Strand | AS# | NO | Antisense Strand |
| AD00117 | AD00117-SS | 673 | g*c*aaaagaAcUuUa | AD00117-AS | 720 | u*U*ucagGuaaaGuU |
| | | | ccugaa*a(GLX-0) | | | cUuuu*g*c |
| AD00118 | AD00118-SS | 674 | g*u*cuuugaGaUuGu | AD00118-AS | 721 | u*U*guuaCacaaUcU |
| | | | guaaca*a(GLX-0) | | | cAaag*a*c |
| AD00119 | AD00119-SS | 675 | g*a*agagaaGuGuAa | AD00119-AS | 722 | u*A*aacaCuuacAcU |
| | | | guguuu*a(GLX-0) | | | uCucu*u*c |
| AD00120 | AD00120-SS | 676 | g*a*agcacaAuUuAu | AD00120-AS | 723 | u*G*uuggUauaaAuU |
| | | | accaac*a(GLX-0) | | | gUgcu*u*c |
| AD00121 | AD00121-SS | 677 | g*a*ucgagaCaUuUa | AD00121-AS | 724 | u*C*cuuuAuaaaUgU |
| | | | uaaagg*a(GLX-0) | | | cUcga*u*c |
| AD00274 | AD00274-SS | 678 | g*g*gauguaGcUuCc | AD00274-AS | 725 | u*G*uacaUggaaGcU |
| | | | auguac*a(GLX-0) | | | aCauc*c*c |
| AD00275 | AD00275-SS | 679 | g*u*aacgugGaAuCu | AD00275-AS | 726 | u*G*aaucCagauUcC |
| | | | ggauuc*a(GLX-0) | | | aCguu*a*c |
| AD00276 | AD00276-SS | 680 | a*g*aaguguAaGuGu | AD00276-AS | 727 | u*U*aagaAacacUuA |
| | | | uucuua*a(GLX-0) | | | cAcuu*c*u |
| AD00277 | AD00277-SS | 681 | a*g*uguaagUgUuUc | AD00277-AS | 728 | u*U*cuuaAgaaaCaC |
| | | | uuaaga*a(GLX-0) | | | uUaca*c*u |
| AD00278 | AD00278-SS | 682 | a*u*acaccuUuCuCa | AD00278-AS | 729 | u*U*auuuGugagAaA |
| | | | caaaua*a(GLX-0) | | | gGugu*a*u |
| AD00279 | AD00279-SS | 683 | g*a*agggaaUcAuGa | AD00279-AS | 730 | u*G*cgauAucauGaU |
| | | | uaucgc*a(GLX-0) | | | uCccu*u*c |
| AD00280 | AD00280-SS | 684 | c*c*aucgagAcAuUu | AD00280-AS | 731 | u*C*uuuaUaaauGuC |
| | | | auaaag*a(GLX-0) | | | uCgau*g*g |
| AD00303 | AD00303-SS | 685 | u*g*guugcuUcUuGa | AD00303-AS | 732 | u*U*auCuuucaaGaA |
| | | | aagaua*a(GLX-0) | | | gCaac*c*a |
| AD00304 | AD00304-SS | 686 | g*g*uuagcaGuGuUg | AD00304-AS | 733 | u*A*uuCuucaacAcU |
| | | | aagaau*a(GLX-0) | | | gCuaa*c*c |
| AD00305 | AD00305-SS | 687 | u*c*cucuacUcCuCa | AD00305-AS | 734 | u*U*uuUcuugagGaG |
| | | | agaaaa*a(GLX-0) | | | uAgag*g*a |
| AD00306 | AD00306-SS | 688 | g*c*cugcaaAaGaAc | AD00306-AS | 735 | a*G*guAaaguucUuU |
| | | | uuuacc*u(GLX-0) | | | uGcag*g*c |
| AD00307 | AD00307-SS | 689 | c*c*ugcaaaAgAaCu | AD00307-AS | 736 | u*A*ggUaaaguuCuU |
| | | | uuaccu*a(GLX-0) | | | uUgca*g*g |
| AD00308 | AD00308-SS | 690 | u*a*ccugaaCcCuGc | AD00308-AS | 737 | u*A*gaAuggcagGgU |
| | | | cauucu*a(GLX-0) | | | uCagg*u*a |
| AD00309 | AD00309-SS | 691 | a*c*uuuggaGgAgAa | AD00309-AS | 738 | u*C*aaUucuucuCcU |
| | | | gaauug*a(GLX-0) | | | cCaaa*g*u |
| AD00310 | AD00310-SS | 692 | g*a*ggagaaGaAuUg | AD00310-AS | 739 | u*C*acAuucaauUcU |
| | | | aaugug*a(GLX-0) | | | uCucc*u*c |
| AD00311 | AD00311-SS | 693 | u*u*cgcuguCaGuUu | AD00311-AS | 740 | u*A*guGaaaaacUgA |
| | | | uucacu*a(GLX-0) | | | cAgcg*a*a |
| AD00312 | AD00312-SS | 694 | c*g*cugucaGuUuUu | AD00312-AS | 741 | u*U*aaGugaaaaAcU |
| | | | cacuua*a(GLX-0) | | | gAcag*c*g |
| AD00313 | AD00313-SS | 695 | u*g*guuacuCuUuGa | AD00313-AS | 742 | u*A*caAucucaaAgA |
| | | | gauugu*a(GLX-0) | | | gUaac*c*a |
| AD00314 | AD00314-SS | 696 | u*g*uguggaGgGuCa | AD00314-AS | 743 | u*U*auGagugacCcU |
| | | | cucaua*a(GLX-0) | | | cCaca*c*a |
| AD00315 | AD00315-SS | 697 | u*c*ugucagAcAuUa | AD00315-AS | 744 | u*C*uuUuguaauGuC |
| | | | caaaag*a(GLX-0) | | | uGaca*g*a |
| AD00398 | AD00398-SS | 698 | (GLS-5)*(Invab)* | AD00398-AS | 745 | u*C*auucUucaaCaC |
| | | | guuagcagUgUuGaag | | | uGcua*a*c |
| | | | aauga*(Invab) | | | |
| AD00399 | AD00399-SS | 699 | (GLS-5)*(Invab)* | AD00399-AS | 746 | u*G*agaaUccagAuU |
| | | | aacguggaAuCuGgau | | | cCacg*u*u |
| | | | ucuca*(Invab) | | | |
| AD00400 | AD00400-SS | 700 | (GLS-5)*(Invab)* | AD00400-AS | 747 | u*C*uucaGugagAaU |
| | | | gaucuggaUuCuCacu | | | cCaga*u*c |
| | | | gaaga*(Invab) | | | |
| AD00401 | AD00401-SS | 701 | (GLS-5)*(Invab)* | AD00401-AS | 748 | u*U*cuuuUgcagGuU |
| | | | gccuuuuaAcCuGcaa | | | aAaag*g*c |
| | | | aagaa*(Invab) | | | |
| AD00402 | AD00402-SS | 702 | (GLS-5)*(Invab)* | AD00402-AS | 749 | u*G*uaaaGuucuUuU |
| | | | aaccugcaAaAgAacu | | | gCagg*u*u |
| | | | uuaca*(Invab) | | | |
| AD00403 | AD00403-SS | 703 | (GLS-5)*(Invab)* | AD00403-AS | 750 | u*A*aacaUucacUcC |
| | | | gguuaaagGaGuGaau | | | uUuaa*c*c |
| | | | guuua*(Invab) | | | |
| AD00404 | AD00404-SS | 704 | (GLS-5)*(Invab)* | AD00404-AS | 751 | u*G*uuacAcaauCuC |
| | | | acucuuugAgAuUgug | | | aAaga*g*u |
| | | | uaaca*(Invab) | | | |
| AD00405 | AD00405-SS | 705 | (GLS-5)*(Invab)* | AD00405-AS | 752 | u*U*ucagUguaaUuC |
| | | | cuccuuugAaUuAcac | | | aAagg*a*g |
| | | | ugaaa*(Invab) | | | |
| AD00406 | AD00406-SS | 706 | (GLS-5)*(Invab)* | AD00406-AS | 753 | u*U*cuucAacacUgC |
| | | | aagguuagCaGuGuug | | | uAacc*u*u |
| | | | aagaa*(Invab) | | | |
| AD00407 | AD00407-SS | 707 | (GLS-5)*(Invab)* | AD00407-AS | 754 | u*A*uuugUgagaAaG |
| | | | gauacaccUuUcUcac | | | gUgua*u*c |
| | | | aaaua*(Invab) | | | |
| AD00408 | AD00408-SS | 708 | (GLS-5)*(Invab)* | AD00408-AS | 755 | u*U*uauuUgugaGaA |
| | | | gacaccuuUcUcAcaa | | | aGgug*u*c |
| | | | auaaa*(Invab) | | | |
| AD00409 | AD00409-SS | 709 | (GLS-5)*(Invab)* | AD00409-AS | 756 | u*U*cuuuUguaaUgU |
| | | | cugucagaCaUuAcaa | | | cUgac*a*g |
| | | | aagaa*(Invab) | | | |
| AD00278-1 | AD00278-1-SS | 710 | (GLS-15)*(Invab) | AD00278-1-AS | 757 | u*U*auuuGugagAaA |
| | | | *auacaccuUuCuCac | | | gGugu*a*u |
| | | | aaauaa*(Invab) | | | |
| AD00311-1 | AD00311-1-SS | 711 | (GLS-15)*(Invab) | AD00311-1-AS | 758 | u*A*guGaaaaacUgA |
| | | | *uucgcuguCaGuUuu | | | cAgcg*a*a |
| | | | ucacua*(Invab) | | | |
| AD00312-1 | AD00312-1-SS | 712 | (GLS-15)*(Invab) | AD00312-1-AS | 759 | u*U*aaGugaaaaAcU |
| | | | *cgcugucaGuUuUuc | | | gAcag*c*g |
| | | | acuuaa*(Invab) | | | |
| AD00407-1 | AD00407-1-SS | 713 | (GLS-15)*(Invab) | AD00407-1-AS | 760 | u*A*uuugUgagaAaG |
| | | | *gauacaccUuUcUca | | | gUgua*u*c |
| | | | caaaua*(Invab) | | | |
| AD00502 | AD00502-SS | 714 | (GLS-15)*(Invab) | AD00502-AS | 761 | u*U*gaaaAacugAcA |
| | | | *cauucgcuGuCaGuu | | | gCgaa*u*g |
| | | | uuucaa*(Invab) | | | |
| AD00503 | AD00503-SS | 715 | (GLS-15)*(Invab) | AD00503-AS | 762 | u*G*ugaaAaacuGaC |
| | | | *cuucgcugUcAgUuu | | | aGcga*a*g |
| | | | uucaca*(Invab) | | | |
| AD00504 | AD00504-SS | 716 | (GLS-15)*(Invab) | AD00504-AS | 763 | u*A*agugAaaaaCuG |
| | | | *ccgcugucAgUuUuu | | | aCagc*g*g |
| | | | cacuua*(Invab) | | | |
| AD00505 | AD00505-SS | 717 | (GLS-15)*(Invab) | AD00505-AS | 764 | u*A*uaagUgaaaAaC |
| | | | *ccugucagUuUuUca | | | uGaca*g*g |
| | | | cuuaua*(Invab) | | | |
| AD00506 | AD00506-SS | 718 | (GLS-15)*(Invab) | AD00506-AS | 765 | u*G*aauaAgugaAaA |
| | | | *cgucaguuUuUcAcu | | | aCuga*c*g |
| | | | uauuca*(Invab) | | | |
| AD00507 | AD00507-SS | 719 | (GLS-15)*(Invab) | AD00507-AS | 766 | u*U*aaucUuaagAaA |
| | | | *cuaaguguUuCuUaa | | | cAcuu*a*g |
| | | | gauuaa*(Invab) | | | |
| AD00559 | AD00559-SS | 1588 | (GLS-15)*(Invab) | AD00559-AS | 1638 | u*A*agaaGgcguUuU |
| | | | *cucuaugaAaAcGcc | | | cAuag*a*g |
| | | | uucuua*(Invab) | | | |
| AD00560 | AD00560-SS | 1589 | (GLS-15)*(Invab) | AD00560-AS | 1639 | u*C*gauaUcaugAuU |
| | | | *agaagggaAuCaUga | | | cCcuu*c*u |
| | | | uaucga*(Invab) | | | |
| AD00561 | AD00561-SS | 1590 | (GLS-15)*(Invab) | AD00561-AS | 1640 | u*C*agaaUuugaCuU |
| | | | *ugaguucaAgUcAaa | | | gAacu*c*a |
| | | | uucuga*(Invab) | | | |
| AD00562 | AD00562-SS | 1591 | (GLS-15)*(Invab) | AD00562-AS | 1641 | u*U*ccuuUauaaAuG |
| | | | *gucgagacAuUuAua | | | uCucg*a*c |
| | | | aaggaa*(Invab) | | | |
| AD00563 | AD00563-SS | 1592 | (GLS-15)*(Invab) | AD00563-AS | 1642 | u*U*ucaaCacugCuA |
| | | | *cuaagguuAgCaGug | | | aCcuu*a*g |
| | | | uugaaa*(Invab) | | | |
| AD00564 | AD00564-SS | 1593 | (GLS-15)*(Invab) | AD00564-AS | 1643 | u*C*aaucUcaaaGaG |
| | | | *cugguuacUcUuUga | | | uAacc*a*g |
| | | | gauuga*(Invab) | | | |
| AD00565 | AD00565-SS | 1594 | (GLS-15)*(Invab) | AD00565-AS | 1644 | u*C*caagAagagUuU |
| | | | *gaggaacaAaCuCuu | | | gUucc*u*c |
| | | | cuugga*(Invab) | | | |
| AD00566 | AD00566-SS | 1595 | (GLS-15)*(Invab) | AD00566-AS | 1645 | u*A*auagCaaacAcC |
| | | | *cacccaagGuGuUug | | | uUggg*u*g |
| | | | cuauua*(Invab) | | | |
| AD00567 | AD00567-SS | 1596 | (GLS-15)*(Invab) | AD00567-AS | 1646 | u*C*uaucUuucaAgA |
| | | | *gguugcuuCuUgAaa | | | aGcaa*c*c |
| | | | gauaga*(Invab) | | | |
| AD00568 | AD00568-SS | 1597 | (GLS-15)*(Invab) | AD00568-AS | 1647 | u*G*cacuUauuuGaU |
| | | | *uguggucaUcAaAua | | | gAcca*c*a |
| | | | agugca*(Invab) | | | |
| AD00569 | AD00569-SS | 1598 | (GLS-15)*(Invab) | AD00569-AS | 1648 | u*U*cuucUgggaGuA |
| | | | *gauucuuuAcUcCca | | | aAgaa*u*c |
| | | | gaagaa*(Invab) | | | |
| AD00570 | AD00570-SS | 1599 | (GLS-15)*(Invab) | AD00570-AS | 1649 | u*G*uaauUcaaaGgA |
| | | | *uccaggcuCcUuUga | | | gCcug*g*a |
| | | | auuaca*(Invab) | | | |
| AD00571 | AD00571-SS | 1600 | (GLS-15)*(Invab) | AD00571-AS | 1650 | u*G*gcauAuuggUuU |
| | | | *guuccaaaAaCcAau | | | uUgga*a*c |
| | | | augcca*(Invab) | | | |
| AD00572 | AD00572-SS | 1601 | (GLS-15)*(Invab) | AD00572-AS | 1651 | u*U*uaccCaacaGuU |
| | | | *guauaccaAcUgUug | | | gGuau*a*c |
| | | | gguaaa*(Invab) | | | |
| AD00573 | AD00573-SS | 1602 | (GLS-15)*(Invab) | AD00573-AS | 1652 | u*U*ugacUccucUcA |
| | | | *guugauauGaGaGga | | | uAuca*a*c |
| | | | gucaaa*(Invab) | | | |
| AD00574 | AD00574-SS | 1603 | (GLS-15)*(Invab) | AD00574-AS | 1653 | u*U*uucuUgaggAgU |
| | | | *uuccucuaCuCcUca | | | aGagg*a*a |
| | | | agaaaa*(Invab) | | | |
| AD00575 | AD00575-SS | 1604 | (GLS-15)*(Invab) | AD00575-AS | 1654 | u*G*aguaAagaaUaA |
| | | | *guuucacuUaUuCuu | | | gUgaa*a*c |
| | | | uacuca*(Invab) | | | |
| AD00576 | AD00576-SS | 1605 | (GLS-15)*(Invab) | AD00576-AS | 1655 | u*A*gaaaCacuuAcA |
| | | | *agagaaguGuAaGug | | | cUucu*c*u |
| | | | uuucua*(Invab) | | | |
| AD00577 | AD00577-SS | 1606 | (GLS-15)*(Invab) | AD00577-AS | 1656 | u*A*gauuUaaaaUgC |
| | | | *gauaguggCaUuUua | | | cAcua*u*c |
| | | | aaucua*(Invab) | | | |
| AD00578 | AD00578-SS | 1607 | (GLS-15)*(Invab) | AD00578-AS | 1657 | u*G*uuuuUggaaUuC |
| | | | *guacacugAaUuCca | | | aGugu*a*c |
| | | | aaaaca*(Invab) | | | |
| AD00586 | AD00586-SS | 1608 | (GLS-15)*(Invab) | AD00586-AS | 1658 | u*A*uuuuAgaauGgC |
| | | | *gaacccugCcAuUcu | | | aGggu*u*c |
| | | | aaaaua*(Invab) | | | |
| AD00587 | AD00587-SS | 1609 | (GLS-15)*(Invab) | AD00587-AS | 1659 | u*U*aaauUuuagAaU |
| | | | *cccugccaUuCuAaa | | | gGcag*g*g |
| | | | auuuaa*(Invab) | | | |
| AD00588 | AD00588-SS | 1610 | (GLS-15)*(Invab) | AD00588-AS | 1660 | u*U*ggcaAacauUcA |
| | | | *gaaggaguGaAuGuu | | | cUccu*u*c |
| | | | ugccaa*(Invab) | | | |
| AD00589 | AD00589-SS | 1611 | (GLS-15)*(Invab) | AD00589-AS | 1661 | u*G*aaaaAcugaCaG |
| | | | *ugauucgcUgUcAgu | | | cGaau*c*a |
| | | | uuuuca*(Invab) | | | |
| AD00590 | AD00590-SS | 1612 | (GLS-15)*(Invab) | AD00590-AS | 1662 | u*A*auaaGugaaAaA |
| | | | *cugucaguUuUuCac | | | cUgac*a*g |
| | | | uuauua*(Invab) | | | |
| AD00591 | AD00591-SS | 1613 | (GLS-15)*(Invab) | AD00591-AS | 1663 | u*U*ccuuAcaguCuU |
| | | | *cucccagaAgAcUgu | | | cUggg*a*g |
| | | | aaggaa*(Invab) | | | |
| AD00592 | AD00592-SS | 1614 | (GLS-15)*(Invab) | AD00592-AS | 1664 | u*A*aucuUaagaAaC |
| | | | *gguaagugUuUcUua | | | aCuua*c*c |
| | | | agauua*(Invab) | | | |
| AD00593 | AD00593-SS | 1615 | (GLS-15)*(Invab) | AD00593-AS | 1665 | u*A*uaauCuuaaGaA |
| | | | *uaaguguuUcUuAag | | | aCacu*u*a |
| | | | auuaua*(Invab) | | | |
| AD00594 | AD00594-SS | 1616 | (GLS-15)*(Invab) | AD00594-AS | 1666 | u*G*auuuAaaauGcC |
| | | | *cuauagugGcAuUuu | | | aCuau*a*g |
| | | | aaauca*(Invab) | | | |
| AD00595 | AD00595-SS | 1617 | (GLS-15)*(Invab) | AD00595-AS | 1667 | u*U*aaugUcugaCaG |
| | | | *guuaaaucUgUcAga | | | aUuua*a*c |
| | | | cauuaa*(Invab) | | | |
| AD00596 | AD00596-SS | 1618 | (GLS-15)*(Invab) | AD00596-AS | 1668 | u*A*caguUgguaUaA |
| | | | *gcacaauuUaUaCca | | | aUugu*g*c |
| | | | acugua*(Invab) | | | |
| AD00597 | AD00597-SS | 1619 | (GLS-15)*(Invab) | AD00597-AS | 1669 | u*U*aaaaUccagUcC |
| | | | *aguacaugGaCuGga | | | aUgua*c*u |
| | | | uuuuaa*(Invab) | | | |
| AD00665 | AD00665-SS | 1620 | (GLS-15)*(Invab) | AD00665-AS | 1670 | u*A*accuUuucuCcA |
| | | | *gaugacauGgAgAaa | | | uGuca*u*c |
| | | | agguu*a*(Invab) | | | |
| AD00666 | AD00666-SS | 1621 | (GLS-15)*(Invab) | AD00666-AS | 1671 | u*A*auugAcuccUcU |
| | | | *ugauaugaGaGgAgu | | | cAuau*c*a |
| | | | caauu*a*(Invab) | | | |
| AD00667 | AD00667-SS | 1622 | (GLS-15)*(Invab) | AD00667-AS | 1672 | u*U*aaaaUugacUcC |
| | | | *uaugagagGaGuCaa | | | uCuca*u*a |
| | | | uuuua*a*(Invab) | | | |
| AD00668 | AD00668-SS | 1623 | (GLS-15)*(Invab) | AD00668-AS | 1673 | u*G*caaaCauucAcU |
| | | | *guaaaggaGuGaAug | | | cCuuu*a*c |
| | | | uuugc*a*(Invab) | | | |
| AD00669 | AD00669-SS | 1624 | (GLS-15)*(Invab) | AD00669-AS | 1674 | u*A*gcgaAucauCuU |
| | | | *gugcacaaAgAuGau | | | uGugc*a*c |
| | | | ucgcu*a*(Invab) | | | |
| AD00670 | AD00670-SS | 1625 | (GLS-15)*(Invab) | AD00670-AS | 1675 | u*A*acugAcagcGaA |
| | | | *gagaugauUcGcUgu | | | uCauc*u*c |
| | | | caguu*a*(Invab) | | | |
| AD00671 | AD00671-SS | 1626 | (GLS-15)*(Invab) | AD00671-AS | 1676 | u*C*uuaaGaaacAcU |
| | | | *aaguguaaGuGuUuc | | | uAcac*u*u |
| | | | uuaag*a*(Invab) | | | |
| AD00672 | AD00672-SS | 1627 | (GLS-15)*(Invab) | AD00672-AS | 1677 | u*U*aguuUugguGaA |
| | | | *guuauuauUcAcCaa | | | uAaua*a*c |
| | | | aacua*a*(Invab) | | | |
| AD00673 | AD00673-SS | 1628 | (GLS-15)*(Invab) | AD00673-AS | 1678 | u*A*uuggUuuuuGgA |
| | | | *acugaauuCcAaAaa | | | aUuca*g*u |
| | | | ccaau*a*(Invab) | | | |
| AD00674 | AD00674-SS | 1629 | (GLS-15)*(Invab) | AD00674-AS | 1679 | u*U*uuucUcuaaAaU |
| | | | *ggacuggaUuUuAga | | | cCagu*c*c |
| | | | gaaaa*a*(Invab) | | | |
| AD00588-1 | AD00588-1-SS | 1630 | (GLS-15)*(Invab) | AD00588-1-AS | 1680 | u*U*ggCaaacauUcA |
| | | | *gaaggaguGaAugUu | | | cucCu*u*c |
| | | | ugcca*a*(Invab) | | | |
| AD00506-1 | AD00506-1-SS | 1631 | (GLS-15)*(Invab) | AD00506-1-AS | 1681 | u*G*aaUaagugaAaA |
| | | | *ugucaguuUuUcaCu | | | acuGa*c*a |
| | | | uauuc*a*(Invab) | | | |
| AD00506-2 | AD00506-2-SS | 1632 | (GLS-15)*(Invab) | AD00506-2-AS | 1682 | u*G*aAuaagugaaaA |
| | | | *ugucaguuUuUcaCu | | | acuga*c*a |
| | | | uauuc*a*(Invab) | | | |
| AD00506-3 | AD00506-3-SS | 1633 | (GLS-15)*(Invab) | AD00506-3-AS | 1683 | u*G*aA*uaagugaaa |
| | | | *ugucaguuU*uUcaC | | | A*acuga*c*a |
| | | | *uuauuc*a*(Invab) | | | |
| AD00506-4 | AD00506-′-SS | 1634 | (GLS-15)*(Invab) | AD00506-4-AS | 1684 | u*G*aauaAgugaAaA |
| | | | *ugucaguuUuUcAcu | | | aCuga*c*a |
| | | | uauuc*a*(Invab) | | | |
| AD00592-1 | AD00592-1-SS | 1635 | (GLS-15)*(Invab) | AD00592-1-AS | 1685 | u*A*auCuuaagaAaC |
| | | | *gguaagugUuUcuUa | | | acuUa*c*c |
| | | | agauu*a*(Invab) | | | |
| AD00570-1 | AD00570-- | 1636 | (GLS-15)*(Invab) | AD00570-1-AS | 1686 | u*G*uaAuucaaaGgA |
| | 1-SS | | *uccaggcuCcUuuGa | | | gccUg*g*a |
| | | | auuac*a*(Invab) | | | |
| AD00561-1 | AD00561- | 1637 | (GLS-15)*(Invab) | AD00561-1-AS | 1687 | u*C*agAauuugaCuU |
| | 1-SS | | *ugaguucaAgUcaAa | | | gaaCu*c*a |
| | | | uucug*a*(Invab) | | | |

In certain embodiments of the invention a dsRNA (also referred to herein as a “duplex”) is one disclosed in one of Tables 1-3. Each row in Tables 1-3 discloses a duplex comprising the sequence of the sense strand and the sequence of the antisense strand in that table row. In addition to the duplexes disclosed in Tables 1-3, it will be understood that in some embodiments, a duplex of the invention may include sense and antisense sequences shown in Tables 1-3, that differ by zero, one, two, or three nucleotides shown in a sequence shown in Tables 1-3. Thus, as non-limiting examples, in some embodiments, an antisense strand in a duplex of the invention may be SEQ ID NO: 225, 251, 258, 295, 325, 417, or 437, with zero, one, two, or three nucleotides differ from those in SEQ ID NO: 225, 251, 258, 295, 325, 417, or 437, respectively.

It will be understood that the sequence of the sense strand and the sequence of the antisense strand in a duplex of the invention may be independently selected. Thus, a dsRNA of the invention may comprise a sense strand and an antisense strand of a duplex disclosed in a row in Tables 1-3. Alternatively, in a dsRNA of the invention, one or both of the selected sense and antisense strand in the dsRNA may include sequences shown in Tables 1-3 but with one or both of the sense and antisense sequences including 1, 2, 3, or more nucleobase substitutions from the parent sequence. The selected sequences may in some embodiments be longer or shorter than their parent sequence. Thus, dsRNA agents included in the invention can but need not include exact sequences of the sense and antisense pairs disclosed as duplexes in Tables 1-3.

In some embodiments, a dsRNA agent comprises a sense strand and an antisense strand, nucleotide positions 2 to 18 in the antisense strand comprising a region of complementarity to a PKK RNA transcript, wherein the region of complementarity comprises at least 15 contiguous nucleotides that differ by 0, 1, 2, or 3 nucleotides from one of the antisense sequences listed in one of Tables 1-3, and optionally comprising a targeting ligand. In some instances, the region of complementarity to the PKK RNA transcript comprises at least 15, 16, 17, 18, or 19 contiguous nucleotides that differ by no more than 3 nucleotides from one of the antisense sequences listed in one of Tables 1-3. In some embodiments of a dsRNA agent of the invention, the antisense strand of the dsRNA is at least substantially complementary to any one of a target region of SEQ ID NO: 767 and is provided in any one of Tables 1-3. In some embodiments, an antisense strand of a dsRNA agent of the invention is fully complementary to any one of a target region of SEQ ID NO: 767 and is provided in any one of Tables 1-3. In some embodiments a dsRNA agent includes a sense strand sequence set forth in any one of Tables 1-3, and the sense strand sequence is at least substantially complementary to the antisense strand sequence in the dsRNA agent. In other embodiments, a dsRNA agent of the invention comprises a sense strand sequence set forth in any one of Tables 1-3, and the sense strand sequence is fully complementary to the antisense strand sequence in the dsRNA agent. In some instances, a dsRNA agent of the invention comprises an antisense strand sequence set forth in any one of Tables 1-3. Some embodiments of a dsRNA agent of the invention comprises the sense and antisense sequences disclosed as duplex in any of Tables 1-3. As described herein, it will be understood that the sense and antisense strands in a duplex of the invention may be independently selected.

### Mismatches

It is known to skilled in art, mismatches are tolerated for efficacy in dsRNA, especially if the mismatches are within terminal region of dsRNA. Certain mismatches are better tolerated in a dsRNA, for example mismatches with wobble base pairs G: U and A: C are tolerated better for efficacy (Du et el., Nucleic Acids Res. 2005 Mar. 21; 3 (5): 1671-7). In some embodiments of methods and compounds of the invention a PKK dsRNA agent may contain one or more mismatches to the PKK target sequence. In some embodiments, PKK dsRNA agent of the invention includes no mismatches. In certain embodiments, PKK dsRNA agent of the invention includes no more than 1, no more than 2, or no more than 3 mismatches to the PKK target sequence. In some embodiments of the invention, an antisense strand of a PKK dsRNA agent contains mismatches to a PKK target sequence that are not located in the center of the region of complementarity. In some embodiments, the antisense strand of the PKK dsRNA agent includes 1, 2, 3, 4, or more mismatches that are within the last 5, 4, 3, 2, or 1 nucleotides from one or both of the 5′ end and the 3′ end of the region of complementarity. Methods described herein and/or methods known in the art can be used to determine whether a PKK dsRNA agent containing a mismatch to a PKK target sequence is effective in inhibiting the expression of the PKK gene.

### Complementarity

As used herein, unless otherwise indicated, the term “complementary,” when used to describe a first nucleotide sequence (e.g., PKK dsRNA agent sense strand) in relation to a second nucleotide sequence (e.g., PKK dsRNA agent antisense strand), means the ability of an oligonucleotide or polynucleotide including the first nucleotide sequence to hybridize [form base pair hydrogen bonds under mammalian physiological conditions (or similar conditions in vitro)] and form a duplex or double helical structure under certain conditions with an oligonucleotide or polynucleotide including the second nucleotide sequence. Other conditions, such as physiologically relevant conditions as can be encountered inside an organism, can apply. A skilled artisan will be able to determine the set of conditions most appropriate for a test of complementarity of two sequences in accordance with the ultimate application of the hybridized nucleotides. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification.

Complementary sequences, for example, within a PKK dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. It will be understood that in embodiments when two oligonucleotides are designed to form, upon hybridization, one or more single-stranded overhangs, such overhangs are not regarded herein as mismatches with regard to the determination of complementarity. For example, a PKK dsRNA agent comprising one oligonucleotide 19 nucleotides in length and another oligonucleotide 20 nucleotides in length, wherein the longer oligonucleotide comprises a sequence of 19 nucleotides that is fully complementary to the shorter oligonucleotide, can yet be referred to as “fully complementary” for the purposes described herein. Thus, as used herein, “fully complementary” means that all (100%) of the bases in a contiguous sequence of a first polynucleotide will hybridize with the same number of bases in a contiguous sequence of a second polynucleotide. The contiguous sequence may comprise all or a part of a first or second nucleotide sequence.

The term “substantially complementary” as used herein means that in a hybridized pair of nucleobase sequences, at least about 85%, 86%, 87%, 88%, 89%, 90%, 91%, 92%, 93%, 94%, 95%, 96%, 97%, 98%, or 99%, but not all, of the bases in a contiguous sequence of a first polynucleotide will hybridize with the same number of bases in a contiguous sequence of a second polynucleotide. The term “substantially complementary” can be used in reference to a first sequence with respect to a second sequence if the two sequences include one or more, for example at least 1, 2, 3, 4, or 5 mismatched base pairs upon hybridization for a duplex up to 15, 16, 17, 18, 19, 20, 21, 22, 23, 24, 25, 26, 27, 28, 29 or 30 base pairs (bp), while retaining the ability to hybridize under the conditions most relevant to their ultimate application, e.g., inhibition of PKK gene expression via a RISC pathway.

The term, “partially complementary” may be used herein in reference to a hybridized pair of nucleobase sequences, in which at least 75%, but not all, of the bases in a contiguous sequence of a first polynucleotide will hybridize with the same number of bases in a contiguous sequence of a second polynucleotide. In some embodiments, “partially complementary” means at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bases in a contiguous sequence of a first polynucleotide will hybridize with the same number of bases in a contiguous sequence of a second polynucleotide

The terms “complementary,” “fully complementary,” “substantially complementary,” and “partially complimentary” are used herein in reference to the base matching between the sense strand and the antisense strand of a PKK dsRNA agent or between the antisense strand of a PKK dsRNA agent and a sequence of a target PKK mRNA. It will be understood that the term “antisense strand of a PKK dsRNA agent” may refer to the same sequence of a “PKK antisense polynucleotide agent”.

As used herein, the term “substantially identical” or “substantial identity” used in reference to a nucleic acid sequence means a nucleic acid sequence comprising a sequence with at least about 85% sequence identity or more, preferably at least 90%, at least 91%, at least 92%, at least 93%, at least 94%, at least 95%, at least 96%, at least 97%, at least 98%, or at least 99%, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompasses nucleotide sequences substantially identical to those disclosed herein, e.g., in Tables 1-3. In some embodiments, the sequences disclosed herein are exactly identical, or at least about 85%, 86%, 87%, 88%, 89%, 90%, 91%, 92%, 93%, 94%, 95%, 96%, 97%, 98%, or 99% percent identical to those disclosed herein, e.g., in Tables 1-3.

As used herein, the term “strand comprising a sequence” means an oligonucleotide comprising a chain of nucleotides that is described by the sequence referred to using the standard nucleotide nomenclature. The term “double-stranded RNA” or “dsRNA,” as used herein, refers to an RNAi that includes an RNA molecule or complex of molecules having a hybridized duplex region comprising two anti-parallel and substantially or fully complementary nucleic acid strands, which are referred to as having “sense” and “antisense” orientations with respect to a target PKK RNA. The duplex region can be of any length that permits specific degradation of a desired target PKK RNA through a RISC pathway, but will typically range from 9 to 30 base pairs in length, e.g., 15-30 base pairs in length. Considering a duplex between 9 and 30 base pairs, the duplex can be any length in this range, for example, 9, 10, 11, 12, 13, 14, 15, 16, 17, 18, 19, 20, 21, 22, 23, 24, 25, 26, 27, 28, 29, or 30, and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. PKK dsRNA agents generated in the cell by processing with Dicer and similar enzymes are generally in the range of 19-22 base pairs in length. One strand of the duplex region of a PKK dsDNA agent comprises a sequence that is substantially complementary to a region of a target PKK RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. In some embodiments of the invention, a hairpin look comprises at least 1, 2, 3, 4, 5, 6, 7, 8, 9, 10, 11, 12, 13, 14, 15, 16, 17, 18, 19, 20, or more unpaired nucleotides. Where the two substantially complementary strands of a PKK dsRNA agent are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA agent as described herein.

In some embodiments of the invention a PKK dsRNA agent may include a sense and antisense sequence that have no-unpaired nucleotides or nucleotide analogs at one or both terminal ends of the dsRNA agent. An end with no unpaired nucleotides is referred to as a “blunt end” and as having no nucleotide overhang. If both ends of a dsRNA agent are blunt, the dsRNA is referred to as “blunt ended.” In some embodiments of the invention, only a first end of a dsRNA agent is blunt, in some embodiments only a second end of a dsRNA agent is blunt, and in certain embodiments of the invention, both ends of a PKK dsRNA agent are blunt.

In some embodiments of dsRNA agents of the invention, the dsRNA does not have one or two blunt ends. In such instances there is at least one unpaired nucleotide at the end of a strand of a dsRNA agent. For example, when a 3′-end of one strand of a dsRNA extends beyond the 5′-end of the other strand, or vice versa, there is a nucleotide overhang. A dsRNA can comprise an overhang of at least 1, 2, 3, 4, 5, 6, or more nucleotides. A nucleotide overhang can comprise or consist of a nucleotide/nucleoside analog, including a deoxynucleotide/nucleoside. It will be understood that in some embodiments a nucleotide overhang is on a sense strand of a dsRNA agent, on an antisense strand of a dsRNA agent, or on both ends of a dsRNA agent and nucleotide(s) of an overhang can be present on the 5′ end, 3′ end or both ends of either an antisense or sense strand of a dsRNA. In certain embodiments of the invention, one or more of the nucleotides in an overhang is replaced with a nucleoside thiophosphate.

As used herein, the term “antisense strand” or “guide strand” refers to the strand of a PKK dsRNA agent that includes a region that is at least substantially complementary to a PKK target sequence. As used herein the term “sense strand,” or “passenger strand” refers to the strand of a PKK dsRNA agent that includes a region that is at least substantially complementary to a region of the antisense strand of the PKK dsRNA agent.

### Modifications

In some embodiments of the invention the RNA of a PKK RNAi agent is chemically modified to enhance stability and/or one or more other beneficial characteristics. Nucleic acids in certain embodiments of the invention may be synthesized and/or modified by methods well established in the art, for example, those described in “Current protocols in Nucleic Acid Chemistry,” Beaucage, S. L. et al. (Eds.), John Wiley & Sons, Inc., New York, N.Y., USA, which is incorporated herein by reference. Modifications that can be present in certain embodiments of PKK dsRNA agents of the invention include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in certain embodiments of PKK dsRNA agents, PKK antisense polynucleotides, and PKK sense polynucleotides of the invention include, but are not limited to RNAs comprising modified backbones or no natural internucleoside linkages. As a non-limiting example, an RNA having a modified backbone may not have a phosphorus atom in the backbone. RNAs that do not have a phosphorus atom in their internucleoside backbone may be referred to as oligonucleosides. In certain embodiments of the invention, a modified RNA has a phosphorus atom in its internucleoside backbone.

It will be understood that the term “RNA molecule” or “RNA” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. The terms “ribonucleoside” and “ribonucleotide” may be used interchangeably herein. An RNA molecule can be modified in the nucleobase structure or in the ribose-phosphate backbone structure, e.g., as described herein below, and molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. In some embodiments of the invention, an RNA molecule comprises at least 1, 2, 3, 4, 5, 6, 7, 8, 9, 10, 11, 12, 13, 14, 15, 16, 17, 18, 19, 20, or up to the full length of the PKK dsRNA agent molecule's ribonucleosides that are modified ribonucleosides. The modifications need not be the same for each of such a plurality of modified ribonucleosides in an RNA molecule. As used herein, the term “plurality” means more than one, so a plurality may be 2, 3, 4, 5, 6, 7, or more of the item to which the term refers. As a non-limiting example, a plurality of modified ribonucleosides in an RNA molecule means 2, 3, 4, 5, 6, 7, 8, 9, 10, 11, 12, 13, 14, 15, 16, 17, 18, 19, 20, or up to the full length of one or both strands of a PKK dsRNA agent molecule's ribonucleosides are modified ribonucleosides. It will be understood that a modified ribonucleosides in the sense and antisense strands may be independently selected.

dsRNA agents, PKK antisense polynucleotides, and/or PKK sense polynucleotides of the invention may, in some embodiments comprise one or more independently selected modified nucleotide and/or one or more independently selected non-phosphodiester linkage. As used herein the term “independently selected” used in reference to a selected element, such as a modified nucleotide, non-phosphodiester linkage, a targeting agent, a dsRNA agent, a duplex of the invention, etc., means that two or more selected elements can but need not be the same as each other.

As used herein, a “nucleotide base,” “nucleotide,” or “nucleobase” is a heterocyclic pyrimidine or purine compound, which is a standard constituent of all nucleic acids, and includes the bases that form the nucleotides adenine (a), guanine (g), cytosine (c), thymine (t), and uracil (u). A nucleobase may further be modified to include, though not intended to be limiting: universal bases, hydrophobic bases, promiscuous bases, size-expanded bases, and fluorinated bases. The term “ribonucleotide” or “nucleotide” may be used herein to refer to an unmodified nucleotide, a modified nucleotide, or a surrogate replacement moiety. Those in the art will recognize that guanine, cytosine, adenine, and uracil can be replaced by other moieties without substantially altering the base pairing properties of an oligonucleotide comprising a nucleotide bearing such replacement moiety.

In one embodiment, modified RNAs contemplated for use in methods and compositions described herein are peptide nucleic acids (PNAs) that have the ability to form the required duplex structure and that permit or mediate the specific degradation of a target RNA via a RISC pathway. In certain embodiments of the invention, a PKK RNA interference agent includes a single stranded RNA that interacts with a target PKK RNA sequence to direct the cleavage of the target PKK RNA.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Means of preparing phosphorus-containing linkages are routinely practiced in the art and such methods can be used to prepare certain modified PKK dsRNA agents, certain modified PKK antisense polynucleotides, and/or certain modified PKK sense polynucleotides of the invention.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts. Means of preparing modified RNA backbones that do not include a phosphorus atom are routinely practiced in the art and such methods can be used to prepare certain modified PKK dsRNA agents, certain modified PKK antisense polynucleotides, and/or certain modified PKK sense polynucleotides of the invention.

In certain embodiments of the invention, RNA mimetics are included in PKK dsRNAs, PKK antisense polynucleotides, and/or PKK sense polynucleotides, such as, but not limited to: replacement of the sugar and the internucleoside linkage, i.e., the backbone, of the nucleotide units with novel groups. In such embodiments, base units are maintained for hybridization with an appropriate PKK nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Means of preparing RNA mimetics are routinely practiced in the art and such methods can be used to prepare certain modified PKK dsRNA agents of the invention.

Some embodiments of the invention include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂-[wherein the native phosphodiester backbone is represented as —O—P—O—CH₂—]. Means of preparing RNAs with phosphorothioate backbones and oligonucleosides with heteroatom backbones are routinely practiced in the art and such methods can be used to prepare certain modified PKK dsRNA agents, certain PKK antisense polynucleotides, and/or certain PKK sense polynucleotides of the invention.

Modified RNAs can also contain one or more substituted sugar moieties. PKK dsRNAs, PKK antisense polynucleotides, and/or PKK sense polynucleotides of the invention may comprise one of the following at the 2′ position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂), OCH₃, O(CH₂)ₙNH₂, O(CH₂)ₙCH₃, O(CH₂)ₙONH₂, and O(CH₂)[ON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a PKK dsRNA agent, or a group for improving the pharmacodynamic properties of a PKK dsRNA agent, PKK antisense polynucleotide, and/or PKK sense polynucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Means of preparing modified RNAs such as those described are routinely practiced in the art and such methods can be used to prepare certain modified PKK dsRNA agents of the invention.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of a PKK dsRNA agent, PKK antisense polynucleotide, and/or PKK sense polynucleotide of the invention, particularly the 3′ position of the sugar on the 3′ terminal nucleotide or in 2′-5′ linked PKK dsRNAs, PKK antisense polynucleotides, or PKK sense polynucleotides, and the 5′ position of 5′ terminal nucleotide. PKK dsRNA agents, PKK antisense polynucleotides, and/or PKK sense polynucleotides may also have sugar mimetics such as cyclobutyl moieties in place of the pentofuranosyl sugar. Means of preparing modified RNAs such as those described are routinely practiced in the art and such methods can be used to prepare certain modified PKK dsRNA agents, PKK antisense polynucleotides, and/or PKK sense polynucleotides of the invention.

A PKK dsRNA agent, PKK antisense polynucleotide, and/or PKK sense polynucleotide may, in some embodiments, include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Additional nucleobases that may be included in certain embodiments of PKK dsRNA agents of the invention are known in the art, see for example: Modified Nucleosides in Biochemistry, Biotechnology and Medicine, Herdewijn, P. Ed. Wiley-VCH, 2008; The Concise Encyclopedia Of Polymer Science And Engineering, pages 858-859, Kroschwitz, J. L, Ed. John Wiley & Sons, 1990, English et al., Angewandte Chemie, International Edition, 1991, 30, 613, Sanghvi, Y S., Chapter 15, dsRNA Research and Applications, pages 289-302, Crooke, S. T. and Lebleu, B., Ed., CRC Press, 1993. Means of preparing dsRNAs, PKK antisense strand polynucleotides and/or PKK sense strand polynucleotides that comprise nucleobase modifications and/or substitutions such as those described herein are routinely practiced in the art and such methods can be used to prepare certain modified PKK dsRNA agents, PKK sense polynucleotides, and/or PKK antisense polynucleotides of the invention.

Certain embodiments of PKK dsRNA agents, PKK antisense polynucleotides, and/or PKK sense polynucleotides of the invention include RNA modified to include one or more locked nucleic acids (LNA). A locked nucleic acid is a nucleotide with a modified ribose moiety comprising an extra bridge connecting the 2′ and 4′ carbons. This structure effectively “locks” the ribose in the 3′-endo structural conformation. The addition of locked nucleic acids in a PKK dsRNA agent, PKK antisense polynucleotides, and/or PKK sense polynucleotides of the invention may increase stability in serum, and to reduce off-target effects (Elmen, J. et al., (2005) Nucleic Acids Research 33 (1): 439-447; Mook, O R. et al., (2007) Mol Canc Ther 6 (3): 833-843; Grunweller, A. et al., (2003) Nucleic Acids Research 31 (12): 3185-3193). Means of preparing dsRNA agents, PKK antisense polynucleotides, and/or PKK sense polynucleotides that comprise locked nucleic acid(s) are routinely practiced in the art and such methods can be used to prepare certain modified PKK dsRNA agents of the invention.

Certain embodiments of PKK dsRNA compounds, sense polynucleotides, and/or antisense polynucleotides of the invention, include at least one modified nucleotide, wherein the at least one modified nucleotide comprises: a 2′-O-methyl nucleotide, 2′-Fluoro nucleotide, 2′-deoxy nucleotide, 2′3′-seco nucleotide mimic, locked nucleotide, 2′-F-Arabino nucleotide, 2′-methoyxyethyl nucleotide, 2′-amino-modified nucleotide, 2′-alkyl-modified nucleotide, morpholino nucleotide, and 3′-OMe nucleotide, a nucleotide comprising a 5′-phosphorothioate group, or a terminal nucleotide linked to a cholesteryl derivative or dodecanoic acid bisdecylamide group, a 2′-amino-modified nucleotide, a phosphoramidate, or a non-natural base comprising nucleotide. In some embodiments, a PKK dsRNA compound includes an E-vinylphosphonate nucleotide at the 5′ end of the antisense strand, also referred to herein as the guide strand.

Certain embodiments of PKK dsRNA compounds, 3′ and 5′ end of sense polynucleotides, and/or 3′ end of antisense polynucleotides of the invention, include at least one modified nucleotide, wherein the at least one modified nucleotide comprises: abasic nucleotide, ribitol, inverted nucleotide, inverted abasic nucleotide, inverted 2′-OMe nucleotide, inverted 2′-deoxy nucleotide. It is known to skilled in art, including an abasic or inverted abasic nucleotide at the end of oligonucleotide enhances stability (Czauderna et al. Nucleic Acids Res. 2003; 31(11):2705-2716. doi:10.1093/nar/gkg393).

Certain embodiments of PKK dsRNA compounds, antisense polynucleotides of the invention, include at least one modified nucleotide, wherein the at least one modified nucleotide comprises unlocked nucleic acid nucleotide (UNA) or/and glycol nucleic acid nucleotide (GNA). It is known to skilled in art, UNA and GNA are thermally destabilizing chemical modifications, can significantly improves the off-target profile of a siRNA compound (Janas, et al., Nat Commun. 2018; 9(1):723. doi:10.1038/s41467-018-02989-4; Laursen et al., Mol BioSyst. 2010; 6:862-70).

Another modification that may be included in the RNA of certain embodiments of PKK dsRNA agents, PKK antisense polynucleotides, and/or PKK sense polynucleotides of the invention, comprises chemically linking to the RNA one or more ligands, moieties or conjugates that enhance one or more characteristics of the PKK dsRNA agent, PKK antisense polynucleotide, and/or PKK sense polynucleotide, respectively. Non-limiting examples of characteristics that may be enhanced are: PKK dsRNA agent, PKK antisense polynucleotide, and/or PKK sense polynucleotide activity, cellular distribution, delivery of a PKK dsRNA agent, pharmacokinetic properties of a PKK dsRNA agent, and cellular uptake of the PKK dsRNA agent. In some embodiments of the invention, a PKK dsRNA agent comprises one or more targeting groups or linking groups, which in certain embodiments of PKK dsRNA agents of the invention are conjugated to the sense strand. A non-limiting example of a targeting group is a compound comprising N-acetyl-galactosamine (GalNAc). The terms “targeting group”, “targeting agent”, “linking agent”, “targeting compound”, and “targeting ligand” may be used interchangeably herein. In certain embodiments of the invention a PKK dsRNA agent comprises a targeting compound that is conjugated to the 5′-terminal end of the sense strand. In certain embodiments of the invention a PKK dsRNA agent comprises a targeting compound that is conjugated to the 3′-terminal end of the sense strand. In some embodiments of the invention, a PKK dsRNA agent comprises a targeting group that comprises GalNAc. In certain embodiments of the invention a PKK dsRNA agent does not include a targeting compound conjugated to one or both of the 3′-terminal end and the 5′-terminal end of the sense strand. In certain embodiments of the invention a PKK dsRNA agent does not include a GalNAc containing targeting compound conjugated to one or both of the 5′-terminal end and the 3′-terminal end of the sense strand.

Additional targeting and linking agents are well known in the art, for example, targeting and linking agents that may be used in certain embodiments of the invention include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Certain embodiments of a composition comprising a PKK dsRNA agent, PKK antisense polynucleotide, and/or PKK sense polynucleotide may comprise a ligand that alters distribution, targeting, or etc. of the PKK dsRNA agent. In some embodiments of a composition comprising a PKK dsRNA agent of the invention, the ligand increases affinity for a selected target, e.g., molecule, cell or cell type, compartment, e.g., a cellular or organ compartment, tissue, organ or region of the body, as, e.g., compared to a species absent such a ligand. A ligand useful in a composition and/or method of the invention may be a naturally occurring substance, such as a protein (e.g., human serum albumin (HSA), low-density lipoprotein (LDL), or globulin); a carbohydrate (e.g., a dextran, pullulan, chitin, chitosan, inulin, cyclodextrin or hyaluronic acid); or a lipid. A ligand may also be a recombinant or synthetic molecule, such as a synthetic polymer, e.g., a synthetic polyamino acid or polyamine. Examples of polyamino acids are a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A ligand included in a composition and/or method of the invention may comprise a targeting group, non-limiting examples of which are a cell or tissue targeting agent, e.g., a lectin, glycoprotein, lipid or protein, e.g., an antibody that binds to a specified cell type such as a kidney cell or a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

A ligand included in a composition and/or method of the invention may be a protein, e.g., glycoprotein, or peptide, for example a molecule with a specific affinity for a co-ligand, or an antibody, for example an antibody, that binds to a specified cell type such as, but not limited to: a liver cell. A ligand useful in an embodiment of a composition and/or method of the invention can be a hormone or hormone receptor. A ligand useful in an embodiment of a composition and/or method of the invention can be a lipid, lectin, carbohydrates, vitamin, cofactor, multivalent lactose, multivalent galactose, N-acetyl-galactosamine, N-acetyl-glucosamine multivalent mannose, or multivalent fucose. A ligand useful in an embodiment of a composition and/or method of the invention can be a substance that can increase uptake of the PKK dsRNA agent into the cell, for example, by disrupting the cell's cytoskeleton, e.g., by disrupting the cell's microtubules, microfilaments, and/or intermediate filaments. Non-limiting examples of this type of agent are: taxon, vincristine, vinblastine, cytochalasin, nocodazole, japlakinolide, latrunculin A, phalloidin, swinholide A, indanocine, and myoservin.

In some embodiments, a ligand attached to a PKK dsRNA agent of the invention functions as a pharmacokinetic (PK) modulator. An example of a PK modulator that may be used in compositions and methods of the invention includes but is not limited to: a lipophile, a bile acid, a steroid, a phospholipid analogue, a peptide, a protein binding agent, PEG, a vitamin, cholesterol, a fatty acid, cholic acid, lithocholic acid, dialkylglycerides, diacylglyceride, a phospholipid, a sphingolipid, naproxen, ibuprofen, vitamin E, biotin, an aptamer that binds a serum protein, etc. Oligonucleotides comprising a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone may also be used in compositions and/or methods of the invention as ligands.

PKK dsRNA Agent Compositions

In some embodiments of the invention, a PKK dsRNA agent is in a composition. A composition of the invention may include one or more PKK dsRNA agent and optionally one or more of a pharmaceutically acceptable carrier, a delivery agent, a targeting agent, detectable label, etc. A non-limiting example of a targeting agent that may be useful according to some embodiments of methods of the invention is an agent that directs a PKK dsRNA agent of the invention to and/or into a cell to be treated. A targeting agent of choice will depend upon such elements as: the nature of the PKK-associated disease or condition, and on the cell type being targeted. In a non-limiting example, in some embodiments of the invention it may be desirable to target a PKK dsRNA agent to and/or into a liver cell, pancreatic cell, kidney cell, testes cell, stomach cell, or lung cell. It will be understood that in some embodiments of methods of the invention, a therapeutic agent comprises a PKK dsRNA agent with only a delivery agent, such as a delivery agent comprising N-Acetylgalactosamine (GalNAc), without any additional attached elements. For example, in some aspects of the invention a PKK dsRNA agent may be attached to a delivery compound comprising GalNAc and included in a composition comprising a pharmaceutically acceptable carrier and administered to a cell or subject without any detectable labels, or targeting agents, etc. attached to the PKK dsRNA agent.

In cases where a PKK dsRNA agent of the invention is administered with and/or attached to one or more delivery agents, targeting agents, labeling agents, etc. a skilled artisan will be aware of and able to select and use suitable agents for use in methods of the invention. Labeling agents may be used in certain methods of the invention to determine the location of a PKK dsRNA agent in cells and tissues and may be used to determine a cell, tissue, or organ location of a treatment composition comprising a PKK dsRNA agent that has been administered in methods of the invention. Procedures for attaching and utilizing labeling agents such as enzymatic labels, dyes, radiolabels, etc. are well known in the art. It will be understood that in some embodiments of compositions and methods of the invention, a labeling agent is attached to one or both of a sense polynucleotide and an antisense polynucleotide included in a PKK dsRNA agent.

Delivery of PKK dsRNA Agents

Certain embodiments of methods of the invention, includes delivery of a PKK dsRNA agent into a cell. As used herein the term, “delivery” means facilitating or effecting uptake or absorption into the cell. Absorption or uptake of a PKK dsRNA agent can occur through unaided diffusive or active cellular processes, or by use of delivery agents, targeting agents, etc. that may be associated with a PKK dsRNA agent of the invention. Delivery means that are suitable for use in methods of the invention include, but are not limited to: in vivo delivery, in which a PKK dsRNA agent is in injected into a tissue site or administered systemically. In some embodiments of the invention, a PKK dsRNA agent is attached to a delivery agent.

Non-limiting examples of methods that can be used to deliver PKK dsRNA agents to cells, tissues and/or subjects include: PKK dsRNA-GalNAc conjugates, PKK dsRNA-GalNAc agent conjugates, SAMiRNA technology, LNP-based delivery methods, and naked RNA delivery. These and other delivery methods have been used successfully in the art to deliver therapeutic RNAi agents for treatment of various diseases and conditions, such as but not limited to: liver diseases, acute intermittent porphyria (AIP), hemophilia, pulmonary fibrosis, etc. Details of various delivery means are found in publications such as: Nikam, R. R. & K. R. Gore (2018) Nucleic Acid Ther, 28 (4), 209-224 August 2018; Springer A. D. & S. F. Dowdy (2018) Nucleic Acid Ther. June 1; 28(3): 109-118; Lee, K. et al., (2018) Arch Pharm Res, 41(9), 867-874; and Nair, J. K. et al., (2014) J. Am. Chem. Soc. 136:16958-16961, the content each of which is incorporated by reference herein.

Some embodiments of the invention comprise use of lipid nanoparticles (LNPs) to deliver a PKK dsRNA agent of the invention to a cell, tissue, and/or subject. LNPs are routinely used for in vivo delivery of PKK dsRNA agents, including therapeutic PKK dsRNA agents. One benefit of using an LNP or other delivery agent is an increased stability of the PKK RNA agent when it is delivered to a subject using the LNP or other delivery agent. In some embodiments of the invention an LNP comprises a cationic LNP that is loaded with one or more PKK RNAi molecules of the invention. The LNP comprising the PKK RNAi molecule(s) is administered to a subject, the LNPs and their attached PKK RNAi molecules are taken up by cells via endocytosis, their presence results in release of RNAi trigger molecules, which mediate RNAi.

Another non-limiting example of a delivery agent that may be used in embodiments of the invention to deliver a PKK dsRNA agent of the invention to a cell, tissue and/or subject is an agent comprising GalNAc that is attached to a PKK dsRNA agent of the invention and delivers the PKK dsRNA agent to a cell, tissue, and/or subject. Examples of certain additional delivery agents comprising GalNAc that can be used in certain embodiments of methods and composition of the invention are disclosed in PCT Application WO2020191183A1 (incorporated herein in its entirety). A non-limiting example of a GalNAc targeting ligand that can be used in compositions and methods of the invention to deliver a PKK dsRNA agent to a cell is a targeting ligand cluster. Examples of targeting ligand clusters that are presented herein are referred to as: GalNAc Ligand with phosphodiester link (GLO) and GalNAc Ligand with phosphorothioate link (GLS). The term “GLX-0” as used herein means a GalNAc ligand that can be any of the disclosed GLS and GLO delivery compounds that can be attached to 3′-end of oligonucleotide during synthesis. For example, though not intended to be limiting: GLX-13 and GLX-14 can be attached to the 3′ end of the oligonucleotide during synthesis. Examples of GalNAc delivery compounds are identified herein as: compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is shown below, with location of attachment of the GalNAc-targeting ligand to an RNAi agent of the invention at far right of each (shown with “ [symbol] ”). In some embodiments GLX-0 is a GalNAc ligand that can be attached to the 3′ end of the oligonucleotide. It will be understood that any RNAi and dsRNA molecule of the invention can be attached to the GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, GLO-1 through GLO-16 and GLS-1 through GLS-16 structures.

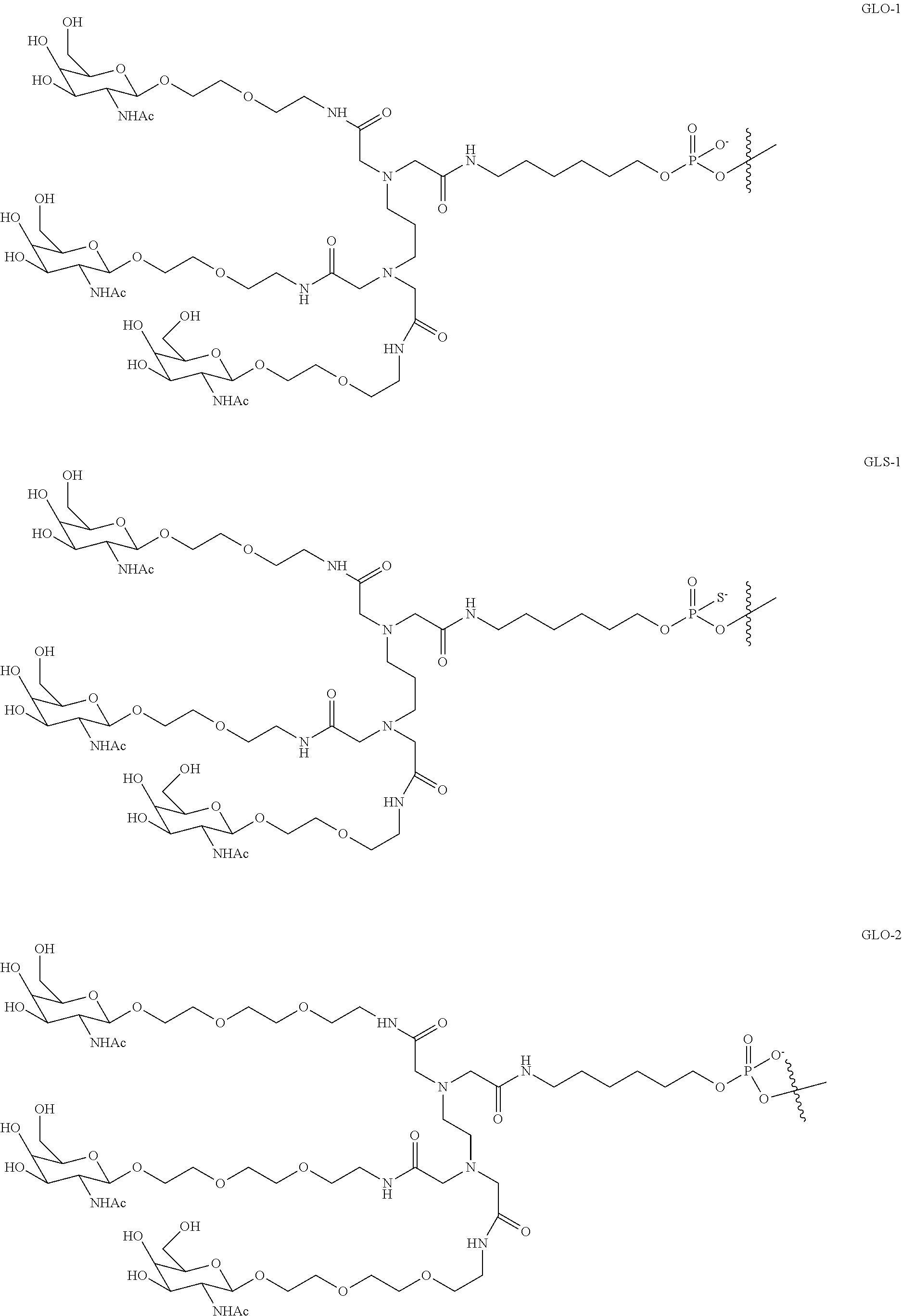 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

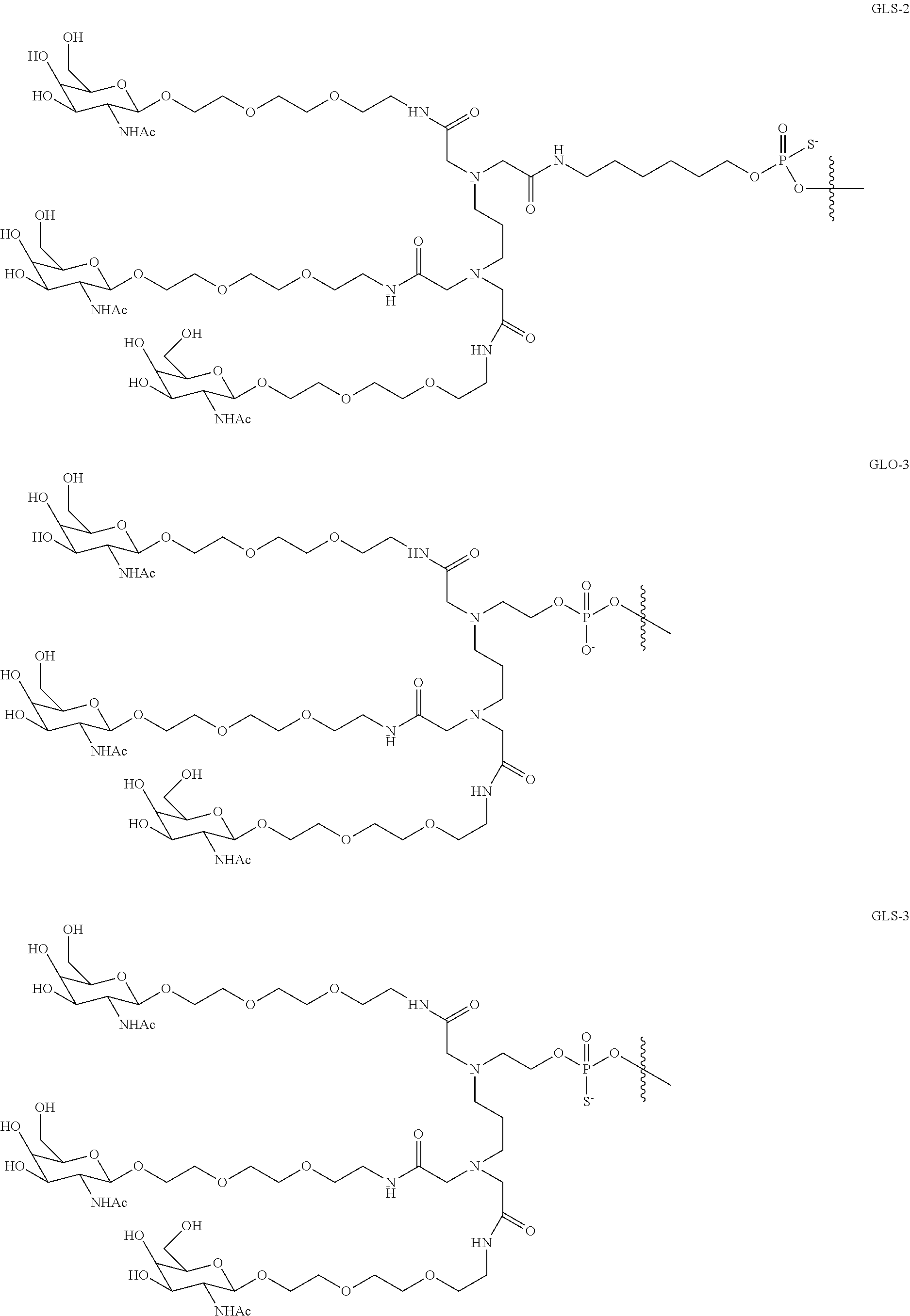 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

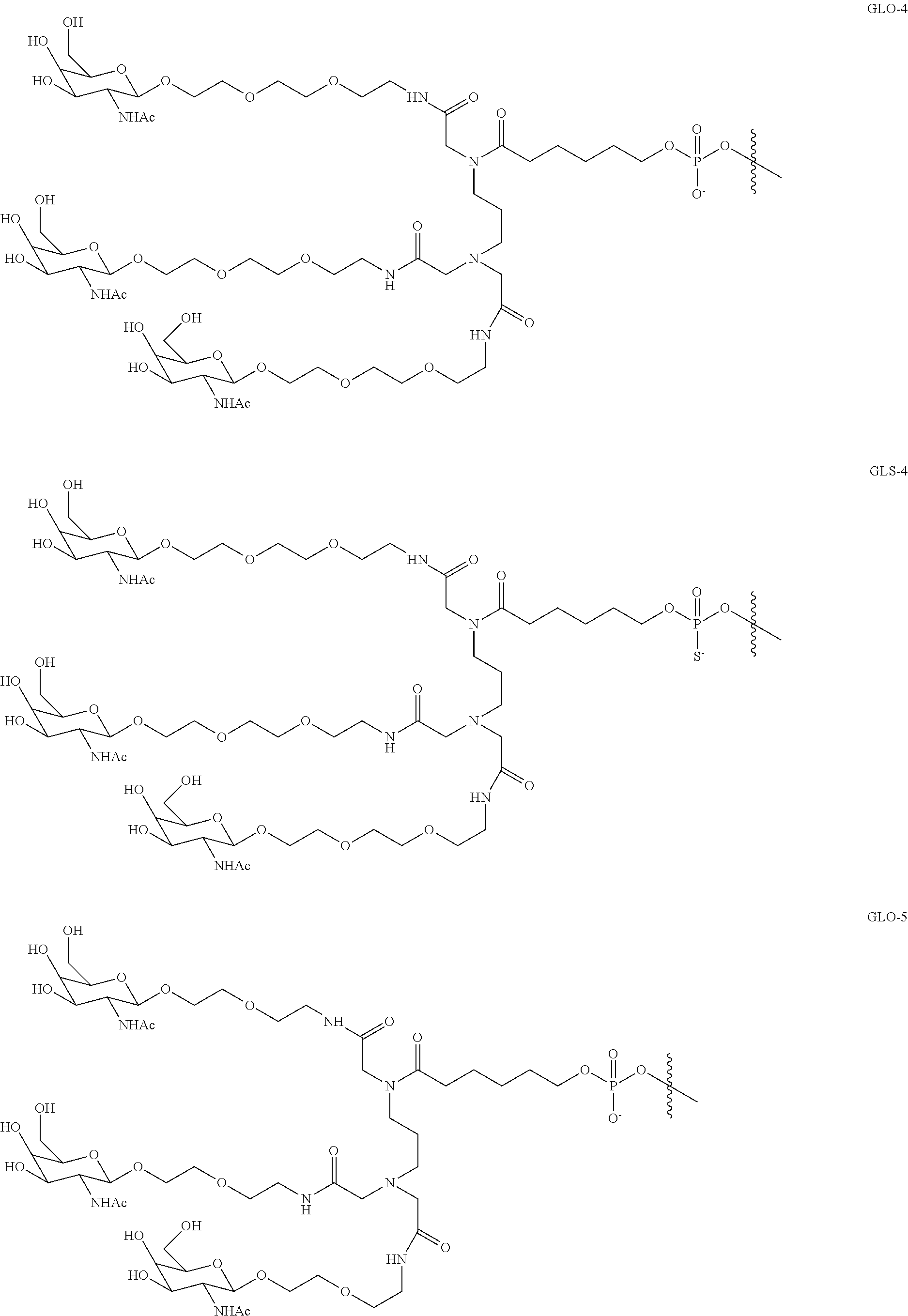 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

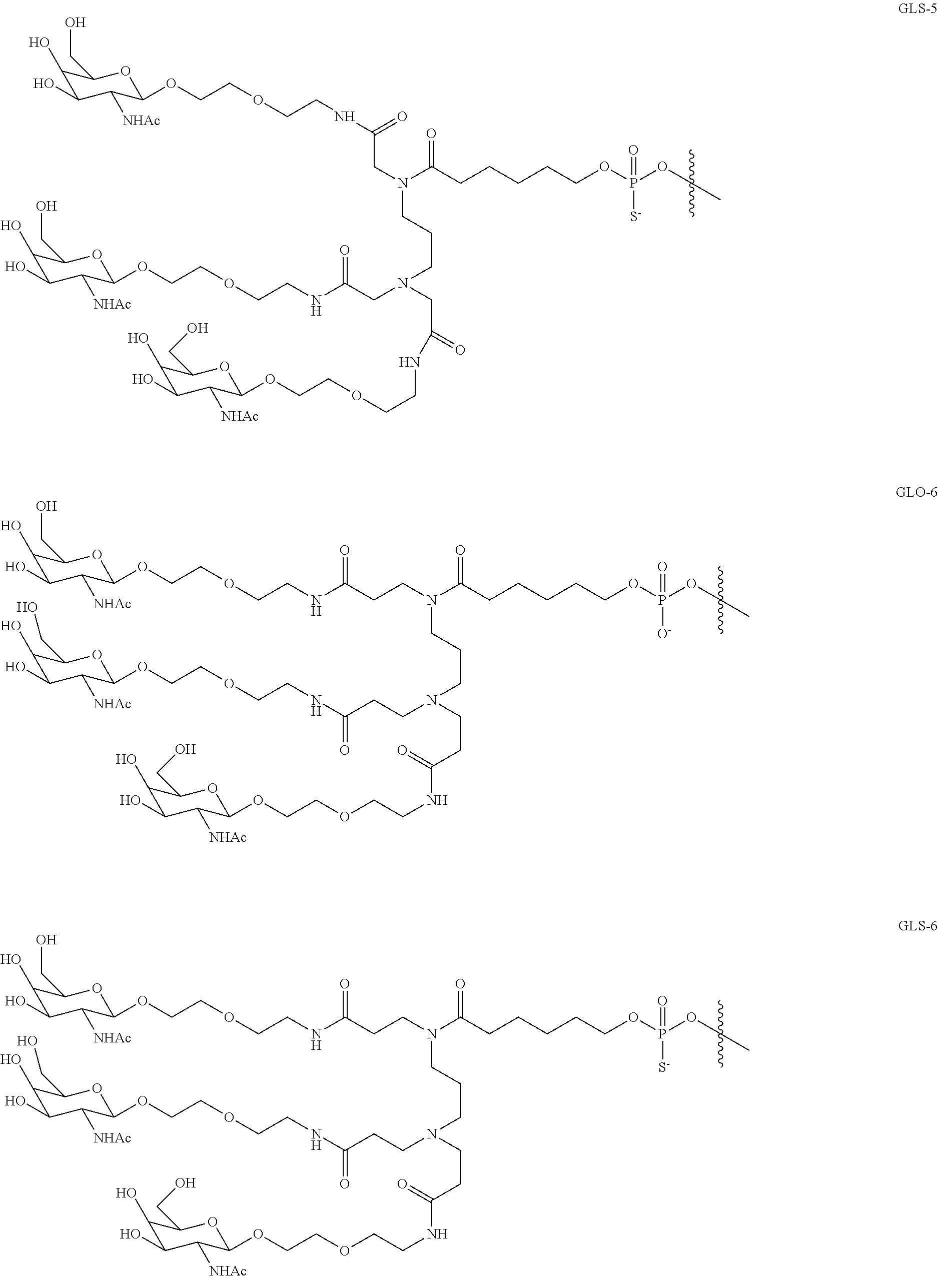 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

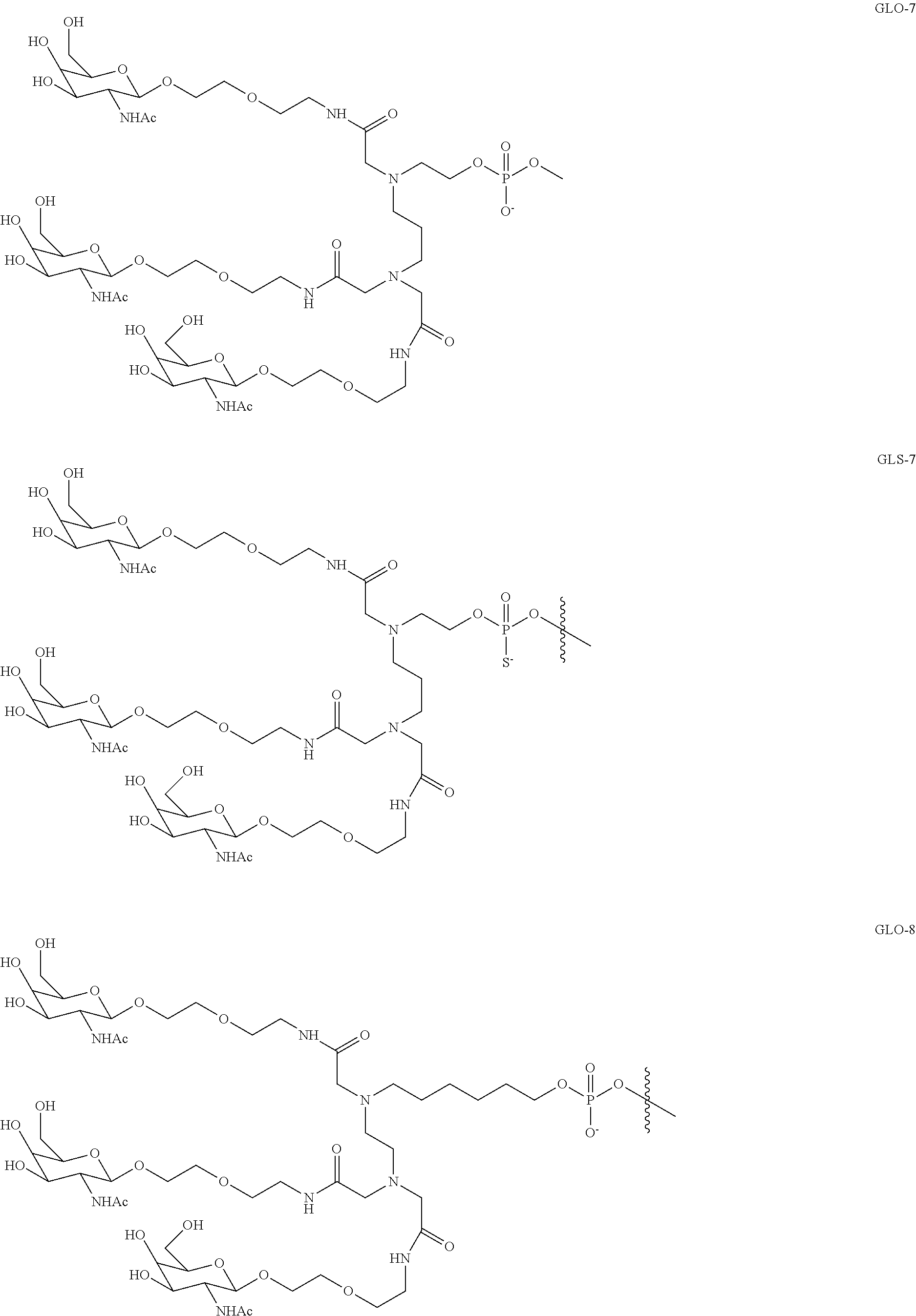 [SMILES: COP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

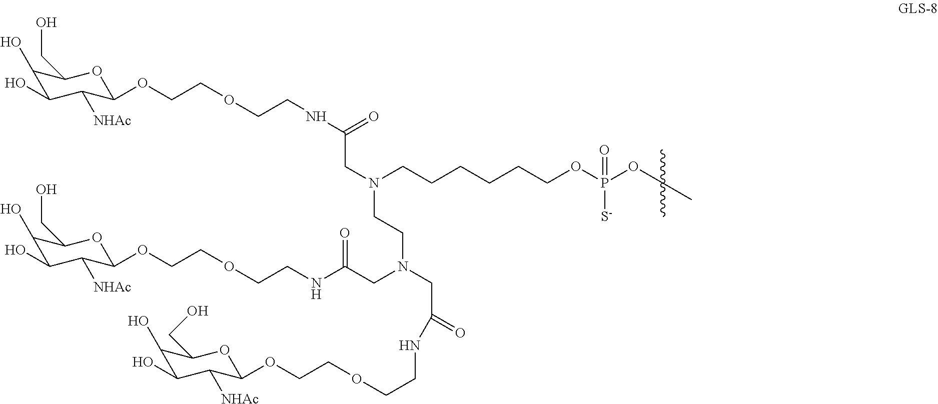 [SMILES: *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

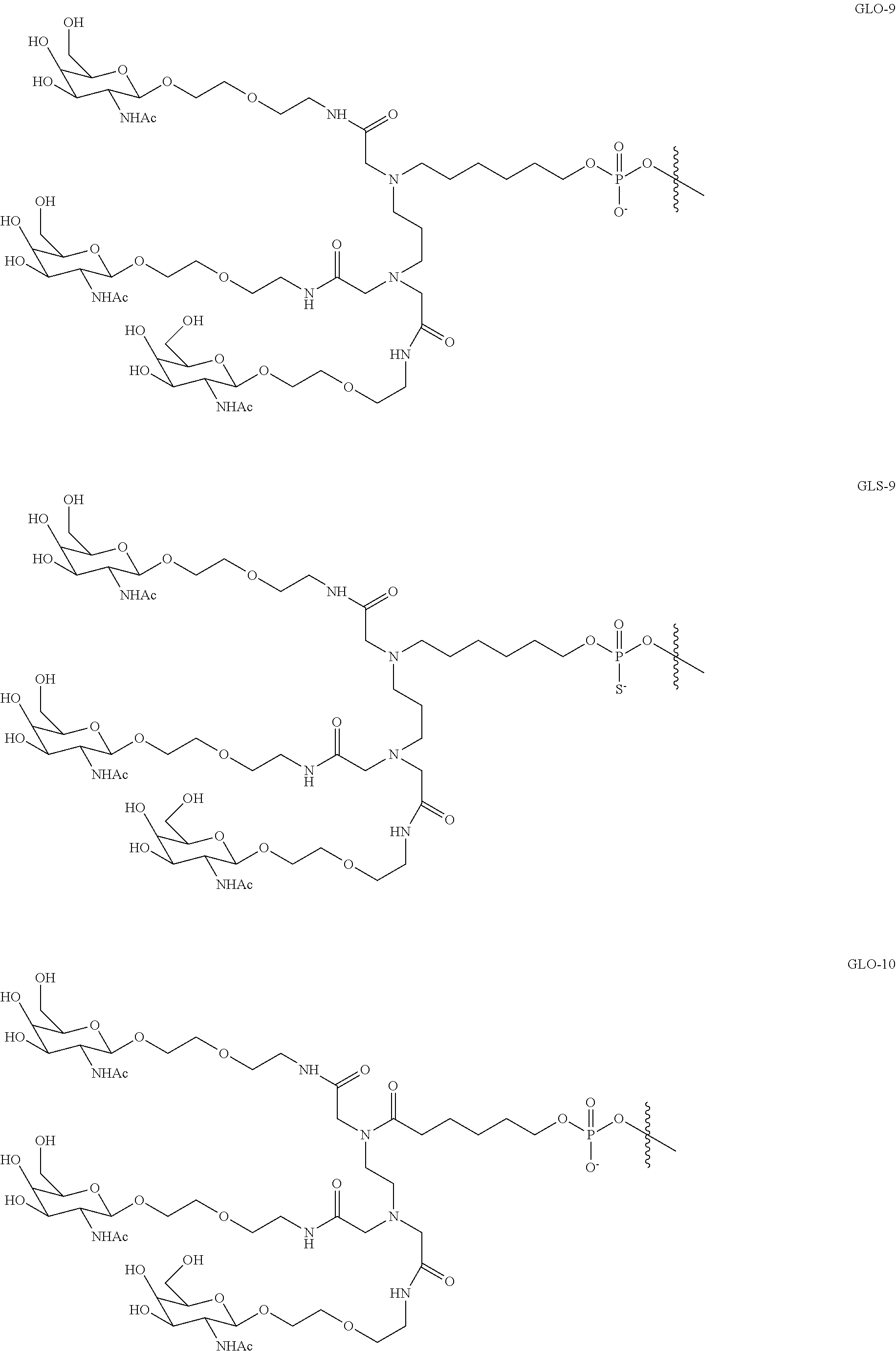 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

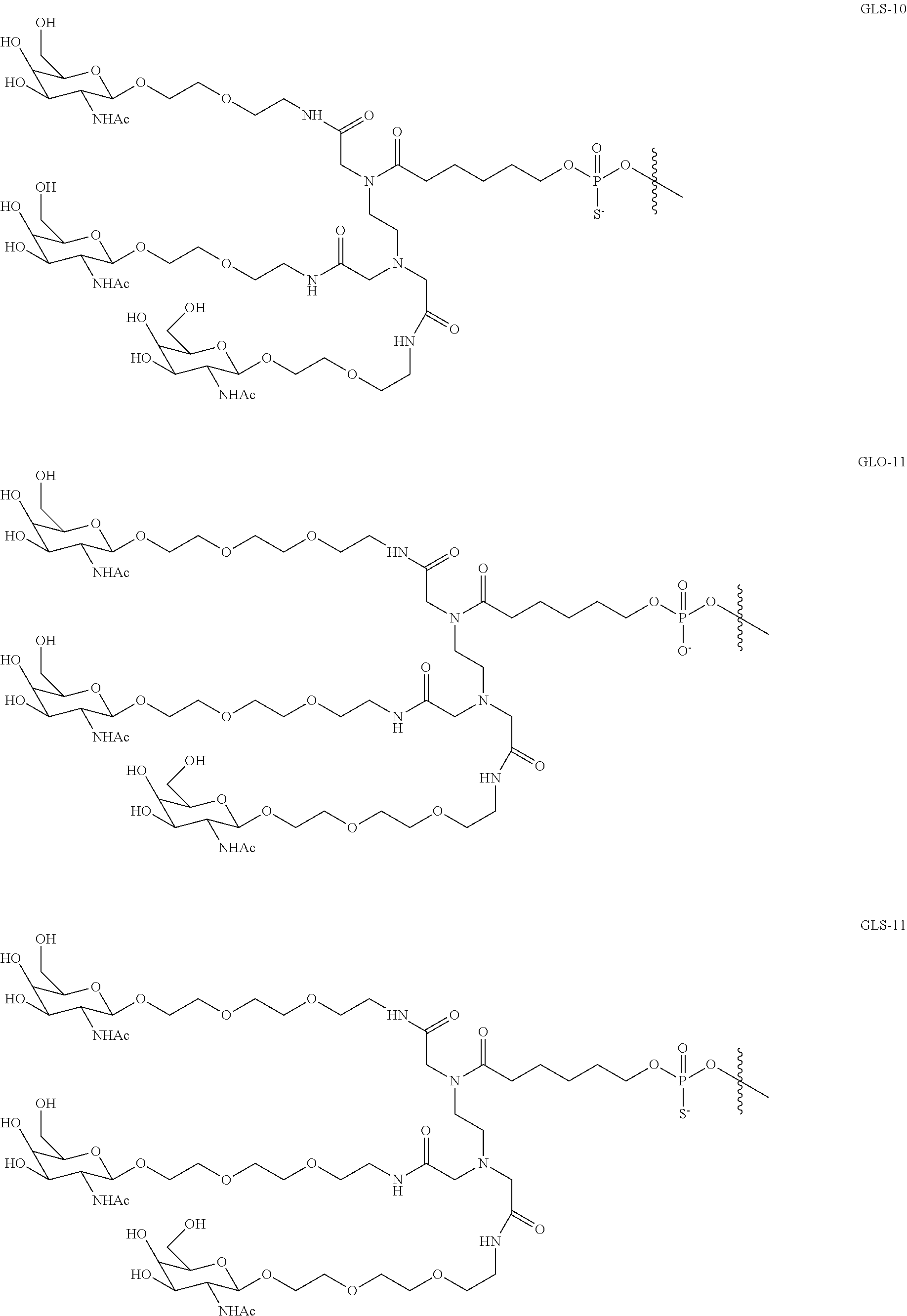 [SMILES: *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

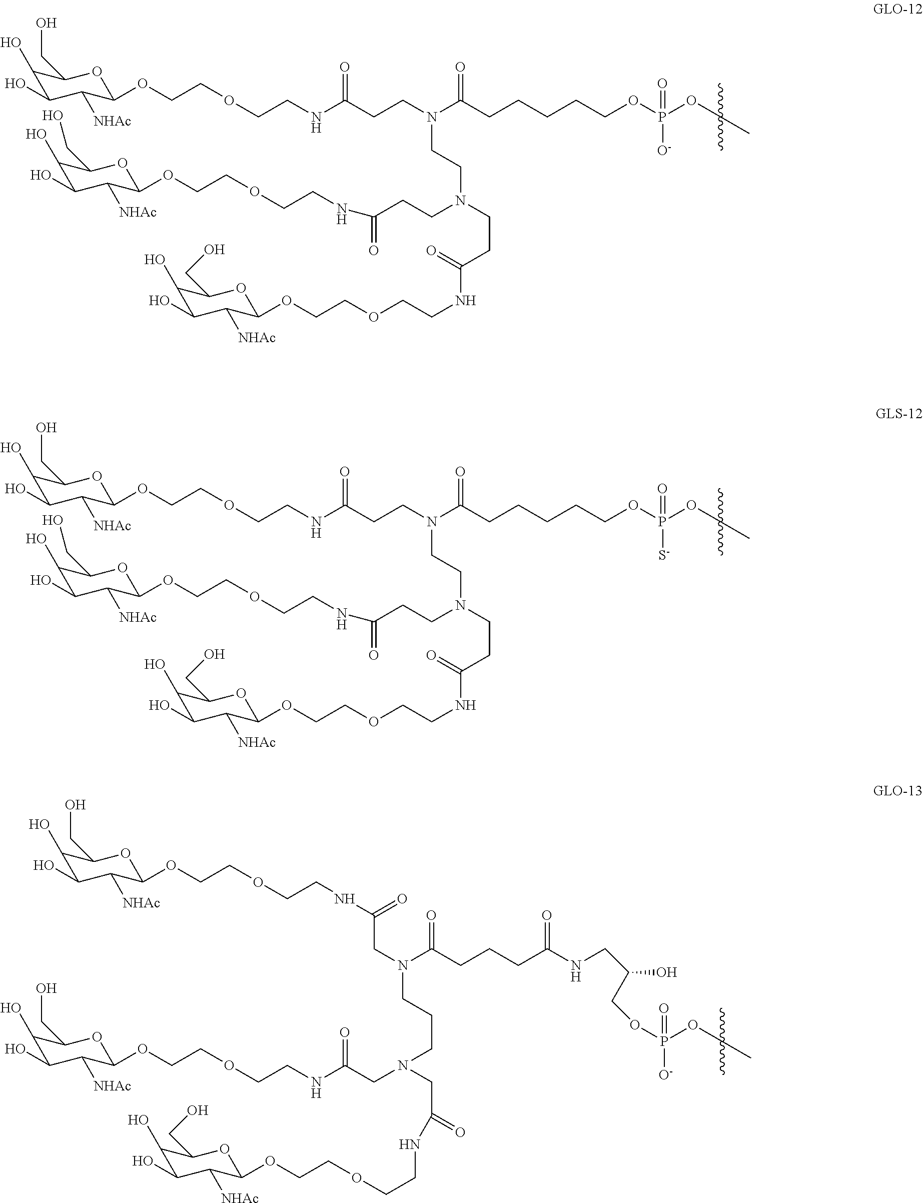 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

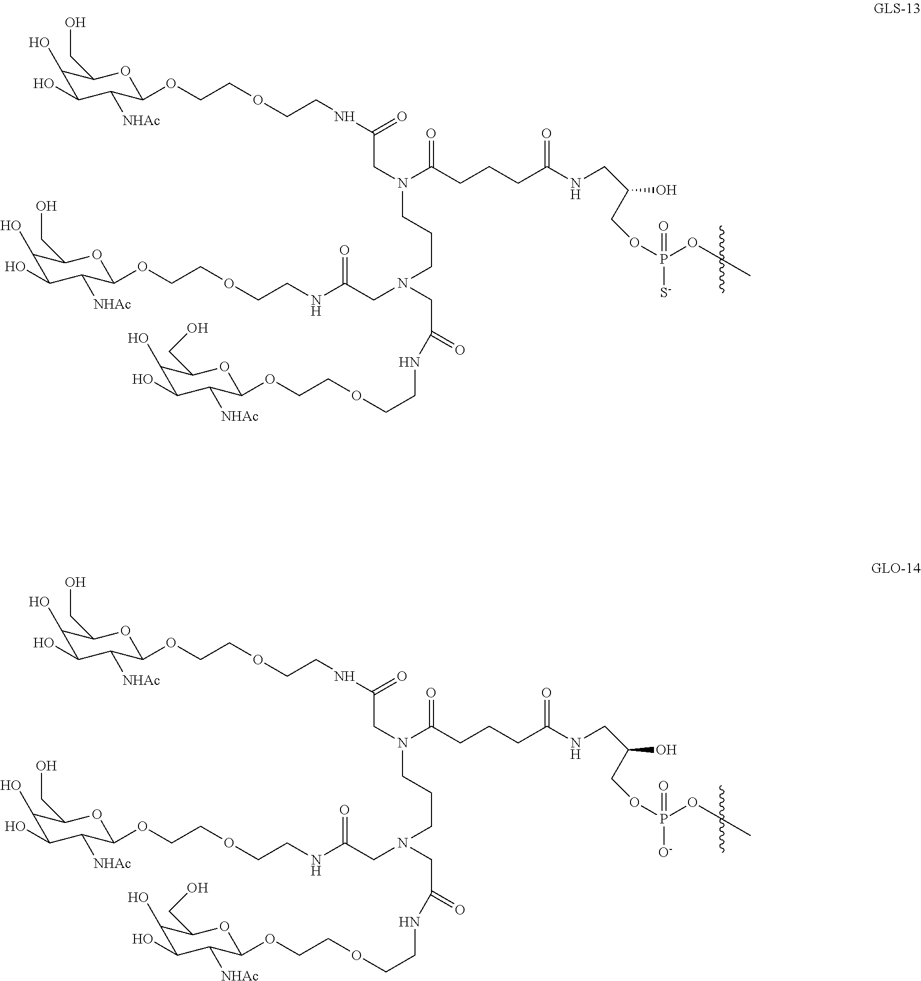 [SMILES: *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

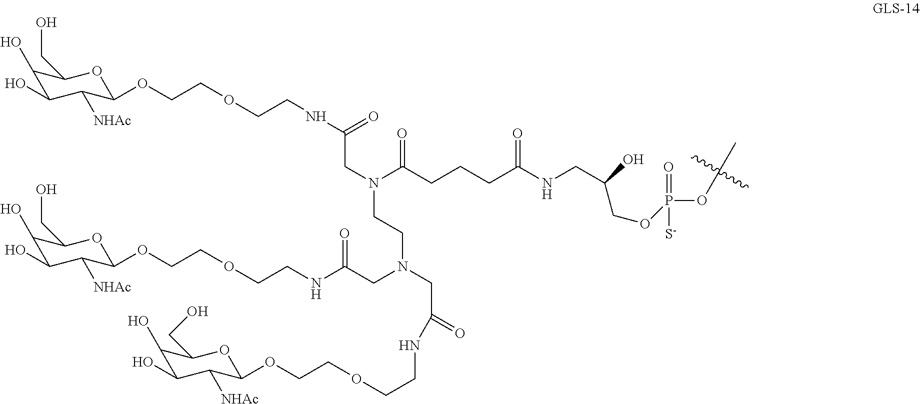 [SMILES: *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

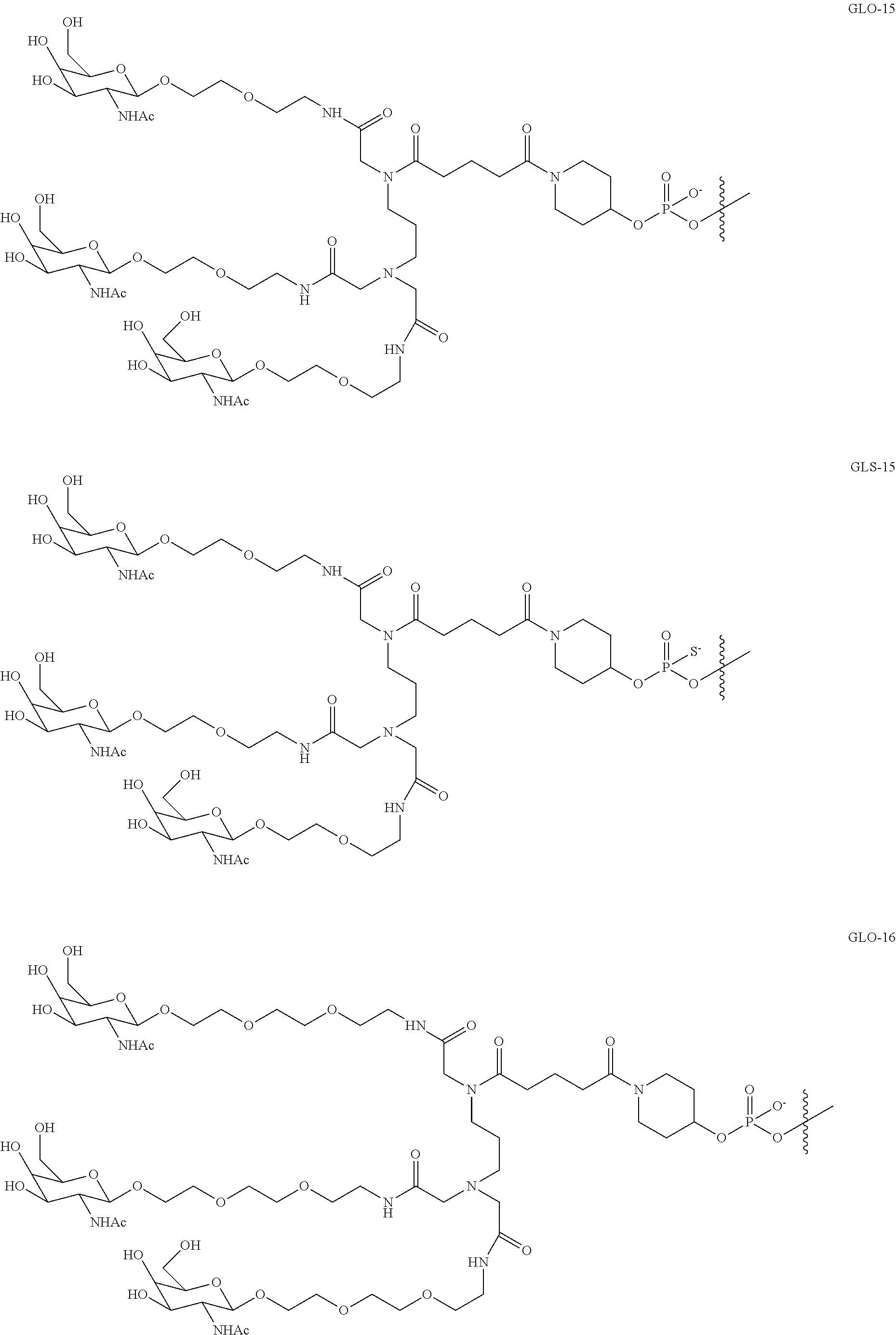 [SMILES: *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

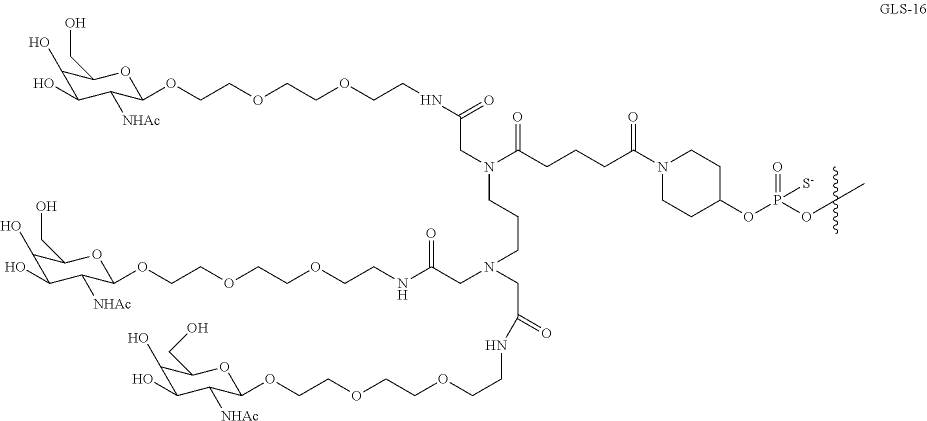 [SMILES: *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

In some embodiments of the invention, in vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction of a PKK RNAi agent into a cell may also be done using art-known methods such as electroporation and lipofection. In certain embodiments of methods of the invention, a PKK dsRNA is delivered without a targeting agent. These RNAs may be delivered as “naked” RNA molecules. As a non-limiting example, a PKK dsRNA of the invention may be administered to a subject to treat a PKK-associated disease or condition in the subject, such as hereditary angioedema, angioedema, hypertension, nephropathy, vascular disease, cardiovascular disease, thrombosis, inflammation in a pharmaceutical composition comprising the RNAi agent, but not including a targeting agent such as a GalNAc targeting compound.

In addition to certain delivery means described herein, it will be understood that RNAi delivery means, such as but not limited to those described herein and those used in the art, can be used in conjunction with embodiments of PKK RNAi agents and treatment methods described herein.

PKK dsRNA agents of the invention may be administered to a subject in an amount and manner effective to reduce a level and activity of PKK polypeptide in a cell and/or subject. The term “activity” as used herein in relation to PKK means activity of the product of PKK cleavage that occurs in cells and subjects. It is known in the art that PKK is cleaved by FXIIa to produce a-kallikrein, which is an active protease responsible for subsequent biological activities. Thus, as used herein the “activity of PKK polypeptide” in a cell means activity of the a-kallikrein cleavage product of the PKK polypeptide. In some embodiments of methods of the invention one or more PKK dsRNA agents are administered to a cell and/or subject to treat a disease or condition associated with PKK expression and activity. Methods of the invention, in some embodiments, include administering one or more independently selected PKK dsRNA agents to a subject in need of such treatment to reduce a disease or condition associated with PKK expression in the subject. PKK dsRNA agents or PKK antisense polynucleotide agents of the invention can be administered to reduce PKK expression and/or activity in one more of in vitro, ex vivo, and in vivo cells.

In some embodiments of the invention, a level, and thus an activity, of PKK polypeptide in a cell is reduced by delivering (e.g. introducing) a PKK dsRNA agent into a cell. Targeting agents and methods may be used to aid in delivery of a PKK dsRNA agent to a specific cell type, cell subtype, organ, spatial region within a subject, and/or to a sub-cellular region within a cell. A PKK dsRNA agent can be administered in certain methods of the invention singly or in combination with one or more additional PKK dsRNA agents. In some embodiments 2, 3, 4, or more independently selected PKK dsRNA agents are administered to a subject. In some embodiments of compositions and methods of the invention, comprise two, three, or more independently selected therapeutic molecules. In some embodiments of methods of the invention, two, three, four, or more PKK siRNAs targeting one, two, or more different positions/regions of KLKB1 mRNA are administered to a cell and/or a subject. In such instances a composition of the invention may comprise two, three, four, or more independently selected PKK siRNAs each conjugated to an independently selected delivery compound. Certain embodiments of agents and/or compositions of the invention comprise divalent siRNA compounds (also referred to herein as linked siRNA compounds), which comprise two independently selected PKK siRNAs that are both conjugated to the same delivery compound.

In certain embodiments of the invention, a PKK dsRNA agent is administered to a subject to treat a PKK-associated disease or condition in conjunction with one or more additional therapeutic regimens for treating the PKK-associated disease or condition. Such combinations may result in a synergistic effect. Non-limiting examples of additional therapeutic regimens are: administering one or more PKK antisense polynucleotides of the invention, administering a non-PKK dsRNA therapeutic agent, and a behavioral modification. An additional therapeutic regimen may be administered at a time that is one or more of: prior to, simultaneous with, and following administration of a PKK dsRNA agent of the invention. It will be understood that simultaneous with as used herein, within five minutes of time zero, within 10 minutes of time zero, within 30 minutes of time zero, within 45 minutes of time zero, and within 60 minutes of time zero, with “time zero” the time of administration of the PKK dsRNA agent of the invention to the subject.

In a non-limiting example, in some embodiments of the invention, an additional therapeutic regimen administered to a subject includes one or more of: one or more PKK antisense polynucleotides, one or more additional PKK dsRNA therapeutic agents, one or more non-PKK dsRNA and/or siRNA therapeutic agent, and a behavioral modification. A non-limiting example of non-PKK dsRNA therapeutic regimen comprises administering to the subject one or more of: a plasma kallikrein inhibitor (including ecallantide, also known by trade name Kalbitor®, and berotralstat, also known by trade name Orladeyo™), a bradykinin B2 receptor antagonist (including icatibant acetate, also known by trade name, Firazyr®), a human monoclonal antibody PKK inhibitor (including lanadelumab-flyo, also known by trade name Takhzyro®), a Factor XII inhibitor, an anabolic androgen (including danazol, stanozolol, and oxandrolone), an antifibrinolytic (including epsilon aminocaproic acid and tranexamic acid), and other therapeutic agents and/or procedures such as but not limited to: oxygen, intravenous fluids, antiemetic agents, and pain control agents (T. Caballero, J Investig Allergol Clin Immunol 31 (1): 1-16 (2021); B. L. Zuraw, All Asth Clin Immunol 6 (1): 23-30 (2010)). Non-limiting examples of behavioral modifications are: a dietary regimen, a rest regimen, an exercise regimen, and counseling. These and other therapeutic agents and behavior modifications are known in the art and used to treat a PKK-associated disease or condition in a subject and may be administered to a subject in combination with the administration of one or more PKK dsRNA agents of the invention to treat the PKK-associated disease or condition. A PKK dsRNA agent of the invention administered to a cell or subject to treat a PKK-associated disease or condition may act in a synergistic manner with one or more other therapeutic agents or activities and increase the effectiveness of the one or more therapeutic agents or activities and/or to increase the effectiveness of the PKK dsRNA agent at treating the PKK-associated disease or condition.

Treatment methods of the invention that include administration of a PKK dsRNA agent can be used in an asymptomatic subject having a PKK-associated disease or condition and/or when one or more symptoms of a PKK-associated disease or condition is present, including at an early stage, mid-stage, and late stage of the disease or condition and all times before and after any of these stages. Methods of the invention may also be to treat subjects who have previously been treated for a PKK-associated disease or condition with one or more other therapeutic agents and/or therapeutic activities that were not successful, were minimally successful, and/or are no longer successful at treating the PKK-associated disease or condition in the subject.

Vector Encoded dsRNAs

In certain embodiments of the invention, a PKK dsRNA agent can be delivered into a cell using a vector. PKK dsRNA agent transcription units can be included in a DNA or RNA vector. Preparation and use of such vectors encoding transgenes for delivering sequences into a cell and or subject are well known in the art. Vectors can be used in methods of the invention that result in transient expression of PKK dsRNA, for example for at least 1, 2, 3, 4, 5, 6, 7, 8, 9, 10 or more hours, 1, 2, 3, 4, 5, 6, 7, 8, 9, 10 or more weeks. The length of the transient expression can be determined using routine methods based on elements such as, but not limited to the specific vector construct selected and the target cell and/or tissue. Such transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

An individual strand or strands of a PKK dsRNA agent can be transcribed from a promoter on an expression vector. Where two separate strands are to be expressed to generate, for example, a dsRNA, two separate expression vectors can be co-introduced to a cell using means such as transfection or infection. In certain embodiments each individual strand of a PKK dsRNA agent of the invention can be transcribed by promoters that are both included on the same expression vector. In certain embodiments of the invention a PKK dsRNA agent is expressed as inverted repeat polynucleotides joined by a linker polynucleotide sequence such that the PKK dsRNA agent has a stem and loop structure.

Non-limiting examples of RNA expression vectors are DNA plasmids or viral vectors. Expression vectors useful in embodiments of the invention can be compatible with eukaryotic cells. Eukaryotic cell expression vectors are routinely used in the art and are available from a number of commercial sources. Delivery of PKK dsRNA expressing vectors can be systemic, such as by intravenous or intramuscular administration, by administration to target cells ex-planted from a subject followed by reintroduction into the subject, or by any other means that allows for introduction into a desired target cell.

Viral vector systems that may be included in an embodiment of a method of the include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Constructs for the recombinant expression of a PKK dsRNA agent may include regulatory elements, such as promoters, enhancers, etc., which may be selected to provide constitutive or regulated/inducible expression. Viral vector systems, and the use of promoters and enhancers, etc. are routine in the art and can be used in conjunction with methods and compositions described herein.

Certain embodiments of the invention include use of viral vectors for delivery of PKK dsRNA agents into cells. Numerous adenovirus-based delivery systems are routinely used in the art for deliver to, for example, lung, liver, the central nervous system, endothelial cells, and muscle. Non-limiting examples of viral vectors that may be used in methods of the invention are: AAV vectors, a pox virus such as a vaccinia virus, a Modified Virus Ankara (MVA), NYVAC, an avipox such as fowl pox or canary pox.

Certain embodiments of the invention include methods of delivering PKK dsRNA agents into cells using a vector and such vectors may be in a pharmaceutically acceptable carrier that may, but need not, include a slow release matrix in which the gene delivery vehicle is imbedded. In some embodiments, a vector for delivering a PKK dsRNA can be produced from a recombinant cell, and a pharmaceutical composition of the invention may include one or more cells that produced the PKK dsRNA delivery system.

Pharmaceutical Compositions Containing PKK dsRNA

Certain embodiments of the invention include use of pharmaceutical compositions containing a PKK dsRNA agent and a pharmaceutically acceptable carrier. The pharmaceutical composition containing the PKK dsRNA agent can be used in methods of the invention to reduce PKK gene expression and serum PKK activity and is useful to treat a PKK-associated disease or condition. Such pharmaceutical compositions can be formulated based on the mode of delivery. Non-limiting examples of formulations for modes of delivery are: a composition formulated for subcutaneous delivery, a composition formulated for systemic administration via parenteral delivery, a composition formulated for intravenous (IV) delivery, a composition formulated for intrathecal delivery, a composition formulated for direct delivery into brain, etc. Administration of a pharmaceutic composition of the invention to deliver a PKK dsRNA agent into a cell may be done using one or more means such as: topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. A PKK dsRNA agent can also be delivered directly to a target tissue, for example directly into the liver, directly into a kidney, etc. It will be understood that “delivering a PKK dsRNA agent” into a cell encompasses delivering a PKK dsRNA agent or PKK antisense polynucleotide agent, respectively, directly as well as expressing a PKK dsRNA agent in a cell from an encoding vector that is delivered into a cell, or by any suitable means with which the PKK dsRNA becomes present in a cell. Preparation and use of formulations and means for delivering inhibitory RNAs are well known and routinely used in the art.

As used herein, a “pharmaceutical composition” comprises a pharmacologically effective amount of a PKK dsRNA agent of the invention and a pharmaceutically acceptable carrier. The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. In some embodiments, a pharmaceutically acceptable carrier include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

As used herein terms such as: “pharmacologically effective amount,” “therapeutically effective amount” and “effective amount” refers to that amount of a PKK dsRNA agent of the invention to produce the intended pharmacological, therapeutic or preventive result. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of a PKK dsRNA agent can reduce PKK polypeptide levels by at least 10%.

### Assessing Efficacy of PKK-Expression Inhibiting Compounds and Compositions

A number of art-known methods can be used to determine presence, absence and/or a level of PKK expression in a cell, tissue, and/or subject before and/or after contact/administration of a PKK inhibiting agent of the invention. In some aspects of the invention such determinations are performed on total RNA obtained from a cell, cells, and/or a biological sample obtained from subject or performed on poly(A)+ mRNA obtained from a cell, cells, and/or a biological sample obtained from a subject. In some embodiments of methods of the invention, a cell and or biological sample obtained from a subject can be analyzed and an amount or level of a PKK-encoding nucleic acid molecule determined using art-known means comprising one or more of: polymerase chain reaction (PCR), Northern blot analysis, and quantitative real-time PCR. RNA analysis can be performed on total cellular RNA or poly(A)+ mRNA.

In some embodiments of methods of the invention, efficacy of a dsRNA agent of the invention to inhibit PKK expression in a cell and/or subject is assessed using art-known means to determine presence, absence and/or level of a PKK polypeptide or one or more of its downstream protein products. For example, though not intended to be limiting, the presence, absence and/or a level of one or more of: a plasma PKK level, a plasma kallikrein level, and a plasma bradykinin generation level can be determined and used to assess efficacy of a dsRNA agent of the invention to reduce PKK expression/activity. Presence, absence, and/or level of a PKK polypeptide activity in a cell, cells, and/or a biological sample obtained from a subject can be determined (also referred to herein as “measured”) before and/or after administration of a dsRNA agent of the invention to the cell and/or subject. A number of art-known methods can be used to determine PKK expression in a cell, tissue and/or subject, non-limiting examples of which are: immunoprecipitation, Western blotting, immunoblotting, enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained commercially, or can be prepared via conventional monoclonal or polyclonal antibody generation methods well known in the art.

### Effective Amounts

Methods of the invention, in some aspects comprise contacting a cell with a PKK dsRNA agent in an effective amount to reduce PKK gene expression in the contacted cell. Certain embodiments of methods of the invention comprise administering a PKK dsRNA agent or a PKK antisense polynucleotide agent to a subject in an amount effective to reduce PKK gene expression and treat a PKK-associated disease or condition in the subject. An “effective amount” used in terms of reducing expression of PKK and/or for treating a PKK-associated disease or condition, is an amount necessary or sufficient to realize a desired biologic effect. For example, an effective amount of a PKK dsRNA agent to treat a PKK-associated disease or condition could be that amount necessary to (i) slow or halt progression of the disease or condition; or (ii) reverse, reduce, or eliminate one or more symptoms of the disease or condition. In some aspects of the invention, an effective amount is that amount of a PKK dsRNA agent that when administered to a subject in need of a treatment of a PKK-associated disease or condition, results in a therapeutic response that prevents and/or treats the disease or condition. According to some aspects of the invention, an effective amount is that amount of a PKK dsRNA agent of the invention that when combined or co-administered with another therapeutic treatment for a PKK-associated disease or condition, results in a therapeutic response that prevents and/or treats the disease or condition. In some embodiments of the invention, a biologic effect of treating a subject with a PKK dsRNA agent of the invention may be the amelioration and or absolute elimination of symptoms resulting from the PKK-associated disease or condition. In some embodiments of the invention, a biologic effect is the complete abrogation of the PKK-associated disease or condition, as evidenced for example, by a diagnostic test that indicates the subject is free of the PKK-associated disease or condition. Additional art-known means of assessing the status of a PKK-associated disease or condition can be used to determine an effect of an agent and/or methods of the invention on a PKK-associated disease or condition.

Typically an effective amount of a PKK dsRNA agent to decrease PKK polypeptide activity to a level to treat a PKK-associated disease or condition will be determined in clinical trials, establishing an effective dose for a test population versus a control population in a blind study. In some embodiments, an effective amount will be that which results in a desired response, e.g., an amount that diminishes a PKK-associated disease or condition in cells, tissues, and/or subjects with the disease or condition. Thus, an effective amount of a PKK dsRNA agent to treat a PKK-associated disease or condition that can be treated by reducing PKK polypeptide activity may be the amount that when administered decreases the amount of PKK polypeptide activity in the subject to an amount that is less than the amount that would be present in the cell, tissue, blood, plasma, and/or subject without the administration of the PKK dsRNA agent or PKK antisense polynucleotide agent. In certain aspects of the invention the level of PKK polypeptide activity, and/or PKK gene expression present in a cell, tissue, and/or subject that has not been contacted with or administered a PKK dsRNA agent of the invention is referred to as a “control” amount. In some embodiments of methods of the invention a control amount for a subject is a pre-treatment amount for the subject, in other words, a level in a subject before administration of a PKK agent can be a control level for that subject and compared to a level of PKK polypeptide activity and/or PKK gene expression in the subject following siRNA administered to the subject. In the case of treating a PKK-associated disease or condition the desired response may be reducing or eliminating one or more symptoms of the disease or condition in the cell, tissue, and/or subject. The reduction or elimination may be temporary or may be permanent.

It will be understood that the status of a PKK-associated disease or condition can be monitored using methods of determining PKK polypeptide activity, PKK gene expression, symptom evaluation, clinical testing, etc. In some aspects of the invention, a desired response to treatment of a PKK-associated disease or condition is delaying the onset or even preventing the onset of the disease or condition. In some aspects of the invention, a desired response to treatment of a PKK-associated disease or condition is reducing the frequency and/or severity with which symptoms of the disease or condition occur.

An effective amount of a compound that decreases PKK polypeptide activity may also be determined by assessing physiological effects of administration of a PKK dsRNA agent on a cell or subject, such as a decrease of a PKK-associated disease or condition following administration. Assays and/or symptomatic monitoring of a subject can be used to determine efficacy of a PKK dsRNA agent of the invention, which may be administered in a pharmaceutical compound of the invention, and to determine the presence or absence of a response to the treatment. A non-limiting example, is that one or more art-known tests of a PKK mRNA transcript level can be used to determine the status of the PKK-associated disease or condition in a subject before and after treatment of the subject with a PKK dsRNA agent of the invention. In this example the disease includes a level of PKK mRNA transcripts and the tests are used to determine a PKK mRNA transcript level in the subject before and after treatment of the subject with a PKK dsRNA agent of the invention. Another non-limiting example, is that one or more art-known tests of plasma PKK and/or plasma kallikrein levels can be used to determine the status of the PKK-associated disease or condition in a subject before and after treatment of the subject with a PKK dsRNA agent of the invention. In this example the disease includes a level of plasma PKK and/or plasma kallikrein and the tests are used to determine a plasma PKK and/or plasma kallikrein level in the subject before and after treatment of the subject with a PKK dsRNA agent of the invention. In another non-limiting example, one or more art-known tests of contact activation are used to determine the status of a PKK-associated disease in a subject. In this example the disease includes a level of contact activation and the tests are used to determine a contact activation level in the subject before and after treatment of the subject with a PKK dsRNA agent of the invention. In yet another non-limiting example, one or more art-known tests of coagulation are used to determine the status of a PKK-associated disease in a subject. In this example the disease includes a level of coagulation and the tests are used to determine a coagulation level in the subject before and after treatment of the subject with a PKK dsRNA agent of the invention. In still another non-limiting example, one or more art-known tests of plasma bradykinin (BK) generation capacity are used to determine the status of a PKK-associated disease in a subject. In this example the disease includes a level of plasma bradykinin and the tests are used to determine a plasma bradykinin level in the subject before and after treatment of the subject with a PKK dsRNA agent of the invention. In another non-limiting example, one or more art-known tests of Factor XIIa (FXIIa) activity are used to determine the status of a PKK-associated disease in a subject. In this example the disease includes levels of plasma Factor XII (FXII) and FXIIa and the tests are used to determine plasma FXII and FXIIa levels in the subject before and after treatment of the subject with a PKK dsRNA agent of the invention. In still another non-limiting example, one or more art-known tests of activated partial thromboplastin time (aPTT) are used to determine the status of a PKK-associated disease in a subject. In this example the disease includes a length of time for a subject to form a blood clot and the tests are used to determine the length of time a subject to form a blood clot before and after treatment of the subject with a PKK dsRNA agent of the invention.

Additional means of determining efficacy of a dsRNA agent of the invention to reduce PKK activity in a cell or subject may include one or more art-known means to evaluate and/or assessing one or more physiological characteristics of a PKK-associated disease or condition in the cell and/or subject, respectively. Non-limiting examples of certain physiological characteristics and symptoms that may be assessed to determine efficacy of a dsRNA agent of the invention in a cell and/or subject are: swelling of skin, swelling of hands and feet, fatigue, headache, muscle aches, tingling of skin, abdominal pain, nausea and vomiting, hoarseness, shortness of breath, mood changes, laryngeal edema, pain, fever, etc. in a subject. Routine means of determining such physiological characteristic are known in the art and include, but are not limited to, blood tests, serum analysis, tissue biopsy, imaging studies, physical examination, etc.

It will be understood that the amount of a PKK dsRNA agent administered to a subject can be modified based, at least in part, on such determinations of disease and/or condition status and/or physiological characteristics determined for a subject. The amount of a treatment may be varied for example by increasing or decreasing the amount of a PKK-dsRNA agent or PKK antisense polynucleotide agent, by changing the composition in which the PKK dsRNA agent or PKK antisense polynucleotide agent, respectively, is administered, by changing the route of administration, by changing the dosage timing and so on. The effective amount of a PKK dsRNA agent will vary with the particular condition being treated, the age and physical condition of the subject being treated; the severity of the condition, the duration of the treatment, the nature of the concurrent therapy (if any), the specific route of administration, and additional factors within the knowledge and expertise of the health practitioner. For example, an effective amount may depend upon the desired level of PKK polypeptide activity and/or PKK gene expression that is effective to treat the PKK-associated disease or condition. A skilled artisan can empirically determine an effective amount of a particular PKK dsRNA agent of the invention for use in methods of the invention without necessitating undue experimentation. Combined with the teachings provided herein, by selecting from among various PKK dsRNA agents or PKK antisense polynucleotide agents of the invention, and weighing factors such as potency, relative bioavailability, patient body weight, severity of adverse side-effects, and preferred mode of administration, an effective prophylactic or therapeutic treatment regimen can be planned that is effective to treat the particular subject. As used in embodiments of the invention, an effective amount of a PKK dsRNA agent of the invention can be that amount that when contacted with a cell results in a desired biological effect in the cell.

It will be recognized that PKK gene silencing may be determined in any cell expressing PKK, either constitutively or by genomic engineering, and by any appropriate assay. In some embodiments of the invention, PKK gene expression is reduced by at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% by administration of a PKK dsRNA agent of the invention. In some embodiments of the invention, PKK gene expression is reduced by at between 5% and 10%, 5% and 25%, 10% and 50%, 10% and 75%, 25% and 75%, 25% and 100%, or 50% and 100% by administration of a PKK dsRNA agent of the invention.

### Dosing

PKK dsRNA agents and PKK antisense polynucleotide agents are delivered in pharmaceutical compositions in dosages sufficient to inhibit expression of a PKK polypeptide. In certain embodiments of the invention, a dose of PKK dsRNA agent is in a range of 0.01 to 200.0 milligrams (mg) per kilogram (kg) body weight of the recipient per day, generally in the range of 1 to 50 mg per kilogram body weight, 5 to 40 mg/kg body weight, 10 to 30 mg/kg body weight, 1 to 20 mg/kg body weight, 1 to 10 mg/kg body weight, 4 to 15 mg/kg body weight per day, inclusive. For example, a PKK dsRNA agent can be administered in an amount that is from about 0.01 mg/kg, 0.05 mg/kg, 0.1 mg/kg, 0.2 mg/kg, 0.3 mg/kg, 0.4 mg/kg, 0.5 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, through 50 mg/kg body per single dose.

Various factors may be considered in the determination of dosage and timing of delivery of a PKK dsRNA agent of the invention. The absolute amount of a PKK dsRNA agent delivered will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment.

Methods of the invention may in some embodiments include administering to a subject 1, 2, 3, 4, 5, 6, 7, 8, 9, 10, or more doses of a PKK dsRNA agent or PKK antisense polynucleotide agent. In some instances, a pharmaceutical compound, (e.g., comprising a PKK dsRNA agent or comprising a PKK antisense polynucleotide agent) can be administered to a subject at least daily, every other day, weekly, every other week, monthly, etc. Doses may be administered once per day or more than once per day, for example, 2, 3, 4, 5, or more times in one 24 hour period. A pharmaceutical composition of the invention may be administered once daily, or the PKK dsRNA agent may be administered as two, three, or more sub-doses at appropriate intervals throughout the day or even using continuous infusion or delivery through a controlled release formulation. In some embodiments of methods of the invention, a pharmaceutical composition of the invention is administered to a subject one or more times per day, one or more times per week, one or more times per month, or one or more times per year.

Methods of the invention, in some aspects, include administration of a pharmaceutical compound alone, in combination with one or more other PKK dsRNA agents or PKK antisense polynucleotide agents, and/or in combination with other drug therapies or treatment activities or regimens that are administered to subjects with a PKK-associated disease or condition. Pharmaceutical compounds may be administered in pharmaceutical compositions. Pharmaceutical compositions used in methods of the invention may be sterile and contain an amount of a PKK dsRNA agent that will reduce activity of a PKK polypeptide to a level sufficient to produce the desired response in a unit of weight or volume suitable for administration to a subject. A dose administered to a subject of a pharmaceutical composition that includes a PKK dsRNA agent to reduce PKK protein activity can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

### Treatment

PKK-associated diseases and conditions in which a decrease in a level and/or activity of PKK polypeptide is effective to treat the disease or condition, can be treated using methods and PKK dsRNA agents of the invention to inhibit PKK expression. Examples of diseases and conditions that may be treated with a PKK dsRNA agent of the invention and a treatment method of the invention, include, but are not limited to: hereditary angioedema, angioedema, hypertension, nephropathy, vascular disease, cardiovascular disease, thrombosis, inflammation. Such diseases and conditions may be referred to herein as “PKK-associated diseases and conditions” and “diseases and conditions caused and/or modulated by PKK.”

In certain aspects of the invention, a subject may be administered a PKK dsRNA agent of the invention at a time that is one or more of before or after diagnosis of a PKK-associated disease or condition. In some aspects of the invention, a subject is at risk of having or developing a PKK-associated disease or condition. A subject at risk of developing a PKK-associated disease or condition is one who has an increased probability of developing the PKK-associated disease or condition, compared to a control risk of developing the PKK-associated disease or condition. In some embodiments of the invention, a level of risk may be statistically significant compared to a control level of risk. Non-limiting examples of a subject at risk may include, a subject who has and/or is at risk of having (1) a preexisting disease and/or abnormality such as, but not limited to a B-cell lymphoproliferative disease, or autoimmune and neoplastic disease that makes the subject more susceptible to developing an acquired angioedema (AAE) disease or condition than a control subject without the preexisting disease or abnormality; (2) a subject having or at risk of developing iatrogenic angioedema, for example, after treatment with angiotensin-converting enzyme (ACE) inhibitors; (3) a subject having a personal medical history of the PKK-associated disease or condition; and (4) a subject who has previously been treated for a PKK-associated disease or condition. It will be understood that a preexisting disease and/or an abnormality that makes the subject more susceptible to a PKK-associated disease or condition, may be a disease or abnormality that when present has been previously identified as having a correlative relation to a higher likelihood of developing a PKK-associated disease or condition.

It will be understood that a PKK dsRNA agent may be administered to a subject based on a medical status of the individual subject. For example, a health-care provided for a subject may assess a PKK transcript level, a plasma PKK level, a plasma kallikrein level, and/or a plasma bradykinin generation level measured in a sample obtained from a subject and determine it is desirable to reduce the subject's PKK level, by administration of a PKK dsRNA agent of the invention. In this example, the PKK transcript, plasma PKK, plasma kallikrein, and plasma bradykinin generation levels may be considered to be non-limiting physiological characteristics of a PKK-associated condition, even if the subject is not diagnosed as having a PKK-associated disease such as one disclosed herein. A healthcare provider may monitor changes in the subject's PKK level, as a measure of efficacy of the administered PKK dsRNA agent of the invention. In a non-limiting example, a biological sample, such as a blood or serum sample is obtained from a subject and a plasma PKK level for the subject is determined in the sample. Upon a finding of the presence of a plasma PKK level in the subject, a PKK dsRNA agent is administered to the subject and a subsequent blood or serum sample is obtained from the subject following the administration and the plasma PKK level determined using the subsequent sample and the results compared to the results determined in the subject's pre-administration (prior) sample. A reduction in the subject's plasma PKK level in the later sample compared to the pre-administration level indicates the administered PKK dsRNA agent efficacy in reducing the plasma PKK level in the subject.

Certain embodiments of methods of the invention include adjusting a treatment that includes administering a PKK dsRNA agent or a PKK antisense polynucleotide agent of the invention to a subject based at least in part on assessment of a change in one or more of the subject's physiological characteristics of a PKK-associated disease or condition resulting from the treatment. For example, in some embodiments of the invention, an effect of an administered dsRNA agent of the invention may be determined for a subject and used to assist in adjusting an amount of a dsRNA agent of the invention subsequently administered to the subject. In a non-limiting example, a subject is administered a dsRNA agent of the invention, the presence and/or level of plasma kallikrein is determined in a biological sample obtained from the subject after the administration, and based at least in part on the determined level, a greater amount of the dsRNA agent is determined to be desirable in order to increase the physiological effect of the administered agent, for example to reduce or further reduce the subject's presence and/or level of plasma kallikrein. In another non-limiting example, a subject is administered a dsRNA agent of the invention, the subject's bradykinin activation level is determined after the administration and based at least in part on the determined bradykinin activation level, a lower amount, higher amount, or same amount of the dsRNA agent is desirable to administer to the subject.

Thus, some embodiments of the invention include assessing a change in one or more physiological characteristics of resulting from a subject's previous treatment to adjust an amount of a dsRNA agent of the invention subsequently administered to the subject. Some embodiments of methods of the invention include 1, 2, 3, 4, 5, 6, or more determinations of a physiological characteristic of a PKK-associated disease or condition to assess and/or monitor the efficacy of an administered PKK dsRNA agent of the invention, and optionally using the determinations to adjust one or more of: a dose, administration regimen, and or administration frequency of a dsRNA agent of the invention to treat a PKK-associated disease or condition in a subject. In some embodiments of methods of the invention, a desired result of administering an effective amount of a dsRNA agent of the invention to a subject is a reduction in one or more of the PKK transcript level, plasma PKK level, plasma kallikrein level, plasma kallikrein activity level, bradykinin generation rate, FXII-to-FXIIa conversion rate, and angioedema attack rate in the subject indicates a reduction of PKK gene expression in the subject.

In certain embodiments of methods of the invention, a desired result of administering an effective amount of a PKK dsRNA agent or PKK antisense polynucleotide agent of the invention to a subject with angioedema and/or hereditary angioedema may be one or more of a reduction in death, a reduction in severity and frequency of instances of angioedema, a reduction of pain, a reduction of swelling, fewer hospital visits, shorter-length hospital stays, and improved life quality compared to these characteristics in a subject not administered the treatment/therapeutic agent or composition of the invention. In certain embodiments of methods of the invention, a desired result of administering an effective amount of a PKK dsRNA agent or PKK antisense polynucleotide agent of the invention to a subject with thrombosis may be one or more of: fewer blood clots, eradication of one or more blood clots, and a reduction in severity and frequency of instances of thrombosis. In certain embodiments of methods of the invention, a desired result of administering an effective amount of a PKK dsRNA agent or PKK antisense polynucleotide agent of the invention to a subject with hypertension, includes but is not limited to a reduction in the hypertension compared to a level of hypertension in a control subject, a non-limiting example of which is a subject not administered the effective amount. In some embodiments of methods of the invention, a desired result of administering an effective amount of a PKK dsRNA agent or PKK antisense polynucleotide agent of the invention to a subject with nephropathy, includes but is not limited to a improved blood pressure control, less protein in the urine; less swelling of feet, ankles, hands or eyes, reduced need to urinate; less confusion or difficulty concentrating, decreased shortness of breath, and less reduction in appetite compared to these physiological characteristics in a control subject, a non-limiting example of which is a subject not administered the effective amount. In certain embodiments of methods of the invention, a desired result of administering an effective amount of a PKK dsRNA agent or PKK antisense polynucleotide agent of the invention to a subject with a vascular disease includes but is not limited to a reduction in presence and/or number of blood clots; reduction in occurrence and/or severity of stroke, aneurysms, lymphedema, varicose veins, peripheral artery disease, intestinal ischemic syndrome, renal artery disease, Raynaud's phenomenon, carotid artery disease, and reduction in blood vessel blockage compared to these physiological characteristics in a control subject, a non-limiting example of which is a subject not administered the effective amount. In some embodiments of methods of the invention, a desired result of administering an effective amount of a PKK dsRNA agent or PKK antisense polynucleotide agent of the invention to a subject with inflammation includes but is not limited to a reduction in presence and/or severity of the inflammation compared to inflammation in a control subject, a non-limiting example of which is a subject not administered the effective amount.

A PKK RNAi as described herein is capable of inhibiting expression of PKK protein. In some embodiments of the invention reducing PKK expression in a cell or subject treats a disease or condition associated with PKK expression in the cell or subject, respectively. Non-limiting examples of diseases and conditions that may be treated by reducing PKK activity are: hereditary angioedema, angioedema, hypertension, nephropathy, vascular disease, cardiovascular disease, thrombosis, inflammation, and/or other diseases for which reducing a level and activity of PKK protein is medically beneficial to a subject, compared to a baseline pre-treatment physiological characteristic of the PKK-associated disease or condition and/or to a control physiological characteristic of the PKK-associated disease or condition, indicates a reduction in PKK gene expression in the subject.

As used herein, the terms “treat”, “treated”, or “treating” when used with respect to a PKK-associated disease or condition may refer to a prophylactic treatment that decreases the likelihood of a subject developing the PKK-associated disease or condition, and also may refer to a treatment after the subject has developed a PKK-associated disease or condition in order to eliminate or reduce the frequency and/or the severity of the PKK-associated disease or condition. In some embodiments a treatment of the invention us used to prevent a PKK-associated disease or condition from becoming more advanced (e.g., more severe), and/or to slow the progression of the PKK-associated disease or condition in a subject compared to a control subject, which in some instances may be the subject in the absence of the therapy to reduce activity in the subject of PKK polypeptide.

Certain embodiments of agents, compositions, and methods of the invention can be used to inhibit PKK gene expression. As used herein in reference to expression of a PKK gene, the terms “inhibit,” “silence,” “reduce,” “down-regulate,” and “knockdown” mean the expression of the PKK gene, as measured by one or more of: a level of RNA transcribed from the gene, a level of activity of PKK expressed, and a level of PKK polypeptide, protein or protein subunit translated from the mRNA in a cell, group of cells, tissue, organ, or subject in which the PKK gene is transcribed, is reduced when the cell, group of cells, tissue, fluid, organ, or subject is contacted with (e.g., treated with) a PKK dsRNA agent of the invention, compared to a control level of RNA transcribed from the PKK gene, a level of activity of expressed PKK, or a level of PKK translated from the mRNA, respectively. In some embodiments, a control level is a level in a cell, tissue, organ or subject that has not been contacted with (e.g. treated with) the PKK dsRNA agent or PKK antisense polynucleotide agent.

### Administration Methods

A variety of administration routes for a PKK dsRNA agent are available for use in methods of the invention. The particular delivery mode selected will depend at least in part, upon the particular condition being treated and the dosage required for therapeutic efficacy. Methods of this invention, generally speaking, may be practiced using any mode of administration that is medically acceptable, meaning any mode that produces effective levels of treatment of a PKK-associated disease or condition without causing clinically unacceptable adverse effects. In some embodiments of the invention, a PKK dsRNA agent may be administered via an oral, enteral, mucosal, subcutaneous, and/or parenteral route. The term “parenteral” includes subcutaneous, intravenous, intrathecal, intramuscular, intraperitoneal, and intrasternal injection, or infusion techniques. Other routes include but are not limited to nasal (e.g., via a gastro-nasal tube), dermal, vaginal, rectal, sublingual, and inhalation. Delivery routes of the invention may include intrathecal, intraventricular, or intracranial. In some embodiments of the invention, a PKK dsRNA agent may be placed within a slow release matrix and administered by placement of the matrix in the subject. In some aspects of the invention, a PKK dsRNA agent may be delivered to a subject cell using nanoparticles coated with a delivery agent that targets a specific cell or organelle. Various delivery means, methods, agents are known in the art. Non-limiting examples of delivery methods and delivery agents are additionally provided elsewhere herein. In some aspects of the invention, the term “delivering” in reference to a PKK dsRNA agent may mean administration to a cell or subject of one or more “naked” PKK dsRNA agent sequences and in certain aspects of the invention “delivering” means administration to a cell or subject via transfection means, delivering a cell comprising a PKK dsRNA agent to a subject, delivering a vector encoding a PKK dsRNA agent into a cell and/or subject, etc. Delivery of a PKK dsRNA agent using a transfection means may include administration of a vector to a cell and/or subject.

In some methods of the invention one or more PKK dsRNA agents or PKK antisense polynucleotide agents may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments of the invention a PKK dsRNA agent may be formulated with another therapeutic agent for simultaneous administration. According to methods of the invention, a PKK dsRNA agent may be administered in a pharmaceutical composition. In general, a pharmaceutical composition comprises a PKK dsRNA agent and optionally, a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients, e.g., the ability of the PKK dsRNA agent to inhibit PKK gene expression in a cell or subject. Numerous methods to administer and deliver dsRNA agents or PKK antisense polynucleotide agents for therapeutic use are known in the art and may be utilized in methods of the invention.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Some embodiments of methods of the invention include administering one or more PKK dsRNA agents or PKK antisense polynucleotide agents directly to a tissue. In some embodiments, the tissue to which the compound is administered is a tissue in which the PKK-associated disease or condition is present or is likely to arise, non-limiting examples of which are the liver or kidney. Direct tissue administration may be achieved by direct injection or other means. Many orally delivered compounds naturally travel to and through the liver and kidneys and some embodiments of treatment methods of the invention include oral administration of one or more PKK dsRNA agents to a subject. PKK dsRNA agents or PKK antisense polynucleotide agents, either alone or in conjunction with other therapeutic agents, may be administered once, or alternatively they may be administered in a plurality of administrations. If administered multiple times, the PKK dsRNA agent may be administered via different routes. For example, though not intended to be limiting, a first (or first several) administrations may be made via subcutaneous means and one or more additional administrations may be oral and/or systemic administrations.

For embodiments of the invention in which it is desirable to administer a PKK dsRNA agent systemically, the PKK dsRNA agent may be formulated for parenteral administration by injection, e.g., by bolus injection or continuous infusion. Formulations for injection may be presented in unit dosage form, e.g., in ampoules or in multi-dose containers, with or without an added preservative. PKK dsRNA agent formulations (also referred to as pharmaceutical compositions) may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more PKK dsRNA agents or PKK antisense polynucleotide agents and to achieve appropriate reduction in PKK protein activity.

In yet other embodiments, methods of the invention include use of a delivery vehicle such as biocompatible microparticle, nanoparticle, or implant suitable for implantation into a recipient, e.g., a subject. Exemplary bioerodible implants that may be useful in accordance with this method are described in PCT Publication No. WO 95/24929 (incorporated by reference herein), which describes a biocompatible, biodegradable polymeric matrix for containing a biological macromolecule.

Both non-biodegradable and biodegradable polymeric matrices can be used in methods of the invention to deliver one or more PKK dsRNA agents or PKK antisense polynucleotide agents to a subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In general, PKK dsRNA agents or PKK antisense polynucleotide agents may be delivered in some embodiments of the invention using the bioerodible implant by way of diffusion, or by degradation of the polymeric matrix. Exemplary synthetic polymers for such use are well known in the art. Biodegradable polymers and non-biodegradable polymers can be used for delivery of PKK dsRNA agents or PKK antisense polynucleotide agents using art-known methods. Bioadhesive polymers such as bioerodible hydrogels (see H. S. Sawhney, C. P. Pathak and J. A. Hubell in Macromolecules, 1993, 26, 581-587, the teachings of which are incorporated by reference herein) may also be used to deliver PKK dsRNA agents or PKK antisense polynucleotide agents for treatment of a PKK-associated disease or condition. Additional suitable delivery systems can include time-release, delayed release or sustained release delivery systems. Such systems can avoid repeated administrations of a PKK dsRNA agent or PKK antisense polynucleotide agent, increasing convenience to the subject and the medical care professional. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation.

Use of a long-term sustained release implant may be suitable for prophylactic treatment of subjects and for subjects at risk of developing a recurrent PKK-associated disease or condition. Long-term release, as used herein, means that the implant is constructed and arranged to deliver a therapeutic level of a PKK dsRNA agent for at least up to 10 days, 20 days, 30 days, 60 days, 90 days, six months, a year, or longer. Long-term sustained release implants are well-known to those of ordinary skill in the art and include some of the release systems described above.

Therapeutic formulations of PKK dsRNA agents or PKK antisense polynucleotide agents may be prepared for storage by mixing the molecule or compound having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

### Cells, Subjects, and Controls

Methods of the invention may be used in conjunction with cells, tissues, organs and/or subjects. In some aspects of the invention a subject is a human or vertebrate mammal including but not limited to a dog, cat, horse, cow, goat, mouse, rat, and primate, e.g., monkey. Thus, the invention can be used to treat PKK-associated diseases or conditions in human and non-human subjects. In some aspects of the invention a subject may be a farm animal, a zoo animal, a domesticated animal or non-domesticated animal and methods of the invention can be used in veterinary prevention and treatment regimens. In some embodiments of the invention, the subject is a human and methods of the invention can be used in human prevention and treatment regimens.

Non-limiting examples of subjects to which the present invention can be applied are subjects who are diagnosed with, suspected of having, or at risk of having a disease or condition associated with a higher than desirable PKK expression and/or activity, also referred to as “elevated levels of PKK expression”. Non-limiting examples of diseases and conditions associated with a higher than desirable levels of PKK expression and/or activity are described elsewhere herein. Methods of the invention may be applied to a subject who, at the time of treatment, has been diagnosed as having the disease or condition associated with a higher than desirable PKK expression and/or activity, or a subject who is considered to be at risk for having or developing a disease or condition associated with a higher than desirable PKK expression and/or activity. In some aspects of the invention a disease or condition associated with a higher than desirable PKK level of expression and/or activity is an acute disease or condition, and in certain aspects of the invention a disease or condition associated with a higher than desirable PKK level of expression and/or activity is a chronic disease or condition.

In a non-limiting example, a PKK dsRNA agent of the invention is administered to a subject diagnosed with, suspected of having, or at risk of having hereditary angioedema, angioedema, hypertension, nephropathy, vascular disease, cardiovascular disease, thrombosis, and/or inflammation, which is a disease or condition in which it is desirable to reduce PKK expression. Methods of the invention may be applied to the subject who, at the time of treatment, has been diagnosed as having the disease or condition, or a subject who is considered to be at risk for having or developing the disease or condition.

In another non-limiting example, a PKK dsRNA agent of the invention is administered to a subject diagnosed with, suspected of having, or at risk of having hereditary angioedema, angioedema, hypertension, nephropathy, vascular disease, cardiovascular disease, thrombosis, and/or inflammation, which is a disease or condition in which it is desirable to reduce PKK expression. Methods of the invention may be applied to the subject who, at the time of treatment, has been diagnosed as having the disease or condition, or a subject who is considered to be at risk for having or developing the disease or condition.

A cell to which methods of the invention may be applied include cells that are in vitro, in vivo, ex vivo cells. Cells may be in a subject, in culture, and/or in suspension, or in any other suitable state or condition. A cell to which a method of the invention may be applied can be a liver cell, a hepatocyte, a cardiac cell, a pancreatic cell, a cardiovascular cell, kidney cell or other type of vertebrate cell, including human and non-human mammalian cells. In certain aspects of the invention, a cell to which methods of the invention may be applied is a healthy, normal cell that is not known to be a disease cell. In certain embodiments of the invention a cell to which methods and compositions of the invention are applied to a liver cell, a hepatocyte, a cardiac cell, a retinal cell, a vascular cell, a pancreatic cell, a cardiovascular cell, and/or a kidney cell. In certain aspects of the invention, a control cell is a normal cell, but it will be understood that a cell having a disease or condition may also serve as a control cell in particular circumstances for example to compare results in a treated cell having a disease or condition versus an untreated cell having the disease or condition, etc. A cell suitable for treatment using an embodiment of the invention can be a cell that expresses a PKK gene, including a cell comprising an expression vector that includes a PKK gene or a portion of a PKK gene.

A level of PKK polypeptide activity can be determined and compared to a control level of PKK polypeptide activity, according to methods of the invention. A control may be a predetermined value, which can take a variety of forms. It can be a single cut-off value, such as a median or mean. It can be established based upon comparative groups, such as in groups having lower levels of PKK polypeptide and/or PKK polypeptide activity and groups having increased levels of PKK polypeptide and/or PKK polypeptide activity. Another non-limiting example of comparative groups may be groups having one or more symptoms of or a diagnosis of a PKK-associated disease or condition; groups without having one or more symptoms of or a diagnosis of the disease or condition; groups of subjects to whom an siRNA treatment of the invention has been administered; groups of subjects to whom an siRNA treatment of the invention has not been administered. Typically, a control may be based on apparently healthy normal individuals in an appropriate age bracket or apparently healthy cells. It will be understood that controls according to the invention may be, in addition to predetermined values, samples of materials tested in parallel with the experimental materials. Examples include samples from control populations or control samples generated through manufacture to be tested in parallel with the experimental samples. In some embodiments of the invention, a control may include a cell or subject not contacted or treated with a PKK dsRNA agent of the invention and in such instances, a control level of PKK polypeptide and/or PKK polypeptide activity can be compared to a level of PKK polypeptide and/or PKK polypeptide activity in a cell or subject contacted with a PKK dsRNA agent of the invention.

In some embodiments of the invention a level of PKK polypeptide determined for a subject can be a control level against which a level of PKK polypeptide determined for the same subject at a different time is compared. In a non-limiting example, a level of PKK is determined in a biological sample obtained from a subject who has not been administered a PKK treatment of the invention. In some embodiments, the biological sample is a serum sample or a tissue sample. The level of PKK polypeptide determined in the sample obtained from the subject can serve as a baseline or control value for the subject. After one or more administrations of a PKK dsRNA agent to the subject in a treatment method of the invention, one or more additional serum samples can be obtained from the subject and the level of PKK polypeptide in the subsequent sample or samples can be compared to the control/baseline level for the subject. Such comparisons can be used to assess onset, progression, or recession of a PKK-associated disease or condition in the subject. For example, a level of PKK polypeptide in the baseline sample obtained from the subject that is higher than a level obtained from the same subject after the subject has been administered a PKK dsRNA agent of the invention indicates regression of the PKK-associated disease or condition and indicates efficacy of the administered PKK dsRNA agent of the invention for treatment of the PKK-associated disease or condition. In some embodiments, a statistically significant decrease of a level of PKK polypeptide in a sample from a subject compared to a level determined in a previously obtained sample from the subject shows regression of the PKK-associated disease or condition.

In some aspects of the invention, values of one or more of a level of PKK polypeptide and/or PKK polypeptide activity determined for a subject may serve as control values for later comparison of levels of PKK polypeptide and/or PKK activity, in that same subject, thus permitting assessment of changes from a “baseline” PKK polypeptide activity in a subject. Thus, an initial PKK polypeptide level and/or initial PKK polypeptide activity level may be present and/or determined in a subject and methods and compounds of the invention may be used to decrease the level of PKK polypeptide and/or PKK polypeptide activity in the subject, with the initial level serving as a control level for that subject.

Using methods of the invention, PKK dsRNA agents of the invention may be administered to a subject. Efficacy of the administration and treatment of the invention can be assessed when a level of PKK polypeptide and/or PKK polypeptide activity in a serum sample and/or tissue sample obtained from a subject is decreased by at least 0.5%, 1%, 5%, 10%, 20%, 30%, 40%, 50%, 60%, 70%, 80%, 90%, 95%, 96%, 97%, 98%, 99%, or 100% compared to a pre-administration level of PKK polypeptide and/or PKK polypeptide activity in a serum sample and/or tissue sample obtained from the subject at a prior time point, or compared to a non-contacted control level, for example a level of PKK polypeptide and/or PKK polypeptide activity in a control serum sample. It will be understood that a level of PKK polypeptide, a level of PKK polypeptide activity, and other physiological characteristics described herein correlate with a level of PKK gene expression. Certain embodiments of methods of the invention comprise administering a PKK dsRNA of the invention to a subject in an amount effective to inhibit PKK gene expression and thereby reduce a level of PKK polypeptide and/or reduce a level of PKK polypeptide activity in the subject.

Some embodiments of the invention, include determining presence, absence, and/or an amount (also referred to herein as a level) of PKK polypeptide in one or more biological samples obtained from one or more subjects. The determination can be used to assess efficacy of a treatment method of the invention. For example, methods and compositions of the invention can be used to determine a level of PKK polypeptide and/or PKK polypeptide activity in a biological sample obtained from a subject previously treated with administration of a PKK dsRNA agent of the invention. A level of PKK polypeptide and/or PKK polypeptide activity determine in the biological sample obtained from the treated subject that is lower by at least 0.5%, 1%, 5%, 10%, 20%, 30%, 40%, 50%, 60%, 70%, 80%, 90%, 95%, 99%, 99.9%, 99.99% or more compared to a pretreatment level of PKK polypeptide and/or PKK polypeptide activity, respectively, determined for the subject, or compared to a non-contacted control biological sample level, indicates a level of efficacy of the treatment administered to the subject. In some embodiments of methods of the invention, contact of a cell (also referred to herein as treatment) with an siRNA agent of the invention results in inhibition of PKK gene expression in the cell by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%, for example, to below the level of detection of the assay.

In some embodiments of the invention a physiological characteristic of a PKK-associated disease or condition determined for a subject can be a control determination against which a determination of the physiological characteristic in the same subject at a different time is compared. In a non-limiting example, a physiological characteristic such as a PKK transcript level, plasma PKK level, plasma kallikrein level, plasma kallikrein activity level, bradykinin generation rate, and/or FXII-to-FXIIa conversion rate is determined in a biological sample obtained from a subject who has not been administered a PKK treatment of the invention. The PKK transcript level, plasma PKK level, plasma kallikrein level, plasma kallikrein activity level, bradykinin generation rate, and/or FXII-to-FXIIa conversion rate (and/or other physiological characteristic of a PKK-associated disease or condition) determined in the sample obtained from the subject can serve as a baseline or control value for the subject. After one or more administrations of a PKK dsRNA agent to the subject in a treatment method of the invention, one or more additional biological samples can be obtained from the subject and the PKK transcript level, plasma PKK level, plasma kallikrein level, plasma kallikrein activity level, bradykinin generation rate, and/or FXII-to-FXIIa conversion rate in the subsequent sample or samples are compared to the control/baseline level and/or ratio, respectively, for the subject. Such comparisons can be used to assess onset, progression, or recession of a PKK-associated disease or condition in the subject. For example, a PKK transcript level, plasma PKK level, plasma kallikrein level, plasma kallikrein activity level, bradykinin generation rate, and/or FXII-to-FXIIa conversion rate in the baseline sample obtained from the subject that is higher than a PKK transcript level, plasma PKK level, plasma kallikrein level, plasma kallikrein activity level, bradykinin generation rate, and/or FXII-to-FXIIa conversion rate determined in a sample obtained from the same subject after the subject has been administered a PKK dsRNA agent of the invention indicates regression of the PKK-associated disease or condition and indicates efficacy of the administered PKK dsRNA agent of the invention for treatment of the PKK-associated disease or condition.

In some aspects of the invention, values of one or more of a physiological characteristic of a PKK-associated disease or condition determined for a subject may serve as control values for later comparison of the physiological characteristics in that same subject, thus permitting assessment of changes from a “baseline” physiological characteristic in a subject. Thus, an initial physiological characteristic may be present and/or determined in a subject and methods and compounds of the invention may be used to decrease the level of PKK polypeptide and/or PKK polypeptide activity in the subject, with the initial physiological characteristic determination serving as a control for that subject.

Using methods of the invention, PKK dsRNA agents of the invention may be administered to a subject in an effective amount to treat a PKK-associated disease or condition. Efficacy of the administration and treatment of the invention can be assessed by determining a change in one or more physiological characteristics of the PKK-associated disease or condition. In non-limiting examples, a PKK transcript level, plasma PKK level, plasma kallikrein level, plasma kallikrein activity level, bradykinin generation rate, and/or FXII-to-FXIIa conversion rate determined in a biological sample obtained from a subject following treatment with a method of the invention, that is lower by at least 0.5%, 1%, 5%, 10%, 20%, 30%, 40%, 50%, 60%, 70%, 80%, 90%, 95%, 99%, 99.9%, 99.99%, or 100% compared to a pre-administration level of the physiological characteristic in a biological sample obtained from the subject at a pre-treatment time point, or compared to the determined physiological characteristic in a non-contacted control, demonstrates efficacy of the treatment administered to the subject. In another non-limiting example, a reduction in an angioedema attack rate determined in a subject following treatment with a method of the invention, that is lower by at least 0.5%, 1%, 5%, 10%, 20%, 30%, 40%, 50%, 60%, 70%, 80%, 90%, 95%, 99%, 99.9%, 99.99%, or 100% compared to a pre-administration attack rate in the subject at a pre-treatment time period, or compared to the determined physiological characteristic in a non-contacted control, demonstrates efficacy of the treatment administered to the subject. It will be understood that a determination of a physiological characteristic of a PKK-associate disease or condition as described herein, correlates with a level of PKK gene expression. Certain embodiments of methods of the invention comprise administering a PKK dsRNA agent of the invention to a subject in an amount effective to inhibit PKK gene expression and thereby reduce a PKK transcript level, plasma PKK level, plasma kallikrein level, plasma kallikrein activity level, bradykinin generation rate, FXII-to-FXIIa conversion rate, and/or angioedema attack rate, or otherwise positively impact a physiological characteristic of a PKK-associated disease or condition in the subject.

Some embodiments of the invention, include determining presence, absence, and/or a change in a physiological characteristic of a PKK-associated disease or condition using methods such as but not limited to: (1) assessing one or more biological samples obtained from one or more subjects for the physiological characteristic; (2) taking a cell and/or tissue biopsy from a subject (for example but not limited to a liver biopsy); (3) taking a serum sample from a subject; (4) taking a blood sample from a subject; and (5) or physical examination of the subject. The determination can be used to assess efficacy of a treatment method of the invention.

### Kits

Also within the scope of the invention are kits that comprise one or more PKK dsRNA agents of the invention and instructions for its use in methods of the invention. Kits of the invention may include one or more of a PKK dsRNA agent, PKK sense polynucleotide, and PKK antisense polynucleotide agent that may be used to treat a PKK-associated disease or condition. Kits containing one or more PKK dsRNA agents, PKK sense polynucleotides, and PKK antisense polynucleotide agents can be prepared for use in treatment methods of the invention. Components of kits of the invention may be packaged either in aqueous medium or in lyophilized form. A kit of the invention may comprise a carrier being compartmentalized to receive in close confinement therein one or more container means or series of container means such as test tubes, vials, flasks, bottles, syringes, or the like. A first container means or series of container means may contain one or more compounds such as a PKK dsRNA agent and/or one or more PKK sense or antisense polynucleotide molecules. A second container means or series of container means may contain a targeting agent, a labelling agent, a delivery agent, etc. that may be included as a portion of a PKK dsRNA agent to be administered in an embodiment of a treatment method of the invention.

A kit of the invention may also include instructions. Instructions typically will be in written form and will provide guidance for carrying-out a treatment embodied by the kit and for making a determination based upon that treatment.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1. Synthesis of PKK RNAi Agents

PKK RNAi agent duplexes shown in Tables 2-3, above, were synthesized in accordance with the following general procedures:

Sense and antisense strand sequences of siRNA were synthesized on oligonucleotide synthesizers using a well-established solid phase synthesis method based on phosphoramidite chemistry. Oligonucleotide chain propagation is achieved through 4-step cycles: a deprotection, a coupling, a capping and an oxidation or a sulfurization step for addition of each nucleotide. Syntheses were performed on a solid support made of controlled pore glass (CPG, 500 or 1000 Å). Monomer phosphoramidites were purchased from commercial sources. Phosphoramidites with GalNAc ligand cluster (GLPA1 and GLPA2 as non-limiting examples) were synthesized according to the procedures of Examples 2-3 herein. For siRNAs used for in vitro screening (Table 2), syntheses were carried out at 20 nmol scale, and for siRNAs used for in vivo testing (Table 3), syntheses were carried out at scale of 1 μmol or larger. In the case where the GalNAc ligand (GLO-0 as a non-limiting example) is attached at 3′-end of sense strand, GalNAc ligand attached CPG solid support was used. In the case where the GalNAc ligand (GLS-1 or GLS-2 as non-limiting example) is attached at 5′-end of sense strand, a GalNAc phosphoramidite (GLPA1 or GLPA2 as a non-limiting example) was used for the last coupling reaction. Trichloroacetic acid (TCA) 3% in dichloromethane was used for deprotection of 4,4′-dimethoxytrityl protecting group (DMT). 5-Ethylthio-1H-tetrazole was used as an activator. I2 in THF/Py/H₂O and phenylacetyl disulfide (PADS) in pyridine/MeCN was used for oxidation and sulfurization reactions, respectively. After the final solid phase synthesis step, cyanoethyl protecting group was removed with 20% diethylamine in acetonitrile. Protecting groups for nucleobase were removed and solid support bound oligomer was cleaved by treating with 28% ammonium hydroxide solution at 65° C. for 2 hours. The aqueous solution contains crude single strand product was concentrated and purified by ion pairing reversed phase HPLC (IP-RP-HPLC). Purified single strand oligonucleotide product from IP-RP-HPLC was converted to sodium salt by dissolving in 1.0 M NaOAc and precipitation by addition of ice cold EtOH. Annealing of equimolar complementary sense stand and antisense strand oligonucleotide in water was performed to form the double strand siRNA product, which was lyophilized to afford a fluffy white solid.

### Example 2. Preparation of Intermediate-A and Intermediate-B

As shown in Scheme 1 below, Intermediate-A was synthesized by treating commercially available galactosamine pentaacetate with trimethylsilyl trifluoromethanesulfonate (TMSOTf) in dichloromethane (DCM). This was followed by glycosylation with Cbz protected 2-(2-aminoethoxy) ethan-1-ol to give Compound II. The Cbz protecting group was removed by hydrogenation to afford Intermediate-A as a trifluoroacetate (TFA) salt. Intermediate B was synthesized based on the same scheme except Cbz protected 2-(2-(2-aminoethoxy) ethoxy) ethan-1-ol was used as the starting material.

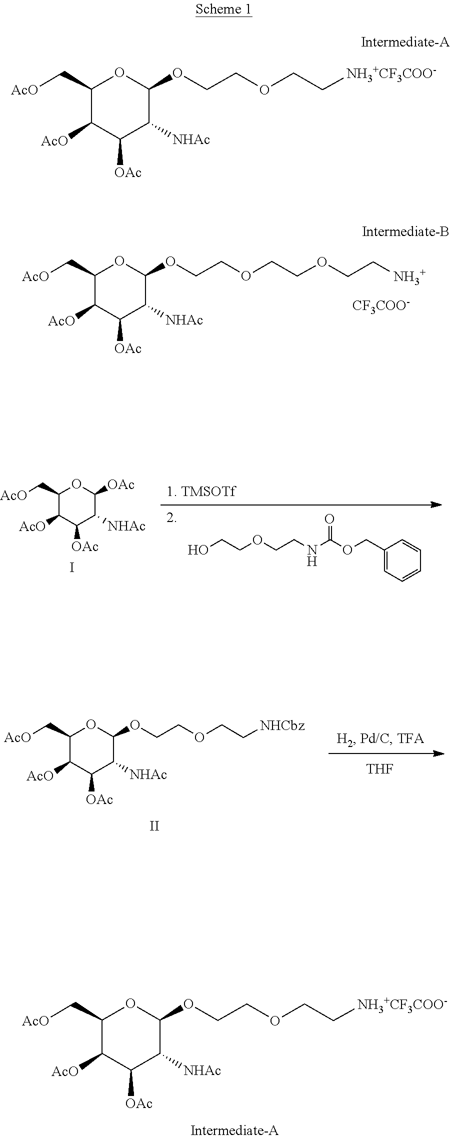

To a solution of Compound I (20.0 g, 51.4 mmol) in 100 mL 1,2-dichloroethane (DCE) was added TMSOTf (17.1 g, 77.2 mmol). The resulting reaction solution was stirred at 60° C. for 2 hrs, and then at 25° C. for 1 hr. Cbz protected 2-(2-aminoethoxy) ethan-1-ol (13.5 g, 56.5 mmol) in DCE (100 mL) dried over 4 Å powder molecular sieves (10 g) was added dropwise to the above mentioned reaction solution at 0° C. under N₂ atmosphere. The resulting reaction mixture was stirred at 25° C. for 16 hrs under N₂ atmosphere. The reaction mixture was filtered and washed with sat. NaHCO₃ (200 mL), water (200 mL) and sat. brine (200 mL). The organic layer was dried over anhydrous Na₂SO₄, filtered and concentrated under reduced pressure to give a crude product, which was triturated with 2-Methyltetrahydrofuran/heptane (5/3, v/v, 1.80 L) for 2 hrs. Resulting mixture was filtered and dried to give Compound II (15.0 g, 50.3% yield) as a white solid.

To a dried and argon purged hydrogenation bottle was carefully added 10% Pd/C (1.50 g), followed by 10 mL tetrahydrofuran (THF) and then a solution of Compound II (15.0 g, 26.4 mmol) in THF (300 mL) and TFA (trifluoroacetic acid, 3.00 g, 26.4 mmol). The resulting mixture was degassed and purged with H₂ three times and stirred at 25° C. for 3 hrs under H₂ (45 psi) atmosphere. Thin-layer chromatography (TLC, solvent: DCM:MeOH=10:1) indicated Compound II was consumed completely. The reaction mixture was filtered and concentrated under reduced pressure. Residue was dissolved in anhydrous DCM (500 mL) and concentrated. This process was repeated 3 times to give Intermediate-A (14.0 g, 96.5% yield) as a foamy white solid. ¹H NMR (400 MHZ DMSO-d₆): δ ppm 7.90 (d, J=9.29 Hz, 1H), 7.78 (br s, 3H), 5.23 (d, J=3.26 Hz, 1H), 4.98 (dd, J=11.29, 3.26 Hz, 1H), 4.56 (d, J=8.53 Hz, 1H), 3.98-4.07 (m, 3H), 3.79-3.93 (m, 2H), 3.55-3.66 (m, 5H), 2.98 (br d, J=4.77 Hz, 2H), 2.11 (s, 3H), 2.00 (s, 3H), 1.90 (s, 3H), 1.76 (s, 3H).

Intermediate-B was synthesized using similar procedures for synthesis of Intermediate-A. ¹H NMR (400 MHZ DMSO-d₆): δ ppm 7.90 (br d, J=9.03 Hz, 4H), 5.21 (d, J=3.51 Hz, 1H), 4.97 (dd, J=11.1 Hz, 1H), 4.54 (d, J=8.53 Hz, 1H), 3.98-4.06 (m, 3H), 3.88 (dt, J=10.9 Hz, 1H), 3.76-3.83 (m, 1H), 3.49-3.61 (m, 9H), 2.97 (br s, 2H), 2.10 (s, 3H), 1.99 (s, 3H), 1.88 (s, 3H), 1.78 (s, 3H). Mass calc. for C₂₀H₃₄N₂O₁₁: 478.22; found: 479.3 (M+H⁺).

### Example 3. Synthesis of GalNAc Ligand Cluster Phosphoramidite GLPA1, GLPA2 and GLPA1

Scheme 2 below was followed to prepare GLPA1 and GLPA2. Starting from benzyl protected propane-1,3-diamine, it was alkylated with tert-butyl 2-bromoacetate to afford triester Compound I. The benzyl protecting group was removed by hydrogenation to afford secondary amine Compound II. Amide coupling with 6-hydroxyhexanoic acid afforded Compound III. tert-Butyl protecting groups were then removed upon treatment of HCl in dioxane to generate triacid Compound IV. Amide coupling between triacid compound IV and Intermediate-A or Intermediate-B was performed to afford Compound Va or Vb. Phosphoramidite GLPA1 or GLPA2 was synthesized by phosphitylation of Compound Va or Vb with 2-Cyanoethyl N,N-diisopropylchlorophosphoramidite and a catalytic amount of 1H-tetrazole.

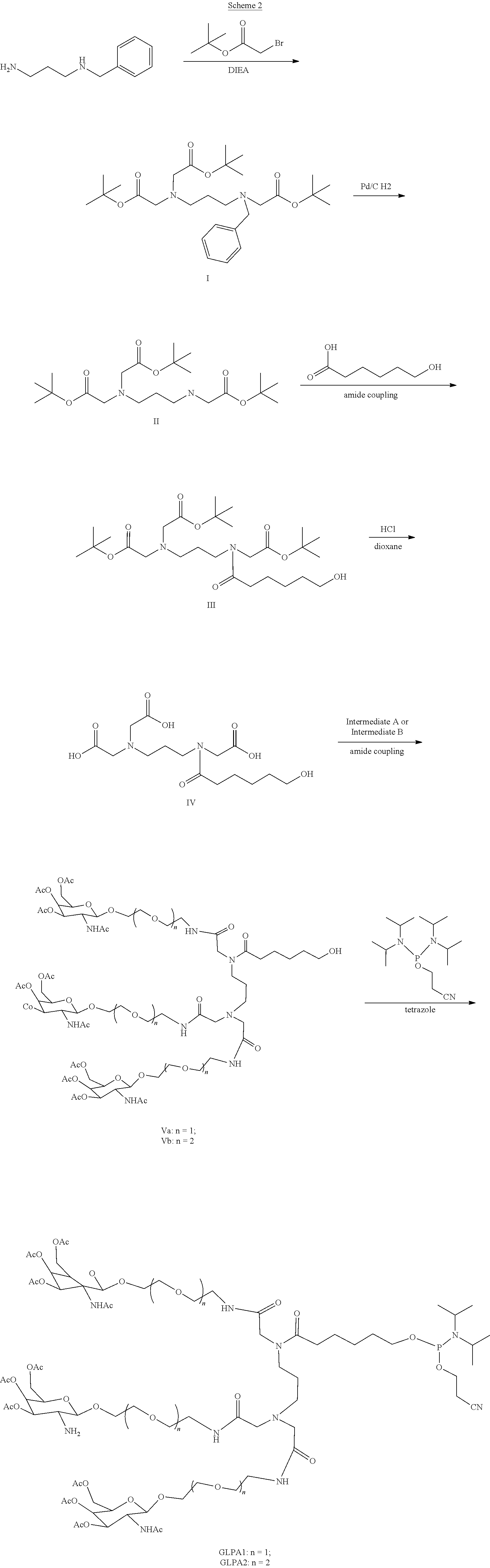

To a solution of N-Benzyl-1,3-propanediamine (5.00 g, 30.4 mmol) in dimethylformamide (DMF, 100 mL) was added tert-butyl 2-bromoacetate (23.7 g, 121 mmol), followed by addition of diisopropylethylamine (DIEA, 23.61 g, 182 mmol) dropwise. The resulting reaction mixture was stirred at 25-30° C. for 16 hrs. LCMS showed N-Benzyl-1,3-propanediamine was consumed completely. Reaction mixture was diluted with H₂O (500 mL) and extracted with EtOAc (500 mL×2). The combined organics were washed with sat. brine (1 L), dried over anhydrous Na₂SO₄, filtered, and concentrated under reduced pressure to give crude product, which was purified by silica gel column chromatography (gradient: petroleum ether:ethyl acetate from 20:1 to 5:1). Compound I (12.1 g, 78.4% yield) was obtained as a colorless oil. ¹H NMR (400 MHZ, CDCl₃): δ ppm 7.26-7.40 (m, 5H), 3.79 (s, 2H), 3.43 (s, 4H), 3.21 (s, 2H), 2.72 (dt, J=16.9, 7.34 Hz, 4H), 1.70 (quin, J=7.2 Hz, 2H), 1.44-1.50 (m, 27H).

A dried hydrogenation bottle was purged with Argon three times. Pd/C (200 mg, 10%) was added, followed by MeOH (5 mL) and then a solution of Compound I (1.00 g, 1.97 mmol) in MeOH (5 mL). The reaction mixture was degassed under vacuum and refilled with H₂. This process was repeated three times. The mixture was stirred at 25° C. for 12 hrs under H₂ (15 psi) atmosphere. LCMS showed Compound I was consumed completely. The reaction mixture was filtered under reduced pressure under N₂ atmosphere. Filtrate was concentrated under reduced pressure to give Compound II (655 mg, 79.7% yield) as yellow oil, which was used for the next step without further purification. ¹H NMR (400 MHZ, CDCl₃): δ ppm 3.44 (s, 4H), 3.31 (s, 2H), 2.78 (t, J=7.1 Hz, 2H), 2.68 (t, J=6.9 Hz, 2H), 1.88 (br s, 1H), 1.69 (quin, J=7.03 Hz, 2H), 1.44-1.50 (s, 27H).

A mixture of Compound II (655 mg, 1.57 mmol), 6-hydroxyhexanoic acid (249 mg, 1.89 mmol), DIEA (1.02 g, 7.86 mmol), 1-ethyl-3-(3-dimethylaminopropyl) carbodiimide hydrochloride (EDCI, 904 mg, 4.72 mmol), and 1-Hydroxybenzotriazole (HOBt, 637 mg, 4.72 mmol) in DMF (6 mL) was degassed and purged with N₂ three times, and then was stirred at 25° C. for 3 hrs under N₂ atmosphere. LCMS indicated desired product. The reaction mixture was diluted with H₂O (10 mL) and extracted with EtOAc 20 mL (10 mL×2). Organics were combined and washed with sat. brine (20 mL), dried over anhydrous Na₂SO₄, filtered, and concentrated to give crude product, which was purified by silica gel column chromatography (gradient:petroleum ether:ethyl acetate from 5:1 to 1:1) to afford Compound III (650 mg, 77.8% yield) as a yellow oil. ¹H NMR (400 MHZ, CDCl₃): δ ppm 3.90-3.95 (s, 2H), 3.63 (t, J=6.40 Hz, 2H), 3.38-3.45 (m, 6H), 2.72 (t, J=6.65 Hz, 2H), 2.40 (t, J=7.28 Hz, 2H), 1.55-1.75 (m, 8H), 1.44 (s, 27H). Mass calc. for C₂₇H₅₀N₂O₈: 530.36; found: 531.3 (M+H⁺).

A mixture of Compound III (5.5 g, 10.3 mmol) in HCl/dioxane (2M, 55 mL) was stirred at 25° C. for 3 hrs. LCMS showed complete consumption of Compound III. Reaction mixture was filtered, washed with EtOAc (50 mL), and dried under reduced pressure to give crude product. It was dissolved in CH₃CN (50 mL), volatiles were removed under vacuum. This process was repeated three times to give Compound IV (2.05 g, 54.5% yield) as a white solid. ¹H NMR (400 MHZ, D₂O): δ ppm 4.21 (s, 1H), 4.07 (d, J=4.5 Hz, 4H), 3.99 (s, 1H), 3.45-3.52 (m, 3H), 3.42 (t, J=6.5 Hz, 1H), 3.32-3.38 (m, 1H), 3.24-3.31 (m, 1H), 2.37 (t, J=7.4 Hz, 1H), 2.24 (t, J=7.4 Hz, 1H), 1.99 (dt, J=15.5, 7.53 Hz, 1H), 1.85-1.94 (m, 1H), 1.85-1.94 (m, 1H), 1.39-1.56 (m, 4H), 1.19-1.31 (m, 2H).

A mixture of Compound IV (500 mg, 1.05 mmol), Intermediate-A (2.02 g, 3.67 mmol), DIEA (813 mg, 6.30 mmol), EDCI (704 mg, 3.67 mmol) and HOBt (496 mg, 3.67 mmol) in DMF (10 mL) was degassed and purged with N₂ for 3 times, and then the mixture was stirred at 25° C. for 3 hrs under N₂ atmosphere. LCMS indicated desired product. The reaction mixture was quenched by addition of H₂O (10 mL), extracted with DCM (10 mL×2). The combined organics were extracted with 10% citric acid (20 mL). The aqueous phase was neutralized with saturated NaHCO₃ solution and re-extracted with DCM (10 mL×2). Organics were dried over sodium sulfate, filtered and concentrated under reduced pressure to give Compound Va (570 mg, 0.281 mmol, 26.8% yield) as a white solid. ¹H NMR: (400 MHZ, CDCl₃) ppm δ 7.84-8.12 (m, 3H), 6.85-7.15 (m, 2H), 6.66-6.81 (m, 1H), 5.36 (br d, J=2.7 Hz, 3H), 5.11-5.27 (m, 3H), 4.63-4.85 (m, 3H), 3.90-4.25 (m, 18H), 3.37-3.75 (m, 28H), 3.15-3.28 (m, 4H), 2.64 (br d, J=6.53 Hz, 2H), 2.30-2.46 (m, 2H), 2.13-2.18 (m, 9H), 2.05 (s, 9H), 1.94-2.03 (m, 18H), 1.68 (br s, 2H), 1.45 (br s, 2H), 1.12 (br t, J=7.0 Hz, 2H).

To a solution of Compound Va (260 mg, 0.161 mmol) in anhydrous DCM (5 mL) was added diisopropylammonium tetrazolide (30.3 mg, 0.177 mmol), followed by dropwise addition of 3-bis(diisopropylamino)phosphanyloxypropanenitrile (194 mg, 0.645 mmol) at ambient temperature under N₂. The reaction mixture was stirred at 20˜25° C. for 2 hrs. LCMS indicated Compound Va was consumed completely. After cooling to −20° C., the reaction mixture was added to a stirred solution of brine/saturated aq. NaHCO₃ (1:1, 5 mL) at 0° C. After stirring for 1 min, DCM (5 mL) was added. Layers were separated. Organics were washed with brine/saturated aq. NaHCO₃ solution (1:1, 5 mL), dried over Na₂SO₄, filtered, and concentrated to ˜1 mL of volume. The residue solution was added dropwise to 20 mL methyl tert-butyl ether (MTBE) with stirring. This resulted in precipitation of white solid. The mixture was centrifuged, and solid was collected. The solid was redissolved in 1 mL of DCM and precipitated by addition of MTBE (20 mL). Solid was again isolated by centrifuge. The solid collected was dissolved in anhydrous CH₃CN. Volatiles were removed. This process was repeated two more times to afford GalNAc ligand phosphoramidite compound GLPA1 (153 mg, 84.4 μmol) as a white solid. ¹H NMR (400 MHZ, CDCl₃): ppm δ 7.71-8.06 (m, 2H), 6.60-7.06 (m, 3H), 5.37 (br d, J=3.0 Hz, 3H), 5.18-5.32 (m, 3H), 4.70-4.86 (m, 3H), 3.92-4.25 (m, 18H), 3.42-3.85 (m, 30H), 3.25 (m, 4H), 2.59-2.75 (m, 4H), 2.27-2.44 (m, 2H), 2.15-2.20 (s, 9H) 2.07 (s, 9H), 1.96-2.03 (m, 18H), 1.65 (br s, 4H), 1.44 (br d, J=7.28 Hz, 2H), 1.14-1.24 (m, 12H). ³¹P NMR (CDCl₃): ppm δ 147.15.

GalNAc ligand phosphoramidite compound GLPA2 was synthesized using the same procedure except Intermediate-B was used. ¹H NMR (400 MHZ, CDCl₃): ppm δ 7.94-8.18 (m, 1H), 7.69 (br s, 1H), 6.66-7.10 (m, 3H), 5.35 (d, J=3.5 Hz, 3H), 5.07-5.25 (m, 3H), 4.76-4.86 (m, 3H), 4.01-4.31 (m, 10H), 3.91-4.01 (m, 8H), 3.74-3.86 (m, 4H), 3.52-3.71 (m, 30H), 3.42-3.50 (m, 6H), 3.15-3.25 (m, 4H), 2.52-2.70 (m, 4H), 2.22-2.45 (m, 2H), 2.15-2.22 (s, 9H), 2.06 (s, 9H), 1.95-2.03 (m, 18H), 1.77 (br s, 2H), 1.58-1.66 (m, 4H), 1.40 (m, 2H), 1.08-1.24 (m, 12H). ³¹P NMR (CDCl₃): ppm δ 147.12.

Scheme 3 below was followed to prepare GLPA15.

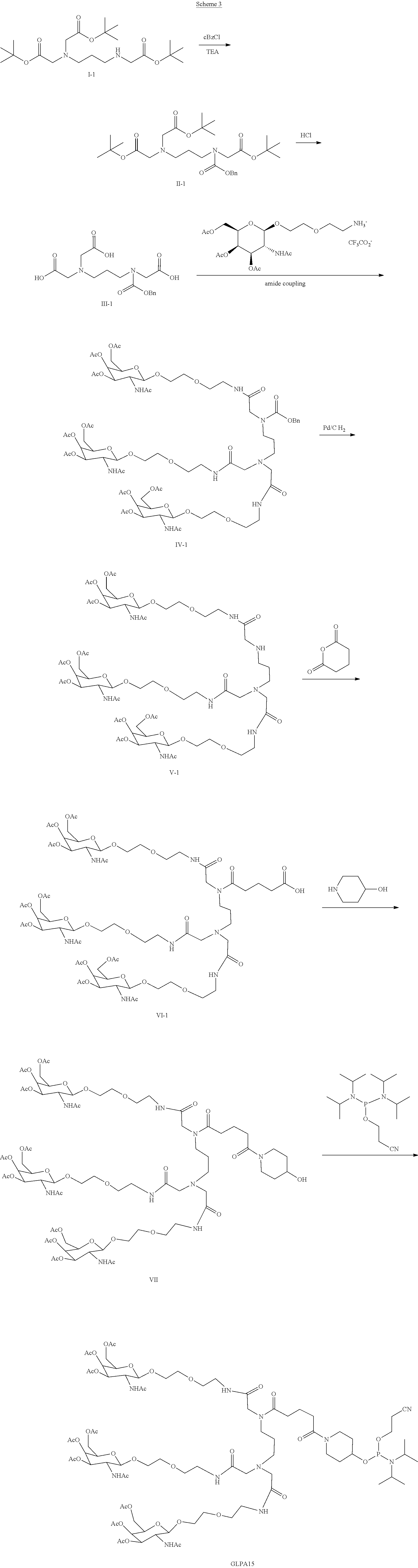

Starting from secondary amine Compound I-1 (Compound II in Scheme 2), Cbz protection was introduced to afford Compound II-1. The tert-Butyl groups of Compound II-1 were removed by treatment with acid to give triacid Compound III-1. Amide coupling of Compound III-1 with Intermediate-A afforded Compound IV-1. The Cbz protecting group of Compound IV-1 was removed by hydrogenation to afford secondary amine Compound V-1, which was reacted with glutaric anhydride to afford carboxylic Compound VI-1. Compound VI-1 reacted with piperidin-4-ol under amide coupling reaction condition to affords Compound VII. Phosphoramidite Compound GLPA15 was synthesized by treating Compound VII with 2-Cyanoethyl N,N diisopropylchlorophosphoramidite and a catalytic amount of 1H-tetrazole.

¹H NMR (400 MHz in DMSO-d6): δ ppm 8.05 (br d, J=6.50 Hz, 2H), 7.81 (br d, J=9.01 Hz, 3H), 5.22 (d, J=3.25 Hz, 3H), 4.98 (dd, J=11.26, 3.25 Hz, 3H), 4.55 (br d, J=8.50 Hz, 3H), 4.03 (s, 9H), 3.64-3.97 (m, 12H), 3.55-3.63 (m, 6H), 3.50 (br s, 5H), 3.40 (br d, J=6.13 Hz, 6H), 3.17-3.30 (m, 9H), 3.07 (br d, J=14.26 Hz, 4H), 2.76 (t, J=5.82 Hz, 2H), 2.18-2.47 (m, 6H), 2.10 (s, 9H), 1.99 (s, 9H), 1.89 (s, 9H), 1.78 (s, 9H), 1.52-1.74 (m, 6H), 1.12-1.19 (m, 12H). ³¹P NMR (DMSO-d6): ppm δ 145.25.

In certain studies, a method used to attach a targeting group comprising GalNAc (also referred to herein as a GalNAc delivery compound) to the 5′-end of a sense strand included use of a GalNAc phosphoramidite (GLPA1) in the last coupling step in the solid phase synthesis, using a synthetic process such as the process used if oligonucleotide chain propagation of adding a nucleotide to the 5′-end of the sense strand is performed.

In some studies a method of attaching a targeting group comprising GalNAc to the 3′-end of a sense strand comprised use of a solid support (CPG) that included a GLO-n. In some studies, a method of attaching a targeting group comprising GalNAc to the 3′-end of a sense strand comprises attaching a GalNAc targeting group to CPG solid support through an ester bond and using the resulting CPG with the attached GalNAc targeting group when synthesizing the sense strand, which results in the GalNAc targeting group attached at the 3′-end of the sense strand.

### Example 4. In Vitro Screening of PKK siRNA Duplexes

Cryopreserved PHHs are thawed and adjusted to appropriate density, and seeded into 96-well plates. Cells are transfected with test siRNAs or a control siRNA using Lipofectamine RNAiMax at the same time of seeding. The test siRNAs were tested at two concentrations in triplicate, 1 nM and 0.2 nM. In a separated experiment, the test siRNAs were tested at 10 nM in triplicate. Results from these two experiments were summarized in Table 4.

The medium was removed and the cells were harvested for RNA extraction, 24 hours after transfection. Total RNA was extracted by RNeasy® 96 Kit (QIAGEN-74182) according to the manual provided by the manufacturer. The cDNA is Synthesized with FastKing RT Kit (With gDNase) (Tiangen-KR116-02) according to the manual provided by the manufacturer.

The PKK cDNA is be detected by qPCR. GAPDH cDNA is detected in parallel as an internal control. Percent of knockdown is calculated by comparing expression level of PKK mRNA from the PKK siRNA treated samples and non-targeted control siRNA treated samples. The duplex sequences used correspond to those shown in Table 2.

TABLE 4
Experimental results of in vitro studies using various
PKK RNAi agents to inhibit PKK expression in primary human
hepatocytes. Studies are described elsewhere herein.

| | Percent inhibition at concentration tested | | |
|---|---|---|---|
| Duplex | 0.2 nM | 1 nM | 10 nM |

| ID | % inh | STD | % inh | STD | % inh | STD |
|---|---|---|---|---|---|---|
| AV00001 | 13.2 | 11.0 | 24.5 | 3.2 | 20.8 | 6.8 |
| AV00003 | 19.2 | 8.8 | 33.3 | 11.5 | 28.5 | 1.8 |
| AV00004 | 4.8 | 6.4 | 23.8 | 5.8 | 14.7 | 2.0 |
| AV00005 | 16.5 | 13.4 | 27.0 | 10.9 | 26.6 | 6.6 |
| AV00006 | 11.6 | 3.8 | 27.8 | 2.0 | 32.0 | 6.8 |
| AV00007 | 9.7 | 11.9 | 13.9 | 14.0 | 17.3 | 4.5 |
| AV00008 | 3.6 | 24.7 | 8.7 | 6.9 | 31.0 | 6.4 |
| AV00009 | −13.1 | 10.5 | 0.4 | 14.6 | 14.9 | 13.6 |
| AV00010 | −6.9 | 44.7 | 5.8 | 19.2 | 19.6 | 14.4 |
| AV00011 | 34.5 | 18.6 | 34.7 | 11.3 | 28.0 | 15.6 |
| AV00012 | 17.5 | 4.3 | 19.4 | 11.2 | 25.1 | 8.2 |
| AV00013 | 17.7 | 32.3 | 21.5 | 8.2 | 23.4 | 3.7 |
| AV00014 | 13.4 | 20.1 | 27.2 | 10.1 | 31.2 | 8.0 |
| AV00015 | 24.7 | 27.7 | 28.2 | 3.9 | 29.6 | 10.5 |
| AV00016 | 25.5 | 18.3 | 25.6 | 8.6 | 24.5 | 11.3 |
| AV00017 | 19.9 | 8.9 | 16.0 | 12.4 | 10.7 | 7.8 |
| AV00018 | 5.9 | 24.4 | 13.8 | 18.8 | 3.8 | 11.5 |
| AV00019 | 26.1 | 11.6 | 38.3 | 4.5 | 18.6 | 6.4 |
| AV00020 | 30.3 | 5.9 | 33.9 | 3.0 | 22.2 | 13.9 |
| AV00021 | 11.4 | 3.0 | 24.7 | 7.0 | 16.3 | 10.0 |
| AV00022 | 19.1 | 16.1 | 10.7 | 8.0 | 35.9 | 3.9 |
| AV00023 | 25.1 | 2.9 | 20.1 | 6.0 | 25.3 | 3.3 |
| AV00024 | 35.9 | 20.3 | 49.7 | 11.6 | 36.8 | 15.6 |
| AV00025 | 32.9 | 13.4 | 17.2 | 2.4 | 32.2 | 13.9 |
| AV00026 | 51.5 | 1.9 | 30.0 | 3.0 | 47.0 | 8.2 |
| AV00027 | 40.3 | 7.8 | 24.1 | 19.3 | 35.2 | 10.3 |
| AV00028 | 39.6 | 13.3 | 47.8 | 4.9 | 35.4 | 11.4 |
| AV00029 | 26.7 | 13.4 | 23.8 | 12.0 | 29.4 | 10.9 |
| AV00030 | 9.9 | 13.1 | 15.3 | 8.4 | 18.4 | 3.3 |
| AV00031 | 10.4 | 18.7 | 32.6 | 3.8 | 35.0 | 8.8 |
| AV00032 | −1.1 | 7.9 | 5.5 | 6.3 | 17.4 | 6.4 |
| AV00033 | 14.1 | 7.5 | 1.6 | 22.4 | 28.2 | 4.1 |
| AV00034 | 18.6 | 7.0 | 25.3 | 14.9 | 30.2 | 7.0 |
| AV00035 | 14.3 | 8.3 | 10.9 | 9.9 | 30.4 | 1.9 |
| AV00036 | 37.7 | 9.5 | −0.4 | 29.8 | 24.9 | 2.4 |
| AV00037 | −8.4 | 17.6 | 12.5 | 11.6 | 31.2 | 16.1 |
| AV00038 | −8.9 | 31.4 | 22.7 | 18.1 | 36.2 | 7.3 |
| AV00039 | −27.1 | 54.0 | −1.2 | 15.5 | 5.7 | 18.9 |
| AV00040 | −24.3 | 84.3 | −3.4 | 11.8 | 15.7 | 7.6 |
| AV00041 | 21.2 | 14.1 | 30.8 | 16.0 | 30.1 | 15.5 |
| AV00042 | 11.5 | 23.4 | 30.0 | 2.9 | 33.1 | 7.9 |
| AV00043 | 23.8 | 5.0 | 29.1 | 4.3 | 36.9 | 11.7 |
| AV00044 | 24.0 | 1.7 | 26.4 | 2.4 | 15.3 | 16.7 |
| AV00045 | 26.7 | 16.5 | 27.9 | 2.5 | 21.1 | 0.9 |
| AV00046 | 14.0 | 15.5 | 1.9 | 18.3 | 28.4 | 12.2 |
| AV00047 | 23.7 | 11.5 | 24.9 | 10.8 | 22.6 | 8.5 |
| AV00048 | 14.5 | 9.4 | 21.5 | 3.3 | 24.9 | 10.0 |
| AV00049 | 24.2 | 14.9 | 14.3 | 15.9 | 21.0 | 5.8 |
| AV00050 | −2.4 | 18.1 | 25.0 | 4.8 | 26.2 | 14.0 |
| AV00051 | −0.6 | 4.0 | 27.7 | 7.1 | 38.0 | 5.7 |
| AV00052 | 24.4 | 8.7 | 41.7 | 0.9 | 13.3 | 2.2 |

### Example 5. In Vivo Screening of PKK siRNA Agents

To assess in vivo activity of PKK siRNAs, mice (4 mice in each group) infected with AAV encoding the human PKK gene were used. At 14 days before dosing of siRNAs, female C57BL/6J mice were infected by intravenous administration of a solution of an adeno-associated virus 8 (AAV8, 2×10¹¹ viral particles) vector encoding the human PKK gene. At day 0, mice were subcutaneously administered a single 3 mg/kg of PKK siRNA agents or PBS. Blood samples were collected at day 0, before dosing of siRNA and at day 7, at termination. Human PKK protein concentration was measured by ELISA assay per manufacturer's recommended protocol (Abcam, Human Prekallikrein 1B ELISA Kit, ab202405). Percent of knockdown was calculated by comparing human PKK level in day 7 mouse plasma samples of siRNA treated group (normalized to PKK expression pre-dosing of siRNA) to PBS treated group. Results are summarized in Table 5, Table 6 and Table 7. In this example, the GLO-0 in said compounds in Table 3 refers to the compound GalNAc3 in Jayaprakash, et al., (2014) J. Am. Chem. Soc., 136, 16958-16961. The duplex sequences used correspond to those shown in Table 3.

TABLE 5
PKK siRNA single 3 mpk subcutaneous dose screening in
AAV-PKK transduced mouse. Percent of reduction of human
PKK in mouse serum was normalized to PKK expression
pre-dosing of siRNA and to the PBS control group.

| | Percent reduction of |
|---|---|
| | human PKK in mouse |
| | serum measured by |
| Duplex ID | ELISA assay |
| AD00188 | 66% |
| AD00117 | 59% |
| AD00118 | 76% |
| AD00119 | 81% |
| AD00120 | 60% |
| AD00121 | 48% |
| AD00274 | 55% |
| AD00275 | 50% |
| AD00276 | 84% |
| AD00277 | 69% |
| AD00278 | 92% |
| AD00279 | 69% |
| AD00280 | 59% |

TABLE 6
PKK siRNA single 3 mpk subcutaneous dose screening in
AAV-PKK transduced mouse. Percent of reduction of human
PKK in mouse serum was normalized to PKK expression
pre-dosing of siRNA and to the PBS control group.

| | Percent reduction of |
|---|---|
| | human PKK in mouse |
| Duplex | serum measured by |
| ID | ELISA assay |
| AD00278 | 91% |
| AD00303 | 70% |
| AD00304 | 70% |
| AD00305 | 59% |
| AD00306 | 13% |
| AD00307 | 36% |
| AD00308 | 47% |
| AD00309 | 37% |
| AD00310 | 53% |
| AD00311 | 85% |
| AD00312 | 83% |
| AD00313 | 62% |
| AD00314 | 31% |
| AD00315 | 53% |

TABLE 7
PKK siRNA single 3 mpk subcutaneous dose screening in
AAV-PKK transduced mouse. Percent of reduction of human
PKK in mouse serum was normalized to PKK expression
pre-dosing of siRNA and to the PBS control group.

| | Percent reduction of |
|---|---|
| | human PKK in mouse |
| | serum measured by |
| Duplex ID | ELISA assay |
| AD00278 | 84% |
| AD00398 | 56% |
| AD00399 | 58% |
| AD00400 | 43% |
| AD00401 | 34% |
| AD00402 | 26% |
| AD00403 | 15% |
| AD00404 | 60% |
| AD00405 | 63% |
| AD00406 | 64% |
| AD00407 | 75% |
| AD00408 | 64% |
| AD00409 | 48% |

### Example 6. In Vivo Testing of PKK siRNA Agents

To assess in vivo activity of PKK siRNAs, mice (6 mice in each group) infected with AAV encoding the human PKK gene were used. At 14 days before dosing of siRNAs, female C57BL/6J mice were infected by intravenous administration of a solution of an adeno-associated virus 8 (AAV8, 2×10¹¹ viral particles) vector encoding the human PKK gene. At day 0, mice were subcutaneously administered a single 3 mg/kg of PKK siRNA agents or PBS. Blood samples were collected at day 0, before dosing of siRNA and at day 7, and day 14 at termination. Human PKK protein concentration was measured by ELISA assay per manufacturer's recommended protocol (Abcam, Human Prekallikrein 1B ELISA Kit, ab202405). Percent of knockdown was calculated by comparing human PKK level in day 7 and day 14 in mouse plasma samples of siRNA treated group (normalized to PKK expression pre-dosing of siRNA) to PBS treated group. Results are summarized in Table 8. In this example, the GLO-0 in said compounds in Table 3 refers to the compound GalNAc3 in Jayaprakash, et al., (2014) J. Am. Chem. Soc., 136, 16958-16961. The duplex sequences used correspond to those shown in Table 3.

TABLE 8
PKK siRNA single 3 mpk subcutaneous dose testing in
AAV-PKK transduced mouse. Percent of reduction of human
PKK in mouse serum was normalized to PKK expression
pre-dosing of siRNA and to the PBS control group

| | Percent reduction of human PKK in mouse | |
|---|---|---|
| | serum measured by ELISA assay normalized to | |
| | day 0 | |
| Duplex | Day 7 | Day 14 |

| ID | Mean | SD | Mean | SD |
|---|---|---|---|---|
| AD00119 | 74% | 0.13 | 79% | 0.06 |
| AD00276 | 75% | 0.06 | 77% | 0.08 |
| AD00278 | 84% | 0.04 | 87% | 0.08 |
| AD00311 | 78% | 0.16 | 90% | 0.03 |
| AD00312 | 75% | 0.12 | 82% | 0.06 |
| AD00407 | 56% | 0.19 | 64% | 0.11 |

### Example 7. In Vitro Screening of PKK siRNA Duplexes

Huh7 cells were trypsinized and adjusted to appropriate density, and seeded into 96-well plates. Cells were transfected with test siRNAs or a control siRNA using Lipofectamine RNAiMax (Invitrogen-13778-150) at the same time of seeding following the protocol according to manufacturer's recommendation. The siRNAs were tested at two concentrations (0.2 nM and 1.0 nM) in triplicate.

Day 0, psiCHECK™-2 Vector Transfection (One Plate)
(1) Transfer 2.5 μg psiCHECK™-2 Vector plasmid into an RNASE free Eppendorf tube (solution mix #1)
(2) Add trypsin to disassociate Huh7 cells in one flask, and count cells using Vi-Cell counting machine, adjust the cell density to 1*10\hat{ }5/ml
(3) Transfer 7.5 μL Fugene-HD into solution mix #1 tube, mix.
(4) Add solution in Step 3 into cell suspension, mix, and dispense suspension into the 96 well plate (100 μl/well)
   3.2 Day 1, siRNAs Transfection
(1) Dilute Lipofectamine® RNAiMAX Reagent with Opti-MEM® Medium.
(2) Dilute the siRNA with RNA-free water to make 12× stock.
(3) Mix equal volume of diluted RNAiMax and siRNA. Incubate the mixture at RT for 15 min to allow complex formation
(4) Add 45 μl/well compound Lipofectamine® RNAiMAX (Opti-MEM) mix into 225 μl/well DMEM fresh medium, and discard the supernatants in assay plate, add 120 μl/well compound mix into 96 well plates no compound control well was defined as cells transfected with psiCHECK™-2 Vector and without siRNA treatment; blank control was cell only wells.

### 3.3 Day 2, Dual-Glo® Luciferase Assay

(1) Add Reagent to assay plate, wait 10 minutes to allow for cell lysis to occur.
(2) Transfer 100 μl cell lysates into a plate, then measure the firefly luminescence.
(3) Add 50 μl of Dual-Glo® Stop & Glo® Reagent to the assay plates and mix, wait 10 minutes, then measure Renilla luminescence.
(4) Calculate the relative expression

### 4. Data Analysis

Ratio of sample well=(sample Renilla luminescence−background blank)/(sample Fireflyluminescence−background blank)

Ratio of no compound control well=(control Renilla luminescence−background blank)/(control sample Fireflyluminescence−background blank)

% inhibition=100−(Ratio of sample well/the average Ratio of no compound control)×100%.

Results are summarized in Table 9, Table 10, Table 11 and Table 12.

TABLE 9
Experimental results of in vitro studies using various
PKK RNAi agents to inhibit PKK expression. The duplex
sequences used correspond to those shown in table 2.

| Percent inhibition at concentration tested | |
|---|---|
| 1 nM | 0.2 nM |

| Duplex ID | % inh | STD | % inh | STD |
|---|---|---|---|---|
| AV00768 | 69.40 | 7.17 | 39.19 | 3.64 |
| AV00769 | 71.54 | 7.12 | 39.01 | 1.72 |
| AV00770 | 76.71 | 5.61 | 59.08 | 3.51 |
| AV00771 | 89.33 | 3.15 | 75.90 | 1.92 |
| AV00772 | 87.42 | 2.27 | 67.86 | 8.12 |
| AV00773 | 79.23 | 2.53 | 63.77 | 4.28 |

TABLE 10
Experimental results of in vitro studies using various
PKK RNAi agents to inhibit PKK expression. The duplex
sequences used correspond to those shown in table 2.

| | Percent inhibition at concentration tested | |
|---|---|---|
| Duplex | 1 nM | 0.2 nM |

| ID | % inh | STD | % inh | STD |
|---|---|---|---|---|
| AV00732 | 66.63 | 1.31 | 38.62 | 3.47 |
| AV00733 | 59.66 | 2.71 | 30.24 | 3.41 |
| AV00734 | 37.08 | 0.54 | 3.67 | 7.50 |
| AV00735 | 75.60 | 1.21 | 45.88 | 3.74 |
| AV00736 | 55.46 | 2.13 | 22.03 | 7.53 |
| AV00737 | 66.33 | 1.82 | 26.38 | 1.96 |
| AV00738 | 49.36 | 3.89 | 14.59 | 3.34 |
| AV00739 | 72.95 | 0.53 | 44.42 | 0.27 |
| AV00740 | 80.47 | 1.40 | 62.57 | 1.59 |
| AV00741 | 72.33 | 3.34 | 36.04 | 4.92 |
| AV00742 | 54.74 | 1.26 | 21.95 | 2.45 |
| AV00744 | 61.74 | 0.30 | 39.87 | 5.41 |
| AV00745 | 72.34 | 2.64 | 55.08 | 4.11 |
| AV00746 | 79.73 | 2.52 | 67.06 | 2.23 |
| AV00747 | 52.49 | 2.18 | 19.27 | 2.69 |
| AV00748 | 34.11 | 1.38 | 4.17 | 5.73 |
| AV00749 | 56.80 | 4.32 | 17.04 | 4.48 |
| AV00750 | 66.72 | 3.26 | 38.85 | 2.67 |
| AV00751 | 82.09 | 2.96 | 54.14 | 1.73 |
| AV00752 | 80.86 | 1.02 | 55.93 | 1.45 |
| AV00753 | 78.35 | 3.67 | 49.66 | 5.47 |
| AV00754 | 66.54 | 2.11 | 37.07 | 7.73 |
| AV00755 | 56.05 | 2.98 | 27.60 | 8.76 |
| AV00756 | 60.64 | 3.11 | 35.59 | 5.20 |
| AV00757 | 85.16 | 0.34 | 65.55 | 4.90 |
| AV00758 | 84.90 | 1.01 | 65.94 | 9.45 |
| AV00759 | 76.10 | 5.06 | 52.89 | 1.44 |
| AV00760 | 74.94 | 1.60 | 51.28 | 1.83 |
| AV00761 | 55.38 | 4.32 | 22.59 | 5.36 |
| AV00762 | 65.76 | 2.04 | 28.41 | 7.63 |
| AV00763 | 63.21 | 1.45 | 29.97 | 7.59 |
| AV00764 | 57.61 | 5.33 | 19.52 | 12.17 |
| AV00766 | 33.89 | 8.60 | 8.85 | 3.81 |
| AV00767 | 57.12 | 3.27 | 26.86 | 4.57 |
| AV00774 | 44.96 | 2.37 | 17.63 | 4.40 |
| AV00775 | 55.71 | 5.20 | 11.24 | 5.29 |
| AV00777 | 66.90 | 3.46 | 36.68 | 4.39 |
| AV00778 | 74.82 | 1.33 | 46.67 | 2.35 |
| AV00779 | 60.62 | 3.51 | 35.71 | 3.49 |
| AV00780 | 40.33 | 6.12 | 4.48 | 5.98 |
| AV00781 | 57.11 | 4.21 | 31.90 | 5.35 |
| AV00782 | 62.18 | 1.89 | 44.05 | 3.17 |
| AV00786 | 49.65 | 2.15 | 32.34 | 6.23 |
| AV00787 | 20.64 | 0.58 | 6.28 | 4.84 |
| AV00788 | 59.45 | 1.85 | 31.36 | 1.80 |
| AV00789 | 64.55 | 3.74 | 49.61 | 3.31 |
| AV00790 | 42.47 | 4.94 | 21.60 | 2.29 |
| AV00791 | 37.82 | 3.76 | 10.52 | 5.25 |
| AV00792 | 56.93 | 1.24 | 27.35 | 4.92 |

TABLE 11
Experimental results of in vitro studies using various
PKK RNAi agents to inhibit PKK expression. The duplex
sequences used correspond to those shown in table 2.

Percent inhibition at concentration tested

| | |
|---|---|
| 1 nM | 0.2 nM |

| | |
|---|---|
| Duplex ID | MEAN ± SD |

| | | |
|---|---|---|
| AV01712 | 55.59 ± 2.10 | 23.18 ± 3.38 |
| AV01714 | 48.04 ± 4.24 | −9.10 ± 9.37 |
| AV01715 | 36.93 ± 10.01 | 3.83 ± 10.72 |
| AV01717 | 31.32 ± 0.90 | −16.64 ± 8.42 |
| AV01718 | 47.58 ± 2.71 | 9.41 ± 12.80 |
| AV01719 | 44.07 ± 3.87 | 14.74 ± 6.18 |
| AV01720 | 51.53 ± 1.60 | −0.31 ± 4.62 |
| AV01721 | 30.13 ± 8.08 | −5.36 ± 2.90 |
| AV01722 | 42.09 ± 6.29 | −9.50 ± 5.67 |
| AV01723 | 50.20 ± 8.32 | −3.73 ± 6.77 |
| AV01724 | 53.52 ± 3.32 | 19.66 ± 8.62 |
| AV01725 | 49.24 ± 1.48 | 7.77 ± 12.75 |
| AV01726 | 63.98 ± 2.74 | 24.78 ± 7.55 |
| AV01727 | 46.18 ± 8.52 | 3.84 ± 9.57 |
| AV01728 | 41.49 ± 0.56 | 14.86 ± 0.69 |
| AV01729 | 50.00 ± 8.91 | 4.63 ± 6.61 |
| AV01730 | 46.14 ± 0.68 | 12.93 ± 9.24 |
| AV01731 | 38.71 ± 1.82 | 7.16 ± 2.71 |
| AV01732 | 55.78 ± 1.81 | 7.83 ± 5.26 |
| AV01733 | 45.45 ± 4.53 | 4.61 ± 3.21 |
| AV01734 | 38.14 ± 13.51 | 4.23 ± 8.32 |
| AV01735 | 57.83 ± 2.69 | 12.37 ± 7.13 |
| AV01736 | 53.07 ± 0.34 | 15.08 ± 12.30 |
| AV01737 | 44.40 ± 0.22 | 12.04 ± 8.81 |
| AV01738 | 32.84 ± 1.89 | 13.26 ± 7.06 |
| AV01739 | 50.57 ± 1.10 | −8.43 ± 3.67 |
| AV01740 | 42.69 ± 1.64 | 4.62 ± 18.73 |
| AV01741 | 18.53 ± 12.45 | 6.47 ± 0.33 |
| AV01742 | 45.50 ± 0.96 | 10.48 ± 11.44 |
| AV01960 | 44.19 ± 9.30 | 23.52 ± 1.61 |
| AV01963 | 52.54 ± 2.50 | 20.76 ± 7.13 |
| AV01964 | 41.08 ± 19.27 | 8.81 ± 2.60 |
| AV01965 | 40.35 ± 7.36 | 0.99 ± 9.39 |
| AV01966 | 37.85 ± 6.77 | 4.75 ± 0.44 |
| AV01967 | 49.31 ± 10.02 | 11.75 ± 6.48 |
| AV01968 | 49.63 ± 4.68 | 18.91 ± 0.39 |
| AV01969 | 56.89 ± 1.81 | 21.63 ± 8.95 |
| AV01970 | 41.68 ± 2.22 | 9.00 ± 0.67 |
| AV01971 | 37.10 ± 2.19 | 12.45 ± 1.25 |
| AV01972 | 57.63 ± 2.70 | 24.78 ± 7.05 |
| AV01973 | 30.66 ± 0.32 | 10.32 ± 5.82 |
| AV01974 | 48.29 ± 3.31 | 18.22 ± 1.79 |
| AV01975 | 48.72 ± 8.83 | 16.66 ± 0.05 |
| AV01976 | 30.65 ± 4.33 | 9.46 ± 4.47 |
| AV01977 | 26.80 ± 8.81 | 4.27 ± 7.71 |
| AV01978 | 27.30 ± 0.63 | 7.45 ± 10.69 |
| AV01980 | 45.57 ± 1.42 | 13.32 ± 2.43 |
| AV01981 | 48.84 ± 13.45 | 12.48 ± 7.82 |
| AV01982 | 41.39 ± 0.24 | 11.44 ± 5.55 |
| AV01983 | 47.08 ± 1.98 | 18.82 ± 1.57 |
| AV01984 | 29.95 ± 3.44 | 9.73 ± 17.68 |
| AV01985 | 57.59 ± 3.15 | 18.00 ± 0.66 |
| AV01986 | 63.44 ± 2.89 | 27.38 ± 21.30 |
| AV01987 | 51.73 ± 2.39 | 17.10 ± 8.03 |
| AV01988 | 43.65 ± 1.69 | 11.20 ± 13.18 |
| AV01989 | 57.28 ± 1.66 | 12.73 ± 0.48 |
| AV02000 | 46.77 ± 1.72 | 2.25 ± 7.74 |
| AV02001 | 35.63 ± 17.99 | 21.60 ± 8.56 |
| AV02002 | 42.52 ± 1.34 | 12.27 ± 3.13 |
| AV02003 | 40.90 ± 0.49 | 0.93 ± 3.77 |
| AV02004 | 40.91 ± 4.57 | 10.28 ± 5.51 |
| AV02005 | 44.41 ± 4.13 | 4.25 ± 6.06 |
| AV02006 | 31.62 ± 4.08 | 3.27 ± 15.83 |
| AV02007 | 38.38 ± 4.91 | 11.80 ± 4.33 |
| AV02008 | 32.20 ± 0.15 | 4.25 ± 2.62 |
| AV02009 | 24.40 ± 4.56 | −0.58 ± 6.23 |

TABLE 12
Experimental results of in vitro studies using various
PKK RNAi agents to inhibit PKK expression. Studies
are described elsewhere herein. The duplex sequences
used correspond to those shown in table 2.

Percent inhibition at concentration tested

| | |
|---|---|
| 1 nM | 0.2 nM |

| | |
|---|---|
| Duplex ID | MEAN ± SD |

| | | |
|---|---|---|
| AV01743 | 59.20 ± 5.92 | 32.69 ± 4.79 |
| AV01744 | 62.63 ± 5.17 | 35.02 ± 13.09 |
| AV01745 | 44.73 ± 2.42 | 9.25 ± 6.21 |
| AV01748 | 39.00 ± 7.95 | −8.15 ± 19.28 |
| AV01749 | 48.44 ± 10.39 | 7.14 ± 11.49 |
| AV01750 | 53.40 ± 5.81 | 19.16 ± 3.14 |
| AV01751 | 60.02 ± 4.93 | 35.38 ± 4.59 |
| AV01752 | 47.88 ± 6.25 | 5.47 ± 7.05 |
| AV01753 | 41.56 ± 8.37 | 14.31 ± 6.59 |
| AV01754 | 39.27 ± 9.36 | 17.94 ± 1.95 |
| AV01755 | 56.09 ± 7.23 | 34.51 ± 7.74 |
| AV01756 | 44.49 ± 4.41 | 25.49 ± 9.15 |
| AV01757 | 61.93 ± 3.12 | 34.36 ± 16.88 |
| AV01758 | 39.14 ± 4.53 | 21.26 ± 7.29 |
| AV01759 | 56.70 ± 9.68 | 26.95 ± 0.90 |
| AV01760 | 65.70 ± 1.97 | 30.47 ± 5.38 |
| AV01761 | 55.90 ± 7.10 | 21.39 ± 4.99 |
| AV01762 | 53.75 ± 1.71 | 27.90 ± 2.81 |
| AV01763 | 33.05 ± 7.25 | 21.34 ± 15.81 |
| AV01764 | 6.76 ± 22.24 | 15.03 ± 4.55 |
| AV01765 | 32.76 ± 13.11 | 20.78 ± 16.36 |
| AV01766 | 37.24 ± 9.01 | 12.65 ± 11.82 |
| AV01767 | 19.06 ± 3.27 | 10.21 ± 5.10 |
| AV01768 | 46.90 ± 9.00 | 33.48 ± 12.67 |
| AV01769 | 47.34 ± 10.46 | 23.19 ± 2.52 |
| AV01770 | 54.05 ± 7.03 | 42.84 ± 10.35 |
| AV01771 | 57.98 ± 10.24 | 26.46 ± 17.70 |
| AV01773 | 49.56 ± 8.46 | 22.14 ± 12.07 |
| AV01774 | 74.07 ± 4.48 | 53.92 ± 3.79 |
| AV01775 | 80.78 ± 1.32 | 62.69 ± 5.20 |
| AV01776 | 78.07 ± 1.84 | 57.09 ± 4.45 |
| AV01777 | 67.78 ± 2.34 | 46.42 ± 8.76 |
| AV01778 | 42.80 ± 6.37 | −8.30 ± 22.18 |
| AV01779 | 54.88 ± 14.70 | 23.85 ± 7.58 |
| AV01780 | 58.65 ± 7.62 | 37.26 ± 18.57 |
| AV01781 | 62.39 ± 6.57 | 33.54 ± 5.76 |
| AV01782 | 62.71 ± 6.12 | 33.09 ± 5.57 |
| AV01783 | 59.62 ± 3.18 | 29.05 ± 3.07 |
| AV01784 | 76.58 ± 6.43 | 66.25 ± 4.15 |
| AV01785 | 73.99 ± 4.16 | 52.48 ± 2.61 |
| AV01786 | 79.58 ± 2.27 | 57.26 ± 1.68 |
| AV01787 | 63.63 ± 5.05 | 32.57 ± 20.27 |
| AV01788 | 80.97 ± 1.17 | 60.79 ± 5.22 |
| AV01789 | 73.25 ± 4.57 | 45.21 ± 4.80 |
| AV01790 | 74.25 ± 2.01 | 57.84 ± 0.79 |
| AV01791 | 77.42 ± 3.19 | 53.30 ± 9.78 |
| AV01792 | 73.80 ± 5.73 | 48.26 ± 10.00 |
| AV01793 | 64.15 ± 4.49 | 22.90 ± 19.58 |
| AV01794 | 57.84 ± 7.54 | 32.58 ± 11.37 |
| AV01795 | 46.28 ± 1.41 | 14.45 ± 7.25 |
| AV01796 | 23.76 ± 3.72 | 5.90 ± 5.68 |
| AV01797 | 69.45 ± 2.98 | 55.33 ± 2.18 |
| AV01798 | 71.58 ± 1.77 | 55.39 ± 9.14 |
| AV01799 | 77.95 ± 3.51 | 61.38 ± 4.29 |
| AV01800 | 73.73 ± 4.10 | 41.72 ± 6.51 |
| AV01801 | 73.11 ± 2.07 | 52.28 ± 5.06 |
| AV01802 | 80.05 ± 2.00 | 63.46 ± 4.94 |
| AV01803 | 52.77 ± 5.48 | 22.00 ± 3.57 |
| AV01804 | 74.86 ± 2.27 | 57.29 ± 6.32 |
| AV01836 | 56.42 ± 12.02 | 22.03 ± 9.84 |
| AV01837 | 69.83 ± 3.38 | 37.19 ± 10.45 |
| AV01838 | 54.37 ± 8.04 | 9.91 ± 3.00 |
| AV01839 | 37.70 ± 0.77 | 6.32 ± 22.05 |
| AV01841 | 31.98 ± 15.49 | 11.61 ± 28.14 |
| AV01842 | 39.04 ± 12.24 | 10.22 ± 3.89 |
| AV01843 | 39.73 ± 6.39 | −19.61 ± 13.39 |
| AV01844 | 47.70 ± 8.79 | 2.32 ± 20.64 |
| AV01845 | 41.82 ± 6.63 | −8.81 ± 1.28 |
| AV01846 | 47.30 ± 0.96 | 11.01 ± 11.22 |
| AV01847 | 49.76 ± 9.15 | 23.47 ± 13.43 |
| AV01848 | 50.78 ± 8.14 | 8.79 ± 5.73 |
| AV01849 | 39.92 ± 11.21 | −3.64 ± 9.49 |
| AV01850 | 47.87 ± 8.54 | −1.80 ± 1.13 |
| AV01851 | 40.75 ± 10.73 | 10.51 ± 4.71 |
| AV01852 | 26.10 ± 10.19 | 8.36 ± 15.76 |
| AV01853 | 47.37 ± 8.35 | 20.40 ± 4.09 |
| AV01854 | 58.36 ± 6.44 | 28.92 ± 12.28 |
| AV01855 | 60.83 ± 8.76 | 21.37 ± 2.42 |
| AV01856 | 47.82 ± 1.19 | 11.71 ± 11.40 |
| AV01857 | 48.40 ± 6.26 | 13.13 ± 8.92 |
| AV01858 | 41.48 ± 5.20 | 3.99 ± 7.49 |
| AV01859 | 57.62 ± 5.05 | 30.29 ± 9.97 |
| AV01860 | 61.37 ± 4.26 | 38.66 ± 6.33 |
| AV01861 | 71.92 ± 6.26 | 38.00 ± 11.39 |
| AV01862 | 65.73 ± 1.64 | 31.73 ± 10.15 |
| AV01863 | 48.50 ± 6.51 | 9.99 ± 5.17 |
| AV01864 | 57.70 ± 5.82 | 20.01 ± 10.78 |
| AV01865 | 55.26 ± 6.10 | 13.41 ± 8.99 |
| AV01866 | 61.53 ± 1.34 | 35.52 ± 2.82 |

### Example 8. In Vivo Testing of PKK siRNA Agents

To assess in vivo activity of PKK siRNAs, mice (4 mice in each group) infected with AAV encoding the human PKK gene were used. At 14 days before dosing of siRNAs, female C57BL/6J mice were infected by intravenous administration of a solution of an adeno-associated virus 8 (AAV8, 2×10¹¹ viral particles) vector encoding the human PKK gene. At day 0, mice were subcutaneously administered a single 3 mg/kg of PKK siRNA agents or PBS. Blood samples were collected at day 0, before dosing of siRNA and at day 7, and day 14 at termination. Human PKK protein concentration was measured by ELISA assay per manufacturer's recommended protocol (Abcam, Human Prekallikrein 1B ELISA Kit, ab202405). Percent of knockdown was calculated by comparing human PKK level in day 7 and day 14 in mouse plasma samples of siRNA treated group (normalized to PKK expression pre-dosing of siRNA) to PBS treated group. Results are summarized in Table 13, Table 14, Table 15, and Table 16. In this example, the GLO-0 in said compounds in Table 3 refers to the compound GalNAc3 in Jayaprakash, et al., (2014) J. Am. Chem. Soc., 136, 16958-16961. The duplex sequences used correspond to those shown in Table 3.

TABLE 13
PKK siRNA single 3 mpk subcutaneous dose testing in
AAV-PKK transduced mouse. Percent of reduction of human
PKK in mouse serum was normalized to PKK expression
pre-dosing of siRNA and to the PBS control group

| | Percent reduction of human PKK in mouse serum | | | |
|---|---|---|---|---|
| | measured by ELISA assay normalized to day 0 | | | |
| | Day 7 | | Day 14 | |
| Duplex ID | Mean | SD | Mean | SD |
| AD00278 | 89% | 0.01 | 85% | 0.04 |
| AD00502 | 61% | 0.04 | NA | |
| AD00503 | 37% | 0.19 | NA | |
| AD00504 | 73% | 0.02 | NA | |
| AD00505 | 76% | 0.08 | 72% | 0.08 |
| AD00506 | 81% | 0.09 | 86% | 0.02 |
| AD00507 | 77% | 0.06 | 79% | 0.03 |
| AD00559 | 58% | 0.12 | NA | |
| AD00560 | 68% | 0.07 | NA | |
| AD00561 | 85% | 0.03 | 86% | 0.02 |
| AD00562 | 48% | 0.2 | NA | |
| AD00563 | 33% | 0.01 | NA | |
| AD00564 | 65% | 0.25 | NA | |
| AD00565 | 51% | 0.09 | NA | |

NA: Not measured

TABLE 14
PKK siRNA single 3 mpk subcutaneous dose testing in
AAV-PKK transduced mouse. Percent of reduction of human
PKK in mouse serum was normalized to PKK expression
pre-dosing of siRNA and to the PBS control group

| | Percent reduction of human PKK in mouse | |
|---|---|---|
| | serum measured by ELISA assay | |
| | normalized to day 0 | |
| Duplex | Day 7 | Day 14 |

| ID | Mean | SD | Mean | SD |
|---|---|---|---|---|
| AD00278 | 92% | 0.02 | 79% | 0.04 |
| AD00566 | 51% | 0.1 | NA | |
| AD00567 | 24% | 0.29 | NA | |
| AD00568 | 23% | 0.1 | NA | |
| AD00569 | 14% | 0.16 | NA | |
| AD00570 | 86% | 0.05 | 76% | 0.07 |
| AD00571 | −9% | 0.23 | NA | |
| AD00572 | −17% | 0.21 | NA | |
| AD00573 | 25% | 0.06 | NA | |
| AD00574 | 22% | 0.12 | NA | |
| AD00575 | 72% | 0.06 | NA | |
| AD00576 | 43% | 0.11 | NA | |
| AD00577 | 69% | 0.08 | NA | |
| AD00578 | 73% | 0.12 | 69% | 0.07 |

NA: Not measured

TABLE 15
PKK siRNA single 3 mpk subcutaneous dose testing in
AAV-PKK transduced mouse. Percent of reduction of human
PKK in mouse serum was normalized to PKK expression
pre-dosing of siRNA and to the PBS control group

| | Percent reduction of human PKK in | |
|---|---|---|
| | mouse serum measured by ELISA | |
| | assay normalized to day 0 | |
| Duplex | Day 7 | Day 14 |

| ID | Mean | SD | Mean | SD |
|---|---|---|---|---|
| AD00278 | 79% | 0.02 | NA | |
| AD00586 | 37% | 0.12 | NA | |
| AD00587 | 61% | 0.05 | NA | |
| AD00588 | 74% | 0.03 | NA | |
| AD00589 | 55% | 0.04 | NA | |
| AD00590 | 46% | 0.05 | NA | |
| AD00591 | 20% | 0.17 | NA | |
| AD00592 | 72% | 0.16 | NA | |
| AD00593 | 69% | 0.03 | NA | |
| AD00594 | 52% | 0.07 | NA | |
| AD00595 | 44% | 0.05 | NA | |
| AD00596 | 17% | 0.2 | NA | |
| AD00597 | −1% | 0.27 | NA | |

NA: Not measured

TABLE 16
PKK siRNA single 3 mpk subcutaneous dose testing in
AAV-PKK transduced mouse. Percent of reduction of human
PKK in mouse serum was normalized to PKK expression
pre-dosing of siRNA and to the PBS control group

| | Percent reduction of human PKK in mouse | |
|---|---|---|
| | serum measured by ELISA assay | |
| | normalized to day 0 | |
| Duplex | Day 7 | Day 14 |

| ID | Mean | SD | Mean | SD |
|---|---|---|---|---|
| AD00278 | 88% | 0.02 | NA | |
| AD00665 | 38% | 0.08 | NA | |
| AD00666 | 21% | 0.12 | NA | |
| AD00667 | 39% | 0.09 | NA | |
| AD00668 | 26% | 0.08 | NA | |
| AD00669 | 71% | 0.18 | NA | |
| AD00670 | 57% | 0.08 | NA | |
| AD00671 | 47% | 0.04 | NA | |
| AD00672 | 69% | 0.03 | NA | |
| AD00673 | 74% | 0.05 | NA | |
| AD00674 | 71% | 0.04 | NA | |

NA: Not measured

### Example 9. In Vivo Testing of PKK siRNA Agents in Cynomolgus Monkeys

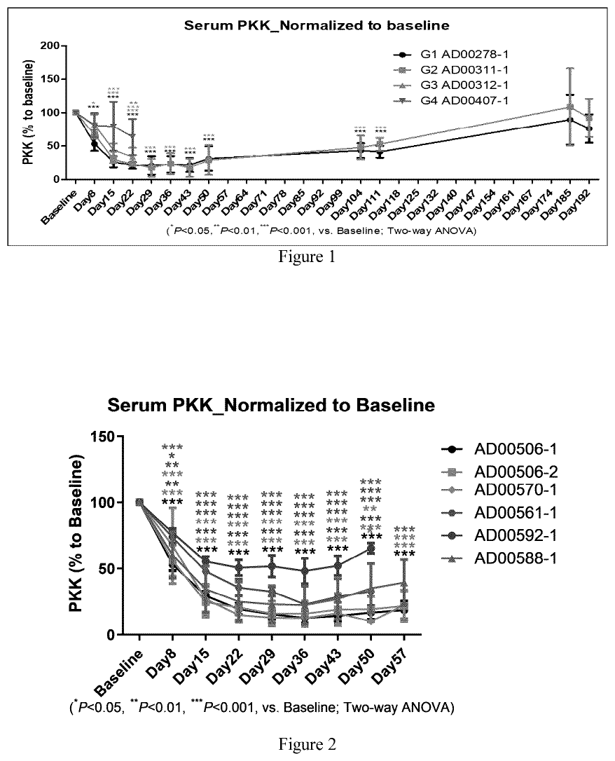

Male cynomolgus monkeys (13-22 years old, 7˜9 kg of weights, 3 monkeys in each group) were enrolled in the study. Each monkey received a subcutaneous injection with one of the testing articles formulated in PBS at 3 mg/kg at day 1 (pre-dosing of siRNA). After overnight fast, blood samples were drawn at day-7 (pre-dose), 1 (pre-dose), 8, 15, 22, 29, 43, 50, 57, 64, 71, 78, 85, 92, 99, 104, 111, 118, 125, 132, 140, 147, 154, 161, 167, 174, 185, and 192; PKK protein concentration in serum were measured by ELISA method (Human Prekallikrein 1B (KLKB1) SimpleStep ELISA® Kit; Abcam; Cat No: ab202405). Percent remaining of PKK (normalized to day 1, pre-dosing of siRNA) for groups dosed with compound AD00278-1, AD00311-1, AD00312-1, and AD00407-1 is shown in FIG. 1.

### Example 10. In Vivo Testing of PKK siRNA Agents in Cynomolgus Monkeys

Male cynomolgus monkeys (3 monkeys in each group) were enrolled in the study. Each monkey received a subcutaneous injection with one of the testing articles formulated in PBS at 3 mg/kg at day 1 (pre-dosing of siRNA). After overnight fast, blood samples were drawn at day −7 (pre-dose), 1 (pre-dose), 8, 15, 22, 29, 43, 50, and 57; PKK protein concentration in serum were measured by ELISA method. Percent remaining of PKK (normalized to day 1, pre-dosing of siRNA) for groups dosed with compound AD00588-1, AD00506-1, AD00506-2, AD00592-1, AD00570-1, AD00561-1 is shown in FIG. 2.

### Example 11. In Vivo Testing of PKK siRNA Agents in Cynomolgus Monkeys

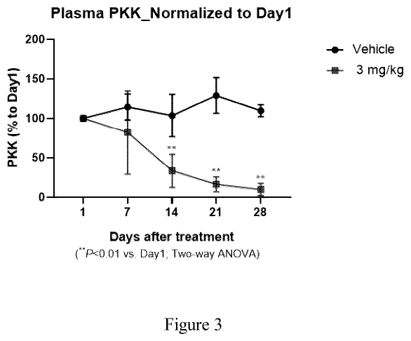

Male cynomolgus monkeys (4 monkeys in each group) were enrolled in the study. Each monkey received a subcutaneous injection with one of the testing articles formulated in PBS at 3 mg/kg AD00506-4 at day 1 (pre-dosing of siRNA). After overnight fast, blood samples were drawn at day-7 (pre-dose), 1 (pre-dose), 7, 14, 21, and 28; PKK protein concentration in serum were measured by ELISA method. Percent remaining of PKK (normalized to day 1, pre-dosing of siRNA) for groups dosed with compound AD00506-4 is shown in FIG. 3.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

1. A double-stranded ribonucleic acid (dsRNA) agent for inhibiting expression of prekallikrein (PKK) in a cell, wherein the dsRNA agent comprises a sense strand and an antisense strand, nucleotide positions 2 to 18 in the antisense strand comprising a region of complementarity to a PKK RNA transcript, wherein the region of complementarity comprises at least 15 contiguous nucleotides that differ by 0, 1, 2, or 3 nucleotides from one of the antisense sequences listed in one of Tables 1-3, and optionally comprising a targeting ligand; or
the region of complementarity to the PKK RNA transcript comprises at least 15, 16, 17, 18, or 19 contiguous nucleotides that differ by no more than 3 nucleotides from one of the antisense sequences listed in one of Tables 1-3.

2. (canceled)

3. The dsRNA agent of claim 1, wherein the antisense strand of dsRNA is at least substantially complementary to a target region of SEQ ID NO: 767 and is provided in any one of Tables 1-3; or
the antisense strand of dsRNA is fully complementary to a target region of SEQ ID NO: 767 and is provided in any one of Tables 1-3.

4. (canceled)

5. The dsRNA agent of claim 1, wherein the dsRNA agent comprises a sense strand sequence set forth in any one of Tables 1-3, wherein the sense strand sequence is at least substantially complementary to the antisense strand sequence in the dsRNA agent; or
the sense strand sequence is fully complementary to the antisense strand sequence in the dsRNA agent.

6. (canceled)

7. The dsRNA agent of claim 1, wherein the dsRNA agent comprises an antisense strand sequence set forth in any one of Tables 1-3; or
the dsRNA agent comprises the sequences set forth as a duplex sequence in any of Tables 1-3.

8. (canceled)

9. The dsRNA of claim 1, wherein the dsRNA agent comprises at least one modified nucleotide; or
all or substantially all of the nucleotides of the antisense strand are modified nucleotides; or
all or substantially all of the nucleotides of the sense strand and the antisense strand are modified nucleotides;
preferably, the dsRNA agent comprises an E-vinylphosphonate nucleotide at the 5′ end of the antisense strand.

10. (canceled)

11. The dsRNA agent of claim 9, wherein the at least one modified nucleotide comprises: a 2′-O-methyl nucleotide, 2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-OMe nucleotide, inverted 2′-deoxy nucleotide, 2′-amino-modified nucleotide, 2′-alkyl-modified nucleotide, morpholino nucleotide, and 3′-OMe nucleotide, a nucleotide comprising a 5′-phosphorothioate group, or a terminal nucleotide linked to a cholesteryl derivative or dodecanoic acid bisdecylamide group, a 2′-amino-modified nucleotide, a phosphoramidate, or a non-natural base comprising nucleotide;
preferably, the modified sense strand is a modified sense strand sequence set forth in one of Tables 2 and 3;
preferably, the modified antisense strand is a modified antisense strand sequence set forth in one of Tables 2 and 3.

12. (canceled)

13. The dsRNA agent of claim 1, wherein the dsRNA agent comprises at least one phosphorothioate internucleoside linkage; or
the sense strand comprises at least one phosphorothioate internucleoside linkage; or
the antisense strand comprises at least one phosphorothioate internucleoside linkage;
preferably, the sense strand comprises 1, 2, 3, 4, 5, or 6 phosphorothioate internucleoside linkages; or
the antisense strand comprises 1, 2, 3, 4, 5, or 6 phosphorothioate internucleoside linkages.

14-20. (canceled)

21. The dsRNA agent of claim 1, wherein the sense strand is complementary or substantially complementary to the antisense strand, and the region of complementarity is between 16 and 23 nucleotides in length; or
the region of complementarity is 19-21 nucleotides in length.

22. (canceled)

23. The dsRNA agent of claim 1, wherein each strand is no more than 30 nucleotides in length; or
each strand is no more than 25 nucleotides in length; or
each strand is no more than 23 nucleotides in length.

24-25. (canceled)

26. The dsRNA agent of claim 1, wherein the dsRNA agent comprises at least one modified nucleotide and further comprises one or more targeting groups or linking groups;
preferably, the one or more targeting groups or linking groups are conjugated to the sense strand;
preferably, the targeting group or linking group comprises N-acetyl-galactosamine (GalNAc).

27-28. (canceled)

29. The dsRNA agent of claim 26, wherein the targeting group has a structure:

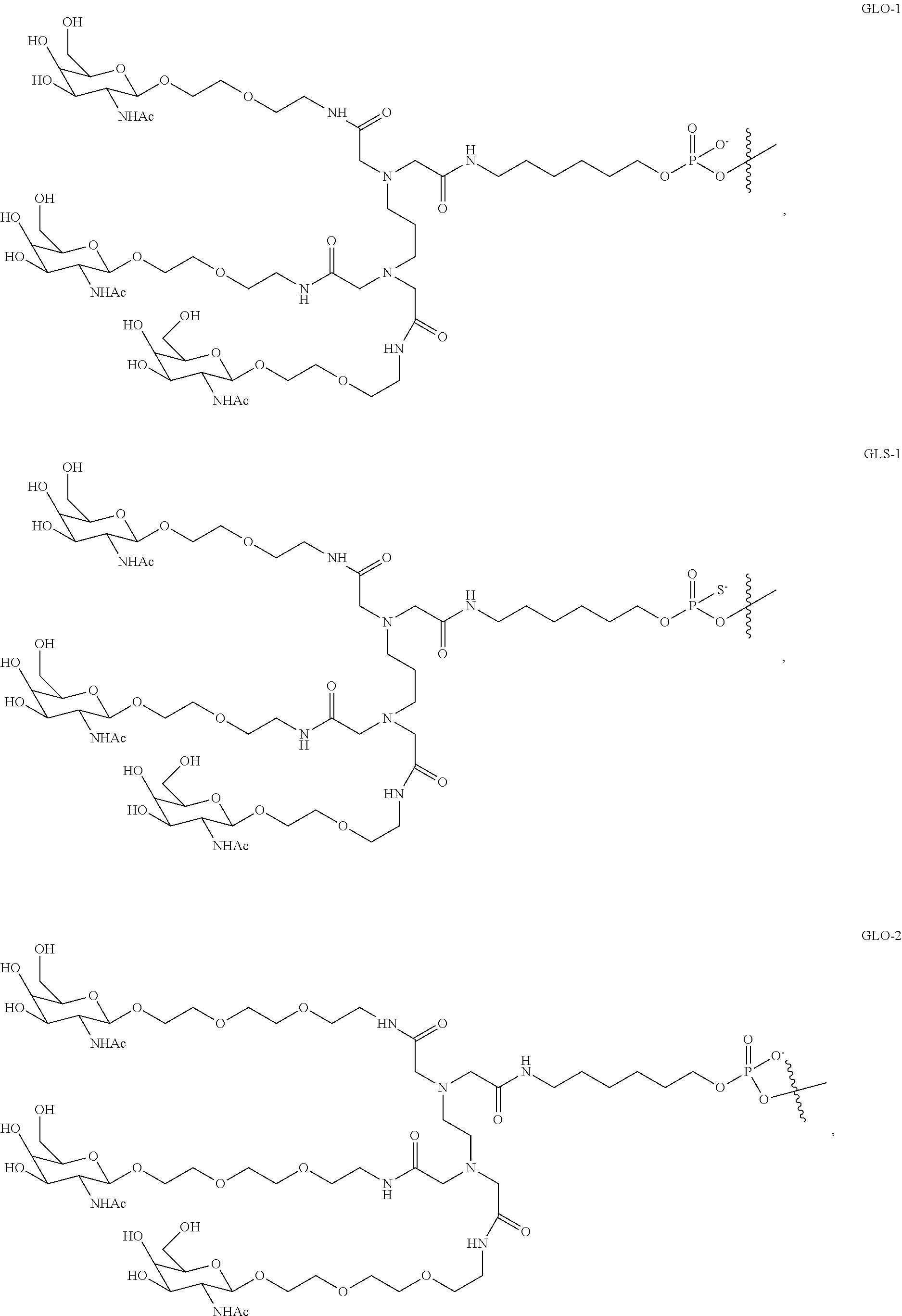 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

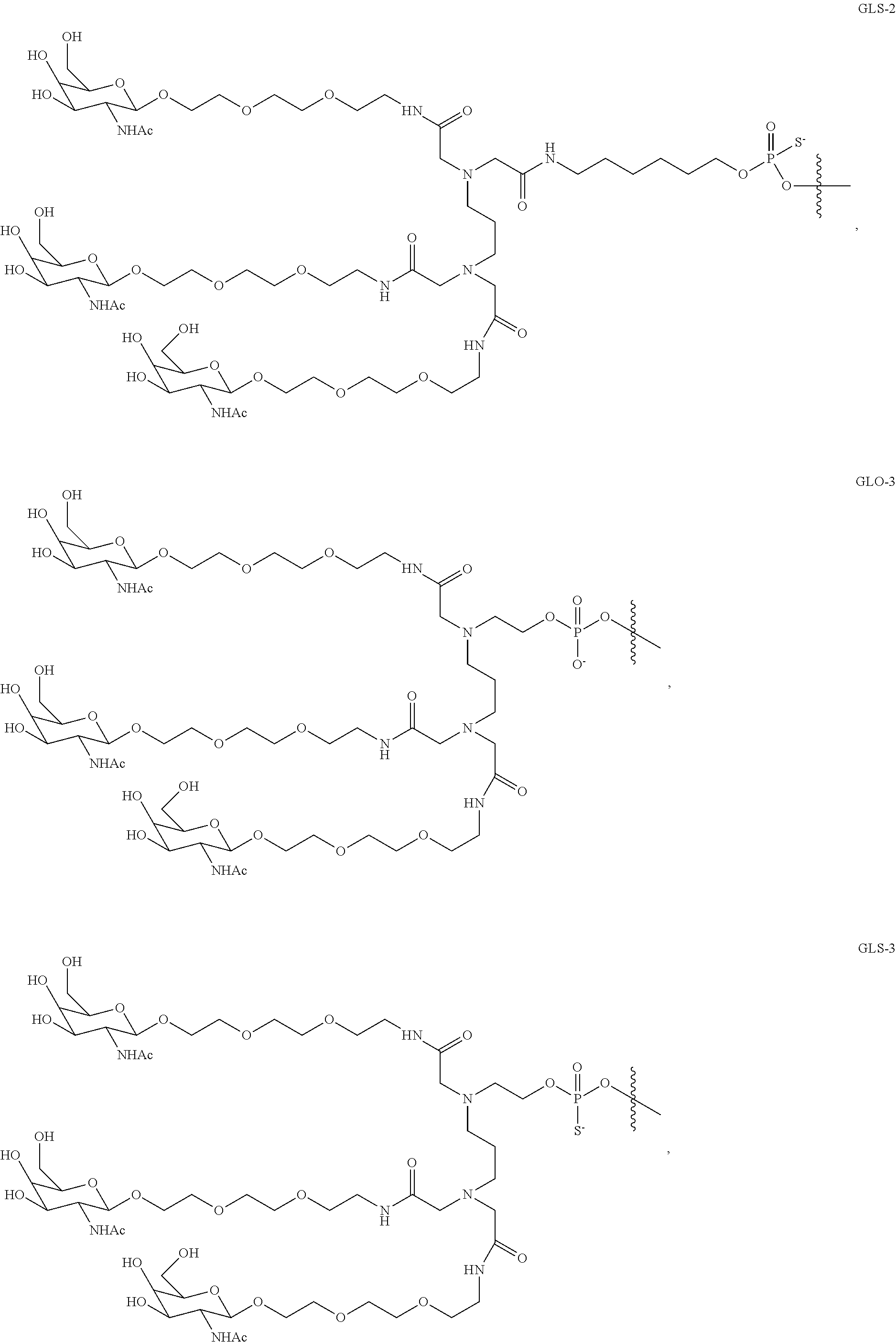 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

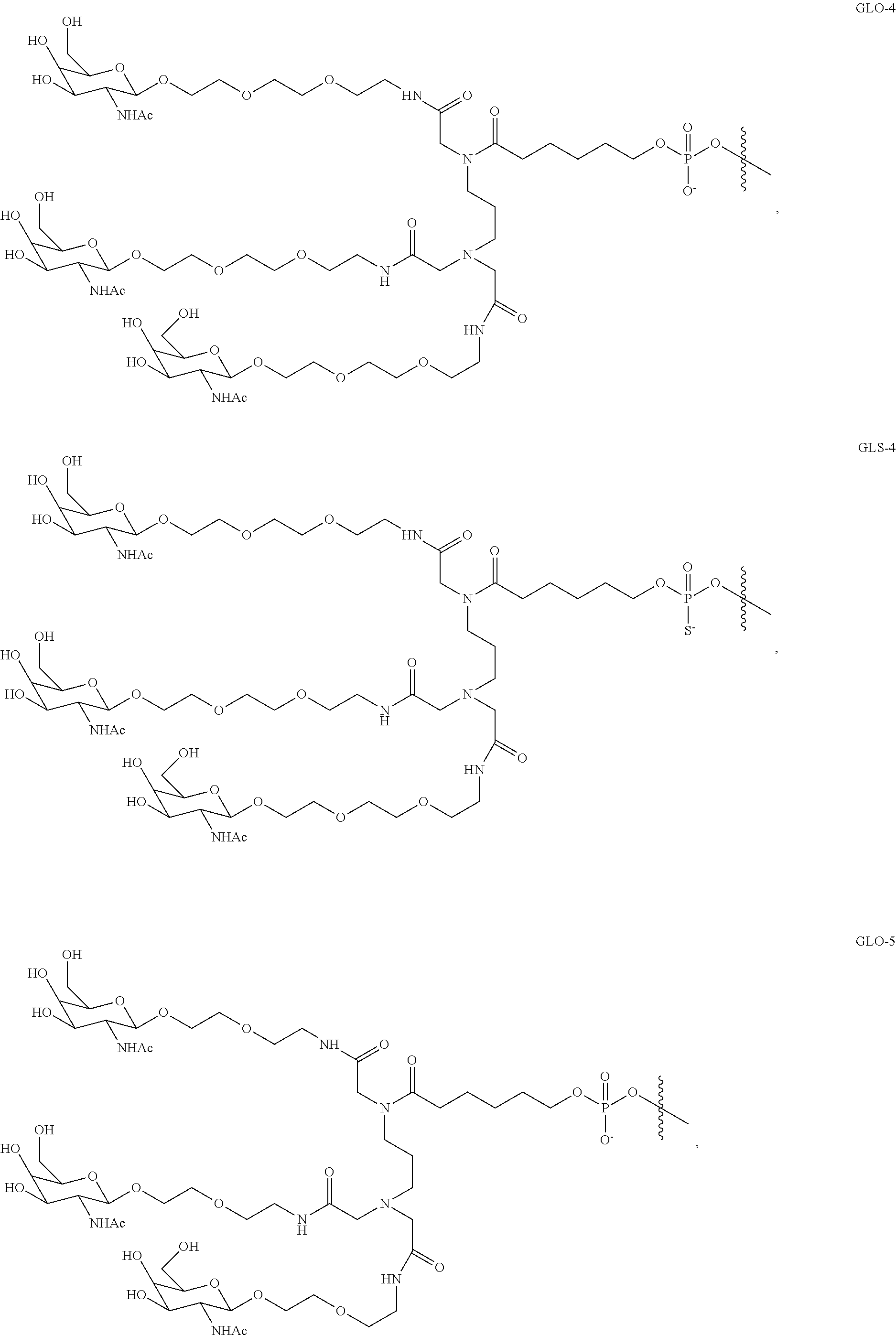 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

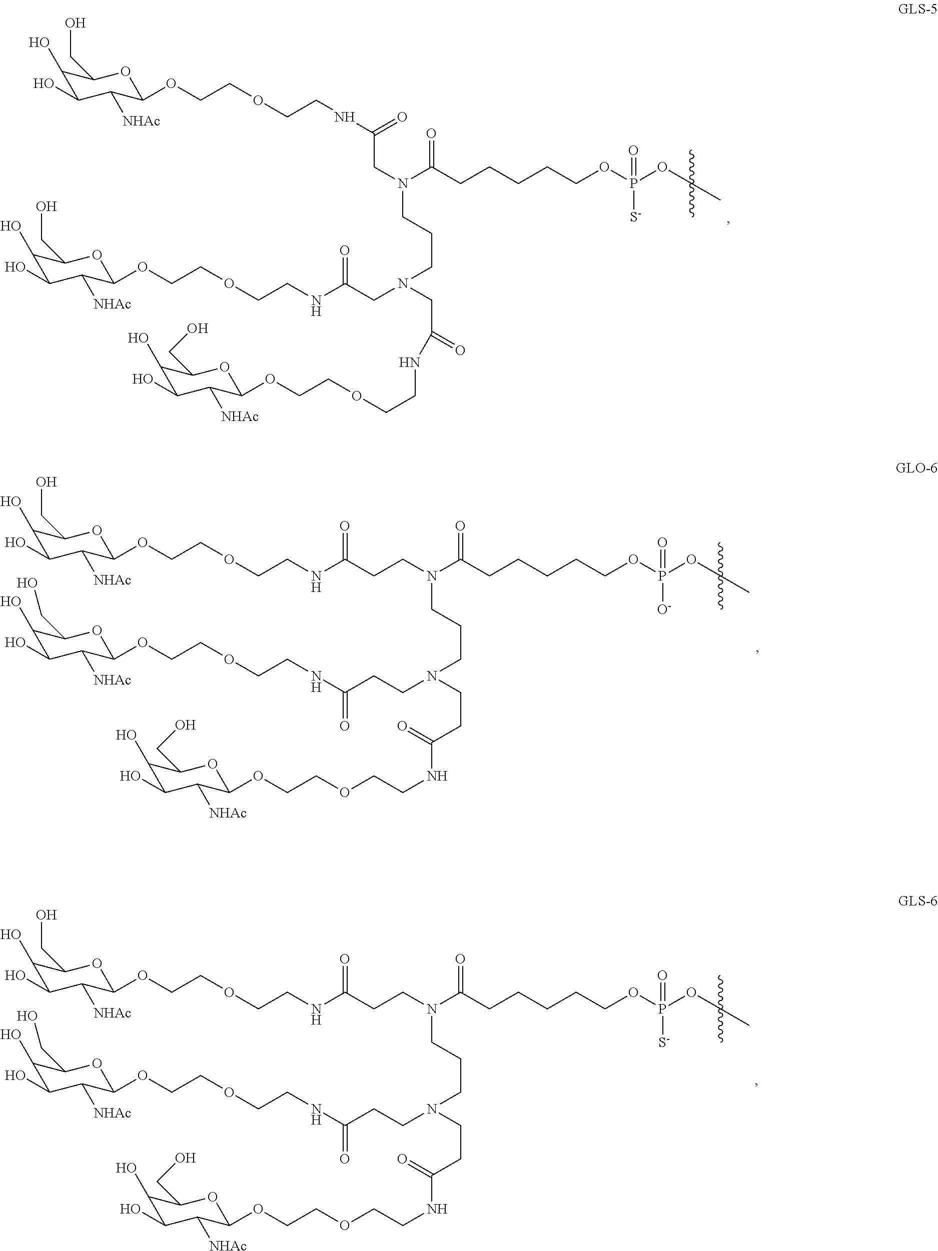 [SMILES: *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

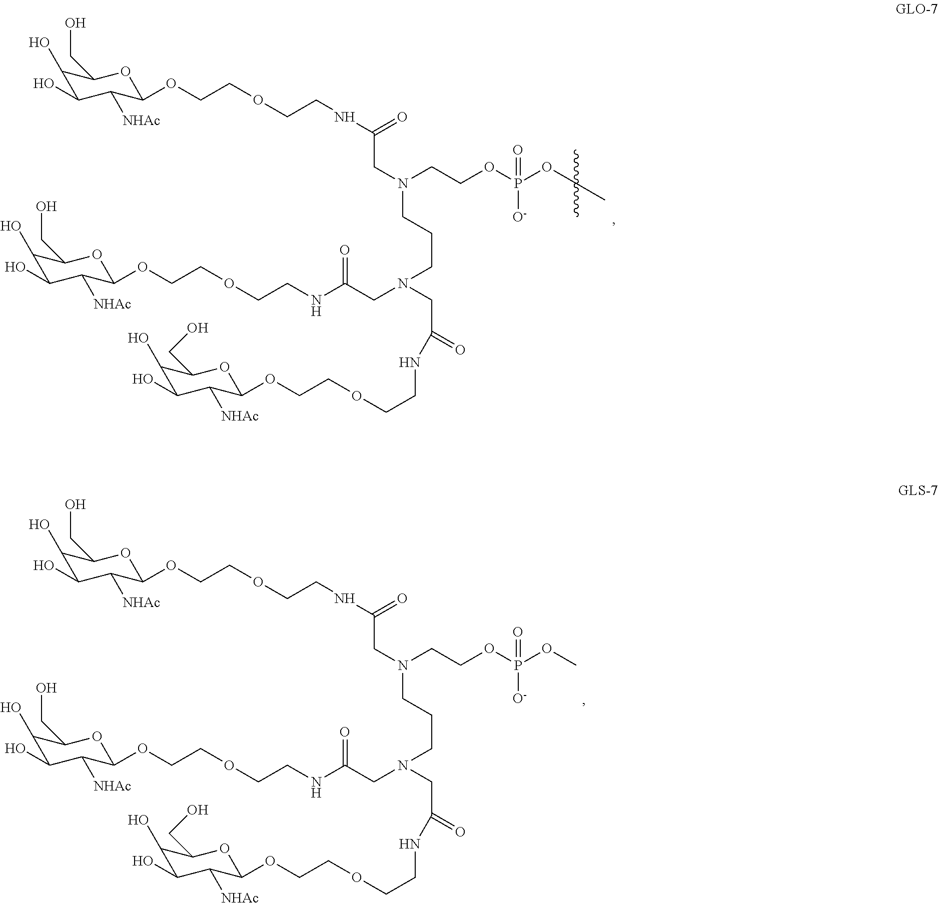 [SMILES: *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; COP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

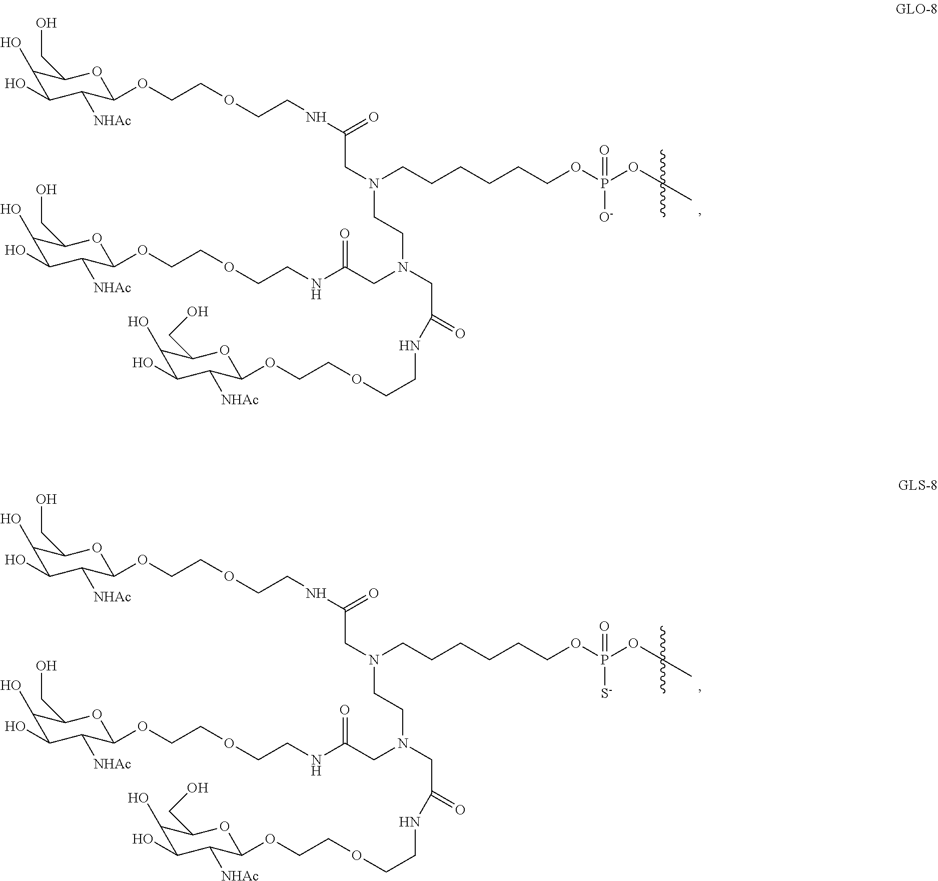 [SMILES: *OP(=O)([O-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

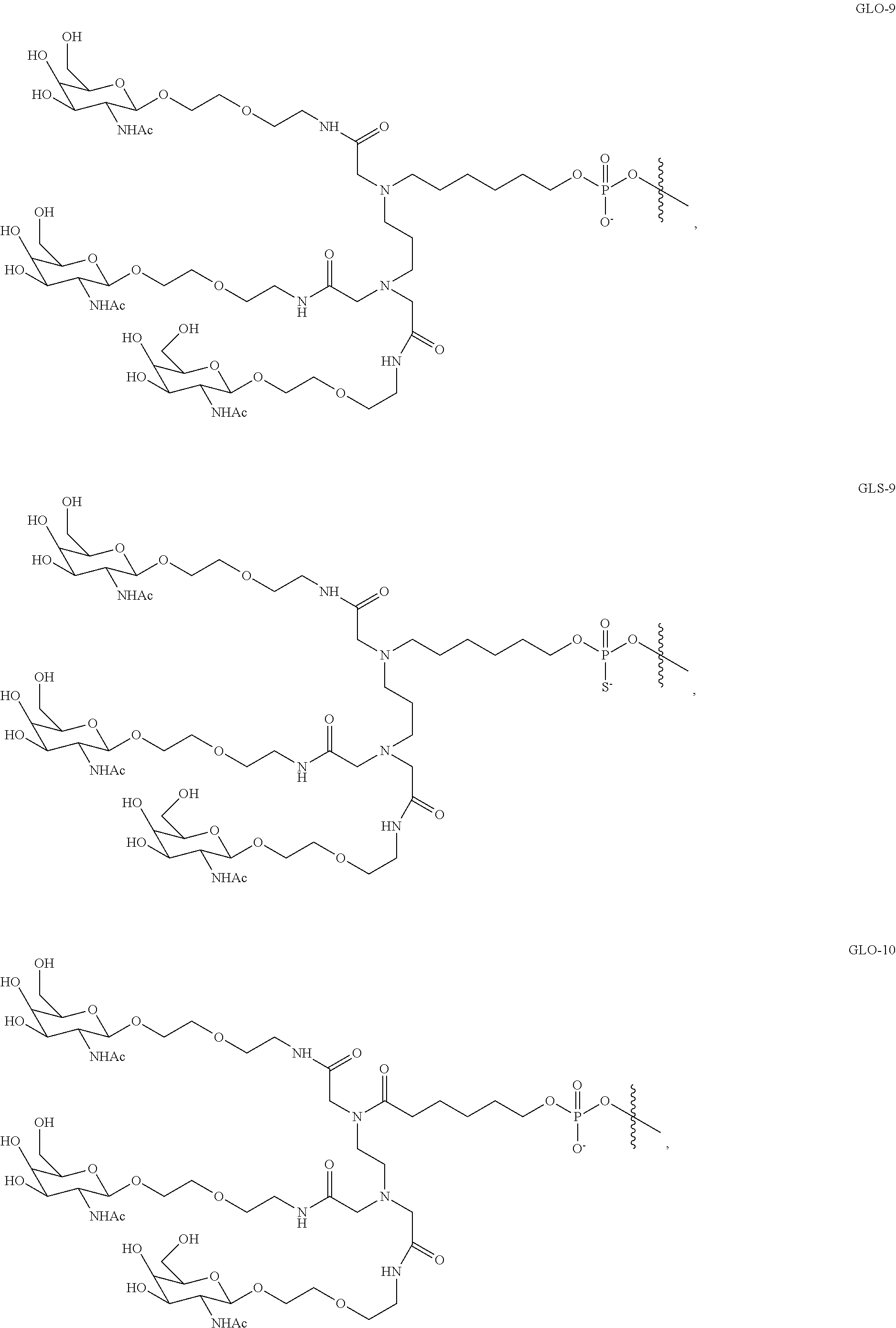 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

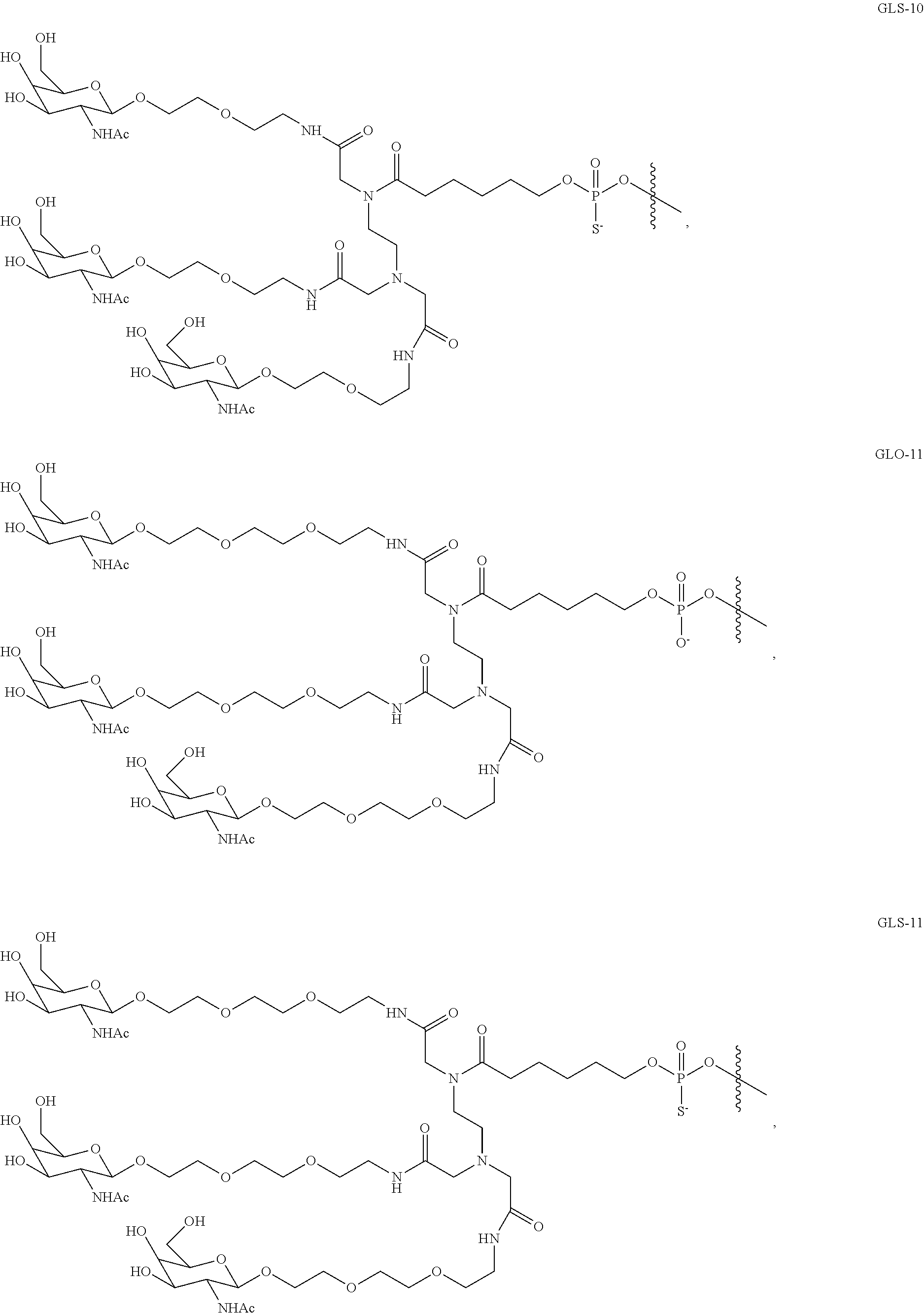 [SMILES: *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

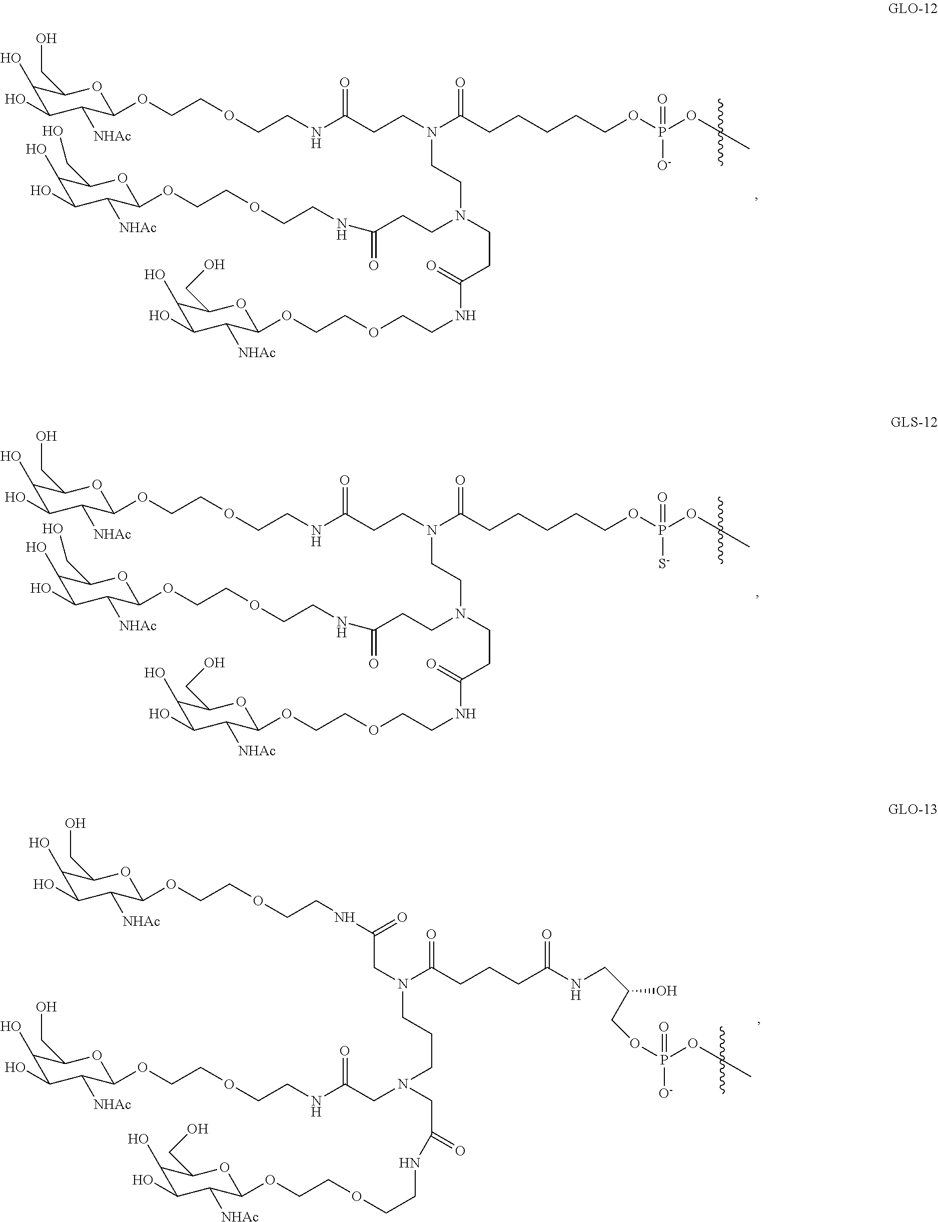 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

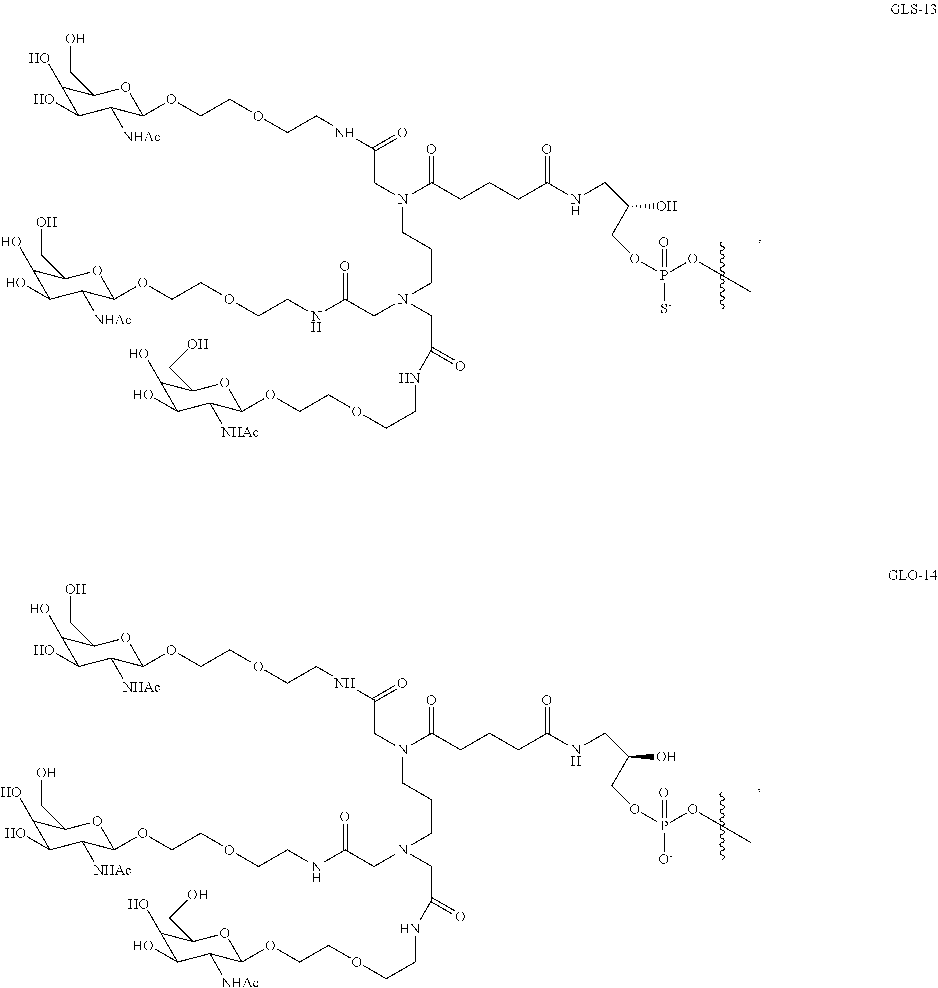 [SMILES: *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

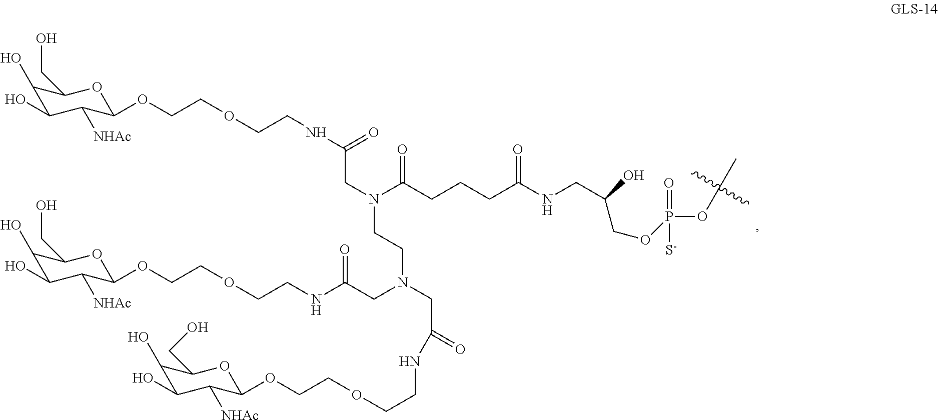 [SMILES: *OP(=O)([S-])OC[C@H](O)CNC(=O)CCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

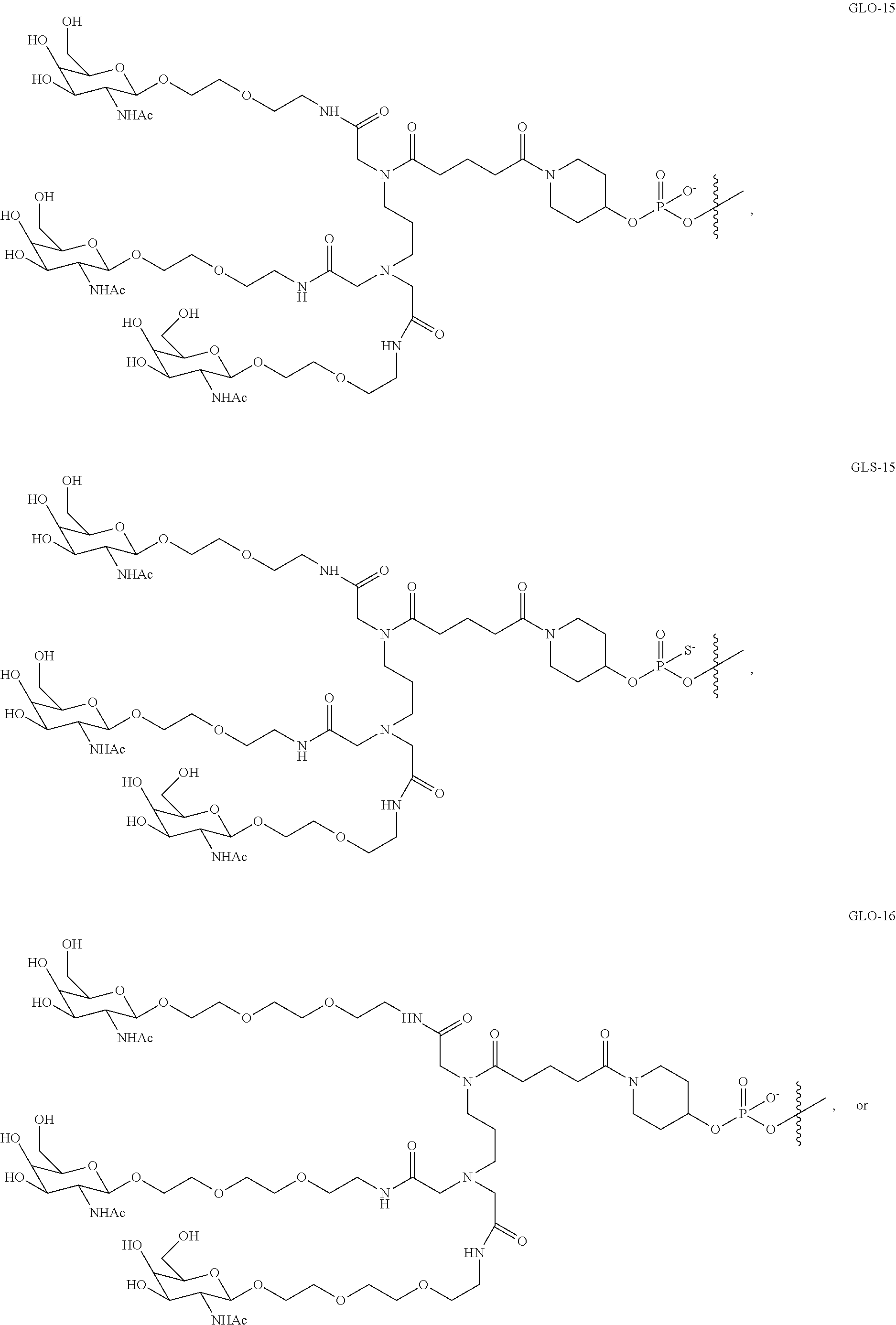 [SMILES: *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

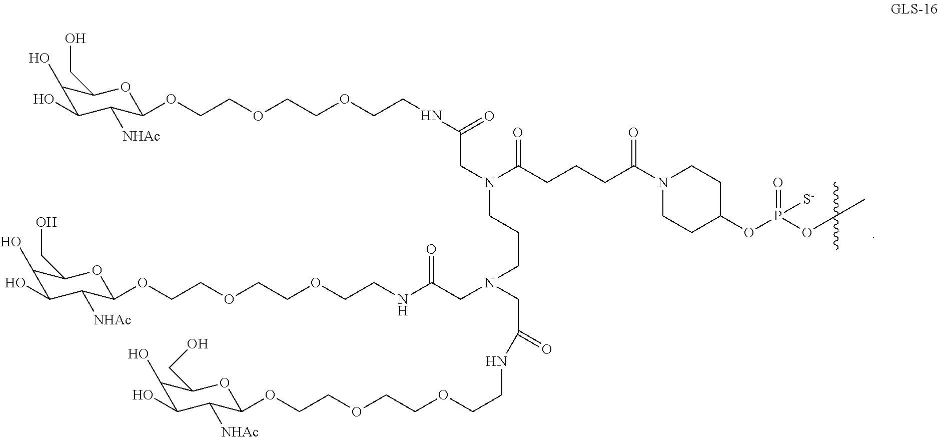 [SMILES: *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

30. The dsRNA agent of claim 1, wherein the dsRNA agent comprises a targeting group that is conjugated to the 5′-terminal end of the sense strand; or
the dsRNA agent comprises a targeting group that is conjugated to the 3′-terminal end of the sense strand.

31. (canceled)

32. The dsRNA agent of claim 1, wherein the antisense strand comprises one inverted abasic residue at 3′-terminal end; or
the sense strand comprises one or two inverted abasic residues at the 3′ and/or the 5′ terminal end.

33. (canceled)

34. The dsRNA agent of claim 1, wherein the dsRNA agent has two blunt ends; or
at least one strand comprises a 3′ overhang of at least 1 or 2 nucleotides.

35-36. (canceled)

37. The dsRNA agent of claim 1, wherein the PKK RNA transcript is SEQ ID NO: 767.

38. A composition comprising one, two, three, or more dsRNA agents of claim 1;
preferably, the composition further comprises a pharmaceutically acceptable carrier;
preferably, the composition further comprises one or more additional therapeutic agents;
preferably, the composition is packaged in a kit, container, pack, dispenser, pre-filled syringe, or vial;
preferably, the composition is formulated for subcutaneous administration or is formulated for intravenous (IV) administration.

39-42. (canceled)

43. A cell comprising a dsRNA agent of claim 1; preferably, the cell is a mammalian cell, optionally a human cell.

44. (canceled)

45. A method of inhibiting the expression of a prekallikrein (PKK) gene in a cell, comprising:
(i) preparing a cell or plurality of cells comprising an effective amount of one or more double-stranded ribonucleic acid (dsRNA) agents of claim 1;
preferably, further comprising (ii) maintaining the cell or plurality of cells prepared in (i) for a time sufficient to obtain degradation of the mRNA transcript of a PKK gene, thereby inhibiting expression of the PKK gene in the cell or plurality of cells and reducing a level of the PKK polypeptide in the cell or plurality of cells.

46-58. (canceled)

59. A method of inhibiting expression of a PKK gene in a subject, the method comprising administering to the subject an effective amount of one or more double-stranded ribonucleic acid (dsRNA) agents of claim 1;
preferably, two, three, four, or more of the dsRNA agents are administered to the subject;
preferably, the dsRNA agent is administered to the subject subcutaneously or by IV administration;
preferably, the method further comprises assessing inhibition of the PKK gene, following the administration of the one or more dsRNA agents, wherein a means for the assessing comprises:
(i) determining one or more physiological characteristics of a PKK-associated disease or condition in the subject and
(ii) comparing the determined physiological characteristic(s) to a baseline pre-treatment physiological characteristic of the PKK-associated disease or condition and/or to a control physiological characteristic of the PKK-associated disease or condition,
wherein the comparison indicates one or more of a presence or absence of inhibition of expression of the PKK gene in the subject;
preferably, the determined physiological characteristic in the subject is one or more of: a level of plasma PKK in the subject; presence, absence, and/or a level of one or more of plasma kallikrein, high-molecular-weight kininogen (HK), and bradykinin in the subject; a physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject;
preferably, the physiological characteristic is determined in a biological sample obtained from the subject;
preferably, the biological sample comprises one or more of: a blood sample, a serum sample, a cell sample, a tissue sample, and a liver sample;
preferably, the determined physiological characteristic in the subject is more normal compared to a baseline pre-treatment physiological characteristic of the PKK-associated disease or condition in the subject and/or to a control physiological characteristic of the PKK-associated disease or condition;
preferably, a level of one or more of: plasma PKK in the subject; plasma kallikrein, high-molecular-weight kininogen (HK), and bradykinin in the subject; determined to be statistically significantly higher, lower, or unchanged compared to a baseline pre-treatment physiological characteristic of the PKK-associated disease or condition and/or to a control physiological characteristic of the PKK-associated disease or condition indicates a status of PKK gene expression in the subject;
preferably, a reduction in one or more of the physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject indicates a reduction of PKK gene expression in the subject;
preferably, the PKK-associated disease or condition is one or more of: hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins.

60-69. (canceled)

70. A method of treating a disease or condition in a subject, wherein
the disease or condition is associated with the presence of a prekallikrein (PKK) protein, the method comprising administering to the subject an effective amount of one or more double-stranded ribonucleic acid (dsRNA) agents of claim 1, to inhibit expression of the PKK gene encoding the PKK protein;
preferably, wherein the method comprises administering two, three, four, or more dsRNA agents to the subject.

71. (canceled)

72. The method of claim 70, wherein the disease or condition is one or more of: hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins.

73. The method of claim 70, further comprising administering an additional therapeutic regimen to the subject;
wherein, the additional therapeutic regimen comprises: administering to the subject one or more: PKK antisense polynucleotides, additional PKK dsRNA therapeutic agent, a non-PKK dsRNA therapeutic agent, a PKK non-dsRNA therapeutic agent, and a behavioral modification;
preferably, the non-PKK dsRNA therapeutic agent is one of more of: a blood thinner, an NSAID, aspirin, an anticoagulant, tissue plasminogen activator (tPA); recombinant tissue plasminogen activator (tPA), a statin, a stent, a cholesterol-lowering therapeutic agent, a vasodilator, cilostazol, Plavix®, an anti-coagulation drug, and an antiplatelet agent;
preferably, the one or more dsRNA agent(s) is administered to the subject by one of both of subcutaneous administration and intravenous (IV) administration.

74-76. (canceled)

77. The method of claim 70, further comprising determining an efficacy of the administered one or more double-stranded ribonucleic acid (dsRNA) agent(s) in the subject;
preferably, a means of determining an efficacy of the treatment in the subject comprises:
(i) determining one or more physiological characteristics of the PKK-associated disease or condition in the subject and
(ii) comparing the determined physiological characteristic(s) to a baseline pre-treatment physiological characteristic of the PKK-associated disease or condition,
wherein the comparison indicates one or more of a presence, absence, and level of efficacy of the administration of the double-stranded ribonucleic acid (dsRNA) agent to the subject;
preferably, the physiological characteristic is determined in a biological sample obtained from the subject;
preferably, the biological sample comprises one or more of: a blood sample, a serum sample, a cell sample, a tissue sample, and a liver sample;
preferably, the determined physiological characteristic in the subject is different compared to the baseline pre-treatment physiological characteristic of the PKK-associated disease or condition in the subject or to a control level of the physiological characteristic.

78. (canceled)

79. The method of claim 77, wherein the determined physiological characteristic in the subject is one or more of: a level of plasma PKK in the subject; presence, absence, and/or a level of one or more of plasma kallikrein, high-molecular-weight kininogen (HK), and bradykinin in the subject; a physical symptom, swelling of skin, swelling of hands and feet, fatigue, headache, muscle aches, tingling of skin, abdominal pain, nausea and vomiting, hoarseness, shortness of breath, mood changes, laryngeal edema, pain, and fever in the subject;
preferably, a level of one or more of the plasma PKK; plasma kallikrein, high-molecular-weight kininogen (HK), and bradykinin; determined to be statistically significantly higher, lower, or unchanged compared to a baseline pre-treatment physiological characteristic of the PKK-associated disease or condition and/or to a control physiological characteristic of the PKK-associated disease or condition indicates a status of PKK gene expression in the subject.

80-83. (canceled)

84. The method of claim 77, wherein the PKK-associated disease or condition is one or more of: hereditary angioedema, angioedema, bradykinin-mediated angioedema, diabetic retinopathy, diabetic macular edema, hypertension, nephropathy, vascular disease, stroke, peripheral artery disease (PAD), abdominal aortic aneurysm (AAA), carotid artery disease (CAD), arteriovenous malformation (AVM), critical limb-threatening ischemia (CLTI), pulmonary embolism (blood clots), deep vein thrombosis (DVT), chronic venous insufficiency (CVI), and varicose veins.

85-89. (canceled)

90. A double-stranded ribonucleic acid (dsRNA) agent for inhibiting expression of prekallikrein (PKK), wherein the dsRNA agent comprises a sense strand and an antisense strand, wherein the sense strand comprises at least 15 contiguous nucleotides that differ by 0, 1, 2, or 3 nucleotides from any one of the nucleotide sequences of nucleotides 1014-1044; 1054-1084; 1059-1089; 1106-1138; 1456-1486; 1543-1573; or 2029-2059 of SEQ ID: 767, and the antisense strand is complementary or substantially complementary to the sense strand sequence in the dsRNA agent, and optionally comprising a targeting ligand;
preferably, the region of complementarity to the PKK RNA transcript comprises at least 15, 16, 17, 18, or 19 contiguous nucleotides that differ by 0, 1, 2, or 3 nucleotides from any one of the nucleotide sequences of nucleotides 1014-1044; 1054-1084; 1059-1089; 1106-1138; 1456-1486; 1543-1573; or 2029-2059 of SEQ ID: 767;
preferably, the antisense strand of dsRNA is at least substantially complementary or fully complementary to a target region of SEQ ID NO: 767; or
the sense strand sequence is at least substantially complementary or fully complementary to the antisense strand sequence in the dsRNA agent;
preferably, the dsRNA agent comprises at least one modified nucleotide;
preferably, all or substantially all of the nucleotides of the antisense strand and sense strand are modified nucleotides;
preferably, the at least one modified nucleotide comprises: a 2′-O-methyl nucleotide, 2′-Fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-OMe nucleotide, inverted 2′-deoxy nucleotide, 2′-amino-modified nucleotide, 2′-alkyl-modified nucleotide, morpholino nucleotide, and 3′-OMe nucleotide, a nucleotide comprising a 5′-phosphorothioate group, or a terminal nucleotide linked to a cholesteryl derivative or dodecanoic acid bisdecylamide group, a 2′-amino-modified nucleotide, a phosphoramidate, or a non-natural base comprising nucleotide;
preferably, the sense strand is complementary or substantially complementary to the antisense strand, and the region of complementarity is between 16 and 23 nucleotides in length;
preferably, the region of complementarity is 19-21 nucleotides in length;
preferably, each strand is no more than 40 nucleotides in length, or each strand is no more than 30 nucleotides in length, or each strand is no more than 25 nucleotides in length, or each strand is no more than 23 nucleotides in length.

91-102. (canceled)


SEQUENCE LISTING (1687 sequences)

SEQ ID NO: 1 - RNA, 21 nt, synthetic construct
  source 1..21: mol_type = other RNA
gggatgtagcttccatgtaca

SEQ ID NO: 2 - RNA, 21 nt, synthetic construct
  source 1..21: mol_type = other RNA
tggttgcttcttgaaagataa

SEQ ID NO: 3 - RNA, 21 nt, synthetic construct
  source 1..21: mol_type = other RNA
cttcttgaaagatagtgttaa

SEQ ID NO: 4 - RNA, 21 nt, synthetic construct
  source 1..21: mol_type = other RNA
caatgtggtcatcaaataaga

SEQ ID NO: 5 - RNA, 21 nt, synthetic construct
  source 1..21: mol_type = other RNA
gtggtcatcaaataagtgcta

SEQ ID NO: 6 - RNA, 21 nt, synthetic construct
  source 1..21: mol_type = other RNA
catcgagacatttataaagga

SEQ ID NO: 7 - RNA, 21 nt, synthetic construct
  source 1..21: mol_type = other RNA
atcgagacatttataaaggaa

SEQ ID NO: 8 - RNA, 21 nt, synthetic construct
  source 1..21: mol_type = other RNA
gttgatatgagaggagtcaaa

SEQ ID NO: 9 - RNA, 21 nt, synthetic construct
  source 1..21: mol_type = other RNA
ctaaggttagcagtgttgaaa

SEQ ID NO: 10 - RNA, 21 nt, synthetic construct
  source 1..21: mol_type = other RNA
ggttagcagtgttgaagaata

SEQ ID NO: 11 - RNA, 21 nt, synthetic construct
  source 1..21: mol_type = other RNA
gttagcagtgttgaagaatga

SEQ ID NO: 12 - RNA, 21 nt, synthetic construct
  source 1..21: mol_type = other RNA
gtaacgtggaatctggattca

SEQ ID NO: 13 - RNA, 21 nt, synthetic construct
  source 1..21: mol_type = other RNA
aacgtggaatctggattctca

SEQ ID NO: 14 - RNA, 21 nt, synthetic construct
  source 1..21: mol_type = other RNA
aatctggattctcactgaaga

SEQ ID NO: 15 - RNA, 21 nt, synthetic construct
  source 1..21: mol_type = other RNA
actccagatgcttttgtgtga

SEQ ID NO: 16 - RNA, 21 nt, synthetic construct
  source 1..21: mol_type = other RNA
ttcttaaaacatctgaaagta

SEQ ID NO: 17 - RNA, 21 nt, synthetic construct
  source 1..21: mol_type = other RNA
ttcctctactcctcaagaaaa

SEQ ID NO: 18 - RNA, 21 nt, synthetic construct
  source 1..21: mol_type = other RNA
tcctctactcctcaagaaaaa

SEQ ID NO: 19 - RNA, 21 nt, synthetic construct
  source 1..21: mol_type = other RNA
gccttttaacctgcaaaagaa

SEQ ID NO: 20 - RNA, 21 nt, synthetic construct
  source 1..21: mol_type = other RNA
aacctgcaaaagaactttaca

SEQ ID NO: 21 - RNA, 21 nt, synthetic construct
  source 1..21: mol_type = other RNA
acctgcaaaagaactttacca

SEQ ID NO: 22 - RNA, 21 nt, synthetic construct
  source 1..21: mol_type = other RNA
cctgcaaaagaactttaccta

SEQ ID NO: 23 - RNA, 21 nt, synthetic construct
  source 1..21: mol_type = other RNA
gcaaaagaactttacctgaaa

SEQ ID NO: 24 - RNA, 21 nt, synthetic construct
  source 1..21: mol_type = other RNA
actttggaggagaagaattga

SEQ ID NO: 25 - RNA, 21 nt, synthetic construct
  source 1..21: mol_type = other RNA
gaggagaagaattgaatgtga

SEQ ID NO: 26 - RNA, 21 nt, synthetic construct
  source 1..21: mol_type = other RNA
tgttaaaggagtgaatgttta

SEQ ID NO: 27 - RNA, 21 nt, synthetic construct
  source 1..21: mol_type = other RNA
ttcgctgtcagtttttcacta

SEQ ID NO: 28 - RNA, 21 nt, synthetic construct
  source 1..21: mol_type = other RNA
cgctgtcagtttttcacttaa

SEQ ID NO: 29 - RNA, 21 nt, synthetic construct
  source 1..21: mol_type = other RNA
ttttcacttattctttactca

SEQ ID NO: 30 - RNA, 21 nt, synthetic construct
  source 1..21: mol_type = other RNA
gaagagaagtgtaagtgttta

SEQ ID NO: 31 - RNA, 21 nt, synthetic construct
  source 1..21: mol_type = other RNA
agagaagtgtaagtgtttcta

SEQ ID NO: 32 - RNA, 21 nt, synthetic construct
  source 1..21: mol_type = other RNA
agaagtgtaagtgtttcttaa

SEQ ID NO: 33 - RNA, 21 nt, synthetic construct
  source 1..21: mol_type = other RNA
agtgtaagtgtttcttaagaa

SEQ ID NO: 34 - RNA, 21 nt, synthetic construct
  source 1..21: mol_type = other RNA
ctggttactctttgagattga

SEQ ID NO: 35 - RNA, 21 nt, synthetic construct
  source 1..21: mol_type = other RNA
tggttactctttgagattgta

SEQ ID NO: 36 - RNA, 21 nt, synthetic construct
  source 1..21: mol_type = other RNA
actctttgagattgtgtaaca

SEQ ID NO: 37 - RNA, 21 nt, synthetic construct
  source 1..21: mol_type = other RNA
ctctttgagattgtgtaacaa

SEQ ID NO: 38 - RNA, 21 nt, synthetic construct
  source 1..21: mol_type = other RNA
tctgtctgcacaacaaaaaca

SEQ ID NO: 39 - RNA, 21 nt, synthetic construct
  source 1..21: mol_type = other RNA
gaggaacaaactcttcttgga

SEQ ID NO: 40 - RNA, 21 nt, synthetic construct
  source 1..21: mol_type = other RNA
tgtgtggagggtcactcataa

SEQ ID NO: 41 - RNA, 21 nt, synthetic construct
  source 1..21: mol_type = other RNA
tatagtggcattttaaatcta

SEQ ID NO: 42 - RNA, 21 nt, synthetic construct
  source 1..21: mol_type = other RNA
tctgtcagacattacaaaaga

SEQ ID NO: 43 - RNA, 21 nt, synthetic construct
  source 1..21: mol_type = other RNA
ctgtcagacattacaaaagaa

SEQ ID NO: 44 - RNA, 21 nt, synthetic construct
  source 1..21: mol_type = other RNA
atacacctttctcacaaataa

SEQ ID NO: 45 - RNA, 21 nt, synthetic construct
  source 1..21: mol_type = other RNA
gaagggaatcatgatatcgca

SEQ ID NO: 46 - RNA, 21 nt, synthetic construct
  source 1..21: mol_type = other RNA
ctcctttgaattacactgaaa

SEQ ID NO: 47 - RNA, 21 nt, synthetic construct
  source 1..21: mol_type = other RNA
ttacactgaattccaaaaaca

SEQ ID NO: 48 - RNA, 21 nt, synthetic construct
  source 1..21: mol_type = other RNA
caagcacaatttataccaaca

SEQ ID NO: 49 - RNA, 21 nt, synthetic construct
  source 1..21: mol_type = other RNA
ccatcgagacatttataaaga

SEQ ID NO: 50 - RNA, 21 nt, synthetic construct
  source 1..21: mol_type = other RNA
tacctgaaccctgccattcta

SEQ ID NO: 51 - RNA, 21 nt, synthetic construct
  source 1..21: mol_type = other RNA
aaggttagcagtgttgaagaa

SEQ ID NO: 52 - RNA, 21 nt, synthetic construct
  source 1..21: mol_type = other RNA
ctctatgaaaacgccttctta

SEQ ID NO: 53 - RNA, 21 nt, synthetic construct
  source 1..21: mol_type = other RNA
cacccaaggtgtttgctatta

SEQ ID NO: 54 - RNA, 21 nt, synthetic construct
  source 1..21: mol_type = other RNA
tcagttttcttccagcaagta

SEQ ID NO: 55 - RNA, 21 nt, synthetic construct
  source 1..21: mol_type = other RNA
aatgacatggagaaaaggtta

SEQ ID NO: 56 - RNA, 21 nt, synthetic construct
  source 1..21: mol_type = other RNA
ggttgcttcttgaaagataga

SEQ ID NO: 57 - RNA, 21 nt, synthetic construct
  source 1..21: mol_type = other RNA
tcttgaaagatagtgttacaa

SEQ ID NO: 58 - RNA, 21 nt, synthetic construct
  source 1..21: mol_type = other RNA
tgtggtcatcaaataagtgca

SEQ ID NO: 59 - RNA, 21 nt, synthetic construct
  source 1..21: mol_type = other RNA
tgatatgagaggagtcaatta

SEQ ID NO: 60 - RNA, 21 nt, synthetic construct
  source 1..21: mol_type = other RNA
tatgagaggagtcaattttaa

SEQ ID NO: 61 - RNA, 21 nt, synthetic construct
  source 1..21: mol_type = other RNA
agtgttgaagaatgccaaaaa

SEQ ID NO: 62 - RNA, 21 nt, synthetic construct
  source 1..21: mol_type = other RNA
agtaacgtggaatctggatta

SEQ ID NO: 63 - RNA, 21 nt, synthetic construct
  source 1..21: mol_type = other RNA
cctgtgccctttcagaaatta

SEQ ID NO: 64 - RNA, 21 nt, synthetic construct
  source 1..21: mol_type = other RNA
ccttttaacctgcaaaagaaa

SEQ ID NO: 65 - RNA, 21 nt, synthetic construct
  source 1..21: mol_type = other RNA
gaaccctgccattctaaaata

SEQ ID NO: 66 - RNA, 21 nt, synthetic construct
  source 1..21: mol_type = other RNA
ccctgccattctaaaatttaa

SEQ ID NO: 67 - RNA, 21 nt, synthetic construct
  source 1..21: mol_type = other RNA
gagaagaattgaatgtgacta

SEQ ID NO: 68 - RNA, 21 nt, synthetic construct
  source 1..21: mol_type = other RNA
ttttgttaaaggagtgaatga

SEQ ID NO: 69 - RNA, 21 nt, synthetic construct
  source 1..21: mol_type = other RNA
gttaaaggagtgaatgtttga

SEQ ID NO: 70 - RNA, 21 nt, synthetic construct
  source 1..21: mol_type = other RNA
ttaaaggagtgaatgtttgca

SEQ ID NO: 71 - RNA, 21 nt, synthetic construct
  source 1..21: mol_type = other RNA
aaaggagtgaatgtttgccaa

SEQ ID NO: 72 - RNA, 21 nt, synthetic construct
  source 1..21: mol_type = other RNA
ttgcacaaagatgattcgcta

SEQ ID NO: 73 - RNA, 21 nt, synthetic construct
  source 1..21: mol_type = other RNA
aagatgattcgctgtcagtta

SEQ ID NO: 74 - RNA, 21 nt, synthetic construct
  source 1..21: mol_type = other RNA
tgattcgctgtcagtttttca

SEQ ID NO: 75 - RNA, 21 nt, synthetic construct
  source 1..21: mol_type = other RNA
cattcgctgtcagtttttcaa

SEQ ID NO: 76 - RNA, 21 nt, synthetic construct
  source 1..21: mol_type = other RNA
cttcgctgtcagtttttcaca

SEQ ID NO: 77 - RNA, 21 nt, synthetic construct
  source 1..21: mol_type = other RNA
ccgctgtcagtttttcactta

SEQ ID NO: 78 - RNA, 21 nt, synthetic construct
  source 1..21: mol_type = other RNA
cctgtcagtttttcacttata

SEQ ID NO: 79 - RNA, 21 nt, synthetic construct
  source 1..21: mol_type = other RNA
ctgtcagtttttcacttatta

SEQ ID NO: 80 - RNA, 21 nt, synthetic construct
  source 1..21: mol_type = other RNA
cgtcagtttttcacttattca

SEQ ID NO: 81 - RNA, 21 nt, synthetic construct
  source 1..21: mol_type = other RNA
gtcagtttttcacttattcta

SEQ ID NO: 82 - RNA, 21 nt, synthetic construct
  source 1..21: mol_type = other RNA
gtttttcacttattctttaca

SEQ ID NO: 83 - RNA, 21 nt, synthetic construct
  source 1..21: mol_type = other RNA
tttttcacttattctttacta

SEQ ID NO: 84 - RNA, 21 nt, synthetic construct
  source 1..21: mol_type = other RNA
tttcacttattctttactcca

SEQ ID NO: 85 - RNA, 21 nt, synthetic construct
  source 1..21: mol_type = other RNA
tattctttactcccagaagaa

SEQ ID NO: 86 - RNA, 21 nt, synthetic construct
  source 1..21: mol_type = other RNA
ttactcccagaagactgtaaa

SEQ ID NO: 87 - RNA, 21 nt, synthetic construct
  source 1..21: mol_type = other RNA
ctcccagaagactgtaaggaa

SEQ ID NO: 88 - RNA, 21 nt, synthetic construct
  source 1..21: mol_type = other RNA
gagaagtgtaagtgtttctta

SEQ ID NO: 89 - RNA, 21 nt, synthetic construct
  source 1..21: mol_type = other RNA
aagtgtaagtgtttcttaaga

SEQ ID NO: 90 - RNA, 21 nt, synthetic construct
  source 1..21: mol_type = other RNA
tgtaagtgtttcttaagatta

SEQ ID NO: 91 - RNA, 21 nt, synthetic construct
  source 1..21: mol_type = other RNA
ctaagtgtttcttaagattaa

SEQ ID NO: 92 - RNA, 21 nt, synthetic construct
  source 1..21: mol_type = other RNA
taagtgtttcttaagattata

SEQ ID NO: 93 - RNA, 21 nt, synthetic construct
  source 1..21: mol_type = other RNA
aagtgtttcttaagattatca

SEQ ID NO: 94 - RNA, 21 nt, synthetic construct
  source 1..21: mol_type = other RNA
gagctctggttactctttgaa

SEQ ID NO: 95 - RNA, 21 nt, synthetic construct
  source 1..21: mol_type = other RNA
ggttactctttgagattgtga

SEQ ID NO: 96 - RNA, 21 nt, synthetic construct
  source 1..21: mol_type = other RNA
cattgttggaggaacaaacta

SEQ ID NO: 97 - RNA, 21 nt, synthetic construct
  source 1..21: mol_type = other RNA
ctatagtggcattttaaatca

SEQ ID NO: 98 - RNA, 21 nt, synthetic construct
  source 1..21: mol_type = other RNA
tttaaatctgtcagacattaa

SEQ ID NO: 99 - RNA, 21 nt, synthetic construct
  source 1..21: mol_type = other RNA
attattattcaccaaaactaa

SEQ ID NO: 100 - RNA, 21 nt, synthetic construct
  source 1..21: mol_type = other RNA
agaagggaatcatgatatcga

SEQ ID NO: 101 - RNA, 21 nt, synthetic construct
  source 1..21: mol_type = other RNA
tccaggctcctttgaattaca

SEQ ID NO: 102 - RNA, 21 nt, synthetic construct
  source 1..21: mol_type = other RNA
actgaattccaaaaaccaata

SEQ ID NO: 103 - RNA, 21 nt, synthetic construct
  source 1..21: mol_type = other RNA
attccaaaaaccaatatgcca

SEQ ID NO: 104 - RNA, 21 nt, synthetic construct
  source 1..21: mol_type = other RNA
gcacaatttataccaactgta

SEQ ID NO: 105 - RNA, 21 nt, synthetic construct
  source 1..21: mol_type = other RNA
ttataccaactgttgggtaaa

SEQ ID NO: 106 - RNA, 21 nt, synthetic construct
  source 1..21: mol_type = other RNA
tacaccaaagtcgctgagtaa

SEQ ID NO: 107 - RNA, 21 nt, synthetic construct
  source 1..21: mol_type = other RNA
agtacatggactggattttaa

SEQ ID NO: 108 - RNA, 21 nt, synthetic construct
  source 1..21: mol_type = other RNA
tggactggattttagagaaaa

SEQ ID NO: 109 - RNA, 21 nt, synthetic construct
  source 1..21: mol_type = other RNA
ggactggattttagagaaaaa

SEQ ID NO: 110 - RNA, 21 nt, synthetic construct
  source 1..21: mol_type = other RNA
acacagagcagtgatggaaaa

SEQ ID NO: 111 - RNA, 21 nt, synthetic construct
  source 1..21: mol_type = other RNA
tgagttcaagtcaaattctga

SEQ ID NO: 112 - RNA, 21 nt, synthetic construct
  source 1..21: mol_type = other RNA
gagttcaagtcaaattctgaa

SEQ ID NO: 113 - RNA, 19 nt, synthetic construct
  source 1..19: mol_type = other RNA
gatgtagcttccatgtaca

SEQ ID NO: 114 - RNA, 19 nt, synthetic construct
  source 1..19: mol_type = other RNA
gttgcttcttgaaagataa

SEQ ID NO: 115 - RNA, 19 nt, synthetic construct
  source 1..19: mol_type = other RNA
tcttgaaagatagtgttaa

SEQ ID NO: 116 - RNA, 19 nt, synthetic construct
  source 1..19: mol_type = other RNA
atgtggtcatcaaataaga

SEQ ID NO: 117 - RNA, 19 nt, synthetic construct
  source 1..19: mol_type = other RNA
ggtcatcaaataagtgcta

SEQ ID NO: 118 - RNA, 19 nt, synthetic construct
  source 1..19: mol_type = other RNA
tcgagacatttataaagga

SEQ ID NO: 119 - RNA, 19 nt, synthetic construct
  source 1..19: mol_type = other RNA
cgagacatttataaaggaa

SEQ ID NO: 120 - RNA, 19 nt, synthetic construct
  source 1..19: mol_type = other RNA
tgatatgagaggagtcaaa

SEQ ID NO: 121 - RNA, 19 nt, synthetic construct
  source 1..19: mol_type = other RNA
aaggttagcagtgttgaaa

SEQ ID NO: 122 - RNA, 19 nt, synthetic construct
  source 1..19: mol_type = other RNA
ttagcagtgttgaagaata

SEQ ID NO: 123 - RNA, 19 nt, synthetic construct
  source 1..19: mol_type = other RNA
tagcagtgttgaagaatga

SEQ ID NO: 124 - RNA, 19 nt, synthetic construct
  source 1..19: mol_type = other RNA
aacgtggaatctggattca

SEQ ID NO: 125 - RNA, 19 nt, synthetic construct
  source 1..19: mol_type = other RNA
cgtggaatctggattctca

SEQ ID NO: 126 - RNA, 19 nt, synthetic construct
  source 1..19: mol_type = other RNA
tctggattctcactgaaga

SEQ ID NO: 127 - RNA, 19 nt, synthetic construct
  source 1..19: mol_type = other RNA
tccagatgcttttgtgtga

SEQ ID NO: 128 - RNA, 19 nt, synthetic construct
  source 1..19: mol_type = other RNA
cttaaaacatctgaaagta

SEQ ID NO: 129 - RNA, 19 nt, synthetic construct
  source 1..19: mol_type = other RNA
cctctactcctcaagaaaa

SEQ ID NO: 130 - RNA, 19 nt, synthetic construct
  source 1..19: mol_type = other RNA
ctctactcctcaagaaaaa

SEQ ID NO: 131 - RNA, 19 nt, synthetic construct
  source 1..19: mol_type = other RNA
cttttaacctgcaaaagaa

SEQ ID NO: 132 - RNA, 19 nt, synthetic construct
  source 1..19: mol_type = other RNA
cctgcaaaagaactttaca

SEQ ID NO: 133 - RNA, 19 nt, synthetic construct
  source 1..19: mol_type = other RNA
ctgcaaaagaactttacca

SEQ ID NO: 134 - RNA, 19 nt, synthetic construct
  source 1..19: mol_type = other RNA
tgcaaaagaactttaccta

SEQ ID NO: 135 - RNA, 19 nt, synthetic construct
  source 1..19: mol_type = other RNA
aaaagaactttacctgaaa

SEQ ID NO: 136 - RNA, 19 nt, synthetic construct
  source 1..19: mol_type = other RNA
tttggaggagaagaattga

SEQ ID NO: 137 - RNA, 19 nt, synthetic construct
  source 1..19: mol_type = other RNA
ggagaagaattgaatgtga

SEQ ID NO: 138 - RNA, 19 nt, synthetic construct
  source 1..19: mol_type = other RNA
ttaaaggagtgaatgttta

SEQ ID NO: 139 - RNA, 19 nt, synthetic construct
  source 1..19: mol_type = other RNA
cgctgtcagtttttcacta

SEQ ID NO: 140 - RNA, 19 nt, synthetic construct
  source 1..19: mol_type = other RNA
ctgtcagtttttcacttaa

SEQ ID NO: 141 - RNA, 19 nt, synthetic construct
  source 1..19: mol_type = other RNA
ttcacttattctttactca

SEQ ID NO: 142 - RNA, 19 nt, synthetic construct
  source 1..19: mol_type = other RNA
agagaagtgtaagtgttta

SEQ ID NO: 143 - RNA, 19 nt, synthetic construct
  source 1..19: mol_type = other RNA
agaagtgtaagtgtttcta

SEQ ID NO: 144 - RNA, 19 nt, synthetic construct
  source 1..19: mol_type = other RNA
aagtgtaagtgtttcttaa

SEQ ID NO: 145 - RNA, 19 nt, synthetic construct
  source 1..19: mol_type = other RNA
tgtaagtgtttcttaagaa

SEQ ID NO: 146 - RNA, 19 nt, synthetic construct
  source 1..19: mol_type = other RNA
ggttactctttgagattga

SEQ ID NO: 147 - RNA, 19 nt, synthetic construct
  source 1..19: mol_type = other RNA
gttactctttgagattgta

SEQ ID NO: 148 - RNA, 19 nt, synthetic construct
  source 1..19: mol_type = other RNA
tctttgagattgtgtaaca

SEQ ID NO: 149 - RNA, 19 nt, synthetic construct
  source 1..19: mol_type = other RNA
ctttgagattgtgtaacaa

SEQ ID NO: 150 - RNA, 19 nt, synthetic construct
  source 1..19: mol_type = other RNA
tgtctgcacaacaaaaaca

SEQ ID NO: 151 - RNA, 19 nt, synthetic construct
  source 1..19: mol_type = other RNA
ggaacaaactcttcttgga

SEQ ID NO: 152 - RNA, 19 nt, synthetic construct
  source 1..19: mol_type = other RNA
tgtggagggtcactcataa

SEQ ID NO: 153 - RNA, 19 nt, synthetic construct
  source 1..19: mol_type = other RNA
tagtggcattttaaatcta

SEQ ID NO: 154 - RNA, 19 nt, synthetic construct
  source 1..19: mol_type = other RNA
tgtcagacattacaaaaga

SEQ ID NO: 155 - RNA, 19 nt, synthetic construct
  source 1..19: mol_type = other RNA
gtcagacattacaaaagaa

SEQ ID NO: 156 - RNA, 19 nt, synthetic construct
  source 1..19: mol_type = other RNA
acacctttctcacaaataa

SEQ ID NO: 157 - RNA, 19 nt, synthetic construct
  source 1..19: mol_type = other RNA
agggaatcatgatatcgca

SEQ ID NO: 158 - RNA, 19 nt, synthetic construct
  source 1..19: mol_type = other RNA
cctttgaattacactgaaa

SEQ ID NO: 159 - RNA, 19 nt, synthetic construct
  source 1..19: mol_type = other RNA
acactgaattccaaaaaca

SEQ ID NO: 160 - RNA, 19 nt, synthetic construct
  source 1..19: mol_type = other RNA
agcacaatttataccaaca

SEQ ID NO: 161 - RNA, 19 nt, synthetic construct
  source 1..19: mol_type = other RNA
atcgagacatttataaaga

SEQ ID NO: 162 - RNA, 19 nt, synthetic construct
  source 1..19: mol_type = other RNA
cctgaaccctgccattcta

SEQ ID NO: 163 - RNA, 19 nt, synthetic construct
  source 1..19: mol_type = other RNA
ggttagcagtgttgaagaa

SEQ ID NO: 164 - RNA, 19 nt, synthetic construct
  source 1..19: mol_type = other RNA
ctatgaaaacgccttctta

SEQ ID NO: 165 - RNA, 19 nt, synthetic construct
  source 1..19: mol_type = other RNA
cccaaggtgtttgctatta

SEQ ID NO: 166 - RNA, 19 nt, synthetic construct
  source 1..19: mol_type = other RNA
agttttcttccagcaagta

SEQ ID NO: 167 - RNA, 19 nt, synthetic construct
  source 1..19: mol_type = other RNA
tgacatggagaaaaggtta

SEQ ID NO: 168 - RNA, 19 nt, synthetic construct
  source 1..19: mol_type = other RNA
ttgcttcttgaaagataga

SEQ ID NO: 169 - RNA, 19 nt, synthetic construct
  source 1..19: mol_type = other RNA
ttgaaagatagtgttacaa

SEQ ID NO: 170 - RNA, 19 nt, synthetic construct
  source 1..19: mol_type = other RNA
tggtcatcaaataagtgca

SEQ ID NO: 171 - RNA, 19 nt, synthetic construct
  source 1..19: mol_type = other RNA
atatgagaggagtcaatta

SEQ ID NO: 172 - RNA, 19 nt, synthetic construct
  source 1..19: mol_type = other RNA
tgagaggagtcaattttaa

SEQ ID NO: 173 - RNA, 19 nt, synthetic construct
  source 1..19: mol_type = other RNA
tgttgaagaatgccaaaaa

SEQ ID NO: 174 - RNA, 19 nt, synthetic construct
  source 1..19: mol_type = other RNA
taacgtggaatctggatta

SEQ ID NO: 175 - RNA, 19 nt, synthetic construct
  source 1..19: mol_type = other RNA
tgtgccctttcagaaatta

SEQ ID NO: 176 - RNA, 19 nt, synthetic construct
  source 1..19: mol_type = other RNA
ttttaacctgcaaaagaaa

SEQ ID NO: 177 - RNA, 19 nt, synthetic construct
  source 1..19: mol_type = other RNA
accctgccattctaaaata

SEQ ID NO: 178 - RNA, 19 nt, synthetic construct
  source 1..19: mol_type = other RNA
ctgccattctaaaatttaa

SEQ ID NO: 179 - RNA, 19 nt, synthetic construct
  source 1..19: mol_type = other RNA
gaagaattgaatgtgacta

SEQ ID NO: 180 - RNA, 19 nt, synthetic construct
  source 1..19: mol_type = other RNA
ttgttaaaggagtgaatga

SEQ ID NO: 181 - RNA, 19 nt, synthetic construct
  source 1..19: mol_type = other RNA
taaaggagtgaatgtttga

SEQ ID NO: 182 - RNA, 19 nt, synthetic construct
  source 1..19: mol_type = other RNA
aaaggagtgaatgtttgca

SEQ ID NO: 183 - RNA, 19 nt, synthetic construct
  source 1..19: mol_type = other RNA
aggagtgaatgtttgccaa

SEQ ID NO: 184 - RNA, 19 nt, synthetic construct
  source 1..19: mol_type = other RNA
gcacaaagatgattcgcta

SEQ ID NO: 185 - RNA, 19 nt, synthetic construct
  source 1..19: mol_type = other RNA
gatgattcgctgtcagtta

SEQ ID NO: 186 - RNA, 19 nt, synthetic construct
  source 1..19: mol_type = other RNA
attcgctgtcagtttttca

SEQ ID NO: 187 - RNA, 19 nt, synthetic construct
  source 1..19: mol_type = other RNA
ttcgctgtcagtttttcaa

SEQ ID NO: 188 - RNA, 19 nt, synthetic construct
  source 1..19: mol_type = other RNA
tcgctgtcagtttttcaca

SEQ ID NO: 189 - RNA, 19 nt, synthetic construct
  source 1..19: mol_type = other RNA
gctgtcagtttttcactta

SEQ ID NO: 190 - RNA, 19 nt, synthetic construct
  source 1..19: mol_type = other RNA
tgtcagtttttcacttata

SEQ ID NO: 191 - RNA, 19 nt, synthetic construct
  source 1..19: mol_type = other RNA
gtcagtttttcacttatta

SEQ ID NO: 192 - RNA, 19 nt, synthetic construct
  source 1..19: mol_type = other RNA
tcagtttttcacttattca

SEQ ID NO: 193 - RNA, 19 nt, synthetic construct
  source 1..19: mol_type = other RNA
cagtttttcacttattcta

SEQ ID NO: 194 - RNA, 19 nt, synthetic construct
  source 1..19: mol_type = other RNA
ttttcacttattctttaca

SEQ ID NO: 195 - RNA, 19 nt, synthetic construct
  source 1..19: mol_type = other RNA
tttcacttattctttacta

SEQ ID NO: 196 - RNA, 19 nt, synthetic construct
  source 1..19: mol_type = other RNA
tcacttattctttactcca

SEQ ID NO: 197 - RNA, 19 nt, synthetic construct
  source 1..19: mol_type = other RNA
ttctttactcccagaagaa

SEQ ID NO: 198 - RNA, 19 nt, synthetic construct
  source 1..19: mol_type = other RNA
actcccagaagactgtaaa

SEQ ID NO: 199 - RNA, 19 nt, synthetic construct
  source 1..19: mol_type = other RNA
cccagaagactgtaaggaa

SEQ ID NO: 200 - RNA, 19 nt, synthetic construct
  source 1..19: mol_type = other RNA
gaagtgtaagtgtttctta

SEQ ID NO: 201 - RNA, 19 nt, synthetic construct
  source 1..19: mol_type = other RNA
gtgtaagtgtttcttaaga

SEQ ID NO: 202 - RNA, 19 nt, synthetic construct
  source 1..19: mol_type = other RNA
taagtgtttcttaagatta

SEQ ID NO: 203 - RNA, 19 nt, synthetic construct
  source 1..19: mol_type = other RNA
aagtgtttcttaagattaa

SEQ ID NO: 204 - RNA, 19 nt, synthetic construct
  source 1..19: mol_type = other RNA
agtgtttcttaagattata

SEQ ID NO: 205 - RNA, 19 nt, synthetic construct
  source 1..19: mol_type = other RNA
gtgtttcttaagattatca

SEQ ID NO: 206 - RNA, 19 nt, synthetic construct
  source 1..19: mol_type = other RNA
gctctggttactctttgaa

SEQ ID NO: 207 - RNA, 19 nt, synthetic construct
  source 1..19: mol_type = other RNA
ttactctttgagattgtga

SEQ ID NO: 208 - RNA, 19 nt, synthetic construct
  source 1..19: mol_type = other RNA
ttgttggaggaacaaacta

SEQ ID NO: 209 - RNA, 19 nt, synthetic construct
  source 1..19: mol_type = other RNA
atagtggcattttaaatca

SEQ ID NO: 210 - RNA, 19 nt, synthetic construct
  source 1..19: mol_type = other RNA
taaatctgtcagacattaa

SEQ ID NO: 211 - RNA, 19 nt, synthetic construct
  source 1..19: mol_type = other RNA
tattattcaccaaaactaa

SEQ ID NO: 212 - RNA, 19 nt, synthetic construct
  source 1..19: mol_type = other RNA
aagggaatcatgatatcga

SEQ ID NO: 213 - RNA, 19 nt, synthetic construct
  source 1..19: mol_type = other RNA
caggctcctttgaattaca

SEQ ID NO: 214 - RNA, 19 nt, synthetic construct
  source 1..19: mol_type = other RNA
tgaattccaaaaaccaata

SEQ ID NO: 215 - RNA, 19 nt, synthetic construct
  source 1..19: mol_type = other RNA
tccaaaaaccaatatgcca

SEQ ID NO: 216 - RNA, 19 nt, synthetic construct
  source 1..19: mol_type = other RNA
acaatttataccaactgta

SEQ ID NO: 217 - RNA, 19 nt, synthetic construct
  source 1..19: mol_type = other RNA
ataccaactgttgggtaaa

SEQ ID NO: 218 - RNA, 19 nt, synthetic construct
  source 1..19: mol_type = other RNA
caccaaagtcgctgagtaa

SEQ ID NO: 219 - RNA, 19 nt, synthetic construct
  source 1..19: mol_type = other RNA
tacatggactggattttaa

SEQ ID NO: 220 - RNA, 19 nt, synthetic construct
  source 1..19: mol_type = other RNA
gactggattttagagaaaa

SEQ ID NO: 221 - RNA, 19 nt, synthetic construct
  source 1..19: mol_type = other RNA
actggattttagagaaaaa

SEQ ID NO: 222 - RNA, 19 nt, synthetic construct
  source 1..19: mol_type = other RNA
acagagcagtgatggaaaa

SEQ ID NO: 223 - RNA, 19 nt, synthetic construct
  source 1..19: mol_type = other RNA
agttcaagtcaaattctga

SEQ ID NO: 224 - RNA, 19 nt, synthetic construct
  source 1..19: mol_type = other RNA
gttcaagtcaaattctgaa

SEQ ID NO: 225 - RNA, 21 nt, synthetic construct
  source 1..21: mol_type = other RNA
tgtacatggaagctacatccc

SEQ ID NO: 226 - RNA, 21 nt, synthetic construct
  source 1..21: mol_type = other RNA
ttatctttcaagaagcaacca

SEQ ID NO: 227 - RNA, 21 nt, synthetic construct
  source 1..21: mol_type = other RNA
ttaacactatctttcaagaag

SEQ ID NO: 228 - RNA, 21 nt, synthetic construct
  source 1..21: mol_type = other RNA
tcttatttgatgaccacattg

SEQ ID NO: 229 - RNA, 21 nt, synthetic construct
  source 1..21: mol_type = other RNA
tagcacttatttgatgaccac

SEQ ID NO: 230 - RNA, 21 nt, synthetic construct
  source 1..21: mol_type = other RNA
tcctttataaatgtctcgatg

SEQ ID NO: 231 - RNA, 21 nt, synthetic construct
  source 1..21: mol_type = other RNA
ttcctttataaatgtctcgat

SEQ ID NO: 232 - RNA, 21 nt, synthetic construct
  source 1..21: mol_type = other RNA
tttgactcctctcatatcaac

SEQ ID NO: 233 - RNA, 21 nt, synthetic construct
  source 1..21: mol_type = other RNA
tttcaacactgctaaccttag

SEQ ID NO: 234 - RNA, 21 nt, synthetic construct
  source 1..21: mol_type = other RNA
tattcttcaacactgctaacc

SEQ ID NO: 235 - RNA, 21 nt, synthetic construct
  source 1..21: mol_type = other RNA
tcattcttcaacactgctaac

SEQ ID NO: 236 - RNA, 21 nt, synthetic construct
  source 1..21: mol_type = other RNA
tgaatccagattccacgttac

SEQ ID NO: 237 - RNA, 21 nt, synthetic construct
  source 1..21: mol_type = other RNA
tgagaatccagattccacgtt

SEQ ID NO: 238 - RNA, 21 nt, synthetic construct
  source 1..21: mol_type = other RNA
tcttcagtgagaatccagatt

SEQ ID NO: 239 - RNA, 21 nt, synthetic construct
  source 1..21: mol_type = other RNA
tcacacaaaagcatctggagt

SEQ ID NO: 240 - RNA, 21 nt, synthetic construct
  source 1..21: mol_type = other RNA
tactttcagatgttttaagaa

SEQ ID NO: 241 - RNA, 21 nt, synthetic construct
  source 1..21: mol_type = other RNA
ttttcttgaggagtagaggaa

SEQ ID NO: 242 - RNA, 21 nt, synthetic construct
  source 1..21: mol_type = other RNA
tttttcttgaggagtagagga

SEQ ID NO: 243 - RNA, 21 nt, synthetic construct
  source 1..21: mol_type = other RNA
ttcttttgcaggttaaaaggc

SEQ ID NO: 244 - RNA, 21 nt, synthetic construct
  source 1..21: mol_type = other RNA
tgtaaagttcttttgcaggtt

SEQ ID NO: 245 - RNA, 21 nt, synthetic construct
  source 1..21: mol_type = other RNA
tggtaaagttcttttgcaggt

SEQ ID NO: 246 - RNA, 21 nt, synthetic construct
  source 1..21: mol_type = other RNA
taggtaaagttcttttgcagg

SEQ ID NO: 247 - RNA, 21 nt, synthetic construct
  source 1..21: mol_type = other RNA
tttcaggtaaagttcttttgc

SEQ ID NO: 248 - RNA, 21 nt, synthetic construct
  source 1..21: mol_type = other RNA
tcaattcttctcctccaaagt

SEQ ID NO: 249 - RNA, 21 nt, synthetic construct
  source 1..21: mol_type = other RNA
tcacattcaattcttctcctc

SEQ ID NO: 250 - RNA, 21 nt, synthetic construct
  source 1..21: mol_type = other RNA
taaacattcactcctttaaca

SEQ ID NO: 251 - RNA, 21 nt, synthetic construct
  source 1..21: mol_type = other RNA
tagtgaaaaactgacagcgaa

SEQ ID NO: 252 - RNA, 21 nt, synthetic construct
  source 1..21: mol_type = other RNA
ttaagtgaaaaactgacagcg

SEQ ID NO: 253 - RNA, 21 nt, synthetic construct
  source 1..21: mol_type = other RNA
tgagtaaagaataagtgaaaa

SEQ ID NO: 254 - RNA, 21 nt, synthetic construct
  source 1..21: mol_type = other RNA
taaacacttacacttctcttc

SEQ ID NO: 255 - RNA, 21 nt, synthetic construct
  source 1..21: mol_type = other RNA
tagaaacacttacacttctct

SEQ ID NO: 256 - RNA, 21 nt, synthetic construct
  source 1..21: mol_type = other RNA
ttaagaaacacttacacttct

SEQ ID NO: 257 - RNA, 21 nt, synthetic construct
  source 1..21: mol_type = other RNA
ttcttaagaaacacttacact

SEQ ID NO: 258 - RNA, 21 nt, synthetic construct
  source 1..21: mol_type = other RNA
tcaatctcaaagagtaaccag

SEQ ID NO: 259 - RNA, 21 nt, synthetic construct
  source 1..21: mol_type = other RNA
tacaatctcaaagagtaacca

SEQ ID NO: 260 - RNA, 21 nt, synthetic construct
  source 1..21: mol_type = other RNA
tgttacacaatctcaaagagt

SEQ ID NO: 261 - RNA, 21 nt, synthetic construct
  source 1..21: mol_type = other RNA
ttgttacacaatctcaaagag

SEQ ID NO: 262 - RNA, 21 nt, synthetic construct
  source 1..21: mol_type = other RNA
tgtttttgttgtgcagacaga

SEQ ID NO: 263 - RNA, 21 nt, synthetic construct
  source 1..21: mol_type = other RNA
tccaagaagagtttgttcctc

SEQ ID NO: 264 - RNA, 21 nt, synthetic construct
  source 1..21: mol_type = other RNA
ttatgagtgaccctccacaca

SEQ ID NO: 265 - RNA, 21 nt, synthetic construct
  source 1..21: mol_type = other RNA
tagatttaaaatgccactata

SEQ ID NO: 266 - RNA, 21 nt, synthetic construct
  source 1..21: mol_type = other RNA
tcttttgtaatgtctgacaga

SEQ ID NO: 267 - RNA, 21 nt, synthetic construct
  source 1..21: mol_type = other RNA
ttcttttgtaatgtctgacag

SEQ ID NO: 268 - RNA, 21 nt, synthetic construct
  source 1..21: mol_type = other RNA
ttatttgtgagaaaggtgtat

SEQ ID NO: 269 - RNA, 21 nt, synthetic construct
  source 1..21: mol_type = other RNA
tgcgatatcatgattcccttc

SEQ ID NO: 270 - RNA, 21 nt, synthetic construct
  source 1..21: mol_type = other RNA
tttcagtgtaattcaaaggag

SEQ ID NO: 271 - RNA, 21 nt, synthetic construct
  source 1..21: mol_type = other RNA
tgtttttggaattcagtgtaa

SEQ ID NO: 272 - RNA, 21 nt, synthetic construct
  source 1..21: mol_type = other RNA
tgttggtataaattgtgcttg

SEQ ID NO: 273 - RNA, 21 nt, synthetic construct
  source 1..21: mol_type = other RNA
tctttataaatgtctcgatgg

SEQ ID NO: 274 - RNA, 21 nt, synthetic construct
  source 1..21: mol_type = other RNA
tagaatggcagggttcaggta

SEQ ID NO: 275 - RNA, 21 nt, synthetic construct
  source 1..21: mol_type = other RNA
ttcttcaacactgctaacctt

SEQ ID NO: 276 - RNA, 21 nt, synthetic construct
  source 1..21: mol_type = other RNA
taagaaggcgttttcatagag

SEQ ID NO: 277 - RNA, 21 nt, synthetic construct
  source 1..21: mol_type = other RNA
taatagcaaacaccttgggtg

SEQ ID NO: 278 - RNA, 21 nt, synthetic construct
  source 1..21: mol_type = other RNA
tacttgctggaagaaaactga

SEQ ID NO: 279 - RNA, 21 nt, synthetic construct
  source 1..21: mol_type = other RNA
taaccttttctccatgtcatt

SEQ ID NO: 280 - RNA, 21 nt, synthetic construct
  source 1..21: mol_type = other RNA
tctatctttcaagaagcaacc

SEQ ID NO: 281 - RNA, 21 nt, synthetic construct
  source 1..21: mol_type = other RNA
ttgtaacactatctttcaaga

SEQ ID NO: 282 - RNA, 21 nt, synthetic construct
  source 1..21: mol_type = other RNA
tgcacttatttgatgaccaca

SEQ ID NO: 283 - RNA, 21 nt, synthetic construct
  source 1..21: mol_type = other RNA
taattgactcctctcatatca

SEQ ID NO: 284 - RNA, 21 nt, synthetic construct
  source 1..21: mol_type = other RNA
ttaaaattgactcctctcata

SEQ ID NO: 285 - RNA, 21 nt, synthetic construct
  source 1..21: mol_type = other RNA
tttttggcattcttcaacact

SEQ ID NO: 286 - RNA, 21 nt, synthetic construct
  source 1..21: mol_type = other RNA
taatccagattccacgttact

SEQ ID NO: 287 - RNA, 21 nt, synthetic construct
  source 1..21: mol_type = other RNA
taatttctgaaagggcacagg

SEQ ID NO: 288 - RNA, 21 nt, synthetic construct
  source 1..21: mol_type = other RNA
tttcttttgcaggttaaaagg

SEQ ID NO: 289 - RNA, 21 nt, synthetic construct
  source 1..21: mol_type = other RNA
tattttagaatggcagggttc

SEQ ID NO: 290 - RNA, 21 nt, synthetic construct
  source 1..21: mol_type = other RNA
ttaaattttagaatggcaggg

SEQ ID NO: 291 - RNA, 21 nt, synthetic construct
  source 1..21: mol_type = other RNA
tagtcacattcaattcttctc

SEQ ID NO: 292 - RNA, 21 nt, synthetic construct
  source 1..21: mol_type = other RNA
tcattcactcctttaacaaaa

SEQ ID NO: 293 - RNA, 21 nt, synthetic construct
  source 1..21: mol_type = other RNA
tcaaacattcactcctttaac

SEQ ID NO: 294 - RNA, 21 nt, synthetic construct
  source 1..21: mol_type = other RNA
tgcaaacattcactcctttaa

SEQ ID NO: 295 - RNA, 21 nt, synthetic construct
  source 1..21: mol_type = other RNA
ttggcaaacattcactccttt

SEQ ID NO: 296 - RNA, 21 nt, synthetic construct
  source 1..21: mol_type = other RNA
tagcgaatcatctttgtgcaa

SEQ ID NO: 297 - RNA, 21 nt, synthetic construct
  source 1..21: mol_type = other RNA
taactgacagcgaatcatctt

SEQ ID NO: 298 - RNA, 21 nt, synthetic construct
  source 1..21: mol_type = other RNA
tgaaaaactgacagcgaatca

SEQ ID NO: 299 - RNA, 21 nt, synthetic construct
  source 1..21: mol_type = other RNA
ttgaaaaactgacagcgaatg

SEQ ID NO: 300 - RNA, 21 nt, synthetic construct
  source 1..21: mol_type = other RNA
tgtgaaaaactgacagcgaag

SEQ ID NO: 301 - RNA, 21 nt, synthetic construct
  source 1..21: mol_type = other RNA
taagtgaaaaactgacagcgg

SEQ ID NO: 302 - RNA, 21 nt, synthetic construct
  source 1..21: mol_type = other RNA
tataagtgaaaaactgacagg

SEQ ID NO: 303 - RNA, 21 nt, synthetic construct
  source 1..21: mol_type = other RNA
taataagtgaaaaactgacag

SEQ ID NO: 304 - RNA, 21 nt, synthetic construct
  source 1..21: mol_type = other RNA
tgaataagtgaaaaactgacg

SEQ ID NO: 305 - RNA, 21 nt, synthetic construct
  source 1..21: mol_type = other RNA
tagaataagtgaaaaactgac

SEQ ID NO: 306 - RNA, 21 nt, synthetic construct
  source 1..21: mol_type = other RNA
tgtaaagaataagtgaaaaac

SEQ ID NO: 307 - RNA, 21 nt, synthetic construct
  source 1..21: mol_type = other RNA
tagtaaagaataagtgaaaaa

SEQ ID NO: 308 - RNA, 21 nt, synthetic construct
  source 1..21: mol_type = other RNA
tggagtaaagaataagtgaaa

SEQ ID NO: 309 - RNA, 21 nt, synthetic construct
  source 1..21: mol_type = other RNA
ttcttctgggagtaaagaata

SEQ ID NO: 310 - RNA, 21 nt, synthetic construct
  source 1..21: mol_type = other RNA
tttacagtcttctgggagtaa

SEQ ID NO: 311 - RNA, 21 nt, synthetic construct
  source 1..21: mol_type = other RNA
ttccttacagtcttctgggag

SEQ ID NO: 312 - RNA, 21 nt, synthetic construct
  source 1..21: mol_type = other RNA
taagaaacacttacacttctc

SEQ ID NO: 313 - RNA, 21 nt, synthetic construct
  source 1..21: mol_type = other RNA
tcttaagaaacacttacactt

SEQ ID NO: 314 - RNA, 21 nt, synthetic construct
  source 1..21: mol_type = other RNA
taatcttaagaaacacttaca

SEQ ID NO: 315 - RNA, 21 nt, synthetic construct
  source 1..21: mol_type = other RNA
ttaatcttaagaaacacttag

SEQ ID NO: 316 - RNA, 21 nt, synthetic construct
  source 1..21: mol_type = other RNA
tataatcttaagaaacactta

SEQ ID NO: 317 - RNA, 21 nt, synthetic construct
  source 1..21: mol_type = other RNA
tgataatcttaagaaacactt

SEQ ID NO: 318 - RNA, 21 nt, synthetic construct
  source 1..21: mol_type = other RNA
ttcaaagagtaaccagagctc

SEQ ID NO: 319 - RNA, 21 nt, synthetic construct
  source 1..21: mol_type = other RNA
tcacaatctcaaagagtaacc

SEQ ID NO: 320 - RNA, 21 nt, synthetic construct
  source 1..21: mol_type = other RNA
tagtttgttcctccaacaatg

SEQ ID NO: 321 - RNA, 21 nt, synthetic construct
  source 1..21: mol_type = other RNA
tgatttaaaatgccactatag

SEQ ID NO: 322 - RNA, 21 nt, synthetic construct
  source 1..21: mol_type = other RNA
ttaatgtctgacagatttaaa

SEQ ID NO: 323 - RNA, 21 nt, synthetic construct
  source 1..21: mol_type = other RNA
ttagttttggtgaataataat

SEQ ID NO: 324 - RNA, 21 nt, synthetic construct
  source 1..21: mol_type = other RNA
tcgatatcatgattcccttct

SEQ ID NO: 325 - RNA, 21 nt, synthetic construct
  source 1..21: mol_type = other RNA
tgtaattcaaaggagcctgga

SEQ ID NO: 326 - RNA, 21 nt, synthetic construct
  source 1..21: mol_type = other RNA
tattggtttttggaattcagt

SEQ ID NO: 327 - RNA, 21 nt, synthetic construct
  source 1..21: mol_type = other RNA
tggcatattggtttttggaat

SEQ ID NO: 328 - RNA, 21 nt, synthetic construct
  source 1..21: mol_type = other RNA
tacagttggtataaattgtgc

SEQ ID NO: 329 - RNA, 21 nt, synthetic construct
  source 1..21: mol_type = other RNA
tttacccaacagttggtataa

SEQ ID NO: 330 - RNA, 21 nt, synthetic construct
  source 1..21: mol_type = other RNA
ttactcagcgactttggtgta

SEQ ID NO: 331 - RNA, 21 nt, synthetic construct
  source 1..21: mol_type = other RNA
ttaaaatccagtccatgtact

SEQ ID NO: 332 - RNA, 21 nt, synthetic construct
  source 1..21: mol_type = other RNA
ttttctctaaaatccagtcca

SEQ ID NO: 333 - RNA, 21 nt, synthetic construct
  source 1..21: mol_type = other RNA
tttttctctaaaatccagtcc

SEQ ID NO: 334 - RNA, 21 nt, synthetic construct
  source 1..21: mol_type = other RNA
ttttccatcactgctctgtgt

SEQ ID NO: 335 - RNA, 21 nt, synthetic construct
  source 1..21: mol_type = other RNA
tcagaatttgacttgaactca

SEQ ID NO: 336 - RNA, 21 nt, synthetic construct
  source 1..21: mol_type = other RNA
ttcagaatttgacttgaactc

SEQ ID NO: 337 - RNA, 19 nt, synthetic construct
  source 1..19: mol_type = other RNA
tgtacatggaagctacatc

SEQ ID NO: 338 - RNA, 19 nt, synthetic construct
  source 1..19: mol_type = other RNA
ttatctttcaagaagcaac

SEQ ID NO: 339 - RNA, 19 nt, synthetic construct
  source 1..19: mol_type = other RNA
ttaacactatctttcaaga

SEQ ID NO: 340 - RNA, 19 nt, synthetic construct
  source 1..19: mol_type = other RNA
tcttatttgatgaccacat

SEQ ID NO: 341 - RNA, 19 nt, synthetic construct
  source 1..19: mol_type = other RNA
tagcacttatttgatgacc

SEQ ID NO: 342 - RNA, 19 nt, synthetic construct
  source 1..19: mol_type = other RNA
tcctttataaatgtctcga

SEQ ID NO: 343 - RNA, 19 nt, synthetic construct
  source 1..19: mol_type = other RNA
ttcctttataaatgtctcg

SEQ ID NO: 344 - RNA, 19 nt, synthetic construct
  source 1..19: mol_type = other RNA
tttgactcctctcatatca

SEQ ID NO: 345 - RNA, 19 nt, synthetic construct
  source 1..19: mol_type = other RNA
tttcaacactgctaacctt

SEQ ID NO: 346 - RNA, 19 nt, synthetic construct
  source 1..19: mol_type = other RNA
tattcttcaacactgctaa

SEQ ID NO: 347 - RNA, 19 nt, synthetic construct
  source 1..19: mol_type = other RNA
tcattcttcaacactgcta

SEQ ID NO: 348 - RNA, 19 nt, synthetic construct
  source 1..19: mol_type = other RNA
tgaatccagattccacgtt

SEQ ID NO: 349 - RNA, 19 nt, synthetic construct
  source 1..19: mol_type = other RNA
tgagaatccagattccacg

SEQ ID NO: 350 - RNA, 19 nt, synthetic construct
  source 1..19: mol_type = other RNA
tcttcagtgagaatccaga

SEQ ID NO: 351 - RNA, 19 nt, synthetic construct
  source 1..19: mol_type = other RNA
tcacacaaaagcatctgga

SEQ ID NO: 352 - RNA, 19 nt, synthetic construct
  source 1..19: mol_type = other RNA
tactttcagatgttttaag

SEQ ID NO: 353 - RNA, 19 nt, synthetic construct
  source 1..19: mol_type = other RNA
ttttcttgaggagtagagg

SEQ ID NO: 354 - RNA, 19 nt, synthetic construct
  source 1..19: mol_type = other RNA
tttttcttgaggagtagag

SEQ ID NO: 355 - RNA, 19 nt, synthetic construct
  source 1..19: mol_type = other RNA
ttcttttgcaggttaaaag

SEQ ID NO: 356 - RNA, 19 nt, synthetic construct
  source 1..19: mol_type = other RNA
tgtaaagttcttttgcagg

SEQ ID NO: 357 - RNA, 19 nt, synthetic construct
  source 1..19: mol_type = other RNA
tggtaaagttcttttgcag

SEQ ID NO: 358 - RNA, 19 nt, synthetic construct
  source 1..19: mol_type = other RNA
taggtaaagttcttttgca

SEQ ID NO: 359 - RNA, 19 nt, synthetic construct
  source 1..19: mol_type = other RNA
tttcaggtaaagttctttt

SEQ ID NO: 360 - RNA, 19 nt, synthetic construct
  source 1..19: mol_type = other RNA
tcaattcttctcctccaaa

SEQ ID NO: 361 - RNA, 19 nt, synthetic construct
  source 1..19: mol_type = other RNA
tcacattcaattcttctcc

SEQ ID NO: 362 - RNA, 19 nt, synthetic construct
  source 1..19: mol_type = other RNA
taaacattcactcctttaa

SEQ ID NO: 363 - RNA, 19 nt, synthetic construct
  source 1..19: mol_type = other RNA
tagtgaaaaactgacagcg

SEQ ID NO: 364 - RNA, 19 nt, synthetic construct
  source 1..19: mol_type = other RNA
ttaagtgaaaaactgacag

SEQ ID NO: 365 - RNA, 19 nt, synthetic construct
  source 1..19: mol_type = other RNA
tgagtaaagaataagtgaa

SEQ ID NO: 366 - RNA, 19 nt, synthetic construct
  source 1..19: mol_type = other RNA
taaacacttacacttctct

SEQ ID NO: 367 - RNA, 19 nt, synthetic construct
  source 1..19: mol_type = other RNA
tagaaacacttacacttct

SEQ ID NO: 368 - RNA, 19 nt, synthetic construct
  source 1..19: mol_type = other RNA
ttaagaaacacttacactt

SEQ ID NO: 369 - RNA, 19 nt, synthetic construct
  source 1..19: mol_type = other RNA
ttcttaagaaacacttaca

SEQ ID NO: 370 - RNA, 19 nt, synthetic construct
  source 1..19: mol_type = other RNA
tcaatctcaaagagtaacc

SEQ ID NO: 371 - RNA, 19 nt, synthetic construct
  source 1..19: mol_type = other RNA
tacaatctcaaagagtaac

SEQ ID NO: 372 - RNA, 19 nt, synthetic construct
  source 1..19: mol_type = other RNA
tgttacacaatctcaaaga

SEQ ID NO: 373 - RNA, 19 nt, synthetic construct
  source 1..19: mol_type = other RNA
ttgttacacaatctcaaag

SEQ ID NO: 374 - RNA, 19 nt, synthetic construct
  source 1..19: mol_type = other RNA
tgtttttgttgtgcagaca

SEQ ID NO: 375 - RNA, 19 nt, synthetic construct
  source 1..19: mol_type = other RNA
tccaagaagagtttgttcc

SEQ ID NO: 376 - RNA, 19 nt, synthetic construct
  source 1..19: mol_type = other RNA
ttatgagtgaccctccaca

SEQ ID NO: 377 - RNA, 19 nt, synthetic construct
  source 1..19: mol_type = other RNA
tagatttaaaatgccacta

SEQ ID NO: 378 - RNA, 19 nt, synthetic construct
  source 1..19: mol_type = other RNA
tcttttgtaatgtctgaca

SEQ ID NO: 379 - RNA, 19 nt, synthetic construct
  source 1..19: mol_type = other RNA
ttcttttgtaatgtctgac

SEQ ID NO: 380 - RNA, 19 nt, synthetic construct
  source 1..19: mol_type = other RNA
ttatttgtgagaaaggtgt

SEQ ID NO: 381 - RNA, 19 nt, synthetic construct
  source 1..19: mol_type = other RNA
tgcgatatcatgattccct

SEQ ID NO: 382 - RNA, 19 nt, synthetic construct
  source 1..19: mol_type = other RNA
tttcagtgtaattcaaagg

SEQ ID NO: 383 - RNA, 19 nt, synthetic construct
  source 1..19: mol_type = other RNA
tgtttttggaattcagtgt

SEQ ID NO: 384 - RNA, 19 nt, synthetic construct
  source 1..19: mol_type = other RNA
tgttggtataaattgtgct

SEQ ID NO: 385 - RNA, 19 nt, synthetic construct
  source 1..19: mol_type = other RNA
tctttataaatgtctcgat

SEQ ID NO: 386 - RNA, 19 nt, synthetic construct
  source 1..19: mol_type = other RNA
tagaatggcagggttcagg

SEQ ID NO: 387 - RNA, 19 nt, synthetic construct
  source 1..19: mol_type = other RNA
ttcttcaacactgctaacc

SEQ ID NO: 388 - RNA, 19 nt, synthetic construct
  source 1..19: mol_type = other RNA
taagaaggcgttttcatag

SEQ ID NO: 389 - RNA, 19 nt, synthetic construct
  source 1..19: mol_type = other RNA
taatagcaaacaccttggg

SEQ ID NO: 390 - RNA, 19 nt, synthetic construct
  source 1..19: mol_type = other RNA
tacttgctggaagaaaact

SEQ ID NO: 391 - RNA, 19 nt, synthetic construct
  source 1..19: mol_type = other RNA
taaccttttctccatgtca

SEQ ID NO: 392 - RNA, 19 nt, synthetic construct
  source 1..19: mol_type = other RNA
tctatctttcaagaagcaa

SEQ ID NO: 393 - RNA, 19 nt, synthetic construct
  source 1..19: mol_type = other RNA
ttgtaacactatctttcaa

SEQ ID NO: 394 - RNA, 19 nt, synthetic construct
  source 1..19: mol_type = other RNA
tgcacttatttgatgacca

SEQ ID NO: 395 - RNA, 19 nt, synthetic construct
  source 1..19: mol_type = other RNA
taattgactcctctcatat

SEQ ID NO: 396 - RNA, 19 nt, synthetic construct
  source 1..19: mol_type = other RNA
ttaaaattgactcctctca

SEQ ID NO: 397 - RNA, 19 nt, synthetic construct
  source 1..19: mol_type = other RNA
tttttggcattcttcaaca

SEQ ID NO: 398 - RNA, 19 nt, synthetic construct
  source 1..19: mol_type = other RNA
taatccagattccacgtta

SEQ ID NO: 399 - RNA, 19 nt, synthetic construct
  source 1..19: mol_type = other RNA
taatttctgaaagggcaca

SEQ ID NO: 400 - RNA, 19 nt, synthetic construct
  source 1..19: mol_type = other RNA
tttcttttgcaggttaaaa

SEQ ID NO: 401 - RNA, 19 nt, synthetic construct
  source 1..19: mol_type = other RNA
tattttagaatggcagggt

SEQ ID NO: 402 - RNA, 19 nt, synthetic construct
  source 1..19: mol_type = other RNA
ttaaattttagaatggcag

SEQ ID NO: 403 - RNA, 19 nt, synthetic construct
  source 1..19: mol_type = other RNA
tagtcacattcaattcttc

SEQ ID NO: 404 - RNA, 19 nt, synthetic construct
  source 1..19: mol_type = other RNA
tcattcactcctttaacaa

SEQ ID NO: 405 - RNA, 19 nt, synthetic construct
  source 1..19: mol_type = other RNA
tcaaacattcactccttta

SEQ ID NO: 406 - RNA, 19 nt, synthetic construct
  source 1..19: mol_type = other RNA
tgcaaacattcactccttt

SEQ ID NO: 407 - RNA, 19 nt, synthetic construct
  source 1..19: mol_type = other RNA
ttggcaaacattcactcct

SEQ ID NO: 408 - RNA, 19 nt, synthetic construct
  source 1..19: mol_type = other RNA
tagcgaatcatctttgtgc

SEQ ID NO: 409 - RNA, 19 nt, synthetic construct
  source 1..19: mol_type = other RNA
taactgacagcgaatcatc

SEQ ID NO: 410 - RNA, 19 nt, synthetic construct
  source 1..19: mol_type = other RNA
tgaaaaactgacagcgaat

SEQ ID NO: 411 - RNA, 19 nt, synthetic construct
  source 1..19: mol_type = other RNA
ttgaaaaactgacagcgaa

SEQ ID NO: 412 - RNA, 19 nt, synthetic construct
  source 1..19: mol_type = other RNA
tgtgaaaaactgacagcga

SEQ ID NO: 413 - RNA, 19 nt, synthetic construct
  source 1..19: mol_type = other RNA
taagtgaaaaactgacagc

SEQ ID NO: 414 - RNA, 19 nt, synthetic construct
  source 1..19: mol_type = other RNA
tataagtgaaaaactgaca

SEQ ID NO: 415 - RNA, 19 nt, synthetic construct
  source 1..19: mol_type = other RNA
taataagtgaaaaactgac

SEQ ID NO: 416 - RNA, 19 nt, synthetic construct
  source 1..19: mol_type = other RNA
tgaataagtgaaaaactga

SEQ ID NO: 417 - RNA, 19 nt, synthetic construct
  source 1..19: mol_type = other RNA
tagaataagtgaaaaactg

SEQ ID NO: 418 - RNA, 19 nt, synthetic construct
  source 1..19: mol_type = other RNA
tgtaaagaataagtgaaaa

SEQ ID NO: 419 - RNA, 19 nt, synthetic construct
  source 1..19: mol_type = other RNA
tagtaaagaataagtgaaa

SEQ ID NO: 420 - RNA, 19 nt, synthetic construct
  source 1..19: mol_type = other RNA
tggagtaaagaataagtga

SEQ ID NO: 421 - RNA, 19 nt, synthetic construct
  source 1..19: mol_type = other RNA
ttcttctgggagtaaagaa

SEQ ID NO: 422 - RNA, 19 nt, synthetic construct
  source 1..19: mol_type = other RNA
tttacagtcttctgggagt

SEQ ID NO: 423 - RNA, 19 nt, synthetic construct
  source 1..19: mol_type = other RNA
ttccttacagtcttctggg

SEQ ID NO: 424 - RNA, 19 nt, synthetic construct
  source 1..19: mol_type = other RNA
taagaaacacttacacttc

SEQ ID NO: 425 - RNA, 19 nt, synthetic construct
  source 1..19: mol_type = other RNA
tcttaagaaacacttacac

SEQ ID NO: 426 - RNA, 19 nt, synthetic construct
  source 1..19: mol_type = other RNA
taatcttaagaaacactta

SEQ ID NO: 427 - RNA, 19 nt, synthetic construct
  source 1..19: mol_type = other RNA
ttaatcttaagaaacactt

SEQ ID NO: 428 - RNA, 19 nt, synthetic construct
  source 1..19: mol_type = other RNA
tataatcttaagaaacact

SEQ ID NO: 429 - RNA, 19 nt, synthetic construct
  source 1..19: mol_type = other RNA
tgataatcttaagaaacac

SEQ ID NO: 430 - RNA, 19 nt, synthetic construct
  source 1..19: mol_type = other RNA
ttcaaagagtaaccagagc

SEQ ID NO: 431 - RNA, 19 nt, synthetic construct
  source 1..19: mol_type = other RNA
tcacaatctcaaagagtaa

SEQ ID NO: 432 - RNA, 19 nt, synthetic construct
  source 1..19: mol_type = other RNA
tagtttgttcctccaacaa

SEQ ID NO: 433 - RNA, 19 nt, synthetic construct
  source 1..19: mol_type = other RNA
tgatttaaaatgccactat

SEQ ID NO: 434 - RNA, 19 nt, synthetic construct
  source 1..19: mol_type = other RNA
ttaatgtctgacagattta

SEQ ID NO: 435 - RNA, 19 nt, synthetic construct
  source 1..19: mol_type = other RNA
ttagttttggtgaataata

SEQ ID NO: 436 - RNA, 19 nt, synthetic construct
  source 1..19: mol_type = other RNA
tcgatatcatgattccctt

SEQ ID NO: 437 - RNA, 19 nt, synthetic construct
  source 1..19: mol_type = other RNA
tgtaattcaaaggagcctg

SEQ ID NO: 438 - RNA, 19 nt, synthetic construct
  source 1..19: mol_type = other RNA
tattggtttttggaattca

SEQ ID NO: 439 - RNA, 19 nt, synthetic construct
  source 1..19: mol_type = other RNA
tggcatattggtttttgga

SEQ ID NO: 440 - RNA, 19 nt, synthetic construct
  source 1..19: mol_type = other RNA
tacagttggtataaattgt

SEQ ID NO: 441 - RNA, 19 nt, synthetic construct
  source 1..19: mol_type = other RNA
tttacccaacagttggtat

SEQ ID NO: 442 - RNA, 19 nt, synthetic construct
  source 1..19: mol_type = other RNA
ttactcagcgactttggtg

SEQ ID NO: 443 - RNA, 19 nt, synthetic construct
  source 1..19: mol_type = other RNA
ttaaaatccagtccatgta

SEQ ID NO: 444 - RNA, 19 nt, synthetic construct
  source 1..19: mol_type = other RNA
ttttctctaaaatccagtc

SEQ ID NO: 445 - RNA, 19 nt, synthetic construct
  source 1..19: mol_type = other RNA
tttttctctaaaatccagt

SEQ ID NO: 446 - RNA, 19 nt, synthetic construct
  source 1..19: mol_type = other RNA
ttttccatcactgctctgt

SEQ ID NO: 447 - RNA, 19 nt, synthetic construct
  source 1..19: mol_type = other RNA
tcagaatttgacttgaact

SEQ ID NO: 448 - RNA, 19 nt, synthetic construct
  source 1..19: mol_type = other RNA
ttcagaatttgacttgaac

SEQ ID NO: 449 - RNA, 21 nt, synthetic construct
  source 1..21: mol_type = other RNA
gggatgtagcttccatgtaca

SEQ ID NO: 450 - RNA, 21 nt, synthetic construct
  source 1..21: mol_type = other RNA
tggttgcttcttgaaagataa

SEQ ID NO: 451 - RNA, 21 nt, synthetic construct
  source 1..21: mol_type = other RNA
cttcttgaaagatagtgttaa

SEQ ID NO: 452 - RNA, 21 nt, synthetic construct
  source 1..21: mol_type = other RNA
caatgtggtcatcaaataaga

SEQ ID NO: 453 - RNA, 21 nt, synthetic construct
  source 1..21: mol_type = other RNA
gtggtcatcaaataagtgcta

SEQ ID NO: 454 - RNA, 21 nt, synthetic construct
  source 1..21: mol_type = other RNA
catcgagacatttataaagga

SEQ ID NO: 455 - RNA, 21 nt, synthetic construct
  source 1..21: mol_type = other RNA
atcgagacatttataaaggaa

SEQ ID NO: 456 - RNA, 21 nt, synthetic construct
  source 1..21: mol_type = other RNA
gttgatatgagaggagtcaaa

SEQ ID NO: 457 - RNA, 21 nt, synthetic construct
  source 1..21: mol_type = other RNA
ctaaggttagcagtgttgaaa

SEQ ID NO: 458 - RNA, 21 nt, synthetic construct
  source 1..21: mol_type = other RNA
ggttagcagtgttgaagaata

SEQ ID NO: 459 - RNA, 21 nt, synthetic construct
  source 1..21: mol_type = other RNA
gttagcagtgttgaagaatga

SEQ ID NO: 460 - RNA, 21 nt, synthetic construct
  source 1..21: mol_type = other RNA
gtaacgtggaatctggattca

SEQ ID NO: 461 - RNA, 21 nt, synthetic construct
  source 1..21: mol_type = other RNA
aacgtggaatctggattctca

SEQ ID NO: 462 - RNA, 21 nt, synthetic construct
  source 1..21: mol_type = other RNA
aatctggattctcactgaaga

SEQ ID NO: 463 - RNA, 21 nt, synthetic construct
  source 1..21: mol_type = other RNA
actccagatgcttttgtgtga

SEQ ID NO: 464 - RNA, 21 nt, synthetic construct
  source 1..21: mol_type = other RNA
ttcttaaaacatctgaaagta

SEQ ID NO: 465 - RNA, 21 nt, synthetic construct
  source 1..21: mol_type = other RNA
ttcctctactcctcaagaaaa

SEQ ID NO: 466 - RNA, 21 nt, synthetic construct
  source 1..21: mol_type = other RNA
tcctctactcctcaagaaaaa

SEQ ID NO: 467 - RNA, 21 nt, synthetic construct
  source 1..21: mol_type = other RNA
gccttttaacctgcaaaagaa

SEQ ID NO: 468 - RNA, 21 nt, synthetic construct
  source 1..21: mol_type = other RNA
aacctgcaaaagaactttaca

SEQ ID NO: 469 - RNA, 21 nt, synthetic construct
  source 1..21: mol_type = other RNA
acctgcaaaagaactttacca

SEQ ID NO: 470 - RNA, 21 nt, synthetic construct
  source 1..21: mol_type = other RNA
cctgcaaaagaactttaccta

SEQ ID NO: 471 - RNA, 21 nt, synthetic construct
  source 1..21: mol_type = other RNA
gcaaaagaactttacctgaaa

SEQ ID NO: 472 - RNA, 21 nt, synthetic construct
  source 1..21: mol_type = other RNA
actttggaggagaagaattga

SEQ ID NO: 473 - RNA, 21 nt, synthetic construct
  source 1..21: mol_type = other RNA
gaggagaagaattgaatgtga

SEQ ID NO: 474 - RNA, 21 nt, synthetic construct
  source 1..21: mol_type = other RNA
tgttaaaggagtgaatgttta

SEQ ID NO: 475 - RNA, 21 nt, synthetic construct
  source 1..21: mol_type = other RNA
ttcgctgtcagtttttcacta

SEQ ID NO: 476 - RNA, 21 nt, synthetic construct
  source 1..21: mol_type = other RNA
cgctgtcagtttttcacttaa

SEQ ID NO: 477 - RNA, 21 nt, synthetic construct
  source 1..21: mol_type = other RNA
ttttcacttattctttactca

SEQ ID NO: 478 - RNA, 21 nt, synthetic construct
  source 1..21: mol_type = other RNA
gaagagaagtgtaagtgttta

SEQ ID NO: 479 - RNA, 21 nt, synthetic construct
  source 1..21: mol_type = other RNA
agagaagtgtaagtgtttcta

SEQ ID NO: 480 - RNA, 21 nt, synthetic construct
  source 1..21: mol_type = other RNA
agaagtgtaagtgtttcttaa

SEQ ID NO: 481 - RNA, 21 nt, synthetic construct
  source 1..21: mol_type = other RNA
agtgtaagtgtttcttaagaa

SEQ ID NO: 482 - RNA, 21 nt, synthetic construct
  source 1..21: mol_type = other RNA
ctggttactctttgagattga

SEQ ID NO: 483 - RNA, 21 nt, synthetic construct
  source 1..21: mol_type = other RNA
tggttactctttgagattgta

SEQ ID NO: 484 - RNA, 21 nt, synthetic construct
  source 1..21: mol_type = other RNA
actctttgagattgtgtaaca

SEQ ID NO: 485 - RNA, 21 nt, synthetic construct
  source 1..21: mol_type = other RNA
ctctttgagattgtgtaacaa

SEQ ID NO: 486 - RNA, 21 nt, synthetic construct
  source 1..21: mol_type = other RNA
tctgtctgcacaacaaaaaca

SEQ ID NO: 487 - RNA, 21 nt, synthetic construct
  source 1..21: mol_type = other RNA
gaggaacaaactcttcttgga

SEQ ID NO: 488 - RNA, 21 nt, synthetic construct
  source 1..21: mol_type = other RNA
tgtgtggagggtcactcataa

SEQ ID NO: 489 - RNA, 21 nt, synthetic construct
  source 1..21: mol_type = other RNA
tatagtggcattttaaatcta

SEQ ID NO: 490 - RNA, 21 nt, synthetic construct
  source 1..21: mol_type = other RNA
tctgtcagacattacaaaaga

SEQ ID NO: 491 - RNA, 21 nt, synthetic construct
  source 1..21: mol_type = other RNA
ctgtcagacattacaaaagaa

SEQ ID NO: 492 - RNA, 21 nt, synthetic construct
  source 1..21: mol_type = other RNA
atacacctttctcacaaataa

SEQ ID NO: 493 - RNA, 21 nt, synthetic construct
  source 1..21: mol_type = other RNA
gaagggaatcatgatatcgca

SEQ ID NO: 494 - RNA, 21 nt, synthetic construct
  source 1..21: mol_type = other RNA
ctcctttgaattacactgaaa

SEQ ID NO: 495 - RNA, 21 nt, synthetic construct
  source 1..21: mol_type = other RNA
ttacactgaattccaaaaaca

SEQ ID NO: 496 - RNA, 21 nt, synthetic construct
  source 1..21: mol_type = other RNA
caagcacaatttataccaaca

SEQ ID NO: 497 - RNA, 21 nt, synthetic construct
  source 1..21: mol_type = other RNA
ccatcgagacatttataaaga

SEQ ID NO: 498 - RNA, 21 nt, synthetic construct
  source 1..21: mol_type = other RNA
tacctgaaccctgccattcta

SEQ ID NO: 499 - RNA, 21 nt, synthetic construct
  source 1..21: mol_type = other RNA
aaggttagcagtgttgaagaa

SEQ ID NO: 500 - RNA, 21 nt, synthetic construct
  source 1..21: mol_type = other RNA
ctctatgaaaacgccttctta

SEQ ID NO: 501 - RNA, 21 nt, synthetic construct
  source 1..21: mol_type = other RNA
cacccaaggtgtttgctatta

SEQ ID NO: 502 - RNA, 21 nt, synthetic construct
  source 1..21: mol_type = other RNA
tcagttttcttccagcaagta

SEQ ID NO: 503 - RNA, 21 nt, synthetic construct
  source 1..21: mol_type = other RNA
aatgacatggagaaaaggtta

SEQ ID NO: 504 - RNA, 21 nt, synthetic construct
  source 1..21: mol_type = other RNA
ggttgcttcttgaaagataga

SEQ ID NO: 505 - RNA, 21 nt, synthetic construct
  source 1..21: mol_type = other RNA
tcttgaaagatagtgttacaa

SEQ ID NO: 506 - RNA, 21 nt, synthetic construct
  source 1..21: mol_type = other RNA
tgtggtcatcaaataagtgca

SEQ ID NO: 507 - RNA, 21 nt, synthetic construct
  source 1..21: mol_type = other RNA
tgatatgagaggagtcaatta

SEQ ID NO: 508 - RNA, 21 nt, synthetic construct
  source 1..21: mol_type = other RNA
tatgagaggagtcaattttaa

SEQ ID NO: 509 - RNA, 21 nt, synthetic construct
  source 1..21: mol_type = other RNA
agtgttgaagaatgccaaaaa

SEQ ID NO: 510 - RNA, 21 nt, synthetic construct
  source 1..21: mol_type = other RNA
agtaacgtggaatctggatta

SEQ ID NO: 511 - RNA, 21 nt, synthetic construct
  source 1..21: mol_type = other RNA
cctgtgccctttcagaaatta

SEQ ID NO: 512 - RNA, 21 nt, synthetic construct
  source 1..21: mol_type = other RNA
ccttttaacctgcaaaagaaa

SEQ ID NO: 513 - RNA, 21 nt, synthetic construct
  source 1..21: mol_type = other RNA
gaaccctgccattctaaaata

SEQ ID NO: 514 - RNA, 21 nt, synthetic construct
  source 1..21: mol_type = other RNA
ccctgccattctaaaatttaa

SEQ ID NO: 515 - RNA, 21 nt, synthetic construct
  source 1..21: mol_type = other RNA
gagaagaattgaatgtgacta

SEQ ID NO: 516 - RNA, 21 nt, synthetic construct
  source 1..21: mol_type = other RNA
ttttgttaaaggagtgaatga

SEQ ID NO: 517 - RNA, 21 nt, synthetic construct
  source 1..21: mol_type = other RNA
gttaaaggagtgaatgtttga

SEQ ID NO: 518 - RNA, 21 nt, synthetic construct
  source 1..21: mol_type = other RNA
ttaaaggagtgaatgtttgca

SEQ ID NO: 519 - RNA, 21 nt, synthetic construct
  source 1..21: mol_type = other RNA
aaaggagtgaatgtttgccaa

SEQ ID NO: 520 - RNA, 21 nt, synthetic construct
  source 1..21: mol_type = other RNA
ttgcacaaagatgattcgcta

SEQ ID NO: 521 - RNA, 21 nt, synthetic construct
  source 1..21: mol_type = other RNA
aagatgattcgctgtcagtta

SEQ ID NO: 522 - RNA, 21 nt, synthetic construct
  source 1..21: mol_type = other RNA
tgattcgctgtcagtttttca

SEQ ID NO: 523 - RNA, 21 nt, synthetic construct
  source 1..21: mol_type = other RNA
cattcgctgtcagtttttcaa

SEQ ID NO: 524 - RNA, 21 nt, synthetic construct
  source 1..21: mol_type = other RNA
cttcgctgtcagtttttcaca

SEQ ID NO: 525 - RNA, 21 nt, synthetic construct
  source 1..21: mol_type = other RNA
ccgctgtcagtttttcactta

SEQ ID NO: 526 - RNA, 21 nt, synthetic construct
  source 1..21: mol_type = other RNA
cctgtcagtttttcacttata

SEQ ID NO: 527 - RNA, 21 nt, synthetic construct
  source 1..21: mol_type = other RNA
ctgtcagtttttcacttatta

SEQ ID NO: 528 - RNA, 21 nt, synthetic construct
  source 1..21: mol_type = other RNA
cgtcagtttttcacttattca

SEQ ID NO: 529 - RNA, 21 nt, synthetic construct
  source 1..21: mol_type = other RNA
gtcagtttttcacttattcta

SEQ ID NO: 530 - RNA, 21 nt, synthetic construct
  source 1..21: mol_type = other RNA
gtttttcacttattctttaca

SEQ ID NO: 531 - RNA, 21 nt, synthetic construct
  source 1..21: mol_type = other RNA
tttttcacttattctttacta

SEQ ID NO: 532 - RNA, 21 nt, synthetic construct
  source 1..21: mol_type = other RNA
tttcacttattctttactcca

SEQ ID NO: 533 - RNA, 21 nt, synthetic construct
  source 1..21: mol_type = other RNA
tattctttactcccagaagaa

SEQ ID NO: 534 - RNA, 21 nt, synthetic construct
  source 1..21: mol_type = other RNA
ttactcccagaagactgtaaa

SEQ ID NO: 535 - RNA, 21 nt, synthetic construct
  source 1..21: mol_type = other RNA
ctcccagaagactgtaaggaa

SEQ ID NO: 536 - RNA, 21 nt, synthetic construct
  source 1..21: mol_type = other RNA
gagaagtgtaagtgtttctta

SEQ ID NO: 537 - RNA, 21 nt, synthetic construct
  source 1..21: mol_type = other RNA
aagtgtaagtgtttcttaaga

SEQ ID NO: 538 - RNA, 21 nt, synthetic construct
  source 1..21: mol_type = other RNA
tgtaagtgtttcttaagatta

SEQ ID NO: 539 - RNA, 21 nt, synthetic construct
  source 1..21: mol_type = other RNA
ctaagtgtttcttaagattaa

SEQ ID NO: 540 - RNA, 21 nt, synthetic construct
  source 1..21: mol_type = other RNA
taagtgtttcttaagattata

SEQ ID NO: 541 - RNA, 21 nt, synthetic construct
  source 1..21: mol_type = other RNA
aagtgtttcttaagattatca

SEQ ID NO: 542 - RNA, 21 nt, synthetic construct
  source 1..21: mol_type = other RNA
gagctctggttactctttgaa

SEQ ID NO: 543 - RNA, 21 nt, synthetic construct
  source 1..21: mol_type = other RNA
ggttactctttgagattgtga

SEQ ID NO: 544 - RNA, 21 nt, synthetic construct
  source 1..21: mol_type = other RNA
cattgttggaggaacaaacta

SEQ ID NO: 545 - RNA, 21 nt, synthetic construct
  source 1..21: mol_type = other RNA
ctatagtggcattttaaatca

SEQ ID NO: 546 - RNA, 21 nt, synthetic construct
  source 1..21: mol_type = other RNA
tttaaatctgtcagacattaa

SEQ ID NO: 547 - RNA, 21 nt, synthetic construct
  source 1..21: mol_type = other RNA
attattattcaccaaaactaa

SEQ ID NO: 548 - RNA, 21 nt, synthetic construct
  source 1..21: mol_type = other RNA
agaagggaatcatgatatcga

SEQ ID NO: 549 - RNA, 21 nt, synthetic construct
  source 1..21: mol_type = other RNA
tccaggctcctttgaattaca

SEQ ID NO: 550 - RNA, 21 nt, synthetic construct
  source 1..21: mol_type = other RNA
actgaattccaaaaaccaata

SEQ ID NO: 551 - RNA, 21 nt, synthetic construct
  source 1..21: mol_type = other RNA
attccaaaaaccaatatgcca

SEQ ID NO: 552 - RNA, 21 nt, synthetic construct
  source 1..21: mol_type = other RNA
gcacaatttataccaactgta

SEQ ID NO: 553 - RNA, 21 nt, synthetic construct
  source 1..21: mol_type = other RNA
ttataccaactgttgggtaaa

SEQ ID NO: 554 - RNA, 21 nt, synthetic construct
  source 1..21: mol_type = other RNA
tacaccaaagtcgctgagtaa

SEQ ID NO: 555 - RNA, 21 nt, synthetic construct
  source 1..21: mol_type = other RNA
agtacatggactggattttaa

SEQ ID NO: 556 - RNA, 21 nt, synthetic construct
  source 1..21: mol_type = other RNA
tggactggattttagagaaaa

SEQ ID NO: 557 - RNA, 21 nt, synthetic construct
  source 1..21: mol_type = other RNA
ggactggattttagagaaaaa

SEQ ID NO: 558 - RNA, 21 nt, synthetic construct
  source 1..21: mol_type = other RNA
acacagagcagtgatggaaaa

SEQ ID NO: 559 - RNA, 21 nt, synthetic construct
  source 1..21: mol_type = other RNA
tgagttcaagtcaaattctga

SEQ ID NO: 560 - RNA, 21 nt, synthetic construct
  source 1..21: mol_type = other RNA
gagttcaagtcaaattctgaa

SEQ ID NO: 561 - RNA, 21 nt, synthetic construct
  source 1..21: mol_type = other RNA
tgtacatggaagctacatccc

SEQ ID NO: 562 - RNA, 21 nt, synthetic construct
  source 1..21: mol_type = other RNA
ttatctttcaagaagcaacca

SEQ ID NO: 563 - RNA, 21 nt, synthetic construct
  source 1..21: mol_type = other RNA
ttaacactatctttcaagaag

SEQ ID NO: 564 - RNA, 21 nt, synthetic construct
  source 1..21: mol_type = other RNA
tcttatttgatgaccacattg

SEQ ID NO: 565 - RNA, 21 nt, synthetic construct
  source 1..21: mol_type = other RNA
tagcacttatttgatgaccac

SEQ ID NO: 566 - RNA, 21 nt, synthetic construct
  source 1..21: mol_type = other RNA
tcctttataaatgtctcgatg

SEQ ID NO: 567 - RNA, 21 nt, synthetic construct
  source 1..21: mol_type = other RNA
ttcctttataaatgtctcgat

SEQ ID NO: 568 - RNA, 21 nt, synthetic construct
  source 1..21: mol_type = other RNA
tttgactcctctcatatcaac

SEQ ID NO: 569 - RNA, 21 nt, synthetic construct
  source 1..21: mol_type = other RNA
tttcaacactgctaaccttag

SEQ ID NO: 570 - RNA, 21 nt, synthetic construct
  source 1..21: mol_type = other RNA
tattcttcaacactgctaacc

SEQ ID NO: 571 - RNA, 21 nt, synthetic construct
  source 1..21: mol_type = other RNA
tcattcttcaacactgctaac

SEQ ID NO: 572 - RNA, 21 nt, synthetic construct
  source 1..21: mol_type = other RNA
tgaatccagattccacgttac

SEQ ID NO: 573 - RNA, 21 nt, synthetic construct
  source 1..21: mol_type = other RNA
tgagaatccagattccacgtt

SEQ ID NO: 574 - RNA, 21 nt, synthetic construct
  source 1..21: mol_type = other RNA
tcttcagtgagaatccagatt

SEQ ID NO: 575 - RNA, 21 nt, synthetic construct
  source 1..21: mol_type = other RNA
tcacacaaaagcatctggagt

SEQ ID NO: 576 - RNA, 21 nt, synthetic construct
  source 1..21: mol_type = other RNA
tactttcagatgttttaagaa

SEQ ID NO: 577 - RNA, 21 nt, synthetic construct
  source 1..21: mol_type = other RNA
ttttcttgaggagtagaggaa

SEQ ID NO: 578 - RNA, 21 nt, synthetic construct
  source 1..21: mol_type = other RNA
tttttcttgaggagtagagga

SEQ ID NO: 579 - RNA, 21 nt, synthetic construct
  source 1..21: mol_type = other RNA
ttcttttgcaggttaaaaggc

SEQ ID NO: 580 - RNA, 21 nt, synthetic construct
  source 1..21: mol_type = other RNA
tgtaaagttcttttgcaggtt

SEQ ID NO: 581 - RNA, 21 nt, synthetic construct
  source 1..21: mol_type = other RNA
tggtaaagttcttttgcaggt

SEQ ID NO: 582 - RNA, 21 nt, synthetic construct
  source 1..21: mol_type = other RNA
taggtaaagttcttttgcagg

SEQ ID NO: 583 - RNA, 21 nt, synthetic construct
  source 1..21: mol_type = other RNA
tttcaggtaaagttcttttgc

SEQ ID NO: 584 - RNA, 21 nt, synthetic construct
  source 1..21: mol_type = other RNA
tcaattcttctcctccaaagt

SEQ ID NO: 585 - RNA, 21 nt, synthetic construct
  source 1..21: mol_type = other RNA
tcacattcaattcttctcctc

SEQ ID NO: 586 - RNA, 21 nt, synthetic construct
  source 1..21: mol_type = other RNA
taaacattcactcctttaaca

SEQ ID NO: 587 - RNA, 21 nt, synthetic construct
  source 1..21: mol_type = other RNA
tagtgaaaaactgacagcgaa

SEQ ID NO: 588 - RNA, 21 nt, synthetic construct
  source 1..21: mol_type = other RNA
ttaagtgaaaaactgacagcg

SEQ ID NO: 589 - RNA, 21 nt, synthetic construct
  source 1..21: mol_type = other RNA
tgagtaaagaataagtgaaaa

SEQ ID NO: 590 - RNA, 21 nt, synthetic construct
  source 1..21: mol_type = other RNA
taaacacttacacttctcttc

SEQ ID NO: 591 - RNA, 21 nt, synthetic construct
  source 1..21: mol_type = other RNA
tagaaacacttacacttctct

SEQ ID NO: 592 - RNA, 21 nt, synthetic construct
  source 1..21: mol_type = other RNA
ttaagaaacacttacacttct

SEQ ID NO: 593 - RNA, 21 nt, synthetic construct
  source 1..21: mol_type = other RNA
ttcttaagaaacacttacact

SEQ ID NO: 594 - RNA, 21 nt, synthetic construct
  source 1..21: mol_type = other RNA
tcaatctcaaagagtaaccag

SEQ ID NO: 595 - RNA, 21 nt, synthetic construct
  source 1..21: mol_type = other RNA
tacaatctcaaagagtaacca

SEQ ID NO: 596 - RNA, 21 nt, synthetic construct
  source 1..21: mol_type = other RNA
tgttacacaatctcaaagagt

SEQ ID NO: 597 - RNA, 21 nt, synthetic construct
  source 1..21: mol_type = other RNA
ttgttacacaatctcaaagag

SEQ ID NO: 598 - RNA, 21 nt, synthetic construct
  source 1..21: mol_type = other RNA
tgtttttgttgtgcagacaga

SEQ ID NO: 599 - RNA, 21 nt, synthetic construct
  source 1..21: mol_type = other RNA
tccaagaagagtttgttcctc

SEQ ID NO: 600 - RNA, 21 nt, synthetic construct
  source 1..21: mol_type = other RNA
ttatgagtgaccctccacaca

SEQ ID NO: 601 - RNA, 21 nt, synthetic construct
  source 1..21: mol_type = other RNA
tagatttaaaatgccactata

SEQ ID NO: 602 - RNA, 21 nt, synthetic construct
  source 1..21: mol_type = other RNA
tcttttgtaatgtctgacaga

SEQ ID NO: 603 - RNA, 21 nt, synthetic construct
  source 1..21: mol_type = other RNA
ttcttttgtaatgtctgacag

SEQ ID NO: 604 - RNA, 21 nt, synthetic construct
  source 1..21: mol_type = other RNA
ttatttgtgagaaaggtgtat

SEQ ID NO: 605 - RNA, 21 nt, synthetic construct
  source 1..21: mol_type = other RNA
tgcgatatcatgattcccttc

SEQ ID NO: 606 - RNA, 21 nt, synthetic construct
  source 1..21: mol_type = other RNA
tttcagtgtaattcaaaggag

SEQ ID NO: 607 - RNA, 21 nt, synthetic construct
  source 1..21: mol_type = other RNA
tgtttttggaattcagtgtaa

SEQ ID NO: 608 - RNA, 21 nt, synthetic construct
  source 1..21: mol_type = other RNA
tgttggtataaattgtgcttg

SEQ ID NO: 609 - RNA, 21 nt, synthetic construct
  source 1..21: mol_type = other RNA
tctttataaatgtctcgatgg

SEQ ID NO: 610 - RNA, 21 nt, synthetic construct
  source 1..21: mol_type = other RNA
tagaatggcagggttcaggta

SEQ ID NO: 611 - RNA, 21 nt, synthetic construct
  source 1..21: mol_type = other RNA
ttcttcaacactgctaacctt

SEQ ID NO: 612 - RNA, 21 nt, synthetic construct
  source 1..21: mol_type = other RNA
taagaaggcgttttcatagag

SEQ ID NO: 613 - RNA, 21 nt, synthetic construct
  source 1..21: mol_type = other RNA
taatagcaaacaccttgggtg

SEQ ID NO: 614 - RNA, 21 nt, synthetic construct
  source 1..21: mol_type = other RNA
tacttgctggaagaaaactga

SEQ ID NO: 615 - RNA, 21 nt, synthetic construct
  source 1..21: mol_type = other RNA
taaccttttctccatgtcatt

SEQ ID NO: 616 - RNA, 21 nt, synthetic construct
  source 1..21: mol_type = other RNA
tctatctttcaagaagcaacc

SEQ ID NO: 617 - RNA, 21 nt, synthetic construct
  source 1..21: mol_type = other RNA
ttgtaacactatctttcaaga

SEQ ID NO: 618 - RNA, 21 nt, synthetic construct
  source 1..21: mol_type = other RNA
tgcacttatttgatgaccaca

SEQ ID NO: 619 - RNA, 21 nt, synthetic construct
  source 1..21: mol_type = other RNA
taattgactcctctcatatca

SEQ ID NO: 620 - RNA, 21 nt, synthetic construct
  source 1..21: mol_type = other RNA
ttaaaattgactcctctcata

SEQ ID NO: 621 - RNA, 21 nt, synthetic construct
  source 1..21: mol_type = other RNA
tttttggcattcttcaacact

SEQ ID NO: 622 - RNA, 21 nt, synthetic construct
  source 1..21: mol_type = other RNA
taatccagattccacgttact

SEQ ID NO: 623 - RNA, 21 nt, synthetic construct
  source 1..21: mol_type = other RNA
taatttctgaaagggcacagg

SEQ ID NO: 624 - RNA, 21 nt, synthetic construct
  source 1..21: mol_type = other RNA
tttcttttgcaggttaaaagg

SEQ ID NO: 625 - RNA, 21 nt, synthetic construct
  source 1..21: mol_type = other RNA
tattttagaatggcagggttc

SEQ ID NO: 626 - RNA, 21 nt, synthetic construct
  source 1..21: mol_type = other RNA
ttaaattttagaatggcaggg

SEQ ID NO: 627 - RNA, 21 nt, synthetic construct
  source 1..21: mol_type = other RNA
tagtcacattcaattcttctc

SEQ ID NO: 628 - RNA, 21 nt, synthetic construct
  source 1..21: mol_type = other RNA
tcattcactcctttaacaaaa

SEQ ID NO: 629 - RNA, 21 nt, synthetic construct
  source 1..21: mol_type = other RNA
tcaaacattcactcctttaac

SEQ ID NO: 630 - RNA, 21 nt, synthetic construct
  source 1..21: mol_type = other RNA
tgcaaacattcactcctttaa

SEQ ID NO: 631 - RNA, 21 nt, synthetic construct
  source 1..21: mol_type = other RNA
ttggcaaacattcactccttt

SEQ ID NO: 632 - RNA, 21 nt, synthetic construct
  source 1..21: mol_type = other RNA
tagcgaatcatctttgtgcaa

SEQ ID NO: 633 - RNA, 21 nt, synthetic construct
  source 1..21: mol_type = other RNA
taactgacagcgaatcatctt

SEQ ID NO: 634 - RNA, 21 nt, synthetic construct
  source 1..21: mol_type = other RNA
tgaaaaactgacagcgaatca

SEQ ID NO: 635 - RNA, 21 nt, synthetic construct
  source 1..21: mol_type = other RNA
ttgaaaaactgacagcgaatg

SEQ ID NO: 636 - RNA, 21 nt, synthetic construct
  source 1..21: mol_type = other RNA
tgtgaaaaactgacagcgaag

SEQ ID NO: 637 - RNA, 21 nt, synthetic construct
  source 1..21: mol_type = other RNA
taagtgaaaaactgacagcgg

SEQ ID NO: 638 - RNA, 21 nt, synthetic construct
  source 1..21: mol_type = other RNA
tataagtgaaaaactgacagg

SEQ ID NO: 639 - RNA, 21 nt, synthetic construct
  source 1..21: mol_type = other RNA
taataagtgaaaaactgacag

SEQ ID NO: 640 - RNA, 21 nt, synthetic construct
  source 1..21: mol_type = other RNA
tgaataagtgaaaaactgacg

SEQ ID NO: 641 - RNA, 21 nt, synthetic construct
  source 1..21: mol_type = other RNA
tagaataagtgaaaaactgac

SEQ ID NO: 642 - RNA, 21 nt, synthetic construct
  source 1..21: mol_type = other RNA
tgtaaagaataagtgaaaaac

SEQ ID NO: 643 - RNA, 21 nt, synthetic construct
  source 1..21: mol_type = other RNA
tagtaaagaataagtgaaaaa

SEQ ID NO: 644 - RNA, 21 nt, synthetic construct
  source 1..21: mol_type = other RNA
tggagtaaagaataagtgaaa

SEQ ID NO: 645 - RNA, 21 nt, synthetic construct
  source 1..21: mol_type = other RNA
ttcttctgggagtaaagaata

SEQ ID NO: 646 - RNA, 21 nt, synthetic construct
  source 1..21: mol_type = other RNA
tttacagtcttctgggagtaa

SEQ ID NO: 647 - RNA, 21 nt, synthetic construct
  source 1..21: mol_type = other RNA
ttccttacagtcttctgggag

SEQ ID NO: 648 - RNA, 21 nt, synthetic construct
  source 1..21: mol_type = other RNA
taagaaacacttacacttctc

SEQ ID NO: 649 - RNA, 21 nt, synthetic construct
  source 1..21: mol_type = other RNA
tcttaagaaacacttacactt

SEQ ID NO: 650 - RNA, 21 nt, synthetic construct
  source 1..21: mol_type = other RNA
taatcttaagaaacacttaca

SEQ ID NO: 651 - RNA, 21 nt, synthetic construct
  source 1..21: mol_type = other RNA
ttaatcttaagaaacacttag

SEQ ID NO: 652 - RNA, 21 nt, synthetic construct
  source 1..21: mol_type = other RNA
tataatcttaagaaacactta

SEQ ID NO: 653 - RNA, 21 nt, synthetic construct
  source 1..21: mol_type = other RNA
tgataatcttaagaaacactt

SEQ ID NO: 654 - RNA, 21 nt, synthetic construct
  source 1..21: mol_type = other RNA
ttcaaagagtaaccagagctc

SEQ ID NO: 655 - RNA, 21 nt, synthetic construct
  source 1..21: mol_type = other RNA
tcacaatctcaaagagtaacc

SEQ ID NO: 656 - RNA, 21 nt, synthetic construct
  source 1..21: mol_type = other RNA
tagtttgttcctccaacaatg

SEQ ID NO: 657 - RNA, 21 nt, synthetic construct
  source 1..21: mol_type = other RNA
tgatttaaaatgccactatag

SEQ ID NO: 658 - RNA, 21 nt, synthetic construct
  source 1..21: mol_type = other RNA
ttaatgtctgacagatttaaa

SEQ ID NO: 659 - RNA, 21 nt, synthetic construct
  source 1..21: mol_type = other RNA
ttagttttggtgaataataat

SEQ ID NO: 660 - RNA, 21 nt, synthetic construct
  source 1..21: mol_type = other RNA
tcgatatcatgattcccttct

SEQ ID NO: 661 - RNA, 21 nt, synthetic construct
  source 1..21: mol_type = other RNA
tgtaattcaaaggagcctgga

SEQ ID NO: 662 - RNA, 21 nt, synthetic construct
  source 1..21: mol_type = other RNA
tattggtttttggaattcagt

SEQ ID NO: 663 - RNA, 21 nt, synthetic construct
  source 1..21: mol_type = other RNA
tggcatattggtttttggaat

SEQ ID NO: 664 - RNA, 21 nt, synthetic construct
  source 1..21: mol_type = other RNA
tacagttggtataaattgtgc

SEQ ID NO: 665 - RNA, 21 nt, synthetic construct
  source 1..21: mol_type = other RNA
tttacccaacagttggtataa

SEQ ID NO: 666 - RNA, 21 nt, synthetic construct
  source 1..21: mol_type = other RNA
ttactcagcgactttggtgta

SEQ ID NO: 667 - RNA, 21 nt, synthetic construct
  source 1..21: mol_type = other RNA
ttaaaatccagtccatgtact

SEQ ID NO: 668 - RNA, 21 nt, synthetic construct
  source 1..21: mol_type = other RNA
ttttctctaaaatccagtcca

SEQ ID NO: 669 - RNA, 21 nt, synthetic construct
  source 1..21: mol_type = other RNA
tttttctctaaaatccagtcc

SEQ ID NO: 670 - RNA, 21 nt, synthetic construct
  source 1..21: mol_type = other RNA
ttttccatcactgctctgtgt

SEQ ID NO: 671 - RNA, 21 nt, synthetic construct
  source 1..21: mol_type = other RNA
tcagaatttgacttgaactca

SEQ ID NO: 672 - RNA, 21 nt, synthetic construct
  source 1..21: mol_type = other RNA
ttcagaatttgacttgaactc

SEQ ID NO: 673 - RNA, 21 nt, synthetic construct
  source 1..21: mol_type = other RNA
gcaaaagaactttacctgaaa

SEQ ID NO: 674 - RNA, 21 nt, synthetic construct
  source 1..21: mol_type = other RNA
gtctttgagattgtgtaacaa

SEQ ID NO: 675 - RNA, 21 nt, synthetic construct
  source 1..21: mol_type = other RNA
gaagagaagtgtaagtgttta

SEQ ID NO: 676 - RNA, 21 nt, synthetic construct
  source 1..21: mol_type = other RNA
gaagcacaatttataccaaca

SEQ ID NO: 677 - RNA, 21 nt, synthetic construct
  source 1..21: mol_type = other RNA
gatcgagacatttataaagga

SEQ ID NO: 678 - RNA, 21 nt, synthetic construct
  source 1..21: mol_type = other RNA
gggatgtagcttccatgtaca

SEQ ID NO: 679 - RNA, 21 nt, synthetic construct
  source 1..21: mol_type = other RNA
gtaacgtggaatctggattca

SEQ ID NO: 680 - RNA, 21 nt, synthetic construct
  source 1..21: mol_type = other RNA
agaagtgtaagtgtttcttaa

SEQ ID NO: 681 - RNA, 21 nt, synthetic construct
  source 1..21: mol_type = other RNA
agtgtaagtgtttcttaagaa

SEQ ID NO: 682 - RNA, 21 nt, synthetic construct
  source 1..21: mol_type = other RNA
atacacctttctcacaaataa

SEQ ID NO: 683 - RNA, 21 nt, synthetic construct
  source 1..21: mol_type = other RNA
gaagggaatcatgatatcgca

SEQ ID NO: 684 - RNA, 21 nt, synthetic construct
  source 1..21: mol_type = other RNA
ccatcgagacatttataaaga

SEQ ID NO: 685 - RNA, 21 nt, synthetic construct
  source 1..21: mol_type = other RNA
tggttgcttcttgaaagataa

SEQ ID NO: 686 - RNA, 21 nt, synthetic construct
  source 1..21: mol_type = other RNA
ggttagcagtgttgaagaata

SEQ ID NO: 687 - RNA, 21 nt, synthetic construct
  source 1..21: mol_type = other RNA
tcctctactcctcaagaaaaa

SEQ ID NO: 688 - RNA, 21 nt, synthetic construct
  source 1..21: mol_type = other RNA
gcctgcaaaagaactttacct

SEQ ID NO: 689 - RNA, 21 nt, synthetic construct
  source 1..21: mol_type = other RNA
cctgcaaaagaactttaccta

SEQ ID NO: 690 - RNA, 21 nt, synthetic construct
  source 1..21: mol_type = other RNA
tacctgaaccctgccattcta

SEQ ID NO: 691 - RNA, 21 nt, synthetic construct
  source 1..21: mol_type = other RNA
actttggaggagaagaattga

SEQ ID NO: 692 - RNA, 21 nt, synthetic construct
  source 1..21: mol_type = other RNA
gaggagaagaattgaatgtga

SEQ ID NO: 693 - RNA, 21 nt, synthetic construct
  source 1..21: mol_type = other RNA
ttcgctgtcagtttttcacta

SEQ ID NO: 694 - RNA, 21 nt, synthetic construct
  source 1..21: mol_type = other RNA
cgctgtcagtttttcacttaa

SEQ ID NO: 695 - RNA, 21 nt, synthetic construct
  source 1..21: mol_type = other RNA
tggttactctttgagattgta

SEQ ID NO: 696 - RNA, 21 nt, synthetic construct
  source 1..21: mol_type = other RNA
tgtgtggagggtcactcataa

SEQ ID NO: 697 - RNA, 21 nt, synthetic construct
  source 1..21: mol_type = other RNA
tctgtcagacattacaaaaga

SEQ ID NO: 698 - RNA, 21 nt, synthetic construct
  source 1..21: mol_type = other RNA
gttagcagtgttgaagaatga

SEQ ID NO: 699 - RNA, 21 nt, synthetic construct
  source 1..21: mol_type = other RNA
aacgtggaatctggattctca

SEQ ID NO: 700 - RNA, 21 nt, synthetic construct
  source 1..21: mol_type = other RNA
gatctggattctcactgaaga

SEQ ID NO: 701 - RNA, 21 nt, synthetic construct
  source 1..21: mol_type = other RNA
gccttttaacctgcaaaagaa

SEQ ID NO: 702 - RNA, 21 nt, synthetic construct
  source 1..21: mol_type = other RNA
aacctgcaaaagaactttaca

SEQ ID NO: 703 - RNA, 21 nt, synthetic construct
  source 1..21: mol_type = other RNA
ggttaaaggagtgaatgttta

SEQ ID NO: 704 - RNA, 21 nt, synthetic construct
  source 1..21: mol_type = other RNA
actctttgagattgtgtaaca

SEQ ID NO: 705 - RNA, 21 nt, synthetic construct
  source 1..21: mol_type = other RNA
ctcctttgaattacactgaaa

SEQ ID NO: 706 - RNA, 21 nt, synthetic construct
  source 1..21: mol_type = other RNA
aaggttagcagtgttgaagaa

SEQ ID NO: 707 - RNA, 21 nt, synthetic construct
  source 1..21: mol_type = other RNA
gatacacctttctcacaaata

SEQ ID NO: 708 - RNA, 21 nt, synthetic construct
  source 1..21: mol_type = other RNA
gacacctttctcacaaataaa

SEQ ID NO: 709 - RNA, 21 nt, synthetic construct
  source 1..21: mol_type = other RNA
ctgtcagacattacaaaagaa

SEQ ID NO: 710 - RNA, 21 nt, synthetic construct
  source 1..21: mol_type = other RNA
atacacctttctcacaaataa

SEQ ID NO: 711 - RNA, 21 nt, synthetic construct
  source 1..21: mol_type = other RNA
ttcgctgtcagtttttcacta

SEQ ID NO: 712 - RNA, 21 nt, synthetic construct
  source 1..21: mol_type = other RNA
cgctgtcagtttttcacttaa

SEQ ID NO: 713 - RNA, 21 nt, synthetic construct
  source 1..21: mol_type = other RNA
gatacacctttctcacaaata

SEQ ID NO: 714 - RNA, 21 nt, synthetic construct
  source 1..21: mol_type = other RNA
cattcgctgtcagtttttcaa

SEQ ID NO: 715 - RNA, 21 nt, synthetic construct
  source 1..21: mol_type = other RNA
cttcgctgtcagtttttcaca

SEQ ID NO: 716 - RNA, 21 nt, synthetic construct
  source 1..21: mol_type = other RNA
ccgctgtcagtttttcactta

SEQ ID NO: 717 - RNA, 21 nt, synthetic construct
  source 1..21: mol_type = other RNA
cctgtcagtttttcacttata

SEQ ID NO: 718 - RNA, 21 nt, synthetic construct
  source 1..21: mol_type = other RNA
cgtcagtttttcacttattca

SEQ ID NO: 719 - RNA, 21 nt, synthetic construct
  source 1..21: mol_type = other RNA
ctaagtgtttcttaagattaa

SEQ ID NO: 720 - RNA, 21 nt, synthetic construct
  source 1..21: mol_type = other RNA
tttcaggtaaagttcttttgc

SEQ ID NO: 721 - RNA, 21 nt, synthetic construct
  source 1..21: mol_type = other RNA
ttgttacacaatctcaaagac

SEQ ID NO: 722 - RNA, 21 nt, synthetic construct
  source 1..21: mol_type = other RNA
taaacacttacacttctcttc

SEQ ID NO: 723 - RNA, 21 nt, synthetic construct
  source 1..21: mol_type = other RNA
tgttggtataaattgtgcttc

SEQ ID NO: 724 - RNA, 21 nt, synthetic construct
  source 1..21: mol_type = other RNA
tcctttataaatgtctcgatc

SEQ ID NO: 725 - RNA, 21 nt, synthetic construct
  source 1..21: mol_type = other RNA
tgtacatggaagctacatccc

SEQ ID NO: 726 - RNA, 21 nt, synthetic construct
  source 1..21: mol_type = other RNA
tgaatccagattccacgttac

SEQ ID NO: 727 - RNA, 21 nt, synthetic construct
  source 1..21: mol_type = other RNA
ttaagaaacacttacacttct

SEQ ID NO: 728 - RNA, 21 nt, synthetic construct
  source 1..21: mol_type = other RNA
ttcttaagaaacacttacact

SEQ ID NO: 729 - RNA, 21 nt, synthetic construct
  source 1..21: mol_type = other RNA
ttatttgtgagaaaggtgtat

SEQ ID NO: 730 - RNA, 21 nt, synthetic construct
  source 1..21: mol_type = other RNA
tgcgatatcatgattcccttc

SEQ ID NO: 731 - RNA, 21 nt, synthetic construct
  source 1..21: mol_type = other RNA
tctttataaatgtctcgatgg

SEQ ID NO: 732 - RNA, 21 nt, synthetic construct
  source 1..21: mol_type = other RNA
ttatctttcaagaagcaacca

SEQ ID NO: 733 - RNA, 21 nt, synthetic construct
  source 1..21: mol_type = other RNA
tattcttcaacactgctaacc

SEQ ID NO: 734 - RNA, 21 nt, synthetic construct
  source 1..21: mol_type = other RNA
tttttcttgaggagtagagga

SEQ ID NO: 735 - RNA, 21 nt, synthetic construct
  source 1..21: mol_type = other RNA
aggtaaagttcttttgcaggc

SEQ ID NO: 736 - RNA, 21 nt, synthetic construct
  source 1..21: mol_type = other RNA
taggtaaagttcttttgcagg

SEQ ID NO: 737 - RNA, 21 nt, synthetic construct
  source 1..21: mol_type = other RNA
tagaatggcagggttcaggta

SEQ ID NO: 738 - RNA, 21 nt, synthetic construct
  source 1..21: mol_type = other RNA
tcaattcttctcctccaaagt

SEQ ID NO: 739 - RNA, 21 nt, synthetic construct
  source 1..21: mol_type = other RNA
tcacattcaattcttctcctc

SEQ ID NO: 740 - RNA, 21 nt, synthetic construct
  source 1..21: mol_type = other RNA
tagtgaaaaactgacagcgaa

SEQ ID NO: 741 - RNA, 21 nt, synthetic construct
  source 1..21: mol_type = other RNA
ttaagtgaaaaactgacagcg

SEQ ID NO: 742 - RNA, 21 nt, synthetic construct
  source 1..21: mol_type = other RNA
tacaatctcaaagagtaacca

SEQ ID NO: 743 - RNA, 21 nt, synthetic construct
  source 1..21: mol_type = other RNA
ttatgagtgaccctccacaca

SEQ ID NO: 744 - RNA, 21 nt, synthetic construct
  source 1..21: mol_type = other RNA
tcttttgtaatgtctgacaga

SEQ ID NO: 745 - RNA, 21 nt, synthetic construct
  source 1..21: mol_type = other RNA
tcattcttcaacactgctaac

SEQ ID NO: 746 - RNA, 21 nt, synthetic construct
  source 1..21: mol_type = other RNA
tgagaatccagattccacgtt

SEQ ID NO: 747 - RNA, 21 nt, synthetic construct
  source 1..21: mol_type = other RNA
tcttcagtgagaatccagatc

SEQ ID NO: 748 - RNA, 21 nt, synthetic construct
  source 1..21: mol_type = other RNA
ttcttttgcaggttaaaaggc

SEQ ID NO: 749 - RNA, 21 nt, synthetic construct
  source 1..21: mol_type = other RNA
tgtaaagttcttttgcaggtt

SEQ ID NO: 750 - RNA, 21 nt, synthetic construct
  source 1..21: mol_type = other RNA
taaacattcactcctttaacc

SEQ ID NO: 751 - RNA, 21 nt, synthetic construct
  source 1..21: mol_type = other RNA
tgttacacaatctcaaagagt

SEQ ID NO: 752 - RNA, 21 nt, synthetic construct
  source 1..21: mol_type = other RNA
tttcagtgtaattcaaaggag

SEQ ID NO: 753 - RNA, 21 nt, synthetic construct
  source 1..21: mol_type = other RNA
ttcttcaacactgctaacctt

SEQ ID NO: 754 - RNA, 21 nt, synthetic construct
  source 1..21: mol_type = other RNA
tatttgtgagaaaggtgtatc

SEQ ID NO: 755 - RNA, 21 nt, synthetic construct
  source 1..21: mol_type = other RNA
tttatttgtgagaaaggtgtc

SEQ ID NO: 756 - RNA, 21 nt, synthetic construct
  source 1..21: mol_type = other RNA
ttcttttgtaatgtctgacag

SEQ ID NO: 757 - RNA, 21 nt, synthetic construct
  source 1..21: mol_type = other RNA
ttatttgtgagaaaggtgtat

SEQ ID NO: 758 - RNA, 21 nt, synthetic construct
  source 1..21: mol_type = other RNA
tagtgaaaaactgacagcgaa

SEQ ID NO: 759 - RNA, 21 nt, synthetic construct
  source 1..21: mol_type = other RNA
ttaagtgaaaaactgacagcg

SEQ ID NO: 760 - RNA, 21 nt, synthetic construct
  source 1..21: mol_type = other RNA
tatttgtgagaaaggtgtatc

SEQ ID NO: 761 - RNA, 21 nt, synthetic construct
  source 1..21: mol_type = other RNA
ttgaaaaactgacagcgaatg

SEQ ID NO: 762 - RNA, 21 nt, synthetic construct
  source 1..21: mol_type = other RNA
tgtgaaaaactgacagcgaag

SEQ ID NO: 763 - RNA, 21 nt, synthetic construct
  source 1..21: mol_type = other RNA
taagtgaaaaactgacagcgg

SEQ ID NO: 764 - RNA, 21 nt, synthetic construct
  source 1..21: mol_type = other RNA
tataagtgaaaaactgacagg

SEQ ID NO: 765 - RNA, 21 nt, synthetic construct
  source 1..21: mol_type = other RNA
tgaataagtgaaaaactgacg

SEQ ID NO: 766 - RNA, 21 nt, synthetic construct
  source 1..21: mol_type = other RNA
ttaatcttaagaaacacttag

SEQ ID NO: 767 - RNA, 2258 nt, Homo sapiens
  source 1..2258: mol_type = mRNA
agtgccacattagaacagcttgaagaccgttcatttttaagtgacaagagactcacctccaagaagcaattgtgttttcagaatgattttattcaagcaagcaacttatttcatttccttgtttgctacagtttcctgtggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaactctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatca

SEQ ID NO: 768 - RNA, 21 nt, synthetic construct
  source 1..21: mol_type = other RNA
tgtcagtttttcacttattca

SEQ ID NO: 769 - RNA, 21 nt, synthetic construct
  source 1..21: mol_type = other RNA
tgtcagtttttcacttattct

SEQ ID NO: 770 - RNA, 21 nt, synthetic construct
  source 1..21: mol_type = other RNA
tgtcagtttttcacttattcc

SEQ ID NO: 771 - RNA, 21 nt, synthetic construct
  source 1..21: mol_type = other RNA
tgtcagtttttcacttattcg

SEQ ID NO: 772 - RNA, 17 nt, synthetic construct
  source 1..17: mol_type = other RNA
agtttttcacttattca

SEQ ID NO: 773 - RNA, 18 nt, synthetic construct
  source 1..18: mol_type = other RNA
cagtttttcacttattca

SEQ ID NO: 774 - RNA, 19 nt, synthetic construct
  source 1..19: mol_type = other RNA
acagtttttcacttattca

SEQ ID NO: 775 - RNA, 19 nt, synthetic construct
  source 1..19: mol_type = other RNA
tcagtttttcacttattca

SEQ ID NO: 776 - RNA, 19 nt, synthetic construct
  source 1..19: mol_type = other RNA
gcagtttttcacttattca

SEQ ID NO: 777 - RNA, 19 nt, synthetic construct
  source 1..19: mol_type = other RNA
ccagtttttcacttattca

SEQ ID NO: 778 - RNA, 20 nt, synthetic construct
  source 1..20: mol_type = other RNA
atcagtttttcacttattca

SEQ ID NO: 779 - RNA, 20 nt, synthetic construct
  source 1..20: mol_type = other RNA
ttcagtttttcacttattca

SEQ ID NO: 780 - RNA, 20 nt, synthetic construct
  source 1..20: mol_type = other RNA
gtcagtttttcacttattca

SEQ ID NO: 781 - RNA, 20 nt, synthetic construct
  source 1..20: mol_type = other RNA
ctcagtttttcacttattca

SEQ ID NO: 782 - RNA, 21 nt, synthetic construct
  source 1..21: mol_type = other RNA
ggtcagtttttcacttattca

SEQ ID NO: 783 - RNA, 21 nt, synthetic construct
  source 1..21: mol_type = other RNA
agtcagtttttcacttattca

SEQ ID NO: 784 - RNA, 21 nt, synthetic construct
  source 1..21: mol_type = other RNA
cgtcagtttttcacttattca

SEQ ID NO: 785 - RNA, 22 nt, synthetic construct
  source 1..22: mol_type = other RNA
ctgtcagtttttcacttattca

SEQ ID NO: 786 - RNA, 23 nt, synthetic construct
  source 1..23: mol_type = other RNA
gctgtcagtttttcacttattca

SEQ ID NO: 787 - RNA, 24 nt, synthetic construct
  source 1..24: mol_type = other RNA
cgctgtcagtttttcacttattca

SEQ ID NO: 788 - RNA, 25 nt, synthetic construct
  source 1..25: mol_type = other RNA
tcgctgtcagtttttcacttattca

SEQ ID NO: 789 - RNA, 26 nt, synthetic construct
  source 1..26: mol_type = other RNA
ttcgctgtcagtttttcacttattca

SEQ ID NO: 790 - RNA, 32 nt, synthetic construct
  source 1..32: mol_type = other RNA
agatgattcgctgtcagtttttcacttattca

SEQ ID NO: 791 - RNA, 21 nt, synthetic construct
  source 1..21: mol_type = other RNA
tgtcagtttttcacttattca

SEQ ID NO: 792 - RNA, 21 nt, synthetic construct
  source 1..21: mol_type = other RNA
tgtcagtttttcacttattca

SEQ ID NO: 793 - RNA, 21 nt, synthetic construct
  source 1..21: mol_type = other RNA
tgtcagtttttcacttattca

SEQ ID NO: 794 - RNA, 21 nt, synthetic construct
  source 1..21: mol_type = other RNA
tgtcagtttttcacttattca

SEQ ID NO: 795 - RNA, 21 nt, synthetic construct
  source 1..21: mol_type = other RNA
tgtcagtttttcacttattca

SEQ ID NO: 796 - RNA, 21 nt, synthetic construct
  source 1..21: mol_type = other RNA
tgtcagtttttcactcattca

SEQ ID NO: 797 - RNA, 21 nt, synthetic construct
  source 1..21: mol_type = other RNA
tgtcagtttttcacttattta

SEQ ID NO: 798 - RNA, 21 nt, synthetic construct
  source 1..21: mol_type = other RNA
tgtcagtttttcacttattca

SEQ ID NO: 799 - RNA, 21 nt, synthetic construct
  source 1..21: mol_type = other RNA
atacacctttctcacaaataa

SEQ ID NO: 800 - RNA, 21 nt, synthetic construct
  source 1..21: mol_type = other RNA
atacacctttctcacaaatat

SEQ ID NO: 801 - RNA, 21 nt, synthetic construct
  source 1..21: mol_type = other RNA
atacacctttctcacaaatac

SEQ ID NO: 802 - RNA, 21 nt, synthetic construct
  source 1..21: mol_type = other RNA
atacacctttctcacaaatag

SEQ ID NO: 803 - RNA, 17 nt, synthetic construct
  source 1..17: mol_type = other RNA
acctttctcacaaataa

SEQ ID NO: 804 - RNA, 18 nt, synthetic construct
  source 1..18: mol_type = other RNA
cacctttctcacaaataa

SEQ ID NO: 805 - RNA, 19 nt, synthetic construct
  source 1..19: mol_type = other RNA
acacctttctcacaaataa

SEQ ID NO: 806 - RNA, 19 nt, synthetic construct
  source 1..19: mol_type = other RNA
tcacctttctcacaaataa

SEQ ID NO: 807 - RNA, 19 nt, synthetic construct
  source 1..19: mol_type = other RNA
gcacctttctcacaaataa

SEQ ID NO: 808 - RNA, 19 nt, synthetic construct
  source 1..19: mol_type = other RNA
ccacctttctcacaaataa

SEQ ID NO: 809 - RNA, 20 nt, synthetic construct
  source 1..20: mol_type = other RNA
aacacctttctcacaaataa

SEQ ID NO: 810 - RNA, 20 nt, synthetic construct
  source 1..20: mol_type = other RNA
tacacctttctcacaaataa

SEQ ID NO: 811 - RNA, 20 nt, synthetic construct
  source 1..20: mol_type = other RNA
gacacctttctcacaaataa

SEQ ID NO: 812 - RNA, 20 nt, synthetic construct
  source 1..20: mol_type = other RNA
cacacctttctcacaaataa

SEQ ID NO: 813 - RNA, 21 nt, synthetic construct
  source 1..21: mol_type = other RNA
gtacacctttctcacaaataa

SEQ ID NO: 814 - RNA, 21 nt, synthetic construct
  source 1..21: mol_type = other RNA
ctacacctttctcacaaataa

SEQ ID NO: 815 - RNA, 21 nt, synthetic construct
  source 1..21: mol_type = other RNA
ttacacctttctcacaaataa

SEQ ID NO: 816 - RNA, 22 nt, synthetic construct
  source 1..22: mol_type = other RNA
gatacacctttctcacaaataa

SEQ ID NO: 817 - RNA, 23 nt, synthetic construct
  source 1..23: mol_type = other RNA
agatacacctttctcacaaataa

SEQ ID NO: 818 - RNA, 24 nt, synthetic construct
  source 1..24: mol_type = other RNA
aagatacacctttctcacaaataa

SEQ ID NO: 819 - RNA, 25 nt, synthetic construct
  source 1..25: mol_type = other RNA
aaagatacacctttctcacaaataa

SEQ ID NO: 820 - RNA, 26 nt, synthetic construct
  source 1..26: mol_type = other RNA
aaaagatacacctttctcacaaataa

SEQ ID NO: 821 - RNA, 32 nt, synthetic construct
  source 1..32: mol_type = other RNA
cattacaaaagatacacctttctcacaaataa

SEQ ID NO: 822 - RNA, 21 nt, synthetic construct
  source 1..21: mol_type = other RNA
atacacctttctcacaaataa

SEQ ID NO: 823 - RNA, 21 nt, synthetic construct
  source 1..21: mol_type = other RNA
atacacctttctcacaaataa

SEQ ID NO: 824 - RNA, 21 nt, synthetic construct
  source 1..21: mol_type = other RNA
atacacctttctcacaaataa

SEQ ID NO: 825 - RNA, 21 nt, synthetic construct
  source 1..21: mol_type = other RNA
atacacctttctcacaaataa

SEQ ID NO: 826 - RNA, 21 nt, synthetic construct
  source 1..21: mol_type = other RNA
atacacctttctcacaaataa

SEQ ID NO: 827 - RNA, 21 nt, synthetic construct
  source 1..21: mol_type = other RNA
atacacctttctcacgaataa

SEQ ID NO: 828 - RNA, 21 nt, synthetic construct
  source 1..21: mol_type = other RNA
atacacctttctcacaaatga

SEQ ID NO: 829 - RNA, 21 nt, synthetic construct
  source 1..21: mol_type = other RNA
atacacctttctcacaaataa

SEQ ID NO: 830 - RNA, 21 nt, synthetic construct
  source 1..21: mol_type = other RNA
ttcgctgtcagtttttcacta

SEQ ID NO: 831 - RNA, 21 nt, synthetic construct
  source 1..21: mol_type = other RNA
ttcgctgtcagtttttcactt

SEQ ID NO: 832 - RNA, 21 nt, synthetic construct
  source 1..21: mol_type = other RNA
ttcgctgtcagtttttcactc

SEQ ID NO: 833 - RNA, 21 nt, synthetic construct
  source 1..21: mol_type = other RNA
ttcgctgtcagtttttcactg

SEQ ID NO: 834 - RNA, 17 nt, synthetic construct
  source 1..17: mol_type = other RNA
ctgtcagtttttcacta

SEQ ID NO: 835 - RNA, 18 nt, synthetic construct
  source 1..18: mol_type = other RNA
gctgtcagtttttcacta

SEQ ID NO: 836 - RNA, 19 nt, synthetic construct
  source 1..19: mol_type = other RNA
agctgtcagtttttcacta

SEQ ID NO: 837 - RNA, 19 nt, synthetic construct
  source 1..19: mol_type = other RNA
tgctgtcagtttttcacta

SEQ ID NO: 838 - RNA, 19 nt, synthetic construct
  source 1..19: mol_type = other RNA
ggctgtcagtttttcacta

SEQ ID NO: 839 - RNA, 19 nt, synthetic construct
  source 1..19: mol_type = other RNA
cgctgtcagtttttcacta

SEQ ID NO: 840 - RNA, 20 nt, synthetic construct
  source 1..20: mol_type = other RNA
acgctgtcagtttttcacta

SEQ ID NO: 841 - RNA, 20 nt, synthetic construct
  source 1..20: mol_type = other RNA
tcgctgtcagtttttcacta

SEQ ID NO: 842 - RNA, 20 nt, synthetic construct
  source 1..20: mol_type = other RNA
gcgctgtcagtttttcacta

SEQ ID NO: 843 - RNA, 20 nt, synthetic construct
  source 1..20: mol_type = other RNA
ccgctgtcagtttttcacta

SEQ ID NO: 844 - RNA, 21 nt, synthetic construct
  source 1..21: mol_type = other RNA
gtcgctgtcagtttttcacta

SEQ ID NO: 845 - RNA, 21 nt, synthetic construct
  source 1..21: mol_type = other RNA
ctcgctgtcagtttttcacta

SEQ ID NO: 846 - RNA, 21 nt, synthetic construct
  source 1..21: mol_type = other RNA
atcgctgtcagtttttcacta

SEQ ID NO: 847 - RNA, 22 nt, synthetic construct
  source 1..22: mol_type = other RNA
attcgctgtcagtttttcacta

SEQ ID NO: 848 - RNA, 23 nt, synthetic construct
  source 1..23: mol_type = other RNA
gattcgctgtcagtttttcacta

SEQ ID NO: 849 - RNA, 24 nt, synthetic construct
  source 1..24: mol_type = other RNA
tgattcgctgtcagtttttcacta

SEQ ID NO: 850 - RNA, 25 nt, synthetic construct
  source 1..25: mol_type = other RNA
atgattcgctgtcagtttttcacta

SEQ ID NO: 851 - RNA, 26 nt, synthetic construct
  source 1..26: mol_type = other RNA
gatgattcgctgtcagtttttcacta

SEQ ID NO: 852 - RNA, 32 nt, synthetic construct
  source 1..32: mol_type = other RNA
cacaaagatgattcgctgtcagtttttcacta

SEQ ID NO: 853 - RNA, 21 nt, synthetic construct
  source 1..21: mol_type = other RNA
ttcgctgtcagtttttcacta

SEQ ID NO: 854 - RNA, 21 nt, synthetic construct
  source 1..21: mol_type = other RNA
ttcgctgtcagtttttcacta

SEQ ID NO: 855 - RNA, 21 nt, synthetic construct
  source 1..21: mol_type = other RNA
ttcgctgtcagtttttcacta

SEQ ID NO: 856 - RNA, 21 nt, synthetic construct
  source 1..21: mol_type = other RNA
ttcgctgtcagtttttcacta

SEQ ID NO: 857 - RNA, 21 nt, synthetic construct
  source 1..21: mol_type = other RNA
ttcgctgtcagtttttcacta

SEQ ID NO: 858 - RNA, 21 nt, synthetic construct
  source 1..21: mol_type = other RNA
ttcgctgtcagttttccacta

SEQ ID NO: 859 - RNA, 21 nt, synthetic construct
  source 1..21: mol_type = other RNA
ttcgctgtcagtttttcacca

SEQ ID NO: 860 - RNA, 21 nt, synthetic construct
  source 1..21: mol_type = other RNA
ttcgctgtcagtttttcacta

SEQ ID NO: 861 - RNA, 21 nt, synthetic construct
  source 1..21: mol_type = other RNA
tccaggctcctttgaattaca

SEQ ID NO: 862 - RNA, 21 nt, synthetic construct
  source 1..21: mol_type = other RNA
tccaggctcctttgaattact

SEQ ID NO: 863 - RNA, 21 nt, synthetic construct
  source 1..21: mol_type = other RNA
tccaggctcctttgaattacc

SEQ ID NO: 864 - RNA, 21 nt, synthetic construct
  source 1..21: mol_type = other RNA
tccaggctcctttgaattacg

SEQ ID NO: 865 - RNA, 17 nt, synthetic construct
  source 1..17: mol_type = other RNA
ggctcctttgaattaca

SEQ ID NO: 866 - RNA, 18 nt, synthetic construct
  source 1..18: mol_type = other RNA
aggctcctttgaattaca

SEQ ID NO: 867 - RNA, 19 nt, synthetic construct
  source 1..19: mol_type = other RNA
aaggctcctttgaattaca

SEQ ID NO: 868 - RNA, 19 nt, synthetic construct
  source 1..19: mol_type = other RNA
taggctcctttgaattaca

SEQ ID NO: 869 - RNA, 19 nt, synthetic construct
  source 1..19: mol_type = other RNA
gaggctcctttgaattaca

SEQ ID NO: 870 - RNA, 19 nt, synthetic construct
  source 1..19: mol_type = other RNA
caggctcctttgaattaca

SEQ ID NO: 871 - RNA, 20 nt, synthetic construct
  source 1..20: mol_type = other RNA
acaggctcctttgaattaca

SEQ ID NO: 872 - RNA, 20 nt, synthetic construct
  source 1..20: mol_type = other RNA
tcaggctcctttgaattaca

SEQ ID NO: 873 - RNA, 20 nt, synthetic construct
  source 1..20: mol_type = other RNA
gcaggctcctttgaattaca

SEQ ID NO: 874 - RNA, 20 nt, synthetic construct
  source 1..20: mol_type = other RNA
ccaggctcctttgaattaca

SEQ ID NO: 875 - RNA, 21 nt, synthetic construct
  source 1..21: mol_type = other RNA
gccaggctcctttgaattaca

SEQ ID NO: 876 - RNA, 21 nt, synthetic construct
  source 1..21: mol_type = other RNA
cccaggctcctttgaattaca

SEQ ID NO: 877 - RNA, 21 nt, synthetic construct
  source 1..21: mol_type = other RNA
accaggctcctttgaattaca

SEQ ID NO: 878 - RNA, 22 nt, synthetic construct
  source 1..22: mol_type = other RNA
ctccaggctcctttgaattaca

SEQ ID NO: 879 - RNA, 23 nt, synthetic construct
  source 1..23: mol_type = other RNA
actccaggctcctttgaattaca

SEQ ID NO: 880 - RNA, 24 nt, synthetic construct
  source 1..24: mol_type = other RNA
aactccaggctcctttgaattaca

SEQ ID NO: 881 - RNA, 25 nt, synthetic construct
  source 1..25: mol_type = other RNA
aaactccaggctcctttgaattaca

SEQ ID NO: 882 - RNA, 26 nt, synthetic construct
  source 1..26: mol_type = other RNA
aaaactccaggctcctttgaattaca

SEQ ID NO: 883 - RNA, 32 nt, synthetic construct
  source 1..32: mol_type = other RNA
cttgataaaactccaggctcctttgaattaca

SEQ ID NO: 884 - RNA, 21 nt, synthetic construct
  source 1..21: mol_type = other RNA
tccaggctcctttgaattaca

SEQ ID NO: 885 - RNA, 21 nt, synthetic construct
  source 1..21: mol_type = other RNA
tccaggctcctttgaattaca

SEQ ID NO: 886 - RNA, 21 nt, synthetic construct
  source 1..21: mol_type = other RNA
tccaggctcctttgaattaca

SEQ ID NO: 887 - RNA, 21 nt, synthetic construct
  source 1..21: mol_type = other RNA
tccaggctcctttgaattaca

SEQ ID NO: 888 - RNA, 21 nt, synthetic construct
  source 1..21: mol_type = other RNA
tccaggctcctttgaattaca

SEQ ID NO: 889 - RNA, 21 nt, synthetic construct
  source 1..21: mol_type = other RNA
tccaggctcctttgagttaca

SEQ ID NO: 890 - RNA, 21 nt, synthetic construct
  source 1..21: mol_type = other RNA
tccaggctcctttgaattata

SEQ ID NO: 891 - RNA, 21 nt, synthetic construct
  source 1..21: mol_type = other RNA
tccaggctcctttgaattaca

SEQ ID NO: 892 - RNA, 21 nt, synthetic construct
  source 1..21: mol_type = other RNA
gaaggagtgaatgtttgccaa

SEQ ID NO: 893 - RNA, 21 nt, synthetic construct
  source 1..21: mol_type = other RNA
gaaggagtgaatgtttgccat

SEQ ID NO: 894 - RNA, 21 nt, synthetic construct
  source 1..21: mol_type = other RNA
gaaggagtgaatgtttgccac

SEQ ID NO: 895 - RNA, 21 nt, synthetic construct
  source 1..21: mol_type = other RNA
gaaggagtgaatgtttgccag

SEQ ID NO: 896 - RNA, 17 nt, synthetic construct
  source 1..17: mol_type = other RNA
gagtgaatgtttgccaa

SEQ ID NO: 897 - RNA, 18 nt, synthetic construct
  source 1..18: mol_type = other RNA
ggagtgaatgtttgccaa

SEQ ID NO: 898 - RNA, 19 nt, synthetic construct
  source 1..19: mol_type = other RNA
aggagtgaatgtttgccaa

SEQ ID NO: 899 - RNA, 19 nt, synthetic construct
  source 1..19: mol_type = other RNA
tggagtgaatgtttgccaa

SEQ ID NO: 900 - RNA, 19 nt, synthetic construct
  source 1..19: mol_type = other RNA
gggagtgaatgtttgccaa

SEQ ID NO: 901 - RNA, 19 nt, synthetic construct
  source 1..19: mol_type = other RNA
cggagtgaatgtttgccaa

SEQ ID NO: 902 - RNA, 20 nt, synthetic construct
  source 1..20: mol_type = other RNA
aaggagtgaatgtttgccaa

SEQ ID NO: 903 - RNA, 20 nt, synthetic construct
  source 1..20: mol_type = other RNA
taggagtgaatgtttgccaa

SEQ ID NO: 904 - RNA, 20 nt, synthetic construct
  source 1..20: mol_type = other RNA
gaggagtgaatgtttgccaa

SEQ ID NO: 905 - RNA, 20 nt, synthetic construct
  source 1..20: mol_type = other RNA
caggagtgaatgtttgccaa

SEQ ID NO: 906 - RNA, 21 nt, synthetic construct
  source 1..21: mol_type = other RNA
taaggagtgaatgtttgccaa

SEQ ID NO: 907 - RNA, 21 nt, synthetic construct
  source 1..21: mol_type = other RNA
caaggagtgaatgtttgccaa

SEQ ID NO: 908 - RNA, 21 nt, synthetic construct
  source 1..21: mol_type = other RNA
aaaggagtgaatgtttgccaa

SEQ ID NO: 909 - RNA, 22 nt, synthetic construct
  source 1..22: mol_type = other RNA
taaaggagtgaatgtttgccaa

SEQ ID NO: 910 - RNA, 23 nt, synthetic construct
  source 1..23: mol_type = other RNA
ttaaaggagtgaatgtttgccaa

SEQ ID NO: 911 - RNA, 24 nt, synthetic construct
  source 1..24: mol_type = other RNA
gttaaaggagtgaatgtttgccaa

SEQ ID NO: 912 - RNA, 25 nt, synthetic construct
  source 1..25: mol_type = other RNA
tgttaaaggagtgaatgtttgccaa

SEQ ID NO: 913 - RNA, 26 nt, synthetic construct
  source 1..26: mol_type = other RNA
ttgttaaaggagtgaatgtttgccaa

SEQ ID NO: 914 - RNA, 32 nt, synthetic construct
  source 1..32: mol_type = other RNA
tgacttttgttaaaggagtgaatgtttgccaa

SEQ ID NO: 915 - RNA, 21 nt, synthetic construct
  source 1..21: mol_type = other RNA
gaaggagtgaatgtttgccaa

SEQ ID NO: 916 - RNA, 21 nt, synthetic construct
  source 1..21: mol_type = other RNA
gaaggagtgaatgtttgccaa

SEQ ID NO: 917 - RNA, 21 nt, synthetic construct
  source 1..21: mol_type = other RNA
gaaggagtgaatgtttgccaa

SEQ ID NO: 918 - RNA, 21 nt, synthetic construct
  source 1..21: mol_type = other RNA
gaaggagtgaatgtttgccaa

SEQ ID NO: 919 - RNA, 21 nt, synthetic construct
  source 1..21: mol_type = other RNA
gaaggagtgaatgtttgccaa

SEQ ID NO: 920 - RNA, 21 nt, synthetic construct
  source 1..21: mol_type = other RNA
gaaggagtgaatgttcgccaa

SEQ ID NO: 921 - RNA, 21 nt, synthetic construct
  source 1..21: mol_type = other RNA
gaaggagtgaatgtttgccga

SEQ ID NO: 922 - RNA, 21 nt, synthetic construct
  source 1..21: mol_type = other RNA
gaaggagtgaatgtttgccaa

SEQ ID NO: 923 - RNA, 21 nt, synthetic construct
  source 1..21: mol_type = other RNA
ttcgctgtcagtttttcacta

SEQ ID NO: 924 - RNA, 21 nt, synthetic construct
  source 1..21: mol_type = other RNA
tcgctgtcagtttttcactta

SEQ ID NO: 925 - RNA, 21 nt, synthetic construct
  source 1..21: mol_type = other RNA
cgctgtcagtttttcacttaa

SEQ ID NO: 926 - RNA, 21 nt, synthetic construct
  source 1..21: mol_type = other RNA
gctgtcagtttttcacttata

SEQ ID NO: 927 - RNA, 21 nt, synthetic construct
  source 1..21: mol_type = other RNA
ctgtcagtttttcacttatta

SEQ ID NO: 928 - RNA, 21 nt, synthetic construct
  source 1..21: mol_type = other RNA
gtcagtttttcacttattcta

SEQ ID NO: 929 - RNA, 21 nt, synthetic construct
  source 1..21: mol_type = other RNA
tcagtttttcacttattctta

SEQ ID NO: 930 - RNA, 21 nt, synthetic construct
  source 1..21: mol_type = other RNA
cagtttttcacttattcttta

SEQ ID NO: 931 - RNA, 21 nt, synthetic construct
  source 1..21: mol_type = other RNA
agtttttcacttattctttaa

SEQ ID NO: 932 - RNA, 21 nt, synthetic construct
  source 1..21: mol_type = other RNA
gtttttcacttattctttaca

SEQ ID NO: 933 - RNA, 21 nt, synthetic construct
  source 1..21: mol_type = other RNA
aaaagatacacctttctcaca

SEQ ID NO: 934 - RNA, 21 nt, synthetic construct
  source 1..21: mol_type = other RNA
aaagatacacctttctcacaa

SEQ ID NO: 935 - RNA, 21 nt, synthetic construct
  source 1..21: mol_type = other RNA
aagatacacctttctcacaaa

SEQ ID NO: 936 - RNA, 21 nt, synthetic construct
  source 1..21: mol_type = other RNA
agatacacctttctcacaaaa

SEQ ID NO: 937 - RNA, 21 nt, synthetic construct
  source 1..21: mol_type = other RNA
gatacacctttctcacaaata

SEQ ID NO: 938 - RNA, 21 nt, synthetic construct
  source 1..21: mol_type = other RNA
tacacctttctcacaaataaa

SEQ ID NO: 939 - RNA, 21 nt, synthetic construct
  source 1..21: mol_type = other RNA
acacctttctcacaaataaaa

SEQ ID NO: 940 - RNA, 21 nt, synthetic construct
  source 1..21: mol_type = other RNA
cacctttctcacaaataaaaa

SEQ ID NO: 941 - RNA, 21 nt, synthetic construct
  source 1..21: mol_type = other RNA
acctttctcacaaataaaaga

SEQ ID NO: 942 - RNA, 21 nt, synthetic construct
  source 1..21: mol_type = other RNA
cctttctcacaaataaaagaa

SEQ ID NO: 943 - RNA, 21 nt, synthetic construct
  source 1..21: mol_type = other RNA
gatgattcgctgtcagtttta

SEQ ID NO: 944 - RNA, 21 nt, synthetic construct
  source 1..21: mol_type = other RNA
atgattcgctgtcagttttta

SEQ ID NO: 945 - RNA, 21 nt, synthetic construct
  source 1..21: mol_type = other RNA
tgattcgctgtcagtttttca

SEQ ID NO: 946 - RNA, 21 nt, synthetic construct
  source 1..21: mol_type = other RNA
gattcgctgtcagtttttcaa

SEQ ID NO: 947 - RNA, 21 nt, synthetic construct
  source 1..21: mol_type = other RNA
attcgctgtcagtttttcaca

SEQ ID NO: 948 - RNA, 21 nt, synthetic construct
  source 1..21: mol_type = other RNA
tcgctgtcagtttttcactta

SEQ ID NO: 949 - RNA, 21 nt, synthetic construct
  source 1..21: mol_type = other RNA
cgctgtcagtttttcacttaa

SEQ ID NO: 950 - RNA, 21 nt, synthetic construct
  source 1..21: mol_type = other RNA
gctgtcagtttttcacttata

SEQ ID NO: 951 - RNA, 21 nt, synthetic construct
  source 1..21: mol_type = other RNA
ctgtcagtttttcacttatta

SEQ ID NO: 952 - RNA, 21 nt, synthetic construct
  source 1..21: mol_type = other RNA
tgtcagtttttcacttattca

SEQ ID NO: 953 - RNA, 21 nt, synthetic construct
  source 1..21: mol_type = other RNA
aaaactccaggctcctttgaa

SEQ ID NO: 954 - RNA, 21 nt, synthetic construct
  source 1..21: mol_type = other RNA
aaactccaggctcctttgaaa

SEQ ID NO: 955 - RNA, 21 nt, synthetic construct
  source 1..21: mol_type = other RNA
aactccaggctcctttgaata

SEQ ID NO: 956 - RNA, 21 nt, synthetic construct
  source 1..21: mol_type = other RNA
actccaggctcctttgaatta

SEQ ID NO: 957 - RNA, 21 nt, synthetic construct
  source 1..21: mol_type = other RNA
ctccaggctcctttgaattaa

SEQ ID NO: 958 - RNA, 21 nt, synthetic construct
  source 1..21: mol_type = other RNA
ccaggctcctttgaattacaa

SEQ ID NO: 959 - RNA, 21 nt, synthetic construct
  source 1..21: mol_type = other RNA
caggctcctttgaattacaca

SEQ ID NO: 960 - RNA, 21 nt, synthetic construct
  source 1..21: mol_type = other RNA
aggctcctttgaattacacta

SEQ ID NO: 961 - RNA, 21 nt, synthetic construct
  source 1..21: mol_type = other RNA
ggctcctttgaattacactga

SEQ ID NO: 962 - RNA, 21 nt, synthetic construct
  source 1..21: mol_type = other RNA
gctcctttgaattacactgaa

SEQ ID NO: 963 - RNA, 21 nt, synthetic construct
  source 1..21: mol_type = other RNA
ttgttaaaggagtgaatgtta

SEQ ID NO: 964 - RNA, 21 nt, synthetic construct
  source 1..21: mol_type = other RNA
tgttaaaggagtgaatgttta

SEQ ID NO: 965 - RNA, 21 nt, synthetic construct
  source 1..21: mol_type = other RNA
gttaaaggagtgaatgtttga

SEQ ID NO: 966 - RNA, 21 nt, synthetic construct
  source 1..21: mol_type = other RNA
ttaaaggagtgaatgtttgca

SEQ ID NO: 967 - RNA, 21 nt, synthetic construct
  source 1..21: mol_type = other RNA
taaaggagtgaatgtttgcca

SEQ ID NO: 968 - RNA, 21 nt, synthetic construct
  source 1..21: mol_type = other RNA
aaggagtgaatgtttgccaaa

SEQ ID NO: 969 - RNA, 21 nt, synthetic construct
  source 1..21: mol_type = other RNA
aggagtgaatgtttgccaaga

SEQ ID NO: 970 - RNA, 21 nt, synthetic construct
  source 1..21: mol_type = other RNA
ggagtgaatgtttgccaagaa

SEQ ID NO: 971 - RNA, 21 nt, synthetic construct
  source 1..21: mol_type = other RNA
gagtgaatgtttgccaagaga

SEQ ID NO: 972 - RNA, 21 nt, synthetic construct
  source 1..21: mol_type = other RNA
agtgaatgtttgccaagagaa

SEQ ID NO: 973 - RNA, 21 nt, synthetic construct
  source 1..21: mol_type = other RNA
tgaataagtgaaaaactgaca

SEQ ID NO: 974 - RNA, 21 nt, synthetic construct
  source 1..21: mol_type = other RNA
agaataagtgaaaaactgaca

SEQ ID NO: 975 - RNA, 21 nt, synthetic construct
  source 1..21: mol_type = other RNA
ggaataagtgaaaaactgaca

SEQ ID NO: 976 - RNA, 21 nt, synthetic construct
  source 1..21: mol_type = other RNA
cgaataagtgaaaaactgaca

SEQ ID NO: 977 - RNA, 17 nt, synthetic construct
  source 1..17: mol_type = other RNA
tgaataagtgaaaaact

SEQ ID NO: 978 - RNA, 18 nt, synthetic construct
  source 1..18: mol_type = other RNA
tgaataagtgaaaaactg

SEQ ID NO: 979 - RNA, 19 nt, synthetic construct
  source 1..19: mol_type = other RNA
tgaataagtgaaaaactgt

SEQ ID NO: 980 - RNA, 19 nt, synthetic construct
  source 1..19: mol_type = other RNA
tgaataagtgaaaaactga

SEQ ID NO: 981 - RNA, 19 nt, synthetic construct
  source 1..19: mol_type = other RNA
tgaataagtgaaaaactgc

SEQ ID NO: 982 - RNA, 19 nt, synthetic construct
  source 1..19: mol_type = other RNA
tgaataagtgaaaaactgg

SEQ ID NO: 983 - RNA, 20 nt, synthetic construct
  source 1..20: mol_type = other RNA
tgaataagtgaaaaactgat

SEQ ID NO: 984 - RNA, 20 nt, synthetic construct
  source 1..20: mol_type = other RNA
tgaataagtgaaaaactgaa

SEQ ID NO: 985 - RNA, 20 nt, synthetic construct
  source 1..20: mol_type = other RNA
tgaataagtgaaaaactgac

SEQ ID NO: 986 - RNA, 20 nt, synthetic construct
  source 1..20: mol_type = other RNA
tgaataagtgaaaaactgag

SEQ ID NO: 987 - RNA, 21 nt, synthetic construct
  source 1..21: mol_type = other RNA
tgaataagtgaaaaactgacc

SEQ ID NO: 988 - RNA, 21 nt, synthetic construct
  source 1..21: mol_type = other RNA
tgaataagtgaaaaactgact

SEQ ID NO: 989 - RNA, 21 nt, synthetic construct
  source 1..21: mol_type = other RNA
tgaataagtgaaaaactgacg

SEQ ID NO: 990 - RNA, 22 nt, synthetic construct
  source 1..22: mol_type = other RNA
tgaataagtgaaaaactgacag

SEQ ID NO: 991 - RNA, 23 nt, synthetic construct
  source 1..23: mol_type = other RNA
tgaataagtgaaaaactgacagc

SEQ ID NO: 992 - RNA, 24 nt, synthetic construct
  source 1..24: mol_type = other RNA
tgaataagtgaaaaactgacagcg

SEQ ID NO: 993 - RNA, 25 nt, synthetic construct
  source 1..25: mol_type = other RNA
tgaataagtgaaaaactgacagcga

SEQ ID NO: 994 - RNA, 26 nt, synthetic construct
  source 1..26: mol_type = other RNA
tgaataagtgaaaaactgacagcgaa

SEQ ID NO: 995 - RNA, 32 nt, synthetic construct
  source 1..32: mol_type = other RNA
tgaataagtgaaaaactgacagcgaatcatct

SEQ ID NO: 996 - RNA, 23 nt, synthetic construct
  source 1..23: mol_type = other RNA
tgaataagtgaaaaactgacatt

SEQ ID NO: 997 - RNA, 23 nt, synthetic construct
  source 1..23: mol_type = other RNA
tgaataagtgaaaaactgacaga

SEQ ID NO: 998 - RNA, 21 nt, synthetic construct
  source 1..21: mol_type = other RNA
tgaataagtgaaaaactgaca

SEQ ID NO: 999 - RNA, 21 nt, synthetic construct
  source 1..21: mol_type = other RNA
tgaataagtgaaaaactgaca

SEQ ID NO: 1000 - RNA, 21 nt, synthetic construct
  source 1..21: mol_type = other RNA
tgaataagtgaaaaactgaca

SEQ ID NO: 1001 - RNA, 21 nt, synthetic construct
  source 1..21: mol_type = other RNA
tgaataagtgaaaaactgaca

SEQ ID NO: 1002 - RNA, 21 nt, synthetic construct
  source 1..21: mol_type = other RNA
taaataagtgaaaaactgaca

SEQ ID NO: 1003 - RNA, 21 nt, synthetic construct
  source 1..21: mol_type = other RNA
tgaataagtgaaaaactgaca

SEQ ID NO: 1004 - RNA, 21 nt, synthetic construct
  source 1..21: mol_type = other RNA
ttatttgtgagaaaggtgtat

SEQ ID NO: 1005 - RNA, 21 nt, synthetic construct
  source 1..21: mol_type = other RNA
atatttgtgagaaaggtgtat

SEQ ID NO: 1006 - RNA, 21 nt, synthetic construct
  source 1..21: mol_type = other RNA
gtatttgtgagaaaggtgtat

SEQ ID NO: 1007 - RNA, 21 nt, synthetic construct
  source 1..21: mol_type = other RNA
ctatttgtgagaaaggtgtat

SEQ ID NO: 1008 - RNA, 17 nt, synthetic construct
  source 1..17: mol_type = other RNA
ttatttgtgagaaaggt

SEQ ID NO: 1009 - RNA, 18 nt, synthetic construct
  source 1..18: mol_type = other RNA
ttatttgtgagaaaggtg

SEQ ID NO: 1010 - RNA, 19 nt, synthetic construct
  source 1..19: mol_type = other RNA
ttatttgtgagaaaggtgt

SEQ ID NO: 1011 - RNA, 19 nt, synthetic construct
  source 1..19: mol_type = other RNA
ttatttgtgagaaaggtga

SEQ ID NO: 1012 - RNA, 19 nt, synthetic construct
  source 1..19: mol_type = other RNA
ttatttgtgagaaaggtgc

SEQ ID NO: 1013 - RNA, 19 nt, synthetic construct
  source 1..19: mol_type = other RNA
ttatttgtgagaaaggtgg

SEQ ID NO: 1014 - RNA, 20 nt, synthetic construct
  source 1..20: mol_type = other RNA
ttatttgtgagaaaggtgtt

SEQ ID NO: 1015 - RNA, 20 nt, synthetic construct
  source 1..20: mol_type = other RNA
ttatttgtgagaaaggtgta

SEQ ID NO: 1016 - RNA, 20 nt, synthetic construct
  source 1..20: mol_type = other RNA
ttatttgtgagaaaggtgtc

SEQ ID NO: 1017 - RNA, 20 nt, synthetic construct
  source 1..20: mol_type = other RNA
ttatttgtgagaaaggtgtg

SEQ ID NO: 1018 - RNA, 21 nt, synthetic construct
  source 1..21: mol_type = other RNA
ttatttgtgagaaaggtgtac

SEQ ID NO: 1019 - RNA, 21 nt, synthetic construct
  source 1..21: mol_type = other RNA
ttatttgtgagaaaggtgtag

SEQ ID NO: 1020 - RNA, 21 nt, synthetic construct
  source 1..21: mol_type = other RNA
ttatttgtgagaaaggtgtaa

SEQ ID NO: 1021 - RNA, 22 nt, synthetic construct
  source 1..22: mol_type = other RNA
ttatttgtgagaaaggtgtatc

SEQ ID NO: 1022 - RNA, 23 nt, synthetic construct
  source 1..23: mol_type = other RNA
ttatttgtgagaaaggtgtatct

SEQ ID NO: 1023 - RNA, 24 nt, synthetic construct
  source 1..24: mol_type = other RNA
ttatttgtgagaaaggtgtatctt

SEQ ID NO: 1024 - RNA, 25 nt, synthetic construct
  source 1..25: mol_type = other RNA
ttatttgtgagaaaggtgtatcttt

SEQ ID NO: 1025 - RNA, 26 nt, synthetic construct
  source 1..26: mol_type = other RNA
ttatttgtgagaaaggtgtatctttt

SEQ ID NO: 1026 - RNA, 32 nt, synthetic construct
  source 1..32: mol_type = other RNA
ttatttgtgagaaaggtgtatcttttgtaatg

SEQ ID NO: 1027 - RNA, 23 nt, synthetic construct
  source 1..23: mol_type = other RNA
ttatttgtgagaaaggtgtattt

SEQ ID NO: 1028 - RNA, 23 nt, synthetic construct
  source 1..23: mol_type = other RNA
ttatttgtgagaaaggtgtatga

SEQ ID NO: 1029 - RNA, 21 nt, synthetic construct
  source 1..21: mol_type = other RNA
ttatttgtgagaaaggtgtat

SEQ ID NO: 1030 - RNA, 21 nt, synthetic construct
  source 1..21: mol_type = other RNA
ttatttgtgagaaaggtgtat

SEQ ID NO: 1031 - RNA, 21 nt, synthetic construct
  source 1..21: mol_type = other RNA
ttatttgtgagaaaggtgtat

SEQ ID NO: 1032 - RNA, 21 nt, synthetic construct
  source 1..21: mol_type = other RNA
ttatttgtgagaaaggtgtat

SEQ ID NO: 1033 - RNA, 21 nt, synthetic construct
  source 1..21: mol_type = other RNA
tcatttgtgagaaaggtgtat

SEQ ID NO: 1034 - RNA, 21 nt, synthetic construct
  source 1..21: mol_type = other RNA
ttatttgtgagaaaggtgtat

SEQ ID NO: 1035 - RNA, 21 nt, synthetic construct
  source 1..21: mol_type = other RNA
tagtgaaaaactgacagcgaa

SEQ ID NO: 1036 - RNA, 21 nt, synthetic construct
  source 1..21: mol_type = other RNA
aagtgaaaaactgacagcgaa

SEQ ID NO: 1037 - RNA, 21 nt, synthetic construct
  source 1..21: mol_type = other RNA
gagtgaaaaactgacagcgaa

SEQ ID NO: 1038 - RNA, 21 nt, synthetic construct
  source 1..21: mol_type = other RNA
cagtgaaaaactgacagcgaa

SEQ ID NO: 1039 - RNA, 17 nt, synthetic construct
  source 1..17: mol_type = other RNA
tagtgaaaaactgacag

SEQ ID NO: 1040 - RNA, 18 nt, synthetic construct
  source 1..18: mol_type = other RNA
tagtgaaaaactgacagc

SEQ ID NO: 1041 - RNA, 19 nt, synthetic construct
  source 1..19: mol_type = other RNA
tagtgaaaaactgacagct

SEQ ID NO: 1042 - RNA, 19 nt, synthetic construct
  source 1..19: mol_type = other RNA
tagtgaaaaactgacagca

SEQ ID NO: 1043 - RNA, 19 nt, synthetic construct
  source 1..19: mol_type = other RNA
tagtgaaaaactgacagcc

SEQ ID NO: 1044 - RNA, 19 nt, synthetic construct
  source 1..19: mol_type = other RNA
tagtgaaaaactgacagcg

SEQ ID NO: 1045 - RNA, 20 nt, synthetic construct
  source 1..20: mol_type = other RNA
tagtgaaaaactgacagcgt

SEQ ID NO: 1046 - RNA, 20 nt, synthetic construct
  source 1..20: mol_type = other RNA
tagtgaaaaactgacagcga

SEQ ID NO: 1047 - RNA, 20 nt, synthetic construct
  source 1..20: mol_type = other RNA
tagtgaaaaactgacagcgc

SEQ ID NO: 1048 - RNA, 20 nt, synthetic construct
  source 1..20: mol_type = other RNA
tagtgaaaaactgacagcgg

SEQ ID NO: 1049 - RNA, 21 nt, synthetic construct
  source 1..21: mol_type = other RNA
tagtgaaaaactgacagcgac

SEQ ID NO: 1050 - RNA, 21 nt, synthetic construct
  source 1..21: mol_type = other RNA
tagtgaaaaactgacagcgag

SEQ ID NO: 1051 - RNA, 21 nt, synthetic construct
  source 1..21: mol_type = other RNA
tagtgaaaaactgacagcgat

SEQ ID NO: 1052 - RNA, 22 nt, synthetic construct
  source 1..22: mol_type = other RNA
tagtgaaaaactgacagcgaat

SEQ ID NO: 1053 - RNA, 23 nt, synthetic construct
  source 1..23: mol_type = other RNA
tagtgaaaaactgacagcgaatc

SEQ ID NO: 1054 - RNA, 24 nt, synthetic construct
  source 1..24: mol_type = other RNA
tagtgaaaaactgacagcgaatca

SEQ ID NO: 1055 - RNA, 25 nt, synthetic construct
  source 1..25: mol_type = other RNA
tagtgaaaaactgacagcgaatcat

SEQ ID NO: 1056 - RNA, 26 nt, synthetic construct
  source 1..26: mol_type = other RNA
tagtgaaaaactgacagcgaatcatc

SEQ ID NO: 1057 - RNA, 32 nt, synthetic construct
  source 1..32: mol_type = other RNA
tagtgaaaaactgacagcgaatcatctttgtg

SEQ ID NO: 1058 - RNA, 21 nt, synthetic construct
  source 1..21: mol_type = other RNA
tagtgaaaaactgacagcgaa

SEQ ID NO: 1059 - RNA, 21 nt, synthetic construct
  source 1..21: mol_type = other RNA
tagtgaaaaactgacagcgaa

SEQ ID NO: 1060 - RNA, 21 nt, synthetic construct
  source 1..21: mol_type = other RNA
tagtgaaaaactgacagcgaa

SEQ ID NO: 1061 - RNA, 21 nt, synthetic construct
  source 1..21: mol_type = other RNA
tagtgaaaaactgacagcgaa

SEQ ID NO: 1062 - RNA, 21 nt, synthetic construct
  source 1..21: mol_type = other RNA
tagtgaaaaactgacagcgaa

SEQ ID NO: 1063 - RNA, 21 nt, synthetic construct
  source 1..21: mol_type = other RNA
tagtgaaaaactgacagcgaa

SEQ ID NO: 1064 - RNA, 21 nt, synthetic construct
  source 1..21: mol_type = other RNA
tggtgaaaaactgacagcgaa

SEQ ID NO: 1065 - RNA, 21 nt, synthetic construct
  source 1..21: mol_type = other RNA
tagtgaaaaactgacagcgaa

SEQ ID NO: 1066 - RNA, 21 nt, synthetic construct
  source 1..21: mol_type = other RNA
tgtaattcaaaggagcctgga

SEQ ID NO: 1067 - RNA, 21 nt, synthetic construct
  source 1..21: mol_type = other RNA
agtaattcaaaggagcctgga

SEQ ID NO: 1068 - RNA, 21 nt, synthetic construct
  source 1..21: mol_type = other RNA
ggtaattcaaaggagcctgga

SEQ ID NO: 1069 - RNA, 21 nt, synthetic construct
  source 1..21: mol_type = other RNA
cgtaattcaaaggagcctgga

SEQ ID NO: 1070 - RNA, 17 nt, synthetic construct
  source 1..17: mol_type = other RNA
tgtaattcaaaggagcc

SEQ ID NO: 1071 - RNA, 18 nt, synthetic construct
  source 1..18: mol_type = other RNA
tgtaattcaaaggagcct

SEQ ID NO: 1072 - RNA, 19 nt, synthetic construct
  source 1..19: mol_type = other RNA
tgtaattcaaaggagcctt

SEQ ID NO: 1073 - RNA, 19 nt, synthetic construct
  source 1..19: mol_type = other RNA
tgtaattcaaaggagccta

SEQ ID NO: 1074 - RNA, 19 nt, synthetic construct
  source 1..19: mol_type = other RNA
tgtaattcaaaggagcctc

SEQ ID NO: 1075 - RNA, 19 nt, synthetic construct
  source 1..19: mol_type = other RNA
tgtaattcaaaggagcctg

SEQ ID NO: 1076 - RNA, 20 nt, synthetic construct
  source 1..20: mol_type = other RNA
tgtaattcaaaggagcctgt

SEQ ID NO: 1077 - RNA, 20 nt, synthetic construct
  source 1..20: mol_type = other RNA
tgtaattcaaaggagcctga

SEQ ID NO: 1078 - RNA, 20 nt, synthetic construct
  source 1..20: mol_type = other RNA
tgtaattcaaaggagcctgc

SEQ ID NO: 1079 - RNA, 20 nt, synthetic construct
  source 1..20: mol_type = other RNA
tgtaattcaaaggagcctgg

SEQ ID NO: 1080 - RNA, 21 nt, synthetic construct
  source 1..21: mol_type = other RNA
tgtaattcaaaggagcctggc

SEQ ID NO: 1081 - RNA, 21 nt, synthetic construct
  source 1..21: mol_type = other RNA
tgtaattcaaaggagcctggg

SEQ ID NO: 1082 - RNA, 21 nt, synthetic construct
  source 1..21: mol_type = other RNA
tgtaattcaaaggagcctggt

SEQ ID NO: 1083 - RNA, 22 nt, synthetic construct
  source 1..22: mol_type = other RNA
tgtaattcaaaggagcctggag

SEQ ID NO: 1084 - RNA, 23 nt, synthetic construct
  source 1..23: mol_type = other RNA
tgtaattcaaaggagcctggagt

SEQ ID NO: 1085 - RNA, 24 nt, synthetic construct
  source 1..24: mol_type = other RNA
tgtaattcaaaggagcctggagtt

SEQ ID NO: 1086 - RNA, 25 nt, synthetic construct
  source 1..25: mol_type = other RNA
tgtaattcaaaggagcctggagttt

SEQ ID NO: 1087 - RNA, 26 nt, synthetic construct
  source 1..26: mol_type = other RNA
tgtaattcaaaggagcctggagtttt

SEQ ID NO: 1088 - RNA, 32 nt, synthetic construct
  source 1..32: mol_type = other RNA
tgtaattcaaaggagcctggagttttatcaag

SEQ ID NO: 1089 - RNA, 23 nt, synthetic construct
  source 1..23: mol_type = other RNA
tgtaattcaaaggagcctggatt

SEQ ID NO: 1090 - RNA, 23 nt, synthetic construct
  source 1..23: mol_type = other RNA
tgtaattcaaaggagcctggaga

SEQ ID NO: 1091 - RNA, 21 nt, synthetic construct
  source 1..21: mol_type = other RNA
tgtaattcaaaggagcctgga

SEQ ID NO: 1092 - RNA, 21 nt, synthetic construct
  source 1..21: mol_type = other RNA
tgtaattcaaaggagcctgga

SEQ ID NO: 1093 - RNA, 21 nt, synthetic construct
  source 1..21: mol_type = other RNA
tgtaattcaaaggagcctgga

SEQ ID NO: 1094 - RNA, 21 nt, synthetic construct
  source 1..21: mol_type = other RNA
tgtaattcaaaggagcctgga

SEQ ID NO: 1095 - RNA, 21 nt, synthetic construct
  source 1..21: mol_type = other RNA
tataattcaaaggagcctgga

SEQ ID NO: 1096 - RNA, 21 nt, synthetic construct
  source 1..21: mol_type = other RNA
tgtaattcaaaggagcctgga

SEQ ID NO: 1097 - RNA, 21 nt, synthetic construct
  source 1..21: mol_type = other RNA
ttggcaaacattcactccttc

SEQ ID NO: 1098 - RNA, 21 nt, synthetic construct
  source 1..21: mol_type = other RNA
atggcaaacattcactccttc

SEQ ID NO: 1099 - RNA, 21 nt, synthetic construct
  source 1..21: mol_type = other RNA
gtggcaaacattcactccttc

SEQ ID NO: 1100 - RNA, 21 nt, synthetic construct
  source 1..21: mol_type = other RNA
ctggcaaacattcactccttc

SEQ ID NO: 1101 - RNA, 17 nt, synthetic construct
  source 1..17: mol_type = other RNA
ttggcaaacattcactc

SEQ ID NO: 1102 - RNA, 18 nt, synthetic construct
  source 1..18: mol_type = other RNA
ttggcaaacattcactcc

SEQ ID NO: 1103 - RNA, 19 nt, synthetic construct
  source 1..19: mol_type = other RNA
ttggcaaacattcactcct

SEQ ID NO: 1104 - RNA, 19 nt, synthetic construct
  source 1..19: mol_type = other RNA
ttggcaaacattcactcca

SEQ ID NO: 1105 - RNA, 19 nt, synthetic construct
  source 1..19: mol_type = other RNA
ttggcaaacattcactccc

SEQ ID NO: 1106 - RNA, 19 nt, synthetic construct
  source 1..19: mol_type = other RNA
ttggcaaacattcactccg

SEQ ID NO: 1107 - RNA, 20 nt, synthetic construct
  source 1..20: mol_type = other RNA
ttggcaaacattcactcctt

SEQ ID NO: 1108 - RNA, 20 nt, synthetic construct
  source 1..20: mol_type = other RNA
ttggcaaacattcactccta

SEQ ID NO: 1109 - RNA, 20 nt, synthetic construct
  source 1..20: mol_type = other RNA
ttggcaaacattcactcctc

SEQ ID NO: 1110 - RNA, 20 nt, synthetic construct
  source 1..20: mol_type = other RNA
ttggcaaacattcactcctg

SEQ ID NO: 1111 - RNA, 21 nt, synthetic construct
  source 1..21: mol_type = other RNA
ttggcaaacattcactcctta

SEQ ID NO: 1112 - RNA, 21 nt, synthetic construct
  source 1..21: mol_type = other RNA
ttggcaaacattcactccttg

SEQ ID NO: 1113 - RNA, 21 nt, synthetic construct
  source 1..21: mol_type = other RNA
ttggcaaacattcactccttt

SEQ ID NO: 1114 - RNA, 22 nt, synthetic construct
  source 1..22: mol_type = other RNA
ttggcaaacattcactccttta

SEQ ID NO: 1115 - RNA, 23 nt, synthetic construct
  source 1..23: mol_type = other RNA
ttggcaaacattcactcctttaa

SEQ ID NO: 1116 - RNA, 24 nt, synthetic construct
  source 1..24: mol_type = other RNA
ttggcaaacattcactcctttaac

SEQ ID NO: 1117 - RNA, 25 nt, synthetic construct
  source 1..25: mol_type = other RNA
ttggcaaacattcactcctttaaca

SEQ ID NO: 1118 - RNA, 26 nt, synthetic construct
  source 1..26: mol_type = other RNA
ttggcaaacattcactcctttaacaa

SEQ ID NO: 1119 - RNA, 32 nt, synthetic construct
  source 1..32: mol_type = other RNA
ttggcaaacattcactcctttaacaaaagtca

SEQ ID NO: 1120 - RNA, 23 nt, synthetic construct
  source 1..23: mol_type = other RNA
ttggcaaacattcactccttctt

SEQ ID NO: 1121 - RNA, 23 nt, synthetic construct
  source 1..23: mol_type = other RNA
ttggcaaacattcactccttcga

SEQ ID NO: 1122 - RNA, 21 nt, synthetic construct
  source 1..21: mol_type = other RNA
ttggcaaacattcactccttc

SEQ ID NO: 1123 - RNA, 21 nt, synthetic construct
  source 1..21: mol_type = other RNA
ttggcaaacattcactccttc

SEQ ID NO: 1124 - RNA, 21 nt, synthetic construct
  source 1..21: mol_type = other RNA
ttggcaaacattcactccttc

SEQ ID NO: 1125 - RNA, 21 nt, synthetic construct
  source 1..21: mol_type = other RNA
ttggcaaacattcactccttc

SEQ ID NO: 1126 - RNA, 21 nt, synthetic construct
  source 1..21: mol_type = other RNA
tcggcaaacattcactccttc

SEQ ID NO: 1127 - RNA, 21 nt, synthetic construct
  source 1..21: mol_type = other RNA
ttggcaaacattcactccttc

SEQ ID NO: 1128 - RNA, 21 nt, synthetic construct
  source 1..21: mol_type = other RNA
tagtgaaaaactgacagcgaa

SEQ ID NO: 1129 - RNA, 21 nt, synthetic construct
  source 1..21: mol_type = other RNA
taagtgaaaaactgacagcga

SEQ ID NO: 1130 - RNA, 21 nt, synthetic construct
  source 1..21: mol_type = other RNA
ttaagtgaaaaactgacagcg

SEQ ID NO: 1131 - RNA, 21 nt, synthetic construct
  source 1..21: mol_type = other RNA
tataagtgaaaaactgacagc

SEQ ID NO: 1132 - RNA, 21 nt, synthetic construct
  source 1..21: mol_type = other RNA
taataagtgaaaaactgacag

SEQ ID NO: 1133 - RNA, 21 nt, synthetic construct
  source 1..21: mol_type = other RNA
tagaataagtgaaaaactgac

SEQ ID NO: 1134 - RNA, 21 nt, synthetic construct
  source 1..21: mol_type = other RNA
taagaataagtgaaaaactga

SEQ ID NO: 1135 - RNA, 21 nt, synthetic construct
  source 1..21: mol_type = other RNA
taaagaataagtgaaaaactg

SEQ ID NO: 1136 - RNA, 21 nt, synthetic construct
  source 1..21: mol_type = other RNA
ttaaagaataagtgaaaaact

SEQ ID NO: 1137 - RNA, 21 nt, synthetic construct
  source 1..21: mol_type = other RNA
tgtaaagaataagtgaaaaac

SEQ ID NO: 1138 - RNA, 21 nt, synthetic construct
  source 1..21: mol_type = other RNA
tgtgagaaaggtgtatctttt

SEQ ID NO: 1139 - RNA, 21 nt, synthetic construct
  source 1..21: mol_type = other RNA
ttgtgagaaaggtgtatcttt

SEQ ID NO: 1140 - RNA, 21 nt, synthetic construct
  source 1..21: mol_type = other RNA
tttgtgagaaaggtgtatctt

SEQ ID NO: 1141 - RNA, 21 nt, synthetic construct
  source 1..21: mol_type = other RNA
ttttgtgagaaaggtgtatct

SEQ ID NO: 1142 - RNA, 21 nt, synthetic construct
  source 1..21: mol_type = other RNA
tatttgtgagaaaggtgtatc

SEQ ID NO: 1143 - RNA, 21 nt, synthetic construct
  source 1..21: mol_type = other RNA
tttatttgtgagaaaggtgta

SEQ ID NO: 1144 - RNA, 21 nt, synthetic construct
  source 1..21: mol_type = other RNA
ttttatttgtgagaaaggtgt

SEQ ID NO: 1145 - RNA, 21 nt, synthetic construct
  source 1..21: mol_type = other RNA
tttttatttgtgagaaaggtg

SEQ ID NO: 1146 - RNA, 21 nt, synthetic construct
  source 1..21: mol_type = other RNA
tcttttatttgtgagaaaggt

SEQ ID NO: 1147 - RNA, 21 nt, synthetic construct
  source 1..21: mol_type = other RNA
ttcttttatttgtgagaaagg

SEQ ID NO: 1148 - RNA, 21 nt, synthetic construct
  source 1..21: mol_type = other RNA
taaaactgacagcgaatcatc

SEQ ID NO: 1149 - RNA, 21 nt, synthetic construct
  source 1..21: mol_type = other RNA
taaaaactgacagcgaatcat

SEQ ID NO: 1150 - RNA, 21 nt, synthetic construct
  source 1..21: mol_type = other RNA
tgaaaaactgacagcgaatca

SEQ ID NO: 1151 - RNA, 21 nt, synthetic construct
  source 1..21: mol_type = other RNA
ttgaaaaactgacagcgaatc

SEQ ID NO: 1152 - RNA, 21 nt, synthetic construct
  source 1..21: mol_type = other RNA
tgtgaaaaactgacagcgaat

SEQ ID NO: 1153 - RNA, 21 nt, synthetic construct
  source 1..21: mol_type = other RNA
taagtgaaaaactgacagcga

SEQ ID NO: 1154 - RNA, 21 nt, synthetic construct
  source 1..21: mol_type = other RNA
ttaagtgaaaaactgacagcg

SEQ ID NO: 1155 - RNA, 21 nt, synthetic construct
  source 1..21: mol_type = other RNA
tataagtgaaaaactgacagc

SEQ ID NO: 1156 - RNA, 21 nt, synthetic construct
  source 1..21: mol_type = other RNA
taataagtgaaaaactgacag

SEQ ID NO: 1157 - RNA, 21 nt, synthetic construct
  source 1..21: mol_type = other RNA
tgaataagtgaaaaactgaca

SEQ ID NO: 1158 - RNA, 21 nt, synthetic construct
  source 1..21: mol_type = other RNA
ttcaaaggagcctggagtttt

SEQ ID NO: 1159 - RNA, 21 nt, synthetic construct
  source 1..21: mol_type = other RNA
tttcaaaggagcctggagttt

SEQ ID NO: 1160 - RNA, 21 nt, synthetic construct
  source 1..21: mol_type = other RNA
tattcaaaggagcctggagtt

SEQ ID NO: 1161 - RNA, 21 nt, synthetic construct
  source 1..21: mol_type = other RNA
taattcaaaggagcctggagt

SEQ ID NO: 1162 - RNA, 21 nt, synthetic construct
  source 1..21: mol_type = other RNA
ttaattcaaaggagcctggag

SEQ ID NO: 1163 - RNA, 21 nt, synthetic construct
  source 1..21: mol_type = other RNA
ttgtaattcaaaggagcctgg

SEQ ID NO: 1164 - RNA, 21 nt, synthetic construct
  source 1..21: mol_type = other RNA
tgtgtaattcaaaggagcctg

SEQ ID NO: 1165 - RNA, 21 nt, synthetic construct
  source 1..21: mol_type = other RNA
tagtgtaattcaaaggagcct

SEQ ID NO: 1166 - RNA, 21 nt, synthetic construct
  source 1..21: mol_type = other RNA
tcagtgtaattcaaaggagcc

SEQ ID NO: 1167 - RNA, 21 nt, synthetic construct
  source 1..21: mol_type = other RNA
ttcagtgtaattcaaaggagc

SEQ ID NO: 1168 - RNA, 21 nt, synthetic construct
  source 1..21: mol_type = other RNA
taacattcactcctttaacaa

SEQ ID NO: 1169 - RNA, 21 nt, synthetic construct
  source 1..21: mol_type = other RNA
taaacattcactcctttaaca

SEQ ID NO: 1170 - RNA, 21 nt, synthetic construct
  source 1..21: mol_type = other RNA
tcaaacattcactcctttaac

SEQ ID NO: 1171 - RNA, 21 nt, synthetic construct
  source 1..21: mol_type = other RNA
tgcaaacattcactcctttaa

SEQ ID NO: 1172 - RNA, 21 nt, synthetic construct
  source 1..21: mol_type = other RNA
tggcaaacattcactccttta

SEQ ID NO: 1173 - RNA, 21 nt, synthetic construct
  source 1..21: mol_type = other RNA
tttggcaaacattcactcctt

SEQ ID NO: 1174 - RNA, 21 nt, synthetic construct
  source 1..21: mol_type = other RNA
tcttggcaaacattcactcct

SEQ ID NO: 1175 - RNA, 21 nt, synthetic construct
  source 1..21: mol_type = other RNA
ttcttggcaaacattcactcc

SEQ ID NO: 1176 - RNA, 21 nt, synthetic construct
  source 1..21: mol_type = other RNA
tctcttggcaaacattcactc

SEQ ID NO: 1177 - RNA, 21 nt, synthetic construct
  source 1..21: mol_type = other RNA
ttctcttggcaaacattcact

SEQ ID NO: 1178 - RNA, 21 nt, synthetic construct
  source 1..21: mol_type = other RNA
tgtcagtttttcacttattca

SEQ ID NO: 1179 - RNA, 21 nt, synthetic construct
  source 1..21: mol_type = other RNA
tgtcagtttttcacttattct

SEQ ID NO: 1180 - RNA, 21 nt, synthetic construct
  source 1..21: mol_type = other RNA
tgtcagtttttcacttattcc

SEQ ID NO: 1181 - RNA, 21 nt, synthetic construct
  source 1..21: mol_type = other RNA
tgtcagtttttcacttattcg

SEQ ID NO: 1182 - RNA, 17 nt, synthetic construct
  source 1..17: mol_type = other RNA
agtttttcacttattca

SEQ ID NO: 1183 - RNA, 18 nt, synthetic construct
  source 1..18: mol_type = other RNA
cagtttttcacttattca

SEQ ID NO: 1184 - RNA, 19 nt, synthetic construct
  source 1..19: mol_type = other RNA
acagtttttcacttattca

SEQ ID NO: 1185 - RNA, 19 nt, synthetic construct
  source 1..19: mol_type = other RNA
tcagtttttcacttattca

SEQ ID NO: 1186 - RNA, 19 nt, synthetic construct
  source 1..19: mol_type = other RNA
gcagtttttcacttattca

SEQ ID NO: 1187 - RNA, 19 nt, synthetic construct
  source 1..19: mol_type = other RNA
ccagtttttcacttattca

SEQ ID NO: 1188 - RNA, 20 nt, synthetic construct
  source 1..20: mol_type = other RNA
atcagtttttcacttattca

SEQ ID NO: 1189 - RNA, 20 nt, synthetic construct
  source 1..20: mol_type = other RNA
ttcagtttttcacttattca

SEQ ID NO: 1190 - RNA, 20 nt, synthetic construct
  source 1..20: mol_type = other RNA
gtcagtttttcacttattca

SEQ ID NO: 1191 - RNA, 20 nt, synthetic construct
  source 1..20: mol_type = other RNA
ctcagtttttcacttattca

SEQ ID NO: 1192 - RNA, 21 nt, synthetic construct
  source 1..21: mol_type = other RNA
ggtcagtttttcacttattca

SEQ ID NO: 1193 - RNA, 21 nt, synthetic construct
  source 1..21: mol_type = other RNA
agtcagtttttcacttattca

SEQ ID NO: 1194 - RNA, 21 nt, synthetic construct
  source 1..21: mol_type = other RNA
cgtcagtttttcacttattca

SEQ ID NO: 1195 - RNA, 22 nt, synthetic construct
  source 1..22: mol_type = other RNA
ctgtcagtttttcacttattca

SEQ ID NO: 1196 - RNA, 23 nt, synthetic construct
  source 1..23: mol_type = other RNA
gctgtcagtttttcacttattca

SEQ ID NO: 1197 - RNA, 24 nt, synthetic construct
  source 1..24: mol_type = other RNA
cgctgtcagtttttcacttattca

SEQ ID NO: 1198 - RNA, 25 nt, synthetic construct
  source 1..25: mol_type = other RNA
tcgctgtcagtttttcacttattca

SEQ ID NO: 1199 - RNA, 26 nt, synthetic construct
  source 1..26: mol_type = other RNA
ttcgctgtcagtttttcacttattca

SEQ ID NO: 1200 - RNA, 32 nt, synthetic construct; identical to SEQ ID NO: 790
  source 1..32: mol_type = other RNA

SEQ ID NO: 1201 - RNA, 21 nt, synthetic construct
  source 1..21: mol_type = other RNA
tgtcagtttttcacttattca

SEQ ID NO: 1202 - RNA, 21 nt, synthetic construct
  source 1..21: mol_type = other RNA
tgtcagtttttcacttattca

SEQ ID NO: 1203 - RNA, 21 nt, synthetic construct
  source 1..21: mol_type = other RNA
tgtcagtttttcacttattca

SEQ ID NO: 1204 - RNA, 21 nt, synthetic construct
  source 1..21: mol_type = other RNA
tgtcagtttttcacttattca

SEQ ID NO: 1205 - RNA, 21 nt, synthetic construct
  source 1..21: mol_type = other RNA
tgtcagtttttcacttattca

SEQ ID NO: 1206 - RNA, 21 nt, synthetic construct
  source 1..21: mol_type = other RNA
tgtcagtttttcactcattca

SEQ ID NO: 1207 - RNA, 21 nt, synthetic construct
  source 1..21: mol_type = other RNA
tgtcagtttttcacttattta

SEQ ID NO: 1208 - RNA, 21 nt, synthetic construct
  source 1..21: mol_type = other RNA
tgtcagtttttcacttattca

SEQ ID NO: 1209 - RNA, 21 nt, synthetic construct
  source 1..21: mol_type = other RNA
atacacctttctcacaaataa

SEQ ID NO: 1210 - RNA, 21 nt, synthetic construct
  source 1..21: mol_type = other RNA
atacacctttctcacaaatat

SEQ ID NO: 1211 - RNA, 21 nt, synthetic construct
  source 1..21: mol_type = other RNA
atacacctttctcacaaatac

SEQ ID NO: 1212 - RNA, 21 nt, synthetic construct
  source 1..21: mol_type = other RNA
atacacctttctcacaaatag

SEQ ID NO: 1213 - RNA, 17 nt, synthetic construct
  source 1..17: mol_type = other RNA
acctttctcacaaataa

SEQ ID NO: 1214 - RNA, 18 nt, synthetic construct
  source 1..18: mol_type = other RNA
cacctttctcacaaataa

SEQ ID NO: 1215 - RNA, 19 nt, synthetic construct
  source 1..19: mol_type = other RNA
acacctttctcacaaataa

SEQ ID NO: 1216 - RNA, 19 nt, synthetic construct
  source 1..19: mol_type = other RNA
tcacctttctcacaaataa

SEQ ID NO: 1217 - RNA, 19 nt, synthetic construct
  source 1..19: mol_type = other RNA
gcacctttctcacaaataa

SEQ ID NO: 1218 - RNA, 19 nt, synthetic construct
  source 1..19: mol_type = other RNA
ccacctttctcacaaataa

SEQ ID NO: 1219 - RNA, 20 nt, synthetic construct
  source 1..20: mol_type = other RNA
aacacctttctcacaaataa

SEQ ID NO: 1220 - RNA, 20 nt, synthetic construct
  source 1..20: mol_type = other RNA
tacacctttctcacaaataa

SEQ ID NO: 1221 - RNA, 20 nt, synthetic construct
  source 1..20: mol_type = other RNA
gacacctttctcacaaataa

SEQ ID NO: 1222 - RNA, 20 nt, synthetic construct
  source 1..20: mol_type = other RNA
cacacctttctcacaaataa

SEQ ID NO: 1223 - RNA, 21 nt, synthetic construct
  source 1..21: mol_type = other RNA
gtacacctttctcacaaataa

SEQ ID NO: 1224 - RNA, 21 nt, synthetic construct
  source 1..21: mol_type = other RNA
ctacacctttctcacaaataa

SEQ ID NO: 1225 - RNA, 21 nt, synthetic construct
  source 1..21: mol_type = other RNA
ttacacctttctcacaaataa

SEQ ID NO: 1226 - RNA, 22 nt, synthetic construct
  source 1..22: mol_type = other RNA
gatacacctttctcacaaataa

SEQ ID NO: 1227 - RNA, 23 nt, synthetic construct
  source 1..23: mol_type = other RNA
agatacacctttctcacaaataa

SEQ ID NO: 1228 - RNA, 24 nt, synthetic construct
  source 1..24: mol_type = other RNA
aagatacacctttctcacaaataa

SEQ ID NO: 1229 - RNA, 25 nt, synthetic construct
  source 1..25: mol_type = other RNA
aaagatacacctttctcacaaataa

SEQ ID NO: 1230 - RNA, 26 nt, synthetic construct
  source 1..26: mol_type = other RNA
aaaagatacacctttctcacaaataa

SEQ ID NO: 1231 - RNA, 32 nt, synthetic construct; identical to SEQ ID NO: 821
  source 1..32: mol_type = other RNA

SEQ ID NO: 1232 - RNA, 21 nt, synthetic construct
  source 1..21: mol_type = other RNA
atacacctttctcacaaataa

SEQ ID NO: 1233 - RNA, 21 nt, synthetic construct
  source 1..21: mol_type = other RNA
atacacctttctcacaaataa

SEQ ID NO: 1234 - RNA, 21 nt, synthetic construct
  source 1..21: mol_type = other RNA
atacacctttctcacaaataa

SEQ ID NO: 1235 - RNA, 21 nt, synthetic construct
  source 1..21: mol_type = other RNA
atacacctttctcacaaataa

SEQ ID NO: 1236 - RNA, 21 nt, synthetic construct
  source 1..21: mol_type = other RNA
atacacctttctcacaaataa

SEQ ID NO: 1237 - RNA, 21 nt, synthetic construct
  source 1..21: mol_type = other RNA
atacacctttctcacgaataa

SEQ ID NO: 1238 - RNA, 21 nt, synthetic construct
  source 1..21: mol_type = other RNA
atacacctttctcacaaatga

SEQ ID NO: 1239 - RNA, 21 nt, synthetic construct
  source 1..21: mol_type = other RNA
atacacctttctcacaaataa

SEQ ID NO: 1240 - RNA, 21 nt, synthetic construct
  source 1..21: mol_type = other RNA
ttcgctgtcagtttttcacta

SEQ ID NO: 1241 - RNA, 21 nt, synthetic construct
  source 1..21: mol_type = other RNA
ttcgctgtcagtttttcactt

SEQ ID NO: 1242 - RNA, 21 nt, synthetic construct
  source 1..21: mol_type = other RNA
ttcgctgtcagtttttcactc

SEQ ID NO: 1243 - RNA, 21 nt, synthetic construct
  source 1..21: mol_type = other RNA
ttcgctgtcagtttttcactg

SEQ ID NO: 1244 - RNA, 17 nt, synthetic construct
  source 1..17: mol_type = other RNA
ctgtcagtttttcacta

SEQ ID NO: 1245 - RNA, 18 nt, synthetic construct
  source 1..18: mol_type = other RNA
gctgtcagtttttcacta

SEQ ID NO: 1246 - RNA, 19 nt, synthetic construct
  source 1..19: mol_type = other RNA
agctgtcagtttttcacta

SEQ ID NO: 1247 - RNA, 19 nt, synthetic construct
  source 1..19: mol_type = other RNA
tgctgtcagtttttcacta

SEQ ID NO: 1248 - RNA, 19 nt, synthetic construct
  source 1..19: mol_type = other RNA
ggctgtcagtttttcacta

SEQ ID NO: 1249 - RNA, 19 nt, synthetic construct
  source 1..19: mol_type = other RNA
cgctgtcagtttttcacta

SEQ ID NO: 1250 - RNA, 20 nt, synthetic construct
  source 1..20: mol_type = other RNA
acgctgtcagtttttcacta

SEQ ID NO: 1251 - RNA, 20 nt, synthetic construct
  source 1..20: mol_type = other RNA
tcgctgtcagtttttcacta

SEQ ID NO: 1252 - RNA, 20 nt, synthetic construct
  source 1..20: mol_type = other RNA
gcgctgtcagtttttcacta

SEQ ID NO: 1253 - RNA, 20 nt, synthetic construct
  source 1..20: mol_type = other RNA
ccgctgtcagtttttcacta

SEQ ID NO: 1254 - RNA, 21 nt, synthetic construct
  source 1..21: mol_type = other RNA
gtcgctgtcagtttttcacta

SEQ ID NO: 1255 - RNA, 21 nt, synthetic construct
  source 1..21: mol_type = other RNA
ctcgctgtcagtttttcacta

SEQ ID NO: 1256 - RNA, 21 nt, synthetic construct
  source 1..21: mol_type = other RNA
atcgctgtcagtttttcacta

SEQ ID NO: 1257 - RNA, 22 nt, synthetic construct
  source 1..22: mol_type = other RNA
attcgctgtcagtttttcacta

SEQ ID NO: 1258 - RNA, 23 nt, synthetic construct
  source 1..23: mol_type = other RNA
gattcgctgtcagtttttcacta

SEQ ID NO: 1259 - RNA, 24 nt, synthetic construct
  source 1..24: mol_type = other RNA
tgattcgctgtcagtttttcacta

SEQ ID NO: 1260 - RNA, 25 nt, synthetic construct
  source 1..25: mol_type = other RNA
atgattcgctgtcagtttttcacta

SEQ ID NO: 1261 - RNA, 26 nt, synthetic construct
  source 1..26: mol_type = other RNA
gatgattcgctgtcagtttttcacta

SEQ ID NO: 1262 - RNA, 32 nt, synthetic construct; identical to SEQ ID NO: 852
  source 1..32: mol_type = other RNA

SEQ ID NO: 1263 - RNA, 21 nt, synthetic construct
  source 1..21: mol_type = other RNA
ttcgctgtcagtttttcacta

SEQ ID NO: 1264 - RNA, 21 nt, synthetic construct
  source 1..21: mol_type = other RNA
ttcgctgtcagtttttcacta

SEQ ID NO: 1265 - RNA, 21 nt, synthetic construct
  source 1..21: mol_type = other RNA
ttcgctgtcagtttttcacta

SEQ ID NO: 1266 - RNA, 21 nt, synthetic construct
  source 1..21: mol_type = other RNA
ttcgctgtcagtttttcacta

SEQ ID NO: 1267 - RNA, 21 nt, synthetic construct
  source 1..21: mol_type = other RNA
ttcgctgtcagtttttcacta

SEQ ID NO: 1268 - RNA, 21 nt, synthetic construct
  source 1..21: mol_type = other RNA
ttcgctgtcagttttccacta

SEQ ID NO: 1269 - RNA, 21 nt, synthetic construct
  source 1..21: mol_type = other RNA
ttcgctgtcagtttttcacca

SEQ ID NO: 1270 - RNA, 21 nt, synthetic construct
  source 1..21: mol_type = other RNA
ttcgctgtcagtttttcacta

SEQ ID NO: 1271 - RNA, 21 nt, synthetic construct
  source 1..21: mol_type = other RNA
tccaggctcctttgaattaca

SEQ ID NO: 1272 - RNA, 21 nt, synthetic construct
  source 1..21: mol_type = other RNA
tccaggctcctttgaattact

SEQ ID NO: 1273 - RNA, 21 nt, synthetic construct
  source 1..21: mol_type = other RNA
tccaggctcctttgaattacc

SEQ ID NO: 1274 - RNA, 21 nt, synthetic construct
  source 1..21: mol_type = other RNA
tccaggctcctttgaattacg

SEQ ID NO: 1275 - RNA, 17 nt, synthetic construct
  source 1..17: mol_type = other RNA
ggctcctttgaattaca

SEQ ID NO: 1276 - RNA, 18 nt, synthetic construct
  source 1..18: mol_type = other RNA
aggctcctttgaattaca

SEQ ID NO: 1277 - RNA, 19 nt, synthetic construct
  source 1..19: mol_type = other RNA
aaggctcctttgaattaca

SEQ ID NO: 1278 - RNA, 19 nt, synthetic construct
  source 1..19: mol_type = other RNA
taggctcctttgaattaca

SEQ ID NO: 1279 - RNA, 19 nt, synthetic construct
  source 1..19: mol_type = other RNA
gaggctcctttgaattaca

SEQ ID NO: 1280 - RNA, 19 nt, synthetic construct
  source 1..19: mol_type = other RNA
caggctcctttgaattaca

SEQ ID NO: 1281 - RNA, 20 nt, synthetic construct
  source 1..20: mol_type = other RNA
acaggctcctttgaattaca

SEQ ID NO: 1282 - RNA, 20 nt, synthetic construct
  source 1..20: mol_type = other RNA
tcaggctcctttgaattaca

SEQ ID NO: 1283 - RNA, 20 nt, synthetic construct
  source 1..20: mol_type = other RNA
gcaggctcctttgaattaca

SEQ ID NO: 1284 - RNA, 20 nt, synthetic construct
  source 1..20: mol_type = other RNA
ccaggctcctttgaattaca

SEQ ID NO: 1285 - RNA, 21 nt, synthetic construct
  source 1..21: mol_type = other RNA
gccaggctcctttgaattaca

SEQ ID NO: 1286 - RNA, 21 nt, synthetic construct
  source 1..21: mol_type = other RNA
cccaggctcctttgaattaca

SEQ ID NO: 1287 - RNA, 21 nt, synthetic construct
  source 1..21: mol_type = other RNA
accaggctcctttgaattaca

SEQ ID NO: 1288 - RNA, 22 nt, synthetic construct
  source 1..22: mol_type = other RNA
ctccaggctcctttgaattaca

SEQ ID NO: 1289 - RNA, 23 nt, synthetic construct
  source 1..23: mol_type = other RNA
actccaggctcctttgaattaca

SEQ ID NO: 1290 - RNA, 24 nt, synthetic construct
  source 1..24: mol_type = other RNA
aactccaggctcctttgaattaca

SEQ ID NO: 1291 - RNA, 25 nt, synthetic construct
  source 1..25: mol_type = other RNA
aaactccaggctcctttgaattaca

SEQ ID NO: 1292 - RNA, 26 nt, synthetic construct
  source 1..26: mol_type = other RNA
aaaactccaggctcctttgaattaca

SEQ ID NO: 1293 - RNA, 32 nt, synthetic construct; identical to SEQ ID NO: 883
  source 1..32: mol_type = other RNA

SEQ ID NO: 1294 - RNA, 21 nt, synthetic construct
  source 1..21: mol_type = other RNA
tccaggctcctttgaattaca

SEQ ID NO: 1295 - RNA, 21 nt, synthetic construct
  source 1..21: mol_type = other RNA
tccaggctcctttgaattaca

SEQ ID NO: 1296 - RNA, 21 nt, synthetic construct
  source 1..21: mol_type = other RNA
tccaggctcctttgaattaca

SEQ ID NO: 1297 - RNA, 21 nt, synthetic construct
  source 1..21: mol_type = other RNA
tccaggctcctttgaattaca

SEQ ID NO: 1298 - RNA, 21 nt, synthetic construct
  source 1..21: mol_type = other RNA
tccaggctcctttgaattaca

SEQ ID NO: 1299 - RNA, 21 nt, synthetic construct
  source 1..21: mol_type = other RNA
tccaggctcctttgagttaca

SEQ ID NO: 1300 - RNA, 21 nt, synthetic construct
  source 1..21: mol_type = other RNA
tccaggctcctttgaattata

SEQ ID NO: 1301 - RNA, 21 nt, synthetic construct
  source 1..21: mol_type = other RNA
tccaggctcctttgaattaca

SEQ ID NO: 1302 - RNA, 21 nt, synthetic construct
  source 1..21: mol_type = other RNA
gaaggagtgaatgtttgccaa

SEQ ID NO: 1303 - RNA, 21 nt, synthetic construct
  source 1..21: mol_type = other RNA
gaaggagtgaatgtttgccat

SEQ ID NO: 1304 - RNA, 21 nt, synthetic construct
  source 1..21: mol_type = other RNA
gaaggagtgaatgtttgccac

SEQ ID NO: 1305 - RNA, 21 nt, synthetic construct
  source 1..21: mol_type = other RNA
gaaggagtgaatgtttgccag

SEQ ID NO: 1306 - RNA, 17 nt, synthetic construct
  source 1..17: mol_type = other RNA
gagtgaatgtttgccaa

SEQ ID NO: 1307 - RNA, 18 nt, synthetic construct
  source 1..18: mol_type = other RNA
ggagtgaatgtttgccaa

SEQ ID NO: 1308 - RNA, 19 nt, synthetic construct
  source 1..19: mol_type = other RNA
aggagtgaatgtttgccaa

SEQ ID NO: 1309 - RNA, 19 nt, synthetic construct
  source 1..19: mol_type = other RNA
tggagtgaatgtttgccaa

SEQ ID NO: 1310 - RNA, 19 nt, synthetic construct
  source 1..19: mol_type = other RNA
gggagtgaatgtttgccaa

SEQ ID NO: 1311 - RNA, 19 nt, synthetic construct
  source 1..19: mol_type = other RNA
cggagtgaatgtttgccaa

SEQ ID NO: 1312 - RNA, 20 nt, synthetic construct
  source 1..20: mol_type = other RNA
aaggagtgaatgtttgccaa

SEQ ID NO: 1313 - RNA, 20 nt, synthetic construct
  source 1..20: mol_type = other RNA
taggagtgaatgtttgccaa

SEQ ID NO: 1314 - RNA, 20 nt, synthetic construct
  source 1..20: mol_type = other RNA
gaggagtgaatgtttgccaa

SEQ ID NO: 1315 - RNA, 20 nt, synthetic construct
  source 1..20: mol_type = other RNA
caggagtgaatgtttgccaa

SEQ ID NO: 1316 - RNA, 21 nt, synthetic construct
  source 1..21: mol_type = other RNA
taaggagtgaatgtttgccaa

SEQ ID NO: 1317 - RNA, 21 nt, synthetic construct
  source 1..21: mol_type = other RNA
caaggagtgaatgtttgccaa

SEQ ID NO: 1318 - RNA, 21 nt, synthetic construct
  source 1..21: mol_type = other RNA
aaaggagtgaatgtttgccaa

SEQ ID NO: 1319 - RNA, 22 nt, synthetic construct
  source 1..22: mol_type = other RNA
taaaggagtgaatgtttgccaa

SEQ ID NO: 1320 - RNA, 23 nt, synthetic construct
  source 1..23: mol_type = other RNA
ttaaaggagtgaatgtttgccaa

SEQ ID NO: 1321 - RNA, 24 nt, synthetic construct
  source 1..24: mol_type = other RNA
gttaaaggagtgaatgtttgccaa

SEQ ID NO: 1322 - RNA, 25 nt, synthetic construct
  source 1..25: mol_type = other RNA
tgttaaaggagtgaatgtttgccaa

SEQ ID NO: 1323 - RNA, 26 nt, synthetic construct
  source 1..26: mol_type = other RNA
ttgttaaaggagtgaatgtttgccaa

SEQ ID NO: 1324 - RNA, 32 nt, synthetic construct; identical to SEQ ID NO: 914
  source 1..32: mol_type = other RNA

SEQ ID NO: 1325 - RNA, 21 nt, synthetic construct
  source 1..21: mol_type = other RNA
gaaggagtgaatgtttgccaa

SEQ ID NO: 1326 - RNA, 21 nt, synthetic construct
  source 1..21: mol_type = other RNA
gaaggagtgaatgtttgccaa

SEQ ID NO: 1327 - RNA, 21 nt, synthetic construct
  source 1..21: mol_type = other RNA
gaaggagtgaatgtttgccaa

SEQ ID NO: 1328 - RNA, 21 nt, synthetic construct
  source 1..21: mol_type = other RNA
gaaggagtgaatgtttgccaa

SEQ ID NO: 1329 - RNA, 21 nt, synthetic construct
  source 1..21: mol_type = other RNA
gaaggagtgaatgtttgccaa

SEQ ID NO: 1330 - RNA, 21 nt, synthetic construct
  source 1..21: mol_type = other RNA
gaaggagtgaatgttcgccaa

SEQ ID NO: 1331 - RNA, 21 nt, synthetic construct
  source 1..21: mol_type = other RNA
gaaggagtgaatgtttgccga

SEQ ID NO: 1332 - RNA, 21 nt, synthetic construct
  source 1..21: mol_type = other RNA
gaaggagtgaatgtttgccaa

SEQ ID NO: 1333 - RNA, 21 nt, synthetic construct
  source 1..21: mol_type = other RNA
ttcgctgtcagtttttcacta

SEQ ID NO: 1334 - RNA, 21 nt, synthetic construct
  source 1..21: mol_type = other RNA
tcgctgtcagtttttcactta

SEQ ID NO: 1335 - RNA, 21 nt, synthetic construct
  source 1..21: mol_type = other RNA
cgctgtcagtttttcacttaa

SEQ ID NO: 1336 - RNA, 21 nt, synthetic construct
  source 1..21: mol_type = other RNA
gctgtcagtttttcacttata

SEQ ID NO: 1337 - RNA, 21 nt, synthetic construct
  source 1..21: mol_type = other RNA
ctgtcagtttttcacttatta

SEQ ID NO: 1338 - RNA, 21 nt, synthetic construct
  source 1..21: mol_type = other RNA
gtcagtttttcacttattcta

SEQ ID NO: 1339 - RNA, 21 nt, synthetic construct
  source 1..21: mol_type = other RNA
tcagtttttcacttattctta

SEQ ID NO: 1340 - RNA, 21 nt, synthetic construct
  source 1..21: mol_type = other RNA
cagtttttcacttattcttta

SEQ ID NO: 1341 - RNA, 21 nt, synthetic construct
  source 1..21: mol_type = other RNA
agtttttcacttattctttaa

SEQ ID NO: 1342 - RNA, 21 nt, synthetic construct
  source 1..21: mol_type = other RNA
gtttttcacttattctttaca

SEQ ID NO: 1343 - RNA, 21 nt, synthetic construct
  source 1..21: mol_type = other RNA
aaaagatacacctttctcaca

SEQ ID NO: 1344 - RNA, 21 nt, synthetic construct
  source 1..21: mol_type = other RNA
aaagatacacctttctcacaa

SEQ ID NO: 1345 - RNA, 21 nt, synthetic construct
  source 1..21: mol_type = other RNA
aagatacacctttctcacaaa

SEQ ID NO: 1346 - RNA, 21 nt, synthetic construct
  source 1..21: mol_type = other RNA
agatacacctttctcacaaaa

SEQ ID NO: 1347 - RNA, 21 nt, synthetic construct
  source 1..21: mol_type = other RNA
gatacacctttctcacaaata

SEQ ID NO: 1348 - RNA, 21 nt, synthetic construct
  source 1..21: mol_type = other RNA
tacacctttctcacaaataaa

SEQ ID NO: 1349 - RNA, 21 nt, synthetic construct
  source 1..21: mol_type = other RNA
acacctttctcacaaataaaa

SEQ ID NO: 1350 - RNA, 21 nt, synthetic construct
  source 1..21: mol_type = other RNA
cacctttctcacaaataaaaa

SEQ ID NO: 1351 - RNA, 21 nt, synthetic construct
  source 1..21: mol_type = other RNA
acctttctcacaaataaaaga

SEQ ID NO: 1352 - RNA, 21 nt, synthetic construct
  source 1..21: mol_type = other RNA
cctttctcacaaataaaagaa

SEQ ID NO: 1353 - RNA, 21 nt, synthetic construct
  source 1..21: mol_type = other RNA
gatgattcgctgtcagtttta

SEQ ID NO: 1354 - RNA, 21 nt, synthetic construct
  source 1..21: mol_type = other RNA
atgattcgctgtcagttttta

SEQ ID NO: 1355 - RNA, 21 nt, synthetic construct
  source 1..21: mol_type = other RNA
tgattcgctgtcagtttttca

SEQ ID NO: 1356 - RNA, 21 nt, synthetic construct
  source 1..21: mol_type = other RNA
gattcgctgtcagtttttcaa

SEQ ID NO: 1357 - RNA, 21 nt, synthetic construct
  source 1..21: mol_type = other RNA
attcgctgtcagtttttcaca

SEQ ID NO: 1358 - RNA, 21 nt, synthetic construct
  source 1..21: mol_type = other RNA
tcgctgtcagtttttcactta

SEQ ID NO: 1359 - RNA, 21 nt, synthetic construct
  source 1..21: mol_type = other RNA
cgctgtcagtttttcacttaa

SEQ ID NO: 1360 - RNA, 21 nt, synthetic construct
  source 1..21: mol_type = other RNA
gctgtcagtttttcacttata

SEQ ID NO: 1361 - RNA, 21 nt, synthetic construct
  source 1..21: mol_type = other RNA
ctgtcagtttttcacttatta

SEQ ID NO: 1362 - RNA, 21 nt, synthetic construct
  source 1..21: mol_type = other RNA
tgtcagtttttcacttattca

SEQ ID NO: 1363 - RNA, 21 nt, synthetic construct
  source 1..21: mol_type = other RNA
aaaactccaggctcctttgaa

SEQ ID NO: 1364 - RNA, 21 nt, synthetic construct
  source 1..21: mol_type = other RNA
aaactccaggctcctttgaaa

SEQ ID NO: 1365 - RNA, 21 nt, synthetic construct
  source 1..21: mol_type = other RNA
aactccaggctcctttgaata

SEQ ID NO: 1366 - RNA, 21 nt, synthetic construct
  source 1..21: mol_type = other RNA
actccaggctcctttgaatta

SEQ ID NO: 1367 - RNA, 21 nt, synthetic construct
  source 1..21: mol_type = other RNA
ctccaggctcctttgaattaa

SEQ ID NO: 1368 - RNA, 21 nt, synthetic construct
  source 1..21: mol_type = other RNA
ccaggctcctttgaattacaa

SEQ ID NO: 1369 - RNA, 21 nt, synthetic construct
  source 1..21: mol_type = other RNA
caggctcctttgaattacaca

SEQ ID NO: 1370 - RNA, 21 nt, synthetic construct
  source 1..21: mol_type = other RNA
aggctcctttgaattacacta

SEQ ID NO: 1371 - RNA, 21 nt, synthetic construct
  source 1..21: mol_type = other RNA
ggctcctttgaattacactga

SEQ ID NO: 1372 - RNA, 21 nt, synthetic construct
  source 1..21: mol_type = other RNA
gctcctttgaattacactgaa

SEQ ID NO: 1373 - RNA, 21 nt, synthetic construct
  source 1..21: mol_type = other RNA
ttgttaaaggagtgaatgtta

SEQ ID NO: 1374 - RNA, 21 nt, synthetic construct
  source 1..21: mol_type = other RNA
tgttaaaggagtgaatgttta

SEQ ID NO: 1375 - RNA, 21 nt, synthetic construct
  source 1..21: mol_type = other RNA
gttaaaggagtgaatgtttga

SEQ ID NO: 1376 - RNA, 21 nt, synthetic construct
  source 1..21: mol_type = other RNA
ttaaaggagtgaatgtttgca

SEQ ID NO: 1377 - RNA, 21 nt, synthetic construct
  source 1..21: mol_type = other RNA
taaaggagtgaatgtttgcca

SEQ ID NO: 1378 - RNA, 21 nt, synthetic construct
  source 1..21: mol_type = other RNA
aaggagtgaatgtttgccaaa

SEQ ID NO: 1379 - RNA, 21 nt, synthetic construct
  source 1..21: mol_type = other RNA
aggagtgaatgtttgccaaga

SEQ ID NO: 1380 - RNA, 21 nt, synthetic construct
  source 1..21: mol_type = other RNA
ggagtgaatgtttgccaagaa

SEQ ID NO: 1381 - RNA, 21 nt, synthetic construct
  source 1..21: mol_type = other RNA
gagtgaatgtttgccaagaga

SEQ ID NO: 1382 - RNA, 21 nt, synthetic construct
  source 1..21: mol_type = other RNA
agtgaatgtttgccaagagaa

SEQ ID NO: 1383 - RNA, 21 nt, synthetic construct
  source 1..21: mol_type = other RNA
tgaataagtgaaaaactgaca

SEQ ID NO: 1384 - RNA, 21 nt, synthetic construct
  source 1..21: mol_type = other RNA
agaataagtgaaaaactgaca

SEQ ID NO: 1385 - RNA, 21 nt, synthetic construct
  source 1..21: mol_type = other RNA
ggaataagtgaaaaactgaca

SEQ ID NO: 1386 - RNA, 21 nt, synthetic construct
  source 1..21: mol_type = other RNA
cgaataagtgaaaaactgaca

SEQ ID NO: 1387 - RNA, 17 nt, synthetic construct
  source 1..17: mol_type = other RNA
tgaataagtgaaaaact

SEQ ID NO: 1388 - RNA, 18 nt, synthetic construct
  source 1..18: mol_type = other RNA
tgaataagtgaaaaactg

SEQ ID NO: 1389 - RNA, 19 nt, synthetic construct
  source 1..19: mol_type = other RNA
tgaataagtgaaaaactgt

SEQ ID NO: 1390 - RNA, 19 nt, synthetic construct
  source 1..19: mol_type = other RNA
tgaataagtgaaaaactga

SEQ ID NO: 1391 - RNA, 19 nt, synthetic construct
  source 1..19: mol_type = other RNA
tgaataagtgaaaaactgc

SEQ ID NO: 1392 - RNA, 19 nt, synthetic construct
  source 1..19: mol_type = other RNA
tgaataagtgaaaaactgg

SEQ ID NO: 1393 - RNA, 20 nt, synthetic construct
  source 1..20: mol_type = other RNA
tgaataagtgaaaaactgat

SEQ ID NO: 1394 - RNA, 20 nt, synthetic construct
  source 1..20: mol_type = other RNA
tgaataagtgaaaaactgaa

SEQ ID NO: 1395 - RNA, 20 nt, synthetic construct
  source 1..20: mol_type = other RNA
tgaataagtgaaaaactgac

SEQ ID NO: 1396 - RNA, 20 nt, synthetic construct
  source 1..20: mol_type = other RNA
tgaataagtgaaaaactgag

SEQ ID NO: 1397 - RNA, 21 nt, synthetic construct
  source 1..21: mol_type = other RNA
tgaataagtgaaaaactgacc

SEQ ID NO: 1398 - RNA, 21 nt, synthetic construct
  source 1..21: mol_type = other RNA
tgaataagtgaaaaactgact

SEQ ID NO: 1399 - RNA, 21 nt, synthetic construct
  source 1..21: mol_type = other RNA
tgaataagtgaaaaactgacg

SEQ ID NO: 1400 - RNA, 22 nt, synthetic construct
  source 1..22: mol_type = other RNA
tgaataagtgaaaaactgacag

SEQ ID NO: 1401 - RNA, 23 nt, synthetic construct
  source 1..23: mol_type = other RNA
tgaataagtgaaaaactgacagc

SEQ ID NO: 1402 - RNA, 24 nt, synthetic construct
  source 1..24: mol_type = other RNA
tgaataagtgaaaaactgacagcg

SEQ ID NO: 1403 - RNA, 25 nt, synthetic construct
  source 1..25: mol_type = other RNA
tgaataagtgaaaaactgacagcga

SEQ ID NO: 1404 - RNA, 26 nt, synthetic construct
  source 1..26: mol_type = other RNA
tgaataagtgaaaaactgacagcgaa

SEQ ID NO: 1405 - RNA, 32 nt, synthetic construct; identical to SEQ ID NO: 995
  source 1..32: mol_type = other RNA

SEQ ID NO: 1406 - RNA, 23 nt, synthetic construct
  source 1..23: mol_type = other RNA
tgaataagtgaaaaactgacatt

SEQ ID NO: 1407 - RNA, 23 nt, synthetic construct
  source 1..23: mol_type = other RNA
tgaataagtgaaaaactgacaga

SEQ ID NO: 1408 - RNA, 21 nt, synthetic construct
  source 1..21: mol_type = other RNA
tgaataagtgaaaaactgaca

SEQ ID NO: 1409 - RNA, 21 nt, synthetic construct
  source 1..21: mol_type = other RNA
tgaataagtgaaaaactgaca

SEQ ID NO: 1410 - RNA, 21 nt, synthetic construct
  source 1..21: mol_type = other RNA
tgaataagtgaaaaactgaca

SEQ ID NO: 1411 - RNA, 21 nt, synthetic construct
  source 1..21: mol_type = other RNA
tgaataagtgaaaaactgaca

SEQ ID NO: 1412 - RNA, 21 nt, synthetic construct
  source 1..21: mol_type = other RNA
taaataagtgaaaaactgaca

SEQ ID NO: 1413 - RNA, 21 nt, synthetic construct
  source 1..21: mol_type = other RNA
tgaataagtgaaaaactgaca

SEQ ID NO: 1414 - RNA, 21 nt, synthetic construct
  source 1..21: mol_type = other RNA
ttatttgtgagaaaggtgtat

SEQ ID NO: 1415 - RNA, 21 nt, synthetic construct
  source 1..21: mol_type = other RNA
atatttgtgagaaaggtgtat

SEQ ID NO: 1416 - RNA, 21 nt, synthetic construct
  source 1..21: mol_type = other RNA
gtatttgtgagaaaggtgtat

SEQ ID NO: 1417 - RNA, 21 nt, synthetic construct
  source 1..21: mol_type = other RNA
ctatttgtgagaaaggtgtat

SEQ ID NO: 1418 - RNA, 17 nt, synthetic construct
  source 1..17: mol_type = other RNA
ttatttgtgagaaaggt

SEQ ID NO: 1419 - RNA, 18 nt, synthetic construct
  source 1..18: mol_type = other RNA
ttatttgtgagaaaggtg

SEQ ID NO: 1420 - RNA, 19 nt, synthetic construct
  source 1..19: mol_type = other RNA
ttatttgtgagaaaggtgt

SEQ ID NO: 1421 - RNA, 19 nt, synthetic construct
  source 1..19: mol_type = other RNA
ttatttgtgagaaaggtga

SEQ ID NO: 1422 - RNA, 19 nt, synthetic construct
  source 1..19: mol_type = other RNA
ttatttgtgagaaaggtgc

SEQ ID NO: 1423 - RNA, 19 nt, synthetic construct
  source 1..19: mol_type = other RNA
ttatttgtgagaaaggtgg

SEQ ID NO: 1424 - RNA, 20 nt, synthetic construct
  source 1..20: mol_type = other RNA
ttatttgtgagaaaggtgtt

SEQ ID NO: 1425 - RNA, 20 nt, synthetic construct
  source 1..20: mol_type = other RNA
ttatttgtgagaaaggtgta

SEQ ID NO: 1426 - RNA, 20 nt, synthetic construct
  source 1..20: mol_type = other RNA
ttatttgtgagaaaggtgtc

SEQ ID NO: 1427 - RNA, 20 nt, synthetic construct
  source 1..20: mol_type = other RNA
ttatttgtgagaaaggtgtg

SEQ ID NO: 1428 - RNA, 21 nt, synthetic construct
  source 1..21: mol_type = other RNA
ttatttgtgagaaaggtgtac

SEQ ID NO: 1429 - RNA, 21 nt, synthetic construct
  source 1..21: mol_type = other RNA
ttatttgtgagaaaggtgtag

SEQ ID NO: 1430 - RNA, 21 nt, synthetic construct
  source 1..21: mol_type = other RNA
ttatttgtgagaaaggtgtaa

SEQ ID NO: 1431 - RNA, 22 nt, synthetic construct
  source 1..22: mol_type = other RNA
ttatttgtgagaaaggtgtatc

SEQ ID NO: 1432 - RNA, 23 nt, synthetic construct
  source 1..23: mol_type = other RNA
ttatttgtgagaaaggtgtatct

SEQ ID NO: 1433 - RNA, 24 nt, synthetic construct
  source 1..24: mol_type = other RNA
ttatttgtgagaaaggtgtatctt

SEQ ID NO: 1434 - RNA, 25 nt, synthetic construct
  source 1..25: mol_type = other RNA
ttatttgtgagaaaggtgtatcttt

SEQ ID NO: 1435 - RNA, 26 nt, synthetic construct
  source 1..26: mol_type = other RNA
ttatttgtgagaaaggtgtatctttt

SEQ ID NO: 1436 - RNA, 32 nt, synthetic construct; identical to SEQ ID NO: 1026
  source 1..32: mol_type = other RNA

SEQ ID NO: 1437 - RNA, 23 nt, synthetic construct
  source 1..23: mol_type = other RNA
ttatttgtgagaaaggtgtattt

SEQ ID NO: 1438 - RNA, 23 nt, synthetic construct
  source 1..23: mol_type = other RNA
ttatttgtgagaaaggtgtatga

SEQ ID NO: 1439 - RNA, 21 nt, synthetic construct
  source 1..21: mol_type = other RNA
ttatttgtgagaaaggtgtat

SEQ ID NO: 1440 - RNA, 21 nt, synthetic construct
  source 1..21: mol_type = other RNA
ttatttgtgagaaaggtgtat

SEQ ID NO: 1441 - RNA, 21 nt, synthetic construct
  source 1..21: mol_type = other RNA
ttatttgtgagaaaggtgtat

SEQ ID NO: 1442 - RNA, 21 nt, synthetic construct
  source 1..21: mol_type = other RNA
ttatttgtgagaaaggtgtat

SEQ ID NO: 1443 - RNA, 21 nt, synthetic construct
  source 1..21: mol_type = other RNA
tcatttgtgagaaaggtgtat

SEQ ID NO: 1444 - RNA, 21 nt, synthetic construct
  source 1..21: mol_type = other RNA
ttatttgtgagaaaggtgtat

SEQ ID NO: 1445 - RNA, 21 nt, synthetic construct
  source 1..21: mol_type = other RNA
tagtgaaaaactgacagcgaa

SEQ ID NO: 1446 - RNA, 21 nt, synthetic construct
  source 1..21: mol_type = other RNA
aagtgaaaaactgacagcgaa

SEQ ID NO: 1447 - RNA, 21 nt, synthetic construct
  source 1..21: mol_type = other RNA
gagtgaaaaactgacagcgaa

SEQ ID NO: 1448 - RNA, 21 nt, synthetic construct
  source 1..21: mol_type = other RNA
cagtgaaaaactgacagcgaa

SEQ ID NO: 1449 - RNA, 17 nt, synthetic construct
  source 1..17: mol_type = other RNA
tagtgaaaaactgacag

SEQ ID NO: 1450 - RNA, 18 nt, synthetic construct
  source 1..18: mol_type = other RNA
tagtgaaaaactgacagc

SEQ ID NO: 1451 - RNA, 19 nt, synthetic construct
  source 1..19: mol_type = other RNA
tagtgaaaaactgacagct

SEQ ID NO: 1452 - RNA, 19 nt, synthetic construct
  source 1..19: mol_type = other RNA
tagtgaaaaactgacagca

SEQ ID NO: 1453 - RNA, 19 nt, synthetic construct
  source 1..19: mol_type = other RNA
tagtgaaaaactgacagcc

SEQ ID NO: 1454 - RNA, 19 nt, synthetic construct
  source 1..19: mol_type = other RNA
tagtgaaaaactgacagcg

SEQ ID NO: 1455 - RNA, 20 nt, synthetic construct
  source 1..20: mol_type = other RNA
tagtgaaaaactgacagcgt

SEQ ID NO: 1456 - RNA, 20 nt, synthetic construct
  source 1..20: mol_type = other RNA
tagtgaaaaactgacagcga

SEQ ID NO: 1457 - RNA, 20 nt, synthetic construct
  source 1..20: mol_type = other RNA
tagtgaaaaactgacagcgc

SEQ ID NO: 1458 - RNA, 20 nt, synthetic construct
  source 1..20: mol_type = other RNA
tagtgaaaaactgacagcgg

SEQ ID NO: 1459 - RNA, 21 nt, synthetic construct
  source 1..21: mol_type = other RNA
tagtgaaaaactgacagcgac

SEQ ID NO: 1460 - RNA, 21 nt, synthetic construct
  source 1..21: mol_type = other RNA
tagtgaaaaactgacagcgag

SEQ ID NO: 1461 - RNA, 21 nt, synthetic construct
  source 1..21: mol_type = other RNA
tagtgaaaaactgacagcgat

SEQ ID NO: 1462 - RNA, 22 nt, synthetic construct
  source 1..22: mol_type = other RNA
tagtgaaaaactgacagcgaat

SEQ ID NO: 1463 - RNA, 23 nt, synthetic construct
  source 1..23: mol_type = other RNA
tagtgaaaaactgacagcgaatc

SEQ ID NO: 1464 - RNA, 24 nt, synthetic construct
  source 1..24: mol_type = other RNA
tagtgaaaaactgacagcgaatca

SEQ ID NO: 1465 - RNA, 25 nt, synthetic construct
  source 1..25: mol_type = other RNA
tagtgaaaaactgacagcgaatcat

SEQ ID NO: 1466 - RNA, 26 nt, synthetic construct
  source 1..26: mol_type = other RNA
tagtgaaaaactgacagcgaatcatc

SEQ ID NO: 1467 - RNA, 32 nt, synthetic construct; identical to SEQ ID NO: 1057
  source 1..32: mol_type = other RNA

SEQ ID NO: 1468 - RNA, 23 nt, synthetic construct
  source 1..23: mol_type = other RNA
tagtgaaaaactgacagcgaatt

SEQ ID NO: 1469 - RNA, 23 nt, synthetic construct
  source 1..23: mol_type = other RNA
tagtgaaaaactgacagcgaaga

SEQ ID NO: 1470 - RNA, 21 nt, synthetic construct
  source 1..21: mol_type = other RNA
tagtgaaaaactgacagcgaa

SEQ ID NO: 1471 - RNA, 21 nt, synthetic construct
  source 1..21: mol_type = other RNA
tagtgaaaaactgacagcgaa

SEQ ID NO: 1472 - RNA, 21 nt, synthetic construct
  source 1..21: mol_type = other RNA
tagtgaaaaactgacagcgaa

SEQ ID NO: 1473 - RNA, 21 nt, synthetic construct
  source 1..21: mol_type = other RNA
tagtgaaaaactgacagcgaa

SEQ ID NO: 1474 - RNA, 21 nt, synthetic construct
  source 1..21: mol_type = other RNA
tggtgaaaaactgacagcgaa

SEQ ID NO: 1475 - RNA, 21 nt, synthetic construct
  source 1..21: mol_type = other RNA
tagtgaaaaactgacagcgaa

SEQ ID NO: 1476 - RNA, 21 nt, synthetic construct
  source 1..21: mol_type = other RNA
tgtaattcaaaggagcctgga

SEQ ID NO: 1477 - RNA, 21 nt, synthetic construct
  source 1..21: mol_type = other RNA
agtaattcaaaggagcctgga

SEQ ID NO: 1478 - RNA, 21 nt, synthetic construct
  source 1..21: mol_type = other RNA
ggtaattcaaaggagcctgga

SEQ ID NO: 1479 - RNA, 21 nt, synthetic construct
  source 1..21: mol_type = other RNA
cgtaattcaaaggagcctgga

SEQ ID NO: 1480 - RNA, 17 nt, synthetic construct
  source 1..17: mol_type = other RNA
tgtaattcaaaggagcc

SEQ ID NO: 1481 - RNA, 18 nt, synthetic construct
  source 1..18: mol_type = other RNA
tgtaattcaaaggagcct

SEQ ID NO: 1482 - RNA, 19 nt, synthetic construct
  source 1..19: mol_type = other RNA
tgtaattcaaaggagcctt

SEQ ID NO: 1483 - RNA, 19 nt, synthetic construct
  source 1..19: mol_type = other RNA
tgtaattcaaaggagccta

SEQ ID NO: 1484 - RNA, 19 nt, synthetic construct
  source 1..19: mol_type = other RNA
tgtaattcaaaggagcctc

SEQ ID NO: 1485 - RNA, 19 nt, synthetic construct
  source 1..19: mol_type = other RNA
tgtaattcaaaggagcctg

SEQ ID NO: 1486 - RNA, 20 nt, synthetic construct
  source 1..20: mol_type = other RNA
tgtaattcaaaggagcctgt

SEQ ID NO: 1487 - RNA, 20 nt, synthetic construct
  source 1..20: mol_type = other RNA
tgtaattcaaaggagcctga

SEQ ID NO: 1488 - RNA, 20 nt, synthetic construct
  source 1..20: mol_type = other RNA
tgtaattcaaaggagcctgc

SEQ ID NO: 1489 - RNA, 20 nt, synthetic construct
  source 1..20: mol_type = other RNA
tgtaattcaaaggagcctgg

SEQ ID NO: 1490 - RNA, 21 nt, synthetic construct
  source 1..21: mol_type = other RNA
tgtaattcaaaggagcctggc

SEQ ID NO: 1491 - RNA, 21 nt, synthetic construct
  source 1..21: mol_type = other RNA
tgtaattcaaaggagcctggg

SEQ ID NO: 1492 - RNA, 21 nt, synthetic construct
  source 1..21: mol_type = other RNA
tgtaattcaaaggagcctggt

SEQ ID NO: 1493 - RNA, 22 nt, synthetic construct
  source 1..22: mol_type = other RNA
tgtaattcaaaggagcctggag

SEQ ID NO: 1494 - RNA, 23 nt, synthetic construct
  source 1..23: mol_type = other RNA
tgtaattcaaaggagcctggagt

SEQ ID NO: 1495 - RNA, 24 nt, synthetic construct
  source 1..24: mol_type = other RNA
tgtaattcaaaggagcctggagtt

SEQ ID NO: 1496 - RNA, 25 nt, synthetic construct
  source 1..25: mol_type = other RNA
tgtaattcaaaggagcctggagttt

SEQ ID NO: 1497 - RNA, 26 nt, synthetic construct
  source 1..26: mol_type = other RNA
tgtaattcaaaggagcctggagtttt

SEQ ID NO: 1498 - RNA, 32 nt, synthetic construct; identical to SEQ ID NO: 1088
  source 1..32: mol_type = other RNA

SEQ ID NO: 1499 - RNA, 23 nt, synthetic construct
  source 1..23: mol_type = other RNA
tgtaattcaaaggagcctggatt

SEQ ID NO: 1500 - RNA, 23 nt, synthetic construct
  source 1..23: mol_type = other RNA
tgtaattcaaaggagcctggaga

SEQ ID NO: 1501 - RNA, 21 nt, synthetic construct
  source 1..21: mol_type = other RNA
tgtaattcaaaggagcctgga

SEQ ID NO: 1502 - RNA, 21 nt, synthetic construct
  source 1..21: mol_type = other RNA
tgtaattcaaaggagcctgga

SEQ ID NO: 1503 - RNA, 21 nt, synthetic construct
  source 1..21: mol_type = other RNA
tgtaattcaaaggagcctgga

SEQ ID NO: 1504 - RNA, 21 nt, synthetic construct
  source 1..21: mol_type = other RNA
tgtaattcaaaggagcctgga

SEQ ID NO: 1505 - RNA, 21 nt, synthetic construct
  source 1..21: mol_type = other RNA
tataattcaaaggagcctgga

SEQ ID NO: 1506 - RNA, 21 nt, synthetic construct
  source 1..21: mol_type = other RNA
tgtaattcaaaggagcctgga

SEQ ID NO: 1507 - RNA, 21 nt, synthetic construct
  source 1..21: mol_type = other RNA
ttggcaaacattcactccttc

SEQ ID NO: 1508 - RNA, 21 nt, synthetic construct
  source 1..21: mol_type = other RNA
atggcaaacattcactccttc

SEQ ID NO: 1509 - RNA, 21 nt, synthetic construct
  source 1..21: mol_type = other RNA
gtggcaaacattcactccttc

SEQ ID NO: 1510 - RNA, 21 nt, synthetic construct
  source 1..21: mol_type = other RNA
ctggcaaacattcactccttc

SEQ ID NO: 1511 - RNA, 17 nt, synthetic construct
  source 1..17: mol_type = other RNA
ttggcaaacattcactc

SEQ ID NO: 1512 - RNA, 18 nt, synthetic construct
  source 1..18: mol_type = other RNA
ttggcaaacattcactcc

SEQ ID NO: 1513 - RNA, 19 nt, synthetic construct
  source 1..19: mol_type = other RNA
ttggcaaacattcactcct

SEQ ID NO: 1514 - RNA, 19 nt, synthetic construct
  source 1..19: mol_type = other RNA
ttggcaaacattcactcca

SEQ ID NO: 1515 - RNA, 19 nt, synthetic construct
  source 1..19: mol_type = other RNA
ttggcaaacattcactccc

SEQ ID NO: 1516 - RNA, 19 nt, synthetic construct
  source 1..19: mol_type = other RNA
ttggcaaacattcactccg

SEQ ID NO: 1517 - RNA, 20 nt, synthetic construct
  source 1..20: mol_type = other RNA
ttggcaaacattcactcctt

SEQ ID NO: 1518 - RNA, 20 nt, synthetic construct
  source 1..20: mol_type = other RNA
ttggcaaacattcactccta

SEQ ID NO: 1519 - RNA, 20 nt, synthetic construct
  source 1..20: mol_type = other RNA
ttggcaaacattcactcctc

SEQ ID NO: 1520 - RNA, 20 nt, synthetic construct
  source 1..20: mol_type = other RNA
ttggcaaacattcactcctg

SEQ ID NO: 1521 - RNA, 21 nt, synthetic construct
  source 1..21: mol_type = other RNA
ttggcaaacattcactcctta

SEQ ID NO: 1522 - RNA, 21 nt, synthetic construct
  source 1..21: mol_type = other RNA
ttggcaaacattcactccttg

SEQ ID NO: 1523 - RNA, 21 nt, synthetic construct
  source 1..21: mol_type = other RNA
ttggcaaacattcactccttt

SEQ ID NO: 1524 - RNA, 22 nt, synthetic construct
  source 1..22: mol_type = other RNA
ttggcaaacattcactccttta

SEQ ID NO: 1525 - RNA, 23 nt, synthetic construct
  source 1..23: mol_type = other RNA
ttggcaaacattcactcctttaa

SEQ ID NO: 1526 - RNA, 24 nt, synthetic construct
  source 1..24: mol_type = other RNA
ttggcaaacattcactcctttaac

SEQ ID NO: 1527 - RNA, 25 nt, synthetic construct
  source 1..25: mol_type = other RNA
ttggcaaacattcactcctttaaca

SEQ ID NO: 1528 - RNA, 26 nt, synthetic construct
  source 1..26: mol_type = other RNA
ttggcaaacattcactcctttaacaa

SEQ ID NO: 1529 - RNA, 32 nt, synthetic construct; identical to SEQ ID NO: 1119
  source 1..32: mol_type = other RNA

SEQ ID NO: 1530 - RNA, 23 nt, synthetic construct
  source 1..23: mol_type = other RNA
ttggcaaacattcactccttctt

SEQ ID NO: 1531 - RNA, 23 nt, synthetic construct
  source 1..23: mol_type = other RNA
ttggcaaacattcactccttcga

SEQ ID NO: 1532 - RNA, 21 nt, synthetic construct
  source 1..21: mol_type = other RNA
ttggcaaacattcactccttc

SEQ ID NO: 1533 - RNA, 21 nt, synthetic construct
  source 1..21: mol_type = other RNA
ttggcaaacattcactccttc

SEQ ID NO: 1534 - RNA, 21 nt, synthetic construct
  source 1..21: mol_type = other RNA
ttggcaaacattcactccttc

SEQ ID NO: 1535 - RNA, 21 nt, synthetic construct
  source 1..21: mol_type = other RNA
ttggcaaacattcactccttc

SEQ ID NO: 1536 - RNA, 21 nt, synthetic construct
  source 1..21: mol_type = other RNA
tcggcaaacattcactccttc

SEQ ID NO: 1537 - RNA, 21 nt, synthetic construct
  source 1..21: mol_type = other RNA
ttggcaaacattcactccttc

SEQ ID NO: 1538 - RNA, 21 nt, synthetic construct
  source 1..21: mol_type = other RNA
tagtgaaaaactgacagcgaa

SEQ ID NO: 1539 - RNA, 21 nt, synthetic construct
  source 1..21: mol_type = other RNA
taagtgaaaaactgacagcga

SEQ ID NO: 1540 - RNA, 21 nt, synthetic construct
  source 1..21: mol_type = other RNA
ttaagtgaaaaactgacagcg

SEQ ID NO: 1541 - RNA, 21 nt, synthetic construct
  source 1..21: mol_type = other RNA
tataagtgaaaaactgacagc

SEQ ID NO: 1542 - RNA, 21 nt, synthetic construct
  source 1..21: mol_type = other RNA
taataagtgaaaaactgacag

SEQ ID NO: 1543 - RNA, 21 nt, synthetic construct
  source 1..21: mol_type = other RNA
tagaataagtgaaaaactgac

SEQ ID NO: 1544 - RNA, 21 nt, synthetic construct
  source 1..21: mol_type = other RNA
taagaataagtgaaaaactga

SEQ ID NO: 1545 - RNA, 21 nt, synthetic construct
  source 1..21: mol_type = other RNA
taaagaataagtgaaaaactg

SEQ ID NO: 1546 - RNA, 21 nt, synthetic construct
  source 1..21: mol_type = other RNA
ttaaagaataagtgaaaaact

SEQ ID NO: 1547 - RNA, 21 nt, synthetic construct
  source 1..21: mol_type = other RNA
tgtaaagaataagtgaaaaac

SEQ ID NO: 1548 - RNA, 21 nt, synthetic construct
  source 1..21: mol_type = other RNA
tgtgagaaaggtgtatctttt

SEQ ID NO: 1549 - RNA, 21 nt, synthetic construct
  source 1..21: mol_type = other RNA
ttgtgagaaaggtgtatcttt

SEQ ID NO: 1550 - RNA, 21 nt, synthetic construct
  source 1..21: mol_type = other RNA
tttgtgagaaaggtgtatctt

SEQ ID NO: 1551 - RNA, 21 nt, synthetic construct
  source 1..21: mol_type = other RNA
ttttgtgagaaaggtgtatct

SEQ ID NO: 1552 - RNA, 21 nt, synthetic construct
  source 1..21: mol_type = other RNA
tatttgtgagaaaggtgtatc

SEQ ID NO: 1553 - RNA, 21 nt, synthetic construct
  source 1..21: mol_type = other RNA
tttatttgtgagaaaggtgta

SEQ ID NO: 1554 - RNA, 21 nt, synthetic construct
  source 1..21: mol_type = other RNA
ttttatttgtgagaaaggtgt

SEQ ID NO: 1555 - RNA, 21 nt, synthetic construct
  source 1..21: mol_type = other RNA
tttttatttgtgagaaaggtg

SEQ ID NO: 1556 - RNA, 21 nt, synthetic construct
  source 1..21: mol_type = other RNA
tcttttatttgtgagaaaggt

SEQ ID NO: 1557 - RNA, 21 nt, synthetic construct
  source 1..21: mol_type = other RNA
ttcttttatttgtgagaaagg

SEQ ID NO: 1558 - RNA, 21 nt, synthetic construct
  source 1..21: mol_type = other RNA
taaaactgacagcgaatcatc

SEQ ID NO: 1559 - RNA, 21 nt, synthetic construct
  source 1..21: mol_type = other RNA
taaaaactgacagcgaatcat

SEQ ID NO: 1560 - RNA, 21 nt, synthetic construct
  source 1..21: mol_type = other RNA
tgaaaaactgacagcgaatca

SEQ ID NO: 1561 - RNA, 21 nt, synthetic construct
  source 1..21: mol_type = other RNA
ttgaaaaactgacagcgaatc

SEQ ID NO: 1562 - RNA, 21 nt, synthetic construct
  source 1..21: mol_type = other RNA
tgtgaaaaactgacagcgaat

SEQ ID NO: 1563 - RNA, 21 nt, synthetic construct
  source 1..21: mol_type = other RNA
taagtgaaaaactgacagcga

SEQ ID NO: 1564 - RNA, 21 nt, synthetic construct
  source 1..21: mol_type = other RNA
ttaagtgaaaaactgacagcg

SEQ ID NO: 1565 - RNA, 21 nt, synthetic construct
  source 1..21: mol_type = other RNA
tataagtgaaaaactgacagc

SEQ ID NO: 1566 - RNA, 21 nt, synthetic construct
  source 1..21: mol_type = other RNA
taataagtgaaaaactgacag

SEQ ID NO: 1567 - RNA, 21 nt, synthetic construct
  source 1..21: mol_type = other RNA
tgaataagtgaaaaactgaca

SEQ ID NO: 1568 - RNA, 21 nt, synthetic construct
  source 1..21: mol_type = other RNA
ttcaaaggagcctggagtttt

SEQ ID NO: 1569 - RNA, 21 nt, synthetic construct
  source 1..21: mol_type = other RNA
tttcaaaggagcctggagttt

SEQ ID NO: 1570 - RNA, 21 nt, synthetic construct
  source 1..21: mol_type = other RNA
tattcaaaggagcctggagtt

SEQ ID NO: 1571 - RNA, 21 nt, synthetic construct
  source 1..21: mol_type = other RNA
taattcaaaggagcctggagt

SEQ ID NO: 1572 - RNA, 21 nt, synthetic construct
  source 1..21: mol_type = other RNA
ttaattcaaaggagcctggag

SEQ ID NO: 1573 - RNA, 21 nt, synthetic construct
  source 1..21: mol_type = other RNA
ttgtaattcaaaggagcctgg

SEQ ID NO: 1574 - RNA, 21 nt, synthetic construct
  source 1..21: mol_type = other RNA
tgtgtaattcaaaggagcctg

SEQ ID NO: 1575 - RNA, 21 nt, synthetic construct
  source 1..21: mol_type = other RNA
tagtgtaattcaaaggagcct

SEQ ID NO: 1576 - RNA, 21 nt, synthetic construct
  source 1..21: mol_type = other RNA
tcagtgtaattcaaaggagcc

SEQ ID NO: 1577 - RNA, 21 nt, synthetic construct
  source 1..21: mol_type = other RNA
ttcagtgtaattcaaaggagc

SEQ ID NO: 1578 - RNA, 21 nt, synthetic construct
  source 1..21: mol_type = other RNA
taacattcactcctttaacaa

SEQ ID NO: 1579 - RNA, 21 nt, synthetic construct
  source 1..21: mol_type = other RNA
taaacattcactcctttaaca

SEQ ID NO: 1580 - RNA, 21 nt, synthetic construct
  source 1..21: mol_type = other RNA
tcaaacattcactcctttaac

SEQ ID NO: 1581 - RNA, 21 nt, synthetic construct
  source 1..21: mol_type = other RNA
tgcaaacattcactcctttaa

SEQ ID NO: 1582 - RNA, 21 nt, synthetic construct
  source 1..21: mol_type = other RNA
tggcaaacattcactccttta

SEQ ID NO: 1583 - RNA, 21 nt, synthetic construct
  source 1..21: mol_type = other RNA
tttggcaaacattcactcctt

SEQ ID NO: 1584 - RNA, 21 nt, synthetic construct
  source 1..21: mol_type = other RNA
tcttggcaaacattcactcct

SEQ ID NO: 1585 - RNA, 21 nt, synthetic construct
  source 1..21: mol_type = other RNA
ttcttggcaaacattcactcc

SEQ ID NO: 1586 - RNA, 21 nt, synthetic construct
  source 1..21: mol_type = other RNA
tctcttggcaaacattcactc

SEQ ID NO: 1587 - RNA, 21 nt, synthetic construct
  source 1..21: mol_type = other RNA
ttctcttggcaaacattcact

SEQ ID NO: 1588 - RNA, 21 nt, synthetic construct
  source 1..21: mol_type = other RNA
ctctatgaaaacgccttctta

SEQ ID NO: 1589 - RNA, 21 nt, synthetic construct
  source 1..21: mol_type = other RNA
agaagggaatcatgatatcga

SEQ ID NO: 1590 - RNA, 21 nt, synthetic construct
  source 1..21: mol_type = other RNA
tgagttcaagtcaaattctga

SEQ ID NO: 1591 - RNA, 21 nt, synthetic construct
  source 1..21: mol_type = other RNA
gtcgagacatttataaaggaa

SEQ ID NO: 1592 - RNA, 21 nt, synthetic construct
  source 1..21: mol_type = other RNA
ctaaggttagcagtgttgaaa

SEQ ID NO: 1593 - RNA, 21 nt, synthetic construct
  source 1..21: mol_type = other RNA
ctggttactctttgagattga

SEQ ID NO: 1594 - RNA, 21 nt, synthetic construct
  source 1..21: mol_type = other RNA
gaggaacaaactcttcttgga

SEQ ID NO: 1595 - RNA, 21 nt, synthetic construct
  source 1..21: mol_type = other RNA
cacccaaggtgtttgctatta

SEQ ID NO: 1596 - RNA, 21 nt, synthetic construct
  source 1..21: mol_type = other RNA
ggttgcttcttgaaagataga

SEQ ID NO: 1597 - RNA, 21 nt, synthetic construct
  source 1..21: mol_type = other RNA
tgtggtcatcaaataagtgca

SEQ ID NO: 1598 - RNA, 21 nt, synthetic construct
  source 1..21: mol_type = other RNA
gattctttactcccagaagaa

SEQ ID NO: 1599 - RNA, 21 nt, synthetic construct
  source 1..21: mol_type = other RNA
tccaggctcctttgaattaca

SEQ ID NO: 1600 - RNA, 21 nt, synthetic construct
  source 1..21: mol_type = other RNA
gttccaaaaaccaatatgcca

SEQ ID NO: 1601 - RNA, 21 nt, synthetic construct
  source 1..21: mol_type = other RNA
gtataccaactgttgggtaaa

SEQ ID NO: 1602 - RNA, 21 nt, synthetic construct
  source 1..21: mol_type = other RNA
gttgatatgagaggagtcaaa

SEQ ID NO: 1603 - RNA, 21 nt, synthetic construct
  source 1..21: mol_type = other RNA
ttcctctactcctcaagaaaa

SEQ ID NO: 1604 - RNA, 21 nt, synthetic construct
  source 1..21: mol_type = other RNA
gtttcacttattctttactca

SEQ ID NO: 1605 - RNA, 21 nt, synthetic construct
  source 1..21: mol_type = other RNA
agagaagtgtaagtgtttcta

SEQ ID NO: 1606 - RNA, 21 nt, synthetic construct
  source 1..21: mol_type = other RNA
gatagtggcattttaaatcta

SEQ ID NO: 1607 - RNA, 21 nt, synthetic construct
  source 1..21: mol_type = other RNA
gtacactgaattccaaaaaca

SEQ ID NO: 1608 - RNA, 21 nt, synthetic construct
  source 1..21: mol_type = other RNA
gaaccctgccattctaaaata

SEQ ID NO: 1609 - RNA, 21 nt, synthetic construct
  source 1..21: mol_type = other RNA
ccctgccattctaaaatttaa

SEQ ID NO: 1610 - RNA, 21 nt, synthetic construct
  source 1..21: mol_type = other RNA
gaaggagtgaatgtttgccaa

SEQ ID NO: 1611 - RNA, 21 nt, synthetic construct
  source 1..21: mol_type = other RNA
tgattcgctgtcagtttttca

SEQ ID NO: 1612 - RNA, 21 nt, synthetic construct
  source 1..21: mol_type = other RNA
ctgtcagtttttcacttatta

SEQ ID NO: 1613 - RNA, 21 nt, synthetic construct
  source 1..21: mol_type = other RNA
ctcccagaagactgtaaggaa

SEQ ID NO: 1614 - RNA, 21 nt, synthetic construct
  source 1..21: mol_type = other RNA
ggtaagtgtttcttaagatta

SEQ ID NO: 1615 - RNA, 21 nt, synthetic construct
  source 1..21: mol_type = other RNA
taagtgtttcttaagattata

SEQ ID NO: 1616 - RNA, 21 nt, synthetic construct
  source 1..21: mol_type = other RNA
ctatagtggcattttaaatca

SEQ ID NO: 1617 - RNA, 21 nt, synthetic construct
  source 1..21: mol_type = other RNA
gttaaatctgtcagacattaa

SEQ ID NO: 1618 - RNA, 21 nt, synthetic construct
  source 1..21: mol_type = other RNA
gcacaatttataccaactgta

SEQ ID NO: 1619 - RNA, 21 nt, synthetic construct
  source 1..21: mol_type = other RNA
agtacatggactggattttaa

SEQ ID NO: 1620 - RNA, 21 nt, synthetic construct
  source 1..21: mol_type = other RNA
gatgacatggagaaaaggtta

SEQ ID NO: 1621 - RNA, 21 nt, synthetic construct
  source 1..21: mol_type = other RNA
tgatatgagaggagtcaatta

SEQ ID NO: 1622 - RNA, 21 nt, synthetic construct
  source 1..21: mol_type = other RNA
tatgagaggagtcaattttaa

SEQ ID NO: 1623 - RNA, 21 nt, synthetic construct
  source 1..21: mol_type = other RNA
gtaaaggagtgaatgtttgca

SEQ ID NO: 1624 - RNA, 21 nt, synthetic construct
  source 1..21: mol_type = other RNA
gtgcacaaagatgattcgcta

SEQ ID NO: 1625 - RNA, 21 nt, synthetic construct
  source 1..21: mol_type = other RNA
gagatgattcgctgtcagtta

SEQ ID NO: 1626 - RNA, 21 nt, synthetic construct
  source 1..21: mol_type = other RNA
aagtgtaagtgtttcttaaga

SEQ ID NO: 1627 - RNA, 21 nt, synthetic construct
  source 1..21: mol_type = other RNA
gttattattcaccaaaactaa

SEQ ID NO: 1628 - RNA, 21 nt, synthetic construct
  source 1..21: mol_type = other RNA
actgaattccaaaaaccaata

SEQ ID NO: 1629 - RNA, 21 nt, synthetic construct
  source 1..21: mol_type = other RNA
ggactggattttagagaaaaa

SEQ ID NO: 1630 - RNA, 21 nt, synthetic construct
  source 1..21: mol_type = other RNA
gaaggagtgaatgtttgccaa

SEQ ID NO: 1631 - RNA, 21 nt, synthetic construct
  source 1..21: mol_type = other RNA
tgtcagtttttcacttattca

SEQ ID NO: 1632 - RNA, 21 nt, synthetic construct
  source 1..21: mol_type = other RNA
tgtcagtttttcacttattca

SEQ ID NO: 1633 - RNA, 21 nt, synthetic construct
  source 1..21: mol_type = other RNA
tgtcagtttttcacttattca

SEQ ID NO: 1634 - RNA, 21 nt, synthetic construct
  source 1..21: mol_type = other RNA
tgtcagtttttcacttattca

SEQ ID NO: 1635 - RNA, 21 nt, synthetic construct
  source 1..21: mol_type = other RNA
ggtaagtgtttcttaagatta

SEQ ID NO: 1636 - RNA, 21 nt, synthetic construct
  source 1..21: mol_type = other RNA
tccaggctcctttgaattaca

SEQ ID NO: 1637 - RNA, 21 nt, synthetic construct
  source 1..21: mol_type = other RNA
tgagttcaagtcaaattctga

SEQ ID NO: 1638 - RNA, 21 nt, synthetic construct
  source 1..21: mol_type = other RNA
taagaaggcgttttcatagag

SEQ ID NO: 1639 - RNA, 21 nt, synthetic construct
  source 1..21: mol_type = other RNA
tcgatatcatgattcccttct

SEQ ID NO: 1640 - RNA, 21 nt, synthetic construct
  source 1..21: mol_type = other RNA
tcagaatttgacttgaactca

SEQ ID NO: 1641 - RNA, 21 nt, synthetic construct
  source 1..21: mol_type = other RNA
ttcctttataaatgtctcgac

SEQ ID NO: 1642 - RNA, 21 nt, synthetic construct
  source 1..21: mol_type = other RNA
tttcaacactgctaaccttag

SEQ ID NO: 1643 - RNA, 21 nt, synthetic construct
  source 1..21: mol_type = other RNA
tcaatctcaaagagtaaccag

SEQ ID NO: 1644 - RNA, 21 nt, synthetic construct
  source 1..21: mol_type = other RNA
tccaagaagagtttgttcctc

SEQ ID NO: 1645 - RNA, 21 nt, synthetic construct
  source 1..21: mol_type = other RNA
taatagcaaacaccttgggtg

SEQ ID NO: 1646 - RNA, 21 nt, synthetic construct
  source 1..21: mol_type = other RNA
tctatctttcaagaagcaacc

SEQ ID NO: 1647 - RNA, 21 nt, synthetic construct
  source 1..21: mol_type = other RNA
tgcacttatttgatgaccaca

SEQ ID NO: 1648 - RNA, 21 nt, synthetic construct
  source 1..21: mol_type = other RNA
ttcttctgggagtaaagaatc

SEQ ID NO: 1649 - RNA, 21 nt, synthetic construct
  source 1..21: mol_type = other RNA
tgtaattcaaaggagcctgga

SEQ ID NO: 1650 - RNA, 21 nt, synthetic construct
  source 1..21: mol_type = other RNA
tggcatattggtttttggaac

SEQ ID NO: 1651 - RNA, 21 nt, synthetic construct
  source 1..21: mol_type = other RNA
tttacccaacagttggtatac

SEQ ID NO: 1652 - RNA, 21 nt, synthetic construct
  source 1..21: mol_type = other RNA
tttgactcctctcatatcaac

SEQ ID NO: 1653 - RNA, 21 nt, synthetic construct
  source 1..21: mol_type = other RNA
ttttcttgaggagtagaggaa

SEQ ID NO: 1654 - RNA, 21 nt, synthetic construct
  source 1..21: mol_type = other RNA
tgagtaaagaataagtgaaac

SEQ ID NO: 1655 - RNA, 21 nt, synthetic construct
  source 1..21: mol_type = other RNA
tagaaacacttacacttctct

SEQ ID NO: 1656 - RNA, 21 nt, synthetic construct
  source 1..21: mol_type = other RNA
tagatttaaaatgccactatc

SEQ ID NO: 1657 - RNA, 21 nt, synthetic construct
  source 1..21: mol_type = other RNA
tgtttttggaattcagtgtac

SEQ ID NO: 1658 - RNA, 21 nt, synthetic construct
  source 1..21: mol_type = other RNA
tattttagaatggcagggttc

SEQ ID NO: 1659 - RNA, 21 nt, synthetic construct
  source 1..21: mol_type = other RNA
ttaaattttagaatggcaggg

SEQ ID NO: 1660 - RNA, 21 nt, synthetic construct
  source 1..21: mol_type = other RNA
ttggcaaacattcactccttc

SEQ ID NO: 1661 - RNA, 21 nt, synthetic construct
  source 1..21: mol_type = other RNA
tgaaaaactgacagcgaatca

SEQ ID NO: 1662 - RNA, 21 nt, synthetic construct
  source 1..21: mol_type = other RNA
taataagtgaaaaactgacag

SEQ ID NO: 1663 - RNA, 21 nt, synthetic construct
  source 1..21: mol_type = other RNA
ttccttacagtcttctgggag

SEQ ID NO: 1664 - RNA, 21 nt, synthetic construct
  source 1..21: mol_type = other RNA
taatcttaagaaacacttacc

SEQ ID NO: 1665 - RNA, 21 nt, synthetic construct
  source 1..21: mol_type = other RNA
tataatcttaagaaacactta

SEQ ID NO: 1666 - RNA, 21 nt, synthetic construct
  source 1..21: mol_type = other RNA
tgatttaaaatgccactatag

SEQ ID NO: 1667 - RNA, 21 nt, synthetic construct
  source 1..21: mol_type = other RNA
ttaatgtctgacagatttaac

SEQ ID NO: 1668 - RNA, 21 nt, synthetic construct
  source 1..21: mol_type = other RNA
tacagttggtataaattgtgc

SEQ ID NO: 1669 - RNA, 21 nt, synthetic construct
  source 1..21: mol_type = other RNA
ttaaaatccagtccatgtact

SEQ ID NO: 1670 - RNA, 21 nt, synthetic construct
  source 1..21: mol_type = other RNA
taaccttttctccatgtcatc

SEQ ID NO: 1671 - RNA, 21 nt, synthetic construct
  source 1..21: mol_type = other RNA
taattgactcctctcatatca

SEQ ID NO: 1672 - RNA, 21 nt, synthetic construct
  source 1..21: mol_type = other RNA
ttaaaattgactcctctcata

SEQ ID NO: 1673 - RNA, 21 nt, synthetic construct
  source 1..21: mol_type = other RNA
tgcaaacattcactcctttac

SEQ ID NO: 1674 - RNA, 21 nt, synthetic construct
  source 1..21: mol_type = other RNA
tagcgaatcatctttgtgcac

SEQ ID NO: 1675 - RNA, 21 nt, synthetic construct
  source 1..21: mol_type = other RNA
taactgacagcgaatcatctc

SEQ ID NO: 1676 - RNA, 21 nt, synthetic construct
  source 1..21: mol_type = other RNA
tcttaagaaacacttacactt

SEQ ID NO: 1677 - RNA, 21 nt, synthetic construct
  source 1..21: mol_type = other RNA
ttagttttggtgaataataac

SEQ ID NO: 1678 - RNA, 21 nt, synthetic construct
  source 1..21: mol_type = other RNA
tattggtttttggaattcagt

SEQ ID NO: 1679 - RNA, 21 nt, synthetic construct
  source 1..21: mol_type = other RNA
tttttctctaaaatccagtcc

SEQ ID NO: 1680 - RNA, 21 nt, synthetic construct
  source 1..21: mol_type = other RNA
ttggcaaacattcactccttc

SEQ ID NO: 1681 - RNA, 21 nt, synthetic construct
  source 1..21: mol_type = other RNA
tgaataagtgaaaaactgaca

SEQ ID NO: 1682 - RNA, 21 nt, synthetic construct
  source 1..21: mol_type = other RNA
tgaataagtgaaaaactgaca

SEQ ID NO: 1683 - RNA, 21 nt, synthetic construct
  source 1..21: mol_type = other RNA
tgaataagtgaaaaactgaca

SEQ ID NO: 1684 - RNA, 21 nt, synthetic construct
  source 1..21: mol_type = other RNA
tgaataagtgaaaaactgaca

SEQ ID NO: 1685 - RNA, 21 nt, synthetic construct
  source 1..21: mol_type = other RNA
taatcttaagaaacacttacc

SEQ ID NO: 1686 - RNA, 21 nt, synthetic construct
  source 1..21: mol_type = other RNA
tgtaattcaaaggagcctgga

SEQ ID NO: 1687 - RNA, 21 nt, synthetic construct
  source 1..21: mol_type = other RNA
tcagaatttgacttgaactca